(19) United States
(12) Patent Application Publication
(10) Pub. No.: US 2025/0032477 A1
(43) Pub. Date: Jan. 30, 2025

(54) COMPOUNDS, PHARMACEUTICAL COMPOSITIONS, AND METHODS FOR THE TREATMENT, PREVENTION, OR MANAGEMENT OF HYPERPROLIFERATIVE DISORDERS

(71) Applicant: VINCERX PHARMA GMBH, Monheim am Rhein (DE)

(72) Inventors: Hans-Georg LERCHEN, Leverkusen (DE); Beatrix STELTE-LUDWIG, Wülfrath (DE); Mareike WIEDMANN, Monheim am Rhein (DE); Anne-Sophie REBSTOCK, Champagne au Mont d'Or (FR); Johannes KOEBBERLING, Neuss (DE); Harvey WONG, Vancouver, BC (CA)

(21) Appl. No.: 18/698,341

(22) PCT Filed: Oct. 3, 2022
(86) PCT No.: PCT/IB2022/000566

§ 371 Date: Apr. 3, 2024

Related U.S. Application Data
(60) Provisional application No. 63/252,122, filed on Oct. 4, 2021.

(51) Int. Cl. A61K 31/4745; A61K 47/65; A61P 35/00

(52) U.S. Cl. CPC A61K 31/4745; A61K 47/65; A61P 35/00

(57) ABSTRACT

Disclosed herein are compounds, pharmaceutical compositions, and methods for treating, preventing or managing diseases and conditions including hyperproliferative disorders such as cancer in humans and other mammals. Compounds disclosed herein are hexacyclic topoisomerase inhibitor prodrugs, conjugated to an integrin binding moiety via cleavable linkers and/or functional spacers.

### CROSS-REFERENCE

This International Patent Application claims the benefit of U.S. Provisional Patent Application No. 63/252,122, filed Oct. 4, 2021, which is incorporated herein by reference in its entirety.

### BACKGROUND OF THE INVENTION

This application relates to compounds, pharmaceutical compositions, processes for preparation thereof, and the use thereof for treating, preventing, or managing diseases and conditions including hyperproliferative disorders, such as, cancer in humans and other mammals.

### SUMMARY OF THE INVENTION

The invention relates to novel compounds (or “conjugates”) comprising one or more binder molecules or derivatives thereof with one or more molecules of an active component, wherein the active component is a hexacyclic cytotoxic or cytostatic ligand (e.g., a topoisomerase inhibitor), which is conjugated to one more binder molecules (e.g., an integrin binder (e.g., an αᵥβ₃ integrin binder)) via a linker peptide EL as described and defined herein, and methods for their preparation, their use for the treatment and/or prophylaxis of disorders, in particular of hyper-proliferative disorders.

The current invention describes conjugates with small molecule binders, which release the drug extracellularly in the tumor microenvironment (TME) upon cleavage by enzymes present in TME. Compounds described herein may comprise small molecule binders (e.g., a cytotoxic or cytostatic ligand and an integrin binder), conjugated via an enzymatically cleavable linker and/or a polymeric linker which, in some embodiments, is retained in an acidic TME.

To increase the therapeutic window of the toxophores described herein, and to achieve a tumor targeting of this efficacious class of anti-tumor compounds, a class of hexacyclic camptothecin derivative prodrugs has been developed and this is described in the current invention. The inventive prodrug compounds may comprise, but are not limited to, one or more αᵥβ₃ integrin binding moieties which are linked to a toxophore via a peptide moiety EL, which is cleaved by proteases present in the tumor microenvironment (TME) to release the parent anti-tumor compound at the site of action. Enzymes present in the TME include, but are not limited to, neutrophil elastase and legumain.

In one aspect, provided herein is a compound, or a pharmaceutically acceptable salt thereof, comprising a hexacyclic cytotoxic or cytostatic ligand (CT) conjugated to one or more integrin binders (IN) via a linker. In some embodiments, the linker is enzymatically-cleavable.

In some embodiments, provided herein is a compound of Formula (A) or Formula (C):

CT-EL-L¹-A¹(L²-IN)(L³-IN) Formula (A)

CT-EL-L⁵-IN Formula (C)
wherein:
CT is a hexacyclic cytotoxic or cytostatic ligand;
EL is an enzymatically-cleavable linker; optionally further comprising a self-immolative group (SIL);
each L¹, L², L³, and L⁵ is independently a bivalent linker;
A¹ is a trivalent linker; and
IN is, in each instance, independently, an integrin binder.

In some embodiments, the compound is cleaved by neutrophil elastase. In some embodiments, EL has the formula: The compound of any one of claims 1-3, or a pharmaceutically acceptable salt thereof, wherein the compound is cleaved by neutrophil elastase. In some embodiments, EL is an enzymatically-cleavable peptide linker of the formula: L-Asp-L-Pro-L-Ala, L-Asn-L-Pro-L-Ala, L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-.

In some embodiments, the compound is of Formula (I-A) or Formula (I-C):

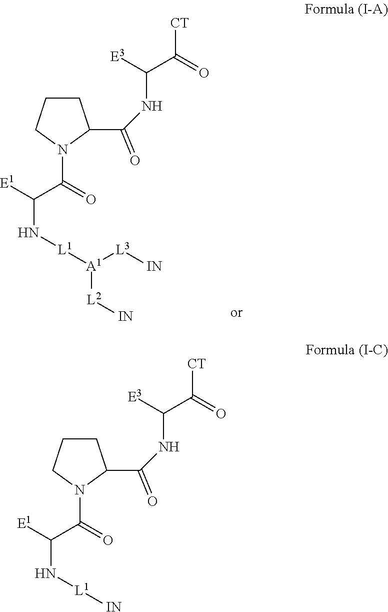

or a stereoisomer or mixture of stereoisomers thereof, wherein:
CT is a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂CH₂C(O)OH; —CH₂C(O)OR¹; or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each L¹, L², L³, and L⁵ is independently a bivalent linker;
R¹ is a substituted or unsubstituted C₁₋₁₂ alkyl; wherein if R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and
IN is, in each instance, independently, an integrin binder.

In some embodiments, IN is, in each instance, a small molecule integrin-binding moiety. In some embodiments, IN is, in each instance, an αᵥβ₃ integrin binder. In some embodiments, IN, in each instance, has the structure:

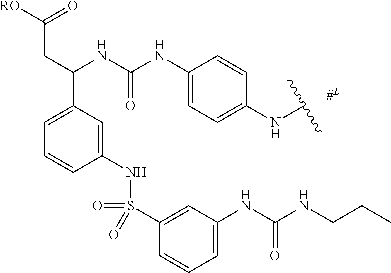

or a pharmaceutically acceptable salt thereof, or a stereoisomer thereof, wherein:
#^{L} denotes a bond to L¹, L², L³, L⁵, or L⁶; and
R is hydrogen or a substituted or unsubstituted C₁₋₁₂ alkyl.

In some embodiments, provided herein is a compound of Formula (II-A) or Formula (II-C):

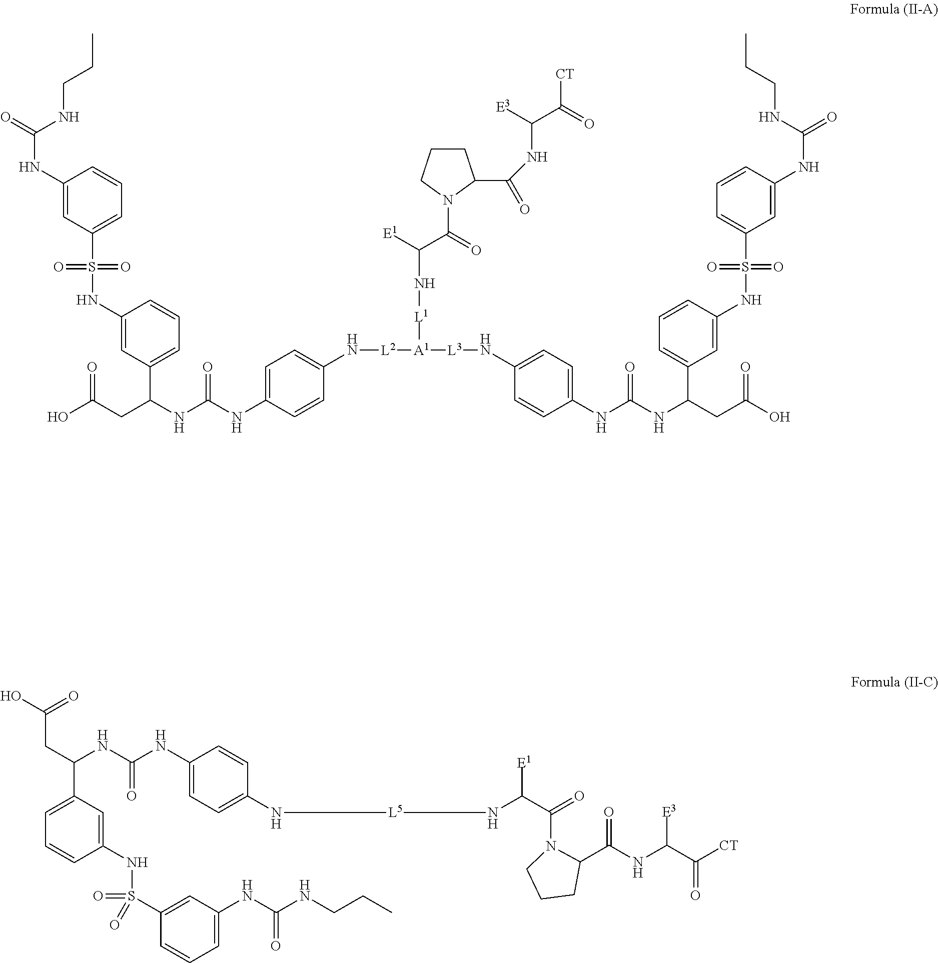

wherein:
CT is a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂CH₂C(O)OH; —CH₂C(O)OR¹; or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each L¹, L², L³, and L⁵ is each independently a bivalent linker; and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and IN is an integrin binder.

In some embodiments, CT is:

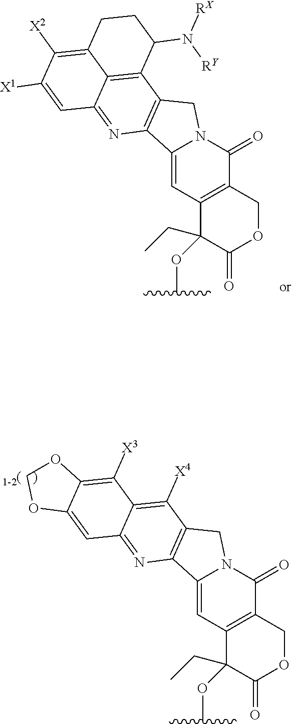

wherein:
X¹ is —F, —Cl, or —CH₃;
X² is —F, —Cl, or —CH₃;
X³ is hydrogen; and
X⁴ is hydrogen, C₁₋₃ alkyl, or C₁₋₃ alkylamine;
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
wherein EL² is an enzymatically-cleavable linker; and
R^{Y} is hydrogen, C₁₋₆ alkyl, —C(O)C₁₋₆ alkyl, —C(O)C₁₋₆ haloalkyl, —C(O)C₁₋₆ alkyl-OH, or —C(O)C₁₋₆ alkyl-OCH₃.
or a pharmaceutically acceptable salt thereof, or a stereoisomer or mixture of stereoisomers thereof.

In some embodiments, provided herein is a compound of Formula (III-A), Formula (III-C), Formula (IV-A), or Formula (IV-C):

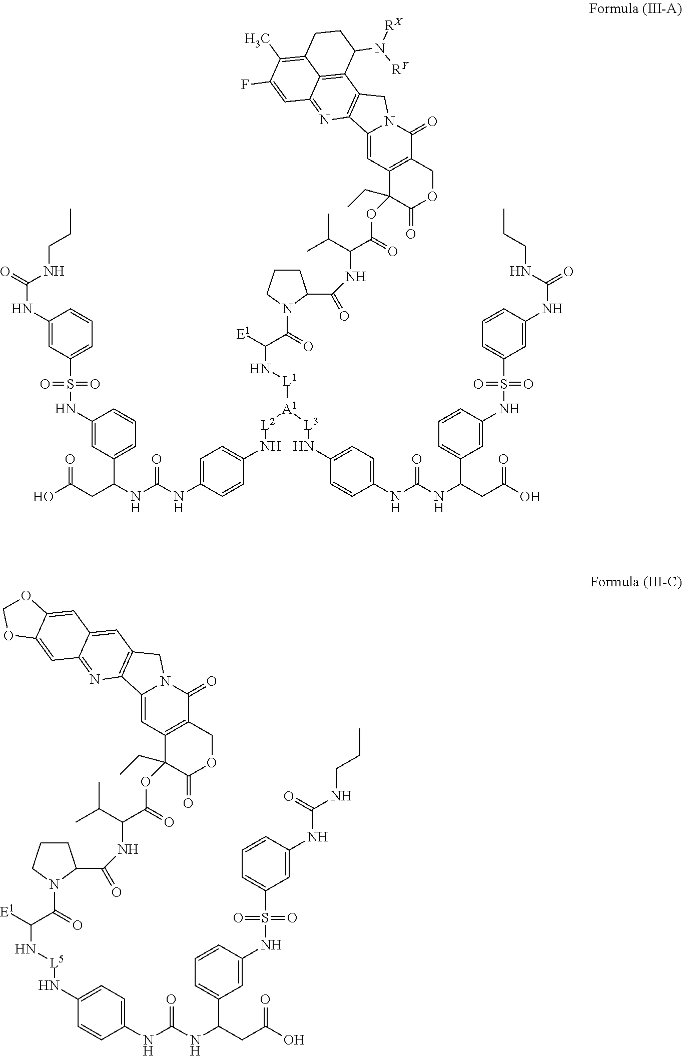

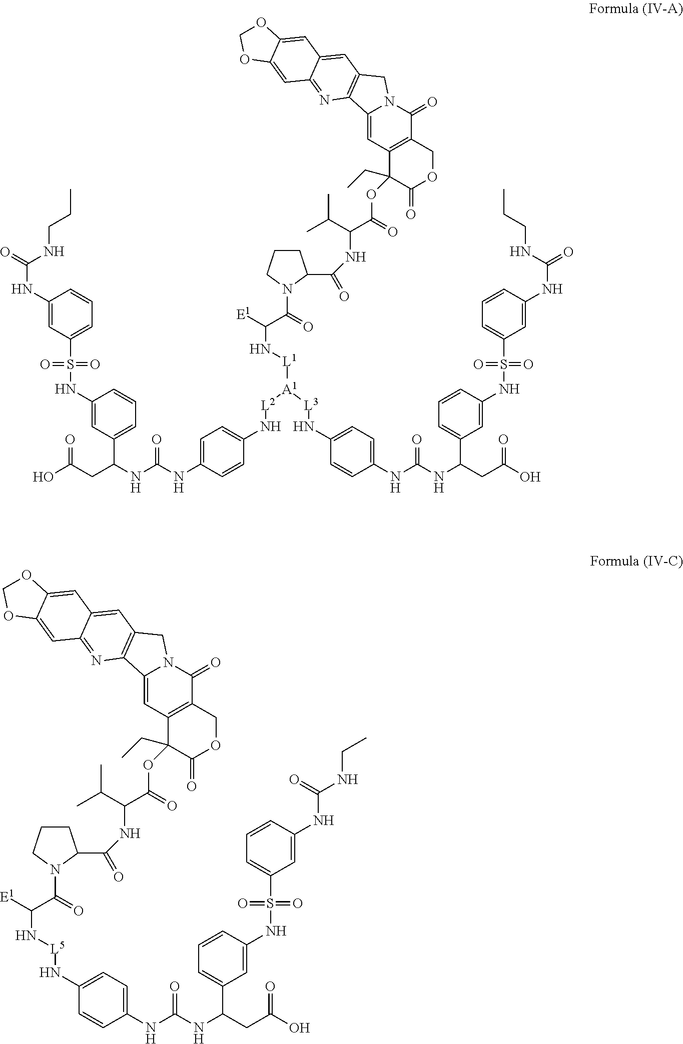

or a pharmaceutically acceptable salt thereof, or a stereoisomer or mixture of stereoisomers thereof; wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
wherein EL² is a legumain-cleavable linker;
R^{Y} is hydrogen, C₁₋₆ alkyl, —C(O)C₁₋₆ alkyl, or —C(O)C₁₋₆ haloalkyl;
E¹ is hydrogen, —CH₂C(O)NH₂ or —CH₂C(O)OH; and
A¹ is a trivalent linker; and
each L¹, L², L³, and L⁵ is independently a bivalent linker.

In still another aspect, described herein is a compound, or a pharmaceutically acceptable salt thereof, or a stereoisomer or mixture of stereoisomers thereof, or a pharmaceutical composition thereof, for use as a medicament. In still another aspect, described herein is a compound, or a pharmaceutically acceptable salt thereof; or a stereoisomer or mixture of stereoisomers thereof, or a pharmaceutical composition thereof, for use in a method of treating a disease or disorder disclosed herein. In some embodiments, the disease or disorder is a hyperproliferative disorder. In some embodiments, the disease or disorder is a cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 panel (a) shows the pH dependent equilibrium reaction between the closed and hydrolyzed open forms of the lactone ring of Compound 1; (b) shows an LC-MS gradient for Compound 1 with acidic eluent; and (c) shows an LC-MS gradient for Compound 1 with basic eluent.

FIG. 2 panel (a) shows the equilibrium reaction between the closed and hydrolyzed open forms of the lactone ring of Compound 2; (b) shows an LC-MS gradient for Compound 2 with acidic eluent; and (c) shows an LC-MS gradient for Compound 2 with basic eluent.

FIG. 3 panel (a) shows the equilibrium reaction between the closed and hydrolyzed open forms of the lactone ring of Compound 4; (b) shows an LC-MS gradient for Compound 4 with acidic eluent; and (c) shows an LC-MS gradient for Compound 4 with basic eluent.

FIG. 4 shows the anti-tumor efficacy of Compounds 4b and 5 in a SCLC mouse xenograft model, compared to untreated (vehicle).

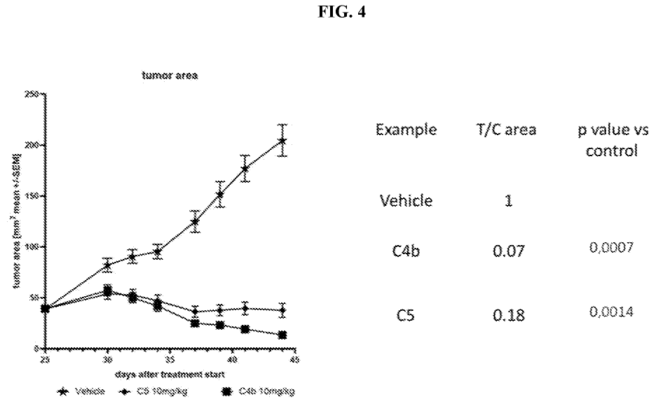

### DETAILED DESCRIPTION OF THE INVENTION

The present invention relates to novel pharmaceutical compounds comprising one or more αᵥβ₃ integrin binders, novel linker units containing one or more N-alkyl amino groups or one or more oxygens at different positions in the linker as well as peptide sequences cleavable by tumor-associated enzymes such as neutrophil elastase, and a cytotoxic component (e.g., a hexacyclic topoisomerase inhibitor), the processes for preparation thereof, to the use thereof for treating, preventing or managing diseases and conditions including hyperproliferative disorders such as cancer in humans and other mammals.

Chemotherapy in cancer is accompanied by usually serious side effects which are to be attributed to the toxic action of chemotherapeutics on proliferating cells of other tissue types rather than tumor tissue. For many years, scientists have occupied themselves with the problem of improving the selectivity of active compounds employed. A frequently followed approach is the synthesis of prodrugs which are released more or less selectively in the target tissue, for example, by change of the pH (DE-A 42 29 903), by enzymes (e.g. glucuronidases; EP-A 511 917 and 595 133) or by antibody-enzyme conjugates (WO 88/07378; U.S. Pat. No. 4,975,278; EP-A 595 133). A problem in these approaches is, inter alia, the lack of stability of the conjugates in other tissues and organs, and, in particular, the ubiquitous active compound distribution which follows the extracellular release of active compound in the tumor tissue.

20(S)—Camptothecin is a pentacyclic alkaloid which was isolated in 1966 by Wall et al. (J. Am. Chem. Soc. 88, 3888 (1966)). It has a high active antitumor potential in numerous in-vitro and in-vivo tests. Unfortunately, however, the realization of the promising potential in the clinical investigation phase failed because of toxicity and solubility problems.

By opening of the E ring lactone and formation of the sodium salt, a water-soluble compound was obtained which is in a pH-dependent equilibrium with the ring-closed form. Here too, clinical studies have not led to success.

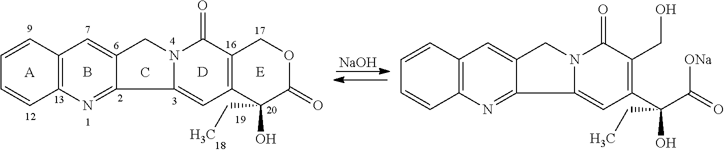

About 20 years later, it was found that the biological activity is to be attributed to enzyme inhibition of topoisomerase I. Since then, the research activities have again been increased in order to find a camptothecin derivative which is more soluble and better tolerated and which is active in-vivo.

For improvement of the water solubility, salts of A-ring- and B-ring-modified camptothecin derivatives and of 20-O-acyl derivatives with ionizable groups have been described (U.S. Pat. No. 4,943,579). The latter prodrug concept was later also transferred to modified camptothecin derivatives (WO 96/02546). The described 20-O-acyl prodrugs, however, have a very short half-life in vivo and are very rapidly cleaved to give the parent structure.

A large number of camptothecin derivatives have been investigated in preclinical and clinical studies; from those, irinotecan, topotecan and belotecan have successfully been approved (Li et al, Am J Cancer Res 2017; 7(12):2350-2394). Some such derivatives (A-W) are listed below.

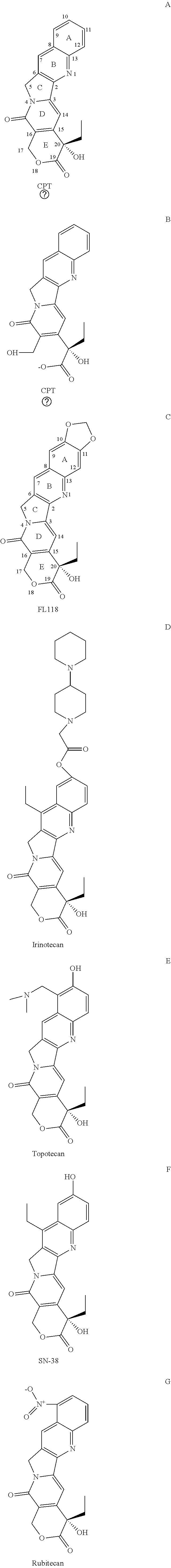

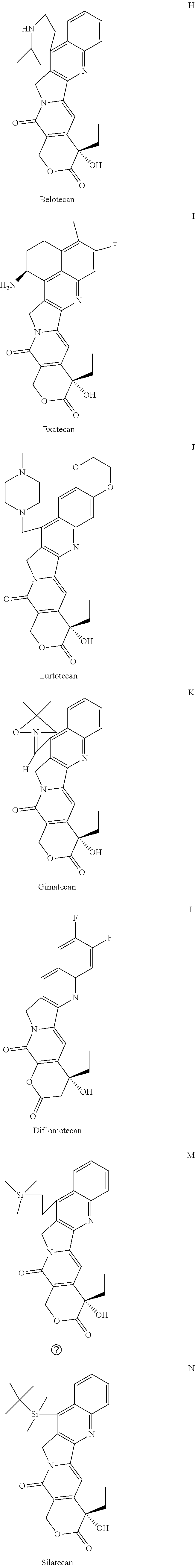

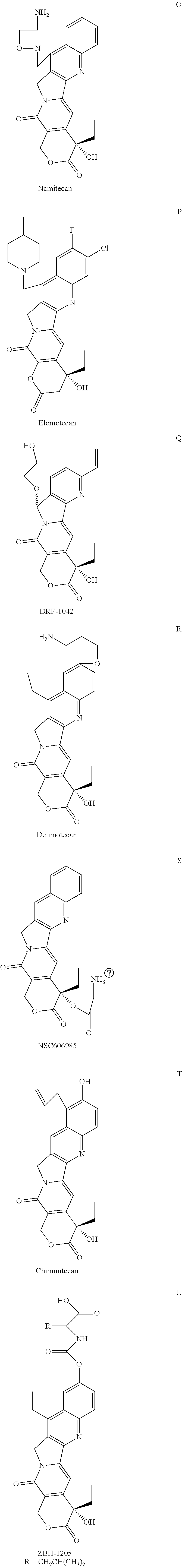

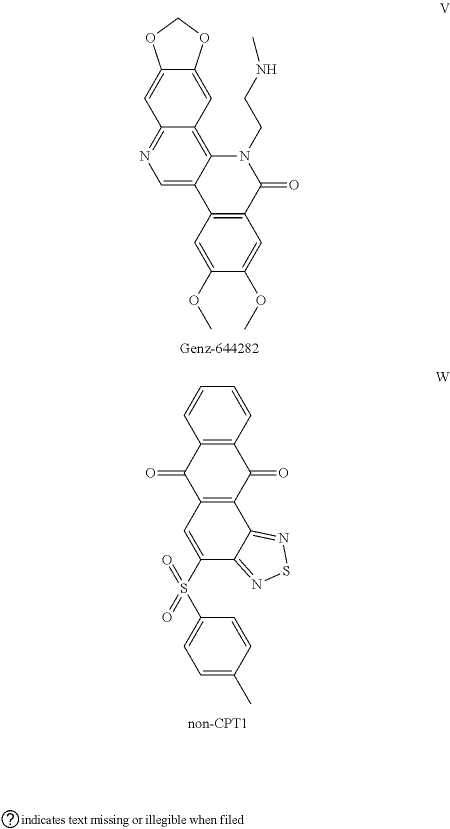 [SMILES: CNCCn1c(=O)c2cc(OC)c(OC)cc2c2cnc3cc4c(cc3c21)OCO4 ; Cc1ccc(S(=O)(=O)c2cc3c(c4nsnc24)C(=O)c2ccccc2C3=O)cc1]

Improving the therapeutic window of cytotoxic agents such as camptothecin and its derivatives remains a challenge.

Integrins are heterodimeric transmembrane proteins found on the surface of cells, which play an important part in the adhesion of the cells to an extracellular matrix. They recognize extracellular glycoproteins such as fibronectin or vitronectin on the extracellular matrix via the RGD sequence occurring in these proteins (RGD is the single-letter code for the amino acid sequence arginine-glycine-aspartate).

In general, integrins such as, for example, the vitronectin receptor, which is also called the αᵥβ₃ receptor, or alternatively the αᵥβ₅ receptor or the GpIIb/IIIa receptor play an important part in biological processes such as cell migration, angiogenesis and cell-matrix adhesion and thus for diseases in which these processes are crucial steps. Cancer, osteoporosis, arteriosclerosis, restenosis and ophthalmia may be mentioned by way of example.

The αᵥβ₃ receptor occurs, for example, in large amounts on growing endothelial cells and makes possible their adhesion to an extracellular matrix. The αᵥβ₃ receptor thus plays an important part in angiogenesis, i. e. the formation of new blood vessels, which is a crucial prerequisite for tumor growth and metastasis formation in carcinomatous disorders.

It was possible to show that the blockade of the above-mentioned receptors is an important starting point for the treatment of disorders of this type. If the adhesion of growing endothelial cells to an extracellular matrix is suppressed by blocking their corresponding integrin receptors, for example, by a cyclic peptide or a monoclonal antibody, angiogenesis does not occur, which leads to a stoppage or regression of tumour growth (cf., for example, Brooks et al. in Cell 79, 1157-1164 (1994)).

In spite of compelling preclinical results demonstrating that the inhibition of integrin has therapeutic potential, clinical trials with integrin inhibitors targeting those integrins have repeatedly failed to demonstrate therapeutic benefits in cancer patients (C. Ruegg et al, Cancers 2019, 11, 978).

WO 1998/010795 describes conjugates in which a molecule targeting tumors is linked to a functional unit such as, for example, a cytostatic or a detectable label such as, for example, a radioactive nuclide. Inter alia, integrin binders such as, for example, peptides having the RGD sequence described above are described as molecules targeting tumor or tumor stroma. Doxorubicin is described as an example of a cytostatic which is linked to a molecule of this type addressing tumors.

In the case of the compounds of WO 1998/010795, the linkage is carried out such that the molecule addressing a tumour and the functional unit are directly bonded to one another with retention of their respective properties (cf., for example, p. 56, 1. 17, to p. 58, 1. 10, and Ex. 6). This has the result that these compounds are indeed selectively concentrated in the immediate vicinity of tumour cells by binding of the entity addressing a tumour (in the case of a radical having αᵥβ₃ integrin-antagonistic action by binding to the αᵥβ₃ integrin receptor which, in particular, is expressed on endothelial cells newly formed by angiogenesis), but on account of the direct combination the functional unit such as, for example, a cytostatic cannot be released into the intracellular space of the tumour tissue.

Fundamentally, the conjugate which on the one hand is selectively concentrated in tumour tissue by the effect of a part addressing αᵥβ₃ or αᵥβ₅ integrin receptors found in the conjugate, but on the other hand comprises a cytostatic which can be released from the conjugate, should have an increased toxophoric effect on tumour tissue due to the possibility of the more direct action of the cytostatic on the tumour cells compared with the conjugates described in WO 1998/010795. In particular, such a toxophoric effect and tumour selectivity should even be higher, if the release of the cytostatic takes place in the immediate vicinity of the tumour tissue or even in the tumour cells.

In WO 2000/069472 enzyme-activated anti-tumour prodrug compounds are disclosed which can be specifically cleaved by collagenase (IV) and elastase. With respect to linking units cleavable by elastase this application describes that the specific tetrapeptide sequences Ala-Ala-Pro-Val and Ala-Ala-Pro-Nva are suitable. Furthermore, in this reference, no conjugates which comprise a moiety addressing αᵥβ₃ integrin receptors and a cytostatic are mentioned. Y. Liu et al. (Mol. Pharmaceutics 2012, 9, 168) describe conjugates of Auristatins linked to an αᵥβ₃ integrin targeting moiety via an legumain-cleavable linker. Gennari et al describe taxane derivatives linked to a cyclopeptidic RGD integrin recognition motif and a diketopiperazine (DKP) scaffold via elastase cleavable linker containing a self-immolative spacer unit (Chem. Eur. J. 2019, 25, 1696-1700).

In EP 1 238 678 conjugates with cytotoxic agents are disclosed which target αᵥβ₃ integrins and have peptide linkers which can be specifically cleaved by elastase. With respect to linking units cleavable by elastase this application describes peptide sequences comprising Pro-Val and Pro-Leu. As toxophore moieties camptothecin and a quinolone carboxylic acid are exemplified.

Particular challenges of such conjugates include
sufficient solubility enabling intravenous administration in appropriate vehicles,
high tumour penetration of intact conjugates,
high stability in plasma to avoid systemic de-conjugation,
efficient binding to the targeted receptors in tumour microenvironment,
efficient cleavage by enzymes present in tumor microenvironment,
high stability and cellular permeability of cleaved toxophore moieties to enhance tumour cell uptake versus re-distribution.

It is therefore one objective of the present invention to develop conjugates which comprise a moiety addressing αᵥβ₃ integrin receptors and a cytostatic which can be released from the conjugate preferably in tumour microenvironment, where the moiety in the conjugate addressing αᵥβ₃ integrin receptors retains its ability to bind to the αᵥβ₃ integrin receptor and therefore provides tissue selectivity to such compounds. In addition, cleavability of the conjugates and drug release should be mediated by enzymes present and active in the tumor environment such as neutrophil elastase. Finally, the profile of the toxophore should match an extracellular cleavage and release mechanism in a way, that it should be highly permeable into tumour cells and tissues and not being a substrate of drug transporters.

In WO2020/094471 αᵥβ₃ integrin conjugates with 7-ethyl camptothecin have been described, which addressed some of these aspects.

A number of camptothecin derivatives have been investigated as a payload in Antibody-Drug-Conjugates, such as DxD in Enhertu (Modi et al, N. Engl. J. Med. 382, 610-621) and SN38 in Trodelvy (Rugo et al., Future Oncol. 16, 705-715), which both got recently approved.

Since small molecule drug conjugates may show favorable features over ADCs such as e. g. higher tumor penetration (Cazzamalli et al., J. Am. Chem. Soc. 2018, 140, 1617) the current invention comprises small molecule drug conjugates with selected camptothecin derivatives. Preferred camptothecin derivatives should show high potency, high membrane penetration properties and a low efflux ratio. Exemplary camptothecin derivatives described herein include 10,11-methylenedioxy-camptothecin (e.g., FL118) and exatecan derivatives.

The present invention relates to pharmaceutical compounds which are conjugates comprising one or more αᵥβ₃ integrin binding moieties, linking units which can be selectively cleaved by tumor associated enzymes such as elastase, a poly N-alkylamine (e.g., polyethyleneimine, or PEI) or polyether (e.g., polyethyleneglycol, or PEG) spacer and a potent cytotoxic agent (i.e., a toxophore (CT)), preferably but not limited to hexacyclic camptothecin derivatives such as those disclosed herein. The conjugates have a tumor-specific action as a result of linkage to αᵥβ₃ integrin binders via preferred linking units which can be selectively cleaved by elastase, i.e. by an enzyme which can especially be found in tumor stroma. The preferred linking units provide sufficient stability of the conjugate in biological media, e.g. culture medium or serum and, at the same time, the desired tumor cell killing as a result of its specific enzymatic or hydrolytic cleavage in a tumor microenvironment and cellular uptake of the released drug moiety.

In particular, the compounds of the present invention show favorable features by different means:
Modified spacers containing one or more oxygen atoms or N-alkyl amino groups at different positions in the linker allowing for high solubility
One or more αᵥβ₃ integrin-binding moieties enabling high affinity
Despite significant steric challenges these novel linkers appear highly susceptible for cleavage by enzymes present in tumor stroma such as neutrophil elastase
Conjugate design in some embodiments but not limited to hydroxy lactone acylation with a beneficial impact on lactone ring stability (Drugs Fut 2002, 27(9), 869)
High stability of the conjugates in plasma and cytotoxic activity which is increased in the presence of elastase
Employment of preferably, but not limited to hexacyclic cytotoxic or cytostatic warheads (e.g., exatecan derivatives or 10,11-methylenedioxy-camptothecin toxophores, 10,11 ethylenedioxy-camptothecin toxophores), which provide enhanced potency over some pentacyclic camptothecin analogs.

Towards this goal, exatecan, 10,11-methylenedioxy-camptothecin, and derivatives thereof, (e.g., those disclosed herein) are particularly advantageous as the toxophore moiety in above mentioned conjugates. Provided herein are conjugates of cytotoxic or cytostatic ligands, e.g., a hexacyclic topoisomerase inhibitor, e.g., exatecan or 10,11-methylenedioxy-camptothecin, that are designed to address the various challenges that have limited clinical viability of compounds and conjugates previously described in the art. Particularly, the present compounds and conjugates provide linking moieties with functional and beneficial effects, including tumor retention, incorporation of multiple integrin binding units, elongated distance between the toxophore and targeting moiety, and prodrug designs which modulate DMPK and toxophore release and thus each of these features enhance on-target toxicity while limiting such toxicity issues at off-target sites (e.g., where neutrophil elastase is less prevalent). The compounds and conjugates disclosed herein are clinically advantageous and provide additional elements of utility for at least those reasons described supra, as well as others that will be apparent to those skilled in the art.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Compounds

Cancer cells or tumor microenvironment overexpress certain enzymes, including, but not limited to neutrophil elastase. Conjugates described herein are generally enzymatically cleavable by neutrophil elastase. In some embodiments, conjugates described herein are selectively cleavable in the microenvironment of cancer cells, with less cleavage taking place in the circulation or in healthy tissues. In some embodiments, a conjugate described herein is cytotoxic after activation by a tumor-associated enzyme, such as, neutrophil elastase, and/or legumain. In some embodiments, a conjugate described herein is non-toxic, in therapeutic concentrations, in the absence of said activation. The cytotoxic agents described herein are inhibitors of topoisomerase. The cytotoxic agent may also be conjugated, e.g., via an enzymatically cleavable linker or a spacer, to an integrin binding moiety. Integrin binding moieties for use in the present disclosure include any agent that is able to bind to a tumor cell or to extracellular matrix. Examples include, but not limited to, small molecules, peptides, proteins, and the like. In some embodiments, the integrin binding moiety binds to an integrin that is alpha-v beta-3 (or alternatively: αᵥβ₃, aᵥb₃, or even avb3). In some embodiments, a conjugate described herein acts as a prodrug, e.g., an enzymatically or hydrolytically cleavable prodrug, and one of the cleaved components is a cytotoxic or cytostatic agent (e.g., exatecan or FL118) described herein. In some embodiments, a conjugate described herein is a compound having the formula (I), as defined herein.

In an aspect, provided herein is a compound, or a pharmaceutically acceptable salt thereof; or a stereoisomer or mixture of stereoisomers thereof, having a structure represented by any one of Formulae (01)-(05):

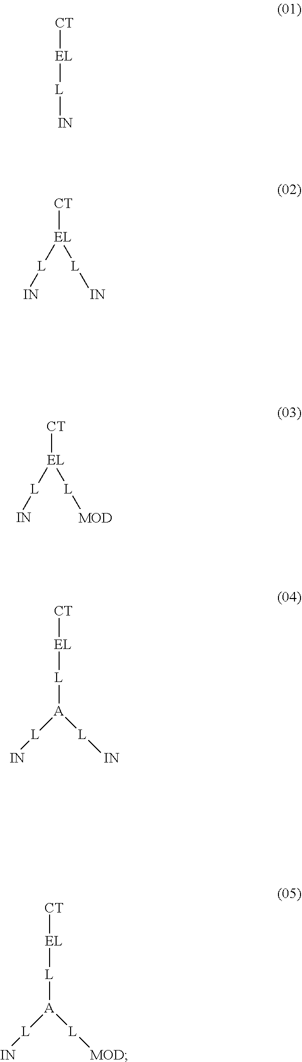

wherein:
CT is a hexacyclic cytotoxic or cytostatic group;
EL is an enzymatically-cleavable linker;
L is a bivalent linker;
A is a trivalent linker;
IN is an integrin binder; and
MOD is a physicochemical or pharmacokinetic modulator group.

In an aspect, provided herein is a compound, or a pharmaceutically acceptable salt thereof, or a stereoisomer or mixture of stereoisomers thereof, having a structure represented by Formula (A), Formula (C), or Formula (E):

CT-EL-L¹-A¹(L²-IN)(L³-IN) Formula (A)

CT-EL-L⁵-IN Formula (C)

CT-EL-L¹-A¹(L²-IN)(L³-MOD)Formula(E)
wherein, in each instance:
CT is a hexacyclic cytotoxic or cytostatic ligand;
EL is an enzymatically-cleavable linker;
each L¹, L², L³, and L⁵ is independently a bivalent linker;
IN is, in each instance, independently, an integrin binder;
MOD is a physicochemical or pharmacokinetic modulating group; and
A¹ is a trivalent linker (e.g., a trivalent radical containing 1 to 100 non-hydrogen atoms, optionally containing alkyl, heteroalkyl, carbocyclic, and/or heterocyclic groups, or any combination thereof).

In some embodiments, L¹, L², L³, and/or L⁵ is a bivalent linker (e.g., a substituted or unsubstituted C₁₋₆₀ alkyl linker, or substituted or unsubstituted 1 to 60-membered heteroalkyl linker, each optionally substituted and/or intersected one or more times by a group selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —NHS(O)₂NH—, —NHS(O)₂NHC(O)—, —NHS(O)₂NHC(O)O—, —O—, —S—, —S(O)—, —S(O)₂—, —S(O)₂NH—, —S(O)₂NHC(O)—, —S(O)₂NHC(O)NH—, —S(O)₂NHC(O)O—, aryl, heteroaryl, aralkyl, heteroaralkyl, or any combination thereof). In some embodiments, L¹, L², L³, and/or L⁵ is a bivalent linker (e.g., simple spacer, a polymeric spacer, In some embodiments, provided herein is a compound, or a pharmaceutically acceptable salt thereof, or a stereoisomer or mixture of stereoisomers thereof, having a structure represented by Formula (I) or Formula (I′):

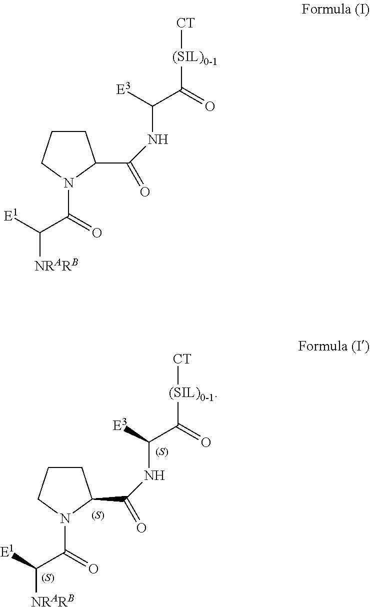

In some embodiments, provided herein is a compound, or a pharmaceutically acceptable salt thereof, or a stereoisomer or mixture of stereoisomers thereof, of Formula (I) or Formula (I′), wherein:
CT is a monovalent radical of a hexacyclic cytotoxic or cytostatic ligand;
SIL is a self-immolative linker;
E¹ is hydrogen, —CH₃, —CH₂CH₃, —CH₂CH₂CH₃, —CH₂C(O)NH₂, —CH₂C(O)OH, —CH₂C(O)OR¹;
E³ is CH₃, CH(CH₃)₂, CH₂CH(CH₃)₂, or CH(CH₃)CH₂CH₃;
R^{A} is hydrogen or C₁₋₆ alkyl;
R^{B} is -L¹-A¹(L²-(IN))(L³-(IN)), -L⁵-IN, or -L¹-A¹(L²-(IN))(L³-(MOD));
   wherein:
   IN is a monovalent radical of an integrin binder;
   A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
   each of L¹, L², L³, and L⁵, is independently a bivalent linker (e.g., a substituted or unsubstituted C₁₋₆₀ alkyl, or substituted or unsubstituted 1-60 heteroalkyl, optionally substituted or intersected by heteroaryl);
   MOD is a physicochemical or pharmacokinetic modulating group (e.g., —COOH); and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl).

In some embodiments, provided herein is a compound, or a pharmaceutically acceptable salt thereof; or a stereoisomer or mixture of stereoisomers thereof, having a structure of Formula (I-A) or Formula (I-A′):

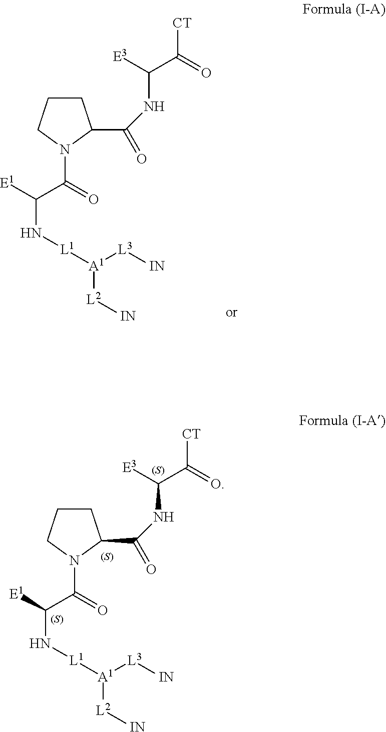

In some embodiments, provided herein is a compound, or a pharmaceutically acceptable salt thereof, or a stereoisomer or mixture of stereoisomers thereof, having a structure of Formula (I-A) or Formula (I-A′), wherein:
CT is a monovalent radical of a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
IN is in each instance, independently, a monovalent radical of an integrin binder;
A¹ is a trivalent radical (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
each of L¹, L², and L³ is a bivalent linker (e.g., a substituted or unsubstituted C₁₋₃₀ alkyl, or substituted or unsubstituted heteroalkyl); and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., a substituted or unsubstituted C₁₋₃₀ alkyl, or substituted or unsubstituted heteroalkyl).

In some embodiments, provided herein is a compound, or a pharmaceutically acceptable salt thereof; or a stereoisomer or mixture of stereoisomers thereof, having a structure of Formula (I-C) or Formula (I-C′):

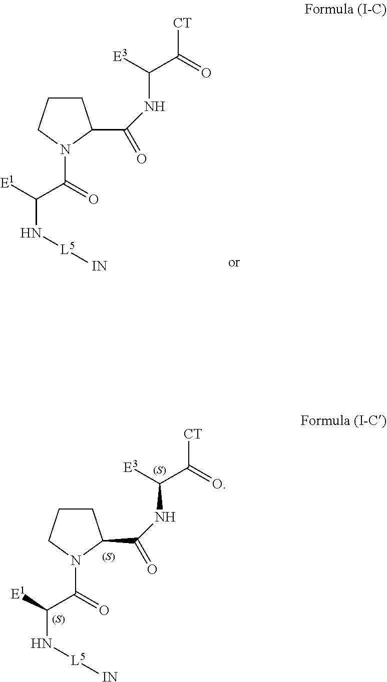

In some embodiments, provided herein is a compound, or a pharmaceutically acceptable salt thereof; or a stereoisomer or mixture of stereoisomers thereof, having a structure of Formula (I-C) or Formula (I-C′), wherein:
CT is a monovalent radical of a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH or —CH₂CH₂C(O)OR¹;
E³ is —CH₃, —CH(CH₃)₂, —CH₂CH(CH₃)₂, or —CH(CH₃)CH₂CH₃;
IN is in each instance, independently, a monovalent radical of an integrin binder;
L⁵ is a bivalent linker (e.g., a substituted or unsubstituted C₁₋₆₀ alkyl linker, or substituted or unsubstituted 1 to 60-membered heteroalkyl linker, each optionally substituted or intersected by aryl or heteroaryl);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
wherein L⁶ is a bivalent linker (e.g., a substituted or unsubstituted C₁₋₃₀ alkyl, or substituted or unsubstituted heteroalkyl).

In another aspect, provided herein is a compound, or a pharmaceutically acceptable salt thereof, or a stereoisomer or mixture of stereoisomers thereof, having a structure represented by Formula (II) or Formula (II′):

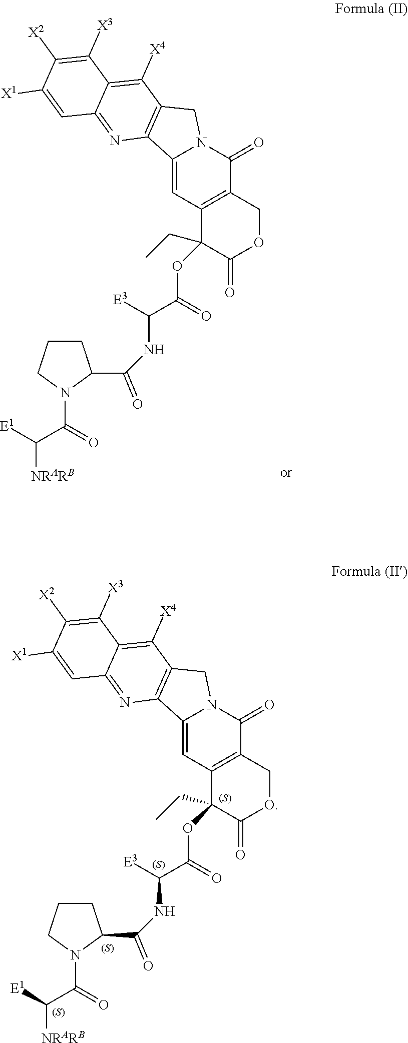

In some embodiments, provided herein is a compound, or a pharmaceutically acceptable salt thereof, or a stereoisomer or mixture of stereoisomers thereof, of Formula (II) or Formula (II′), wherein:
(i) X¹ and X² taken together form an ethylenedioxy or methylenedioxy heterocycle; and X³ and X⁴ are each independently hydrogen, halogen, C₁₋₆ haloalkyl, C₁₋₆ alkyl, or C₁₋₆ alkylamine; or
(ii) X¹ and X² are each independently hydrogen, halogen, C₁₋₄ alkyl, or C₁₋₄ haloalkyl, and X³ and X⁴ taken together form a carbocyclic ring A substituted with —NR^{X}R^{Y};
wherein:
   R^{X} is hydrogen, C₁₋₆ alkyl, —C(O)CH₃, —C(O)CF₃, —C(O)OBn,
      —C(O)CH₂(pyridine), -Asn-C(O)CH₂(pyridine), or
      -EL²-C(O)CH₂(pyridine);
      wherein:
      EL² is an enzymatically-cleavable linker, (e.g., cleavable by a tumor-associated protease (e.g., legumain), e.g., a di- or tripeptide linker, e.g., (-L-Asp)-(L-Pro)-(L-Val-), (-L-Asn)-(L-Pro)-(L-Val-), (-L-Ala)-(L-Ala)-(L-Asn-), or (-L-Ala)-(L-N-Me-Ala)-(L-Asn-);
R^{Y} is hydrogen, substituted or unsubstituted alkyl, substituted or unsubstituted heteroalkyl, substituted or unsubstituted carbocycle, or a substituted or unsubstituted heterocycle; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
E¹ is hydrogen, —CH₃, —CH₂CH₃, —CH₂CH₂CH₃, —CH₂C(O)NH₂, —CH₂C(O)OH, or —CH₂C(O)OR¹;
E³ is CH₃, CH(CH₃)₂, CH₂CH(CH₃)₂, or CH(CH₃)CH₂CH₃;
R^{A} is hydrogen or C₁₋₆ alkyl;
R^{B} is -L¹-A¹(L²IN)(L³IN), -L⁵-IN, or -L¹-A¹(L²IN)(L³MOD);
   wherein:
   IN is a monovalent radical of an integrin binder;
   MOD is a physicochemical modulating group as defined herein (e.g., —COOH);
   A¹ is a trivalent radical (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
   each of L¹, L², L³, and L⁵ is independently a bivalent linker (e.g., a substituted or unsubstituted C₁₋₆₀ alkyl linker, or substituted or unsubstituted 1 to 60-membered heteroalkyl linker, each optionally substituted or intersected by aryl or heteroaryl); and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., a substituted or unsubstituted C₁₋₃₀ alkyl, or substituted or unsubstituted heteroalkyl).

In some embodiments, provided herein is a compound, or a pharmaceutically acceptable salt thereof; or a stereoisomer or mixture of stereoisomers thereof, having a structure of Formula (II-A), Formula (II-C), or Formula (II-E):

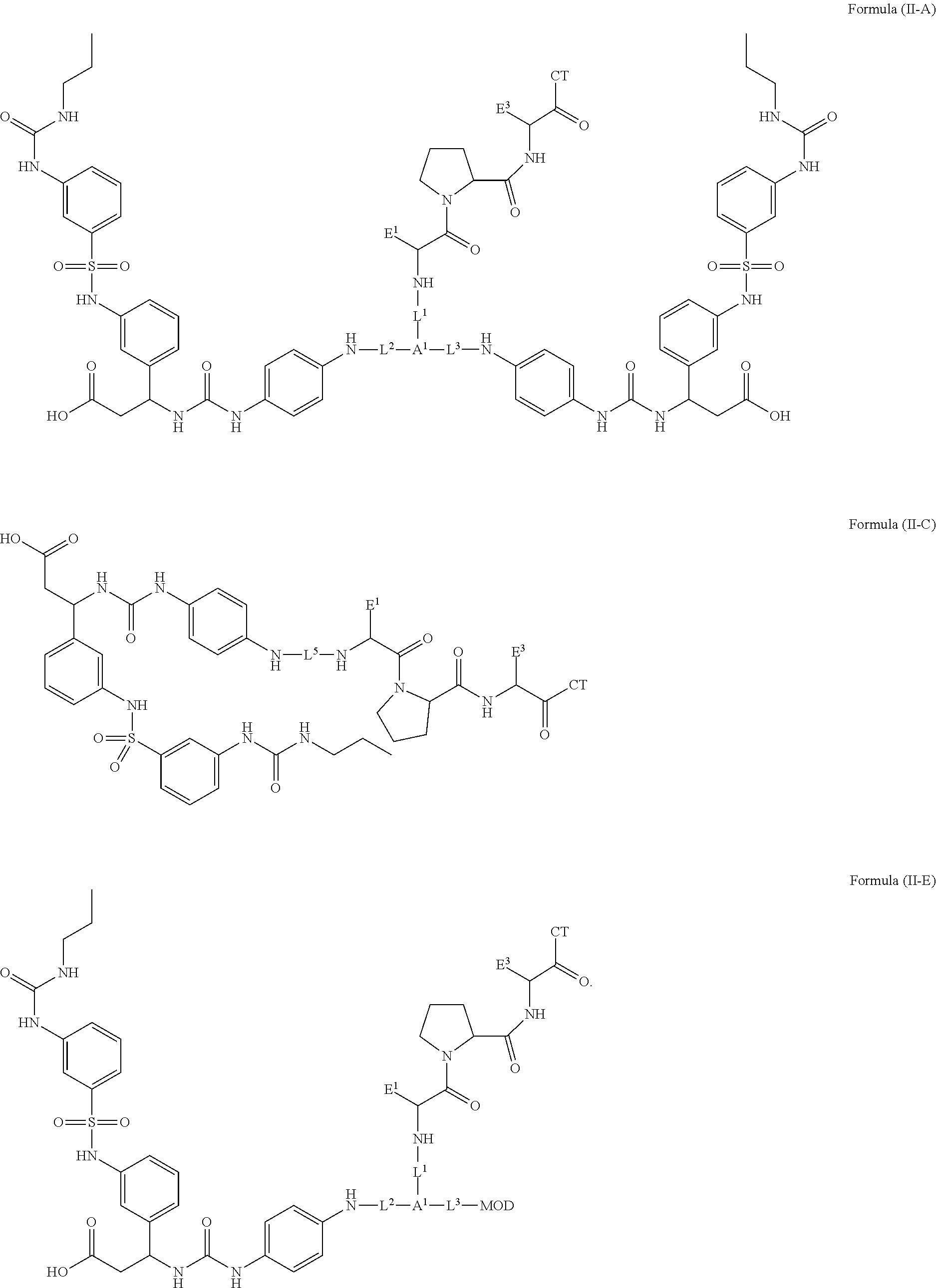

In some embodiments, A¹ is a trivalent radical containing 1 to 100 atoms, optionally containing alkyl, heteroalkyl, carbocyclic, or heterocyclic groups, or any combination thereof; and each of L¹, L², and L³ is independently substituted or unsubstituted heteroalkyl.

In some embodiments:
A¹ is a trivalent linker (e.g., a trivalent linker group, optionally comprising heteroalkyl, alkyl, aralkyl, heteroaralkyl, aryl, heteroaryl, cycloalkyl, and/or heterocycloalkyl groups);
E¹ is hydrogen, —CH₃, —CH₂C(O)NH₂, —CH₂C(O)OH;
E³ is CH₃, CH(CH₃)₂, CH₂CH(CH₃)₂, or CH(CH₃)CH₂CH₃;
L¹, L², L³, and L⁵ are each bivalent linkers (e.g., independently selected from (i), (ii), (iii), and (iv):
   (i) —C(O)—,
   (ii) —(CO)ₘ(CH₂)ₙ(OC₂₋₆ alkyl)ₒ(NH)ₚ(CO)_{q}—,
   (iii) —(CO)ᵣ(CH₂)ₛ(NR¹⁰C₁₋₆ alkyl)ₜ(NR¹¹)ᵤ(CO)ᵥ—, or
   (iv) —(CO)ᵣ(CH₂)ₛ(NR¹⁰C(O)C₁₋₆ alkyl)ₜ(NR¹¹)ᵤ(CO)ᵥ—,
   wherein:
   R¹⁰ is, in each instance, independently hydrogen or C₁₋₃ alkyl;
   R¹¹ is, in each instance, independently hydrogen or C₁₋₃ alkyl;
   m is, in each instance, independently 0 or 1;
   n is in each instance, independently an integer from 0 to 10;
   is in each instance, independently an integer from 0 to 10;
   p is in each instance, independently 0 or 1;
   q is in each instance, independently 0 or 1;
   r is in each instance, independently 0 or 1;
   s is in each instance, independently an integer from 0 to 10;
   t is in each instance, independently an integer from 0 to 10;
   u is in each instance, independently 0 or 1; and
   v is in each instance, independently 0 or 1); and
MOD is a physicochemical or pharmacokinetic modulator (e.g., —COOH).

In some embodiments, A¹ is a trivalent heteroalkyl radical; and each of L¹, L², and L³ is substituted or unsubstituted heteroalkyl.

In some embodiments, L⁵ is substituted or unsubstituted heteroalkyl. In some embodiments, L⁵ is a linker having a structure represented by the formula:
(i) —(CO)ₘ(CH₂)ₙ(OC₂₋₆ alkyl)ₒ(NIH)ₚ(CO)_{q}— or
(ii) —(CO)ᵣ(CH₂)ₛ(NR¹⁰C₁₋₆ alkyl)ₜ(NR¹¹)ᵤ(CO)ᵥ—; or
(iii) —(CO)ᵣ(CH₂)ₛ(NR¹⁰C(O)C₁₋₆ alkyl)ₜ(NR¹¹)ᵤ(CO)ᵥ—; or
wherein:
R¹⁰ is hydrogen or C₁₋₃ alkyl;
R¹¹ is hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and
v is 0 or 1.

In some embodiments, provided herein is a compound or a pharmaceutically acceptable salt thereof, or a stereoisomer or mixture of stereoisomers thereof, wherein:
X¹ and X² taken together form a methylenedioxy heterocycle;
X³ is hydrogen; and X⁴ is hydrogen, C₁₋₆ alkyl, or C₁₋₆ alkylamine.

In some embodiments, provided herein is a compound or a pharmaceutically acceptable salt thereof, or a stereoisomer or mixture of stereoisomers thereof, wherein:
X¹ and X² are each independently halogen or —CH₃; and
X³ and X⁴ taken together form a cyclohexyl ring substituted with —NR^{X}R^{Y};
wherein:
R^{X} is hydrogen, C₁₋₆ alkyl, —C(O)CH₃, —C(O)CF₃, —C(O)OBn, —C(O)CH₂(pyridine), -Asn-C(O)CH₂(pyridine), or -(-L-Asn)-(N-Me-L-Ala)-(L-Ala-)-C(O)CH₂-(pyridine); and
R^{Y} is hydrogen or C₁₋₆alkyl.

In some embodiments, provided herein is a compound or a pharmaceutically acceptable salt thereof, or a stereoisomer or mixture of stereoisomers thereof, having a structure represented by Formula (III) or Formula (IV):

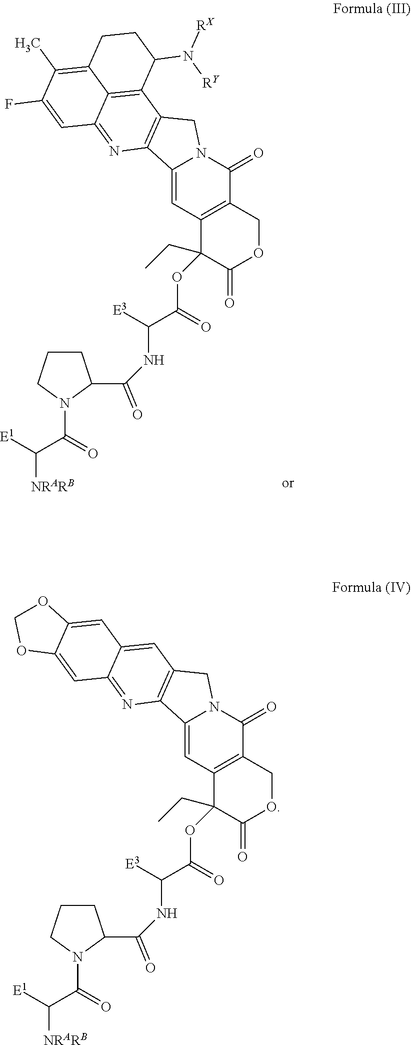

In some embodiments, provided herein is a compound or a pharmaceutically acceptable salt thereof, or a stereoisomer or mixture of stereoisomers thereof, having a structure represented by Formula (III′) or Formula (IV′):

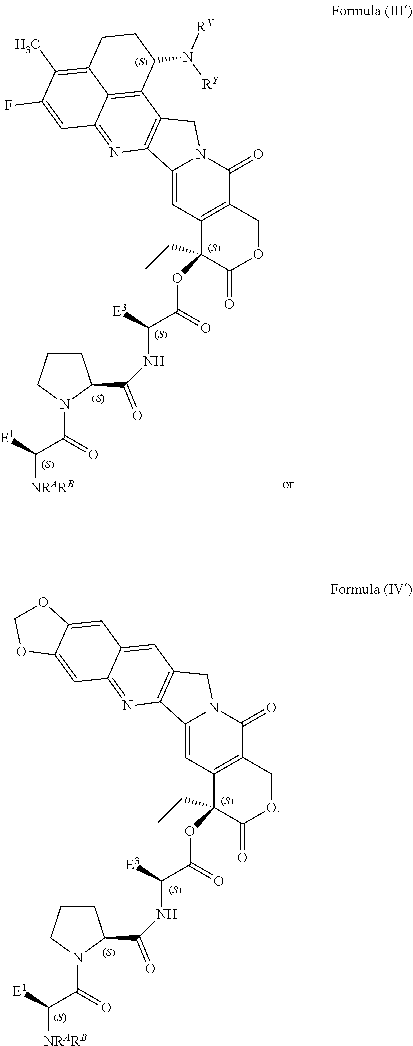

In some embodiments, provided herein is a compound of Formula (III-A), Formula (III-C), Formula (IV-A), or Formula (IV-C):

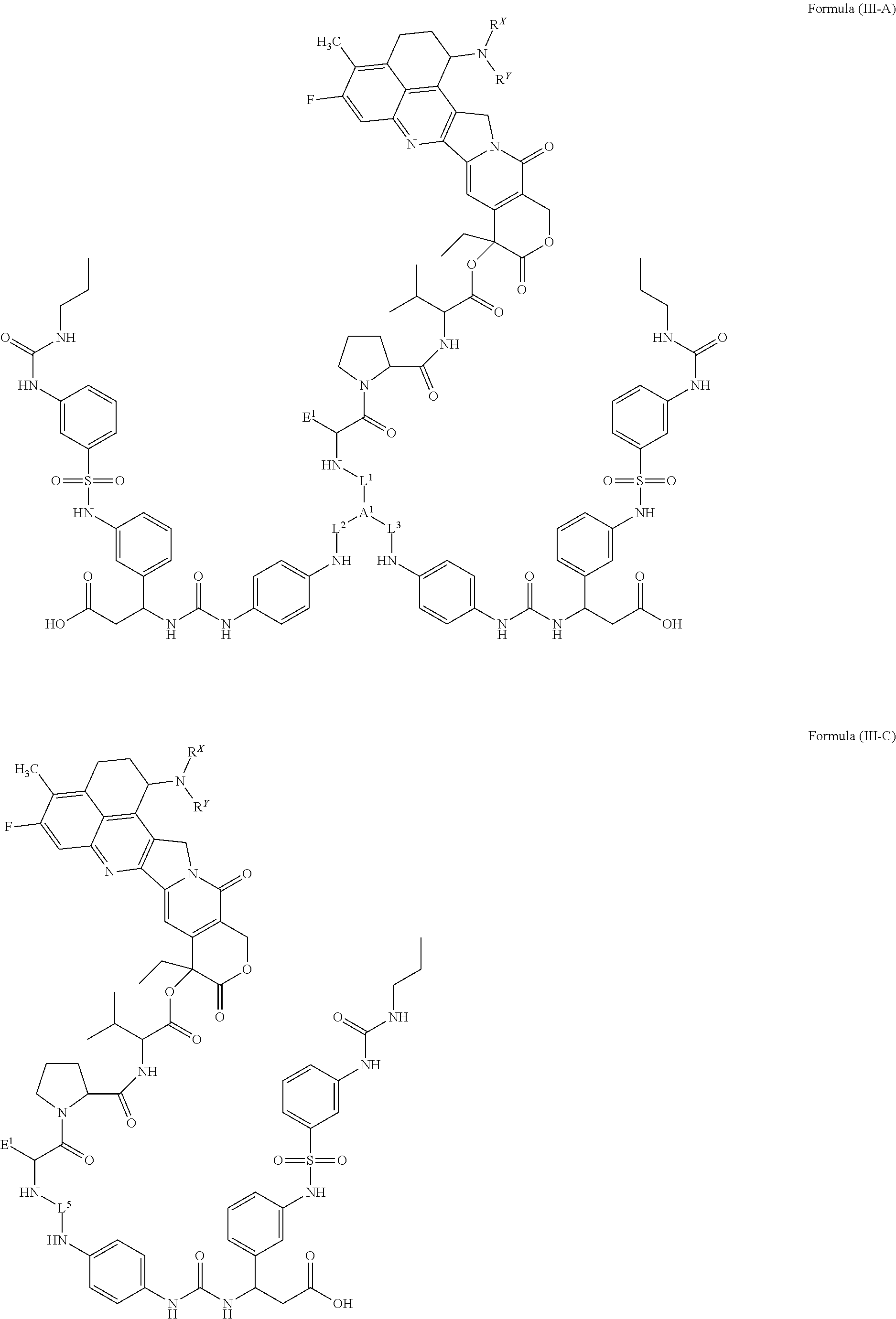

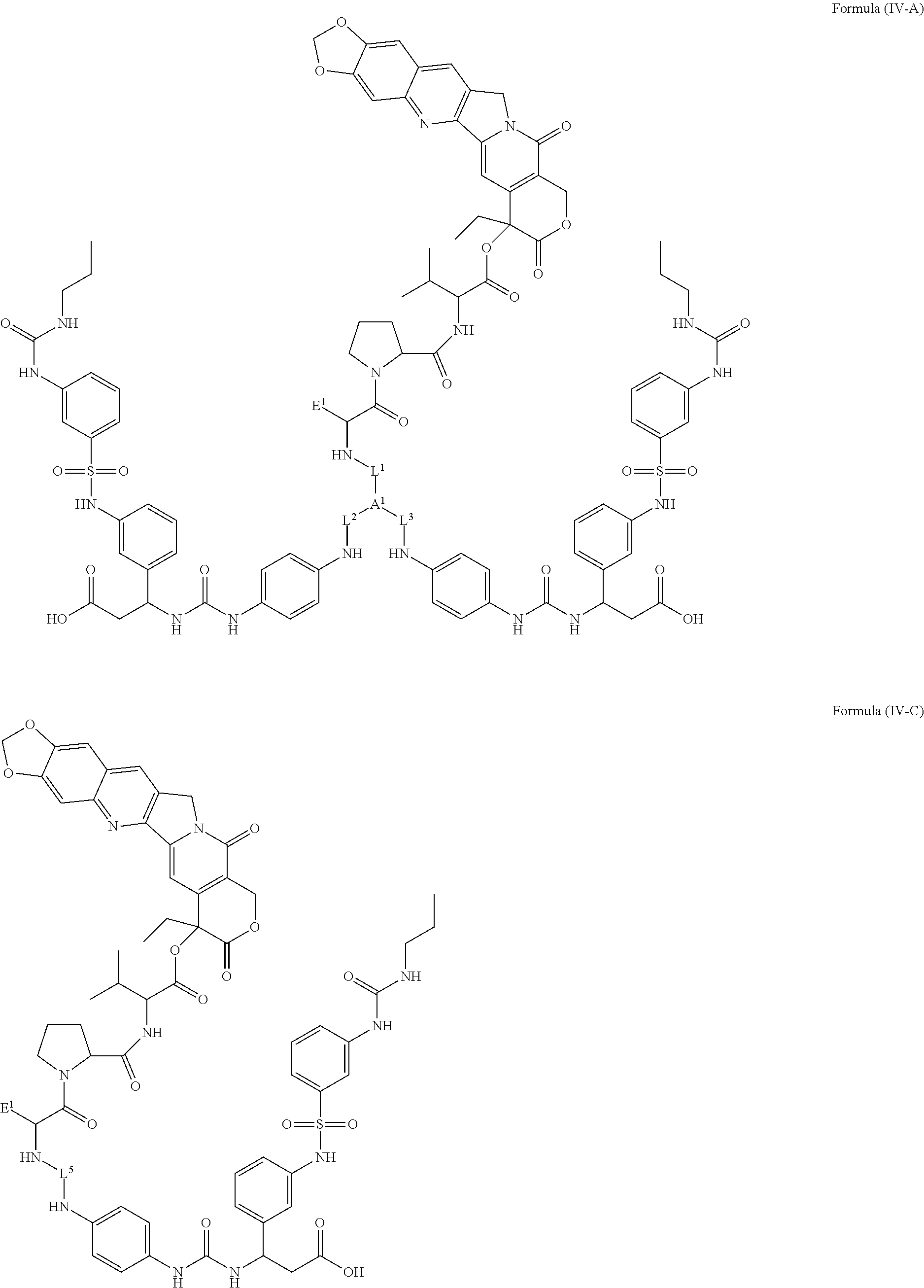

or a pharmaceutically acceptable salt thereof, or a stereoisomer or mixture of stereoisomers thereof.

In some embodiments:
R^{X} is hydrogen, C₁₋₆ alkyl, —C(O)CH₃, —C(O)CF₃, —C(O)OBn (i.e., —Cbz), —C(O)CH₂(pyridine), -Asn-C(O)CH₂(pyridine), or -EL²-C(O)CH₂(pyridine);
   wherein:
   EL² is (-L-Asp)-(L-Pro)-(L-Val-), (-L-Asn)-(L-Pro)-(L-Val-), (-L-Ala)-(L-Ala)-(L-Asn-), or (-L-Ala)-(N-Me-L-Ala)-(L-Asn-);
R^{Y} is hydrogen, substituted or unsubstituted alkyl, substituted or unsubstituted heteroalkyl, substituted or unsubstituted carbocycle, or a substituted or unsubstituted heterocycle; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂;
E¹ is hydrogen, —CH₂C(O)NH₂, —CH₂C(O)OH, or —CH₂C(O)OR¹;
R¹ is a C₁₋₆ alkyl group that is substituted with -L⁶-IN, —NH₂, —NH(C₁₋₆alkyl), —NHL⁶-IN, —N(C₁₋₆alkyl)₂, or —N(C₁₋₆alkyl)₃⁺; IN is an integrin binder; and L⁶ is —C(O)—;
A¹ is a trivalent heteroalkyl radical;
each of L¹, L², L³, and L⁵, is independently a bivalent linker (e.g., a substituted or unsubstituted C₁₋₆₀ alkyl, or substituted or unsubstituted 1-60 heteroalkyl, optionally substituted or intersected by heteroaryl);

In some embodiments, each of L¹, L², L³, and L⁵ is independently a simple spacer (defined herein) or a polymeric spacer (defined herein).

In some embodiments, provided herein is a compound of Formula (III-A) or Formula (IV-A), or a pharmaceutically acceptable salt thereof, or a stereoisomer or mixture of stereoisomers thereof, wherein:
R^{X} is hydrogen, —C₁₋₆ alkyl, —C(O)CH₃, or —C(O)CF₃;
R^{Y} is hydrogen or —C₁₋₆ alkyl;
E¹ is hydrogen, —CH₂C(O)NH₂, or —CH₂C(O)OH;
A¹ is a trivalent linker (e.g., a substituted or unsubstituted amino acid); and
L¹, L², L³, and L⁵ are each independently selected from (i), (ii), or (iii), or (iv):
   (i) —C(O)—,
   (ii) —(CO)ₘ(CH₂)ₙ(OC₂₋₆ alkyl)ₒ(NH)ₚ(CO)_{q}—,
   (iii) —(CO)ᵣ(CH₂)ₛ(NR¹⁰C₂₋₆ alkyl)ₜ(NR¹¹)ᵤ(CO)ᵥ—; or
   (iv) —(CO)ᵣ—(CH₂)ₛ—(NR¹⁰C(O)C₁₋₆ alkyl)ₜ-(NR¹¹)ᵤ—(CO)ᵥ—;
   wherein:
   R¹⁰ is, in each instance, independently hydrogen or C₁₋₃ alkyl;
   R¹¹ is, in each instance, independently hydrogen or C₁₋₃ alkyl;
   m is, in each instance, independently 0 or 1;
   n is in each instance, independently an integer from 0 to 10;
   is in each instance, independently an integer from 0 to 10;
   p is in each instance, independently 0 or 1;
   q is in each instance, independently 0 or 1;
   r is in each instance, independently 0 or 1;
   s is in each instance, independently an integer from 0 to 10;
   t is in each instance, independently an integer from 0 to 10;
   u is in each instance, independently 0 or 1; and
   v is in each instance, independently 0 or 1.

In some embodiments, provided herein is a compound of Formula (III-A) or Formula (IV-A), wherein:
R^{X} is hydrogen, —C₁₋₆ alkyl, or —C(O)CH₃;
R^{Y} is hydrogen or —C₁₋₆ alkyl;
E¹ is hydrogen, —CH₂C(O)OH, or —CH₂C(O)NH₂;
A¹ is a substituted or unsubstituted amino acid (e.g., a substituted or unsubstituted lysine or a substituted or unsubstituted glutamine);
L¹ is —C(O)—C₂₋₄ alkyl-[O—C₂₋₆ alkyl]₁₋₈-NH; or
   —C(O)—C₁₋₄ alkyl-[N(CH₃)—C₂₋₆ alkyl]₁₋₈-NH—,
L² is —C(O)—C₂₋₄ alkyl-[O—C₂₋₆ alkyl]₁₋₈-NHC(O);
   —C(O)—C₁₋₄ alkyl-[N(CH₃)—C₂₋₆ alkyl]₁₋₈-NHC(O)—, or
   —C(O)—C₁₋₄ alkyl-[N(CH₃)C(O)—C₁₋₆ alkyl]₁₋₈-N(CH₃)—, and
L³ is —C(O)—C₂₋₄ alkyl-[O—C₂₋₆ alkyl]₁₋₈-NHC(O);
   —C(O)—C₁₋₄ alkyl-[N(CH₃)—C₂₋₆ alkyl]₁₋₈-NHC(O)—; or
   —C(O)—C₁₋₄ alkyl-[N(CH₃)C(O)—C₁₋₆ alkyl]₁₋₈-N(CH₃)—.

In some embodiments, provided herein is a compound of Formula (III-A) or Formula (IV-A), having the structure:

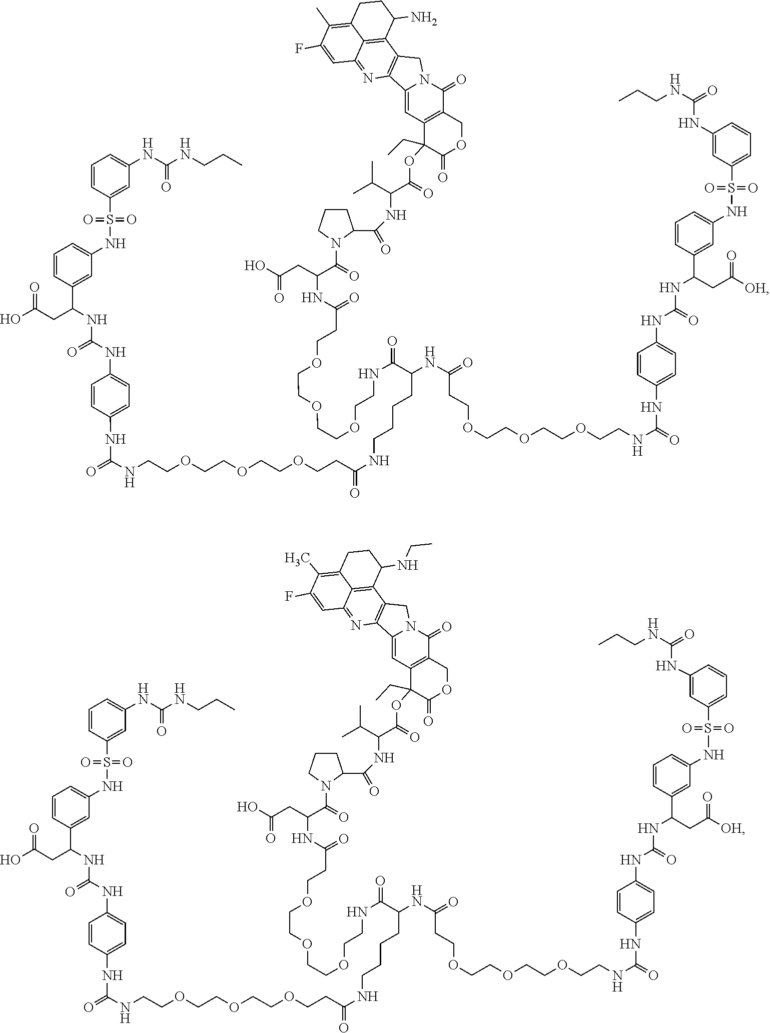

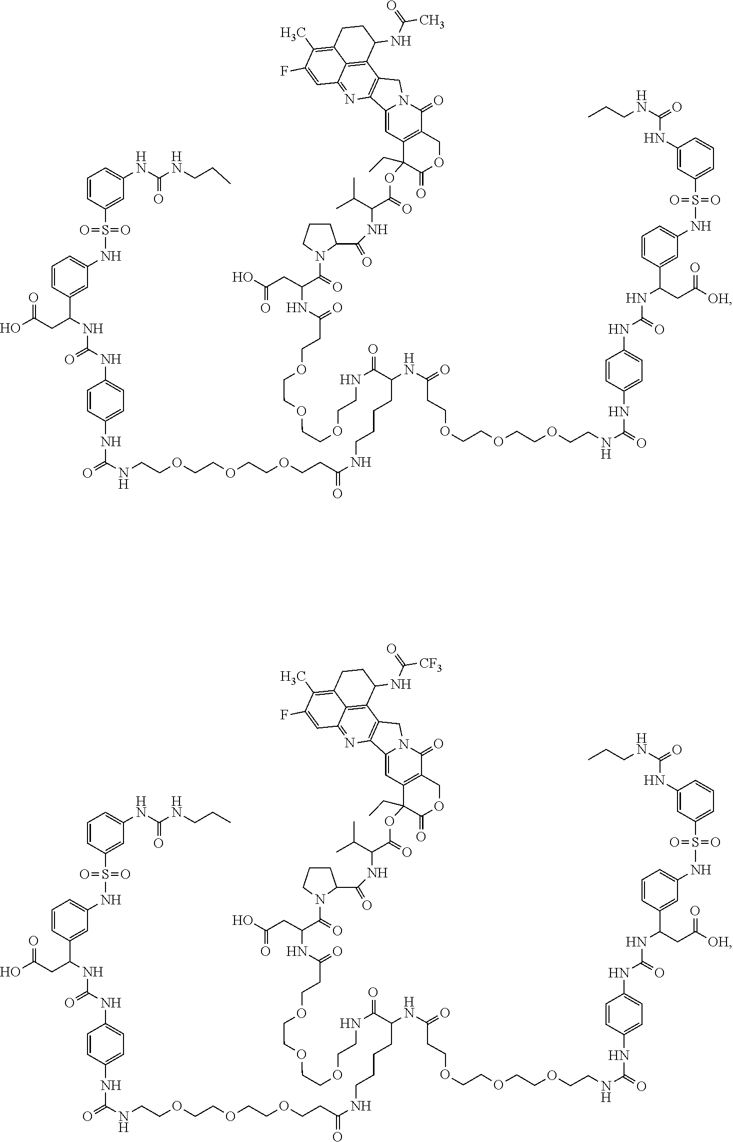

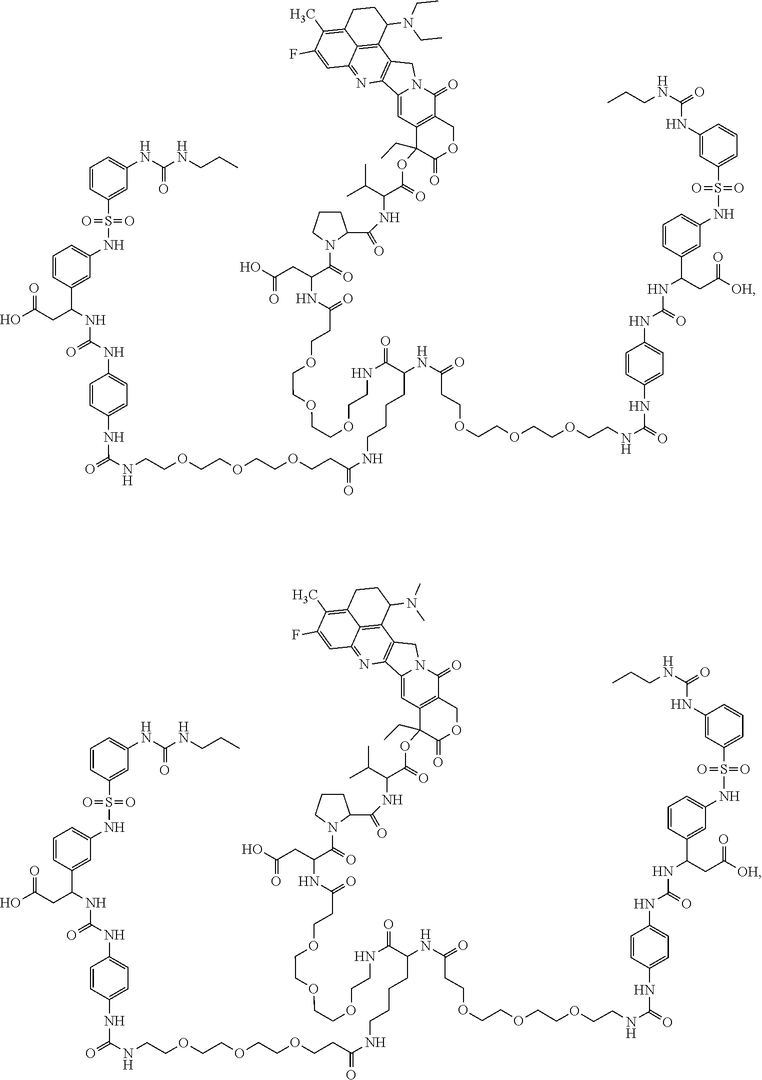

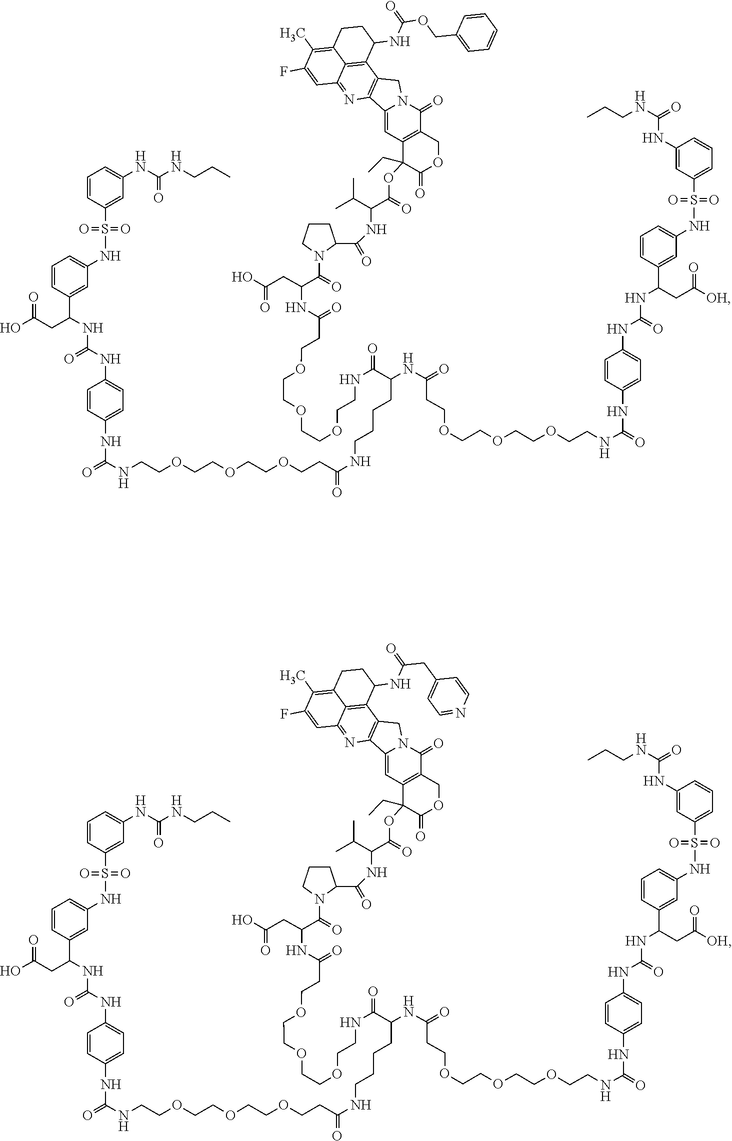

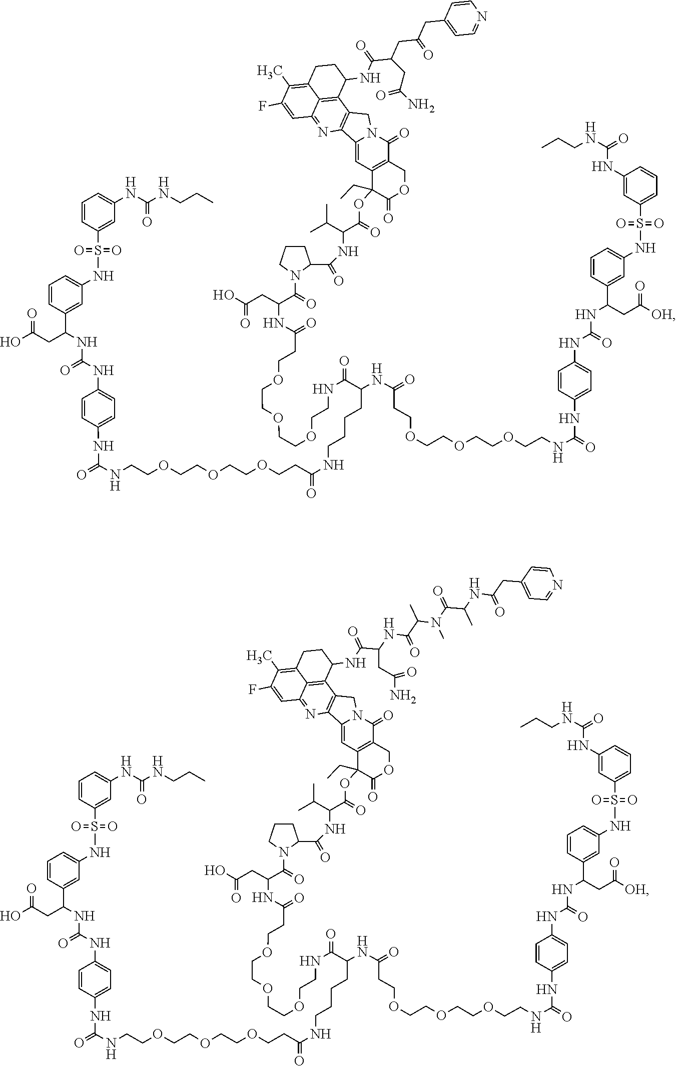

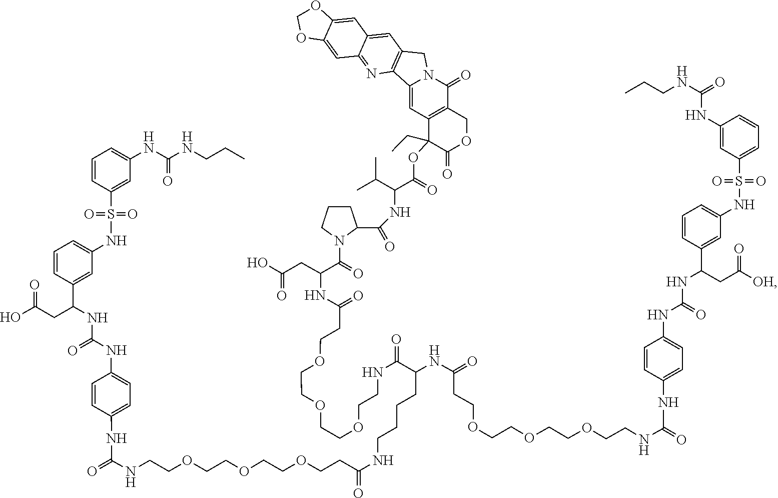 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCCCC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCOCCOCCOCCC(=O)NC(CC(=O)O)C(=O)N4CCCC4C(=O)NC(C(=O)OC4(CC)C(=O)OCc5c4cc4n(c5=O)Cc5cc6cc7c(cc6nc5-4)OCO7)C(C)C)cc3)c2)c1]

or a pharmaceutically acceptable salt thereof, or a stereoisomer or mixture of stereoisomers thereof.

In some embodiments, provided herein is a compound of Formula (III-A) or Formula (IV-A), having the structure:

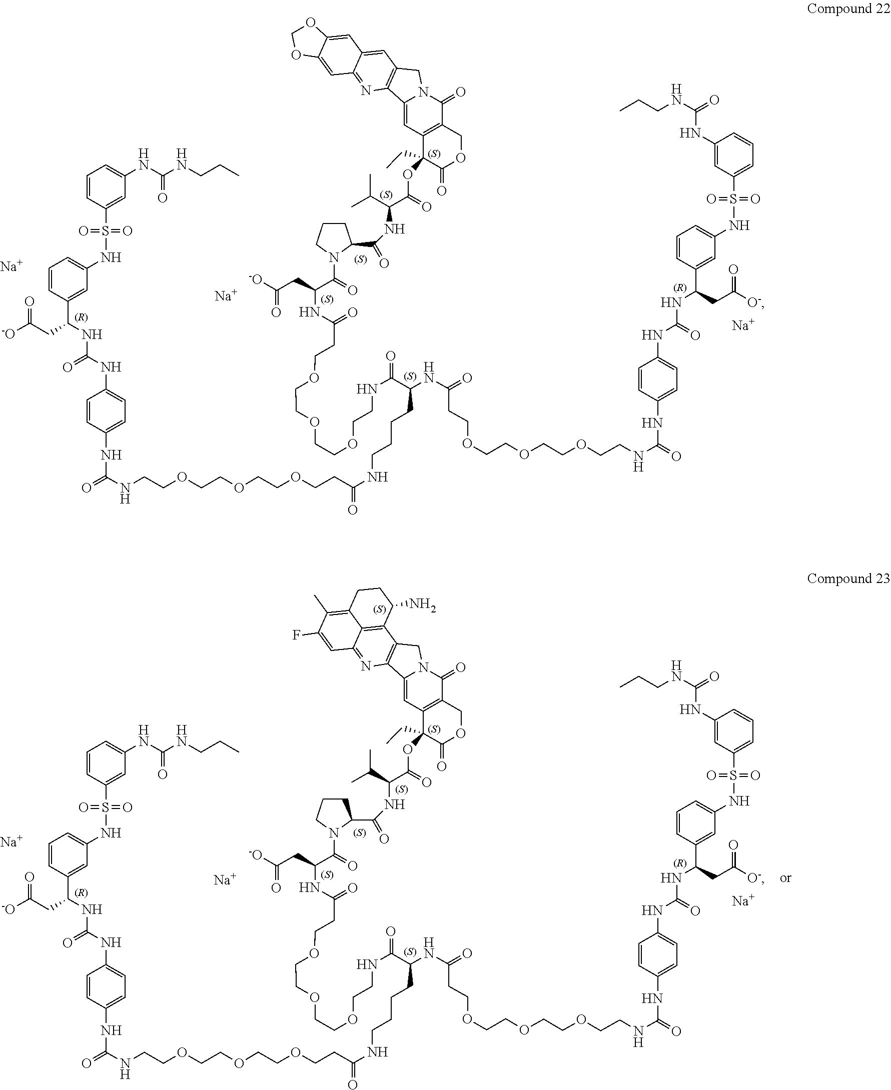

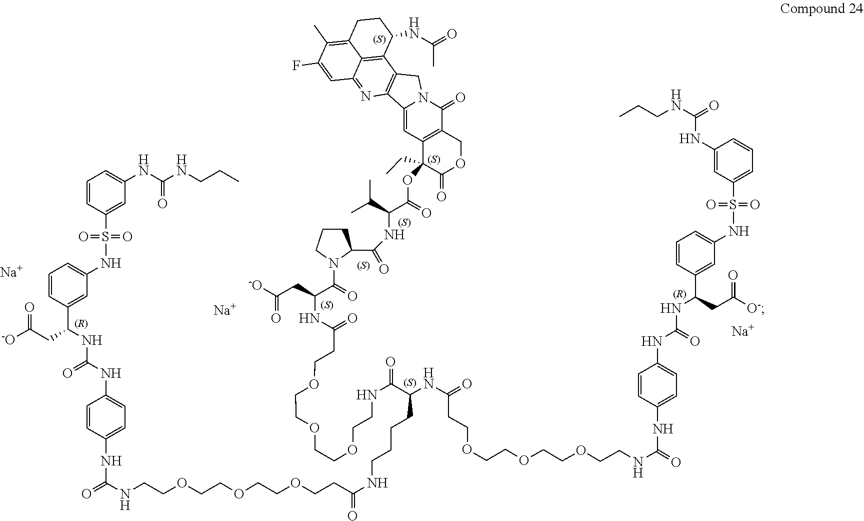

In some embodiments, provided herein is a compound of Formula (III-C) or Formula (IV-C):

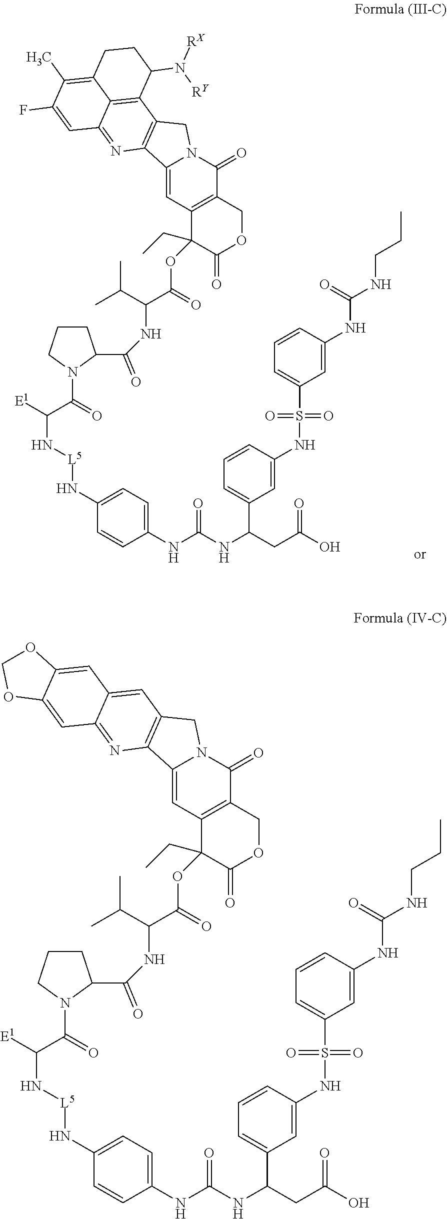

or a pharmaceutically acceptable salt thereof, or a stereoisomer or mixture of stereoisomers thereof.

In some embodiments, the compound is of Formula (III-C′) or Formula (IV-C′):

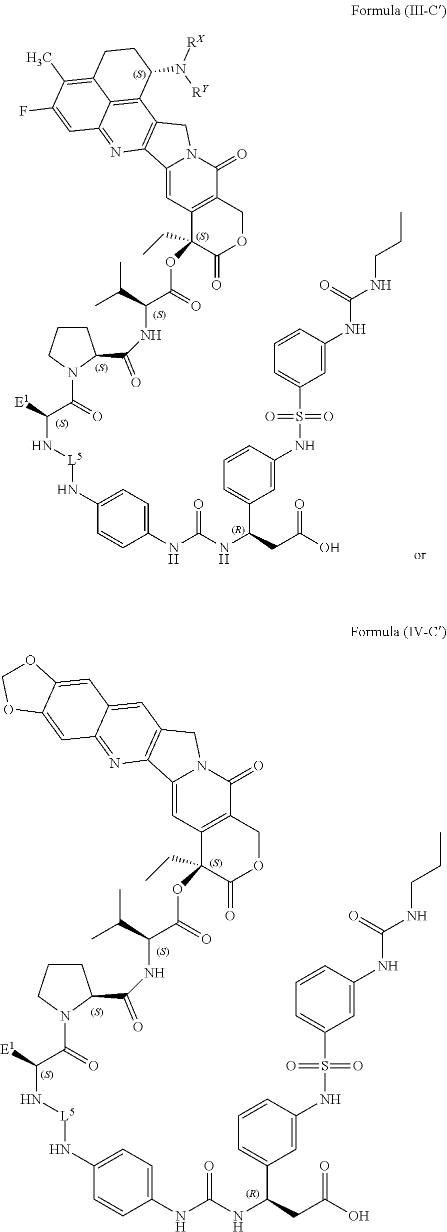

or a pharmaceutically acceptable salt thereof.

In some embodiments, provided herein is a compound of Formula (III-C), Formula (IV-C), Formula (III-C′), or Formula (IV-C′) wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
   wherein:
   EL² is an enzymatically-cleavable linker (e.g., -(L-Asn)(NMe-L-Ala)(L-Ala)- or -(L-Asn)(L-Ala)(L-Ala)-);
R^{Y} is hydrogen, C₁₋₆alkyl, —C(O)C₁₋₆alkyl, or —C(O)C₁₋₆haloalkyl;
E¹ is hydrogen, —CH₂C(O)NH₂ or —CH₂C(O)OH; and
L⁵ is —C(O)—C₁₋₆ alkyl-NHC(O)—,
   —C(O)—C₁₋₆ alkyl-N(CH₃)C(O)—,
   —C(O)—C₂₋₆ alkyl-[OC₂₋₆ alkyl]₁₋₈-NHC(O)—,
   —C(O)—C₂₋₆ alkyl-[OC₂₋₆ alkyl]₁₋₈-N(CH₃)C(O)—,
   —C(O)—C₁₋₆ alkyl-[NHC₁₋₆ alkyl]₁₋₈-NHC(O)—,
   —C(O)—C₁₋₆ alkyl-[NHC₁₋₆ alkyl]₁₋₈-N(CH₃)C(O)—,
   —C(O)—C₁₋₆ alkyl-[N(CH₃)C₁₋₆ alkyl]₁₋₈-NHC(O)—, or
   —C(O)—C₁₋₆ alkyl-[N(CH₃)C₁₋₆ alkyl]₁₋₈-N(CH₃)C(O)—.

In some embodiments, provided herein is a compound or a pharmaceutically acceptable salt thereof, or a stereoisomer or mixture of stereoisomers thereof, having the structure of Formula (III-C) or Formula (IV-C), wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -(L-Asn)-(N-Me-L-Ala)-(L-Ala)-C(O)CH₂-(pyridine)
R^{Y} is hydrogen, C₁₋₆alkyl, —C(O)C₁₋₆alkyl, or —C(O)C₁₋₆haloalkyl;
E¹ is hydrogen, —CH₂C(O)NH₂ or —CH₂C(O)OH;
L⁵ is —C(O)—C₂₋₆ alkyl-[OCH₂CH₂]₂₋₄—NHC(O)—, or
   —C(O)—C₁₋₆ alkyl-[N(CH₃)CH₂CH₂]₂₋₄—NHC(O)—.

In some embodiments, E¹ is hydrogen. In some embodiments, E¹ is hydrogen; and L⁵ is —C(O)—C₂₋₄ alkyl-[OC₂₋₆ alkyl]₁₋₈-NHC(O)—. In some embodiments, E¹ is hydrogen; and L⁵ is —C(O)—C₂₋₄ alkyl-[OC₂₋₄ alkyl]₂₋₆—NHC(O)— or —C(O)—C₁₋₄ alkyl-[N(CH₃)—C₂₋₄ alkyl]₂₋₆—NHC(O)—.

In some embodiments, E¹ is —CH₂C(O)NH₂. In some embodiments, L⁵ is —C(O)—C₁₋₆ alkyl-[N(CH₃)—C₂₋₆ alkyl]₁₋₈-NHC(O)—. In some embodiments, E¹ is —CH₂C(O)NH₂ and L⁵ is —C(O)—C₁₋₆ alkyl-[N(CH₃)—C₂₋₆ alkyl]₁₋₈-NHC(O)—. In some embodiments, E¹ is —CH₂C(O)OH. In some embodiments, L⁵ is —C(O)—C₂₋₄ alkyl-[OC₂₋₆ alkyl]₁₋₈-NHC(O)—. In some embodiments, E¹ is —CH₂C(O)OH; and L⁵ is —C(O)—C₂₋₄ alkyl-[OC₂₋₆ alkyl]₁₋₈-NHC(O)—. In some embodiments, L⁵ is —C(O)—C₂₋₄ alkyl-[OC₂₋₄ alkyl]₂₋₆—NHC(O)— or —C(O)—C₁₋₄ alkyl-[N(CH₃)—C₂₋₄ alkyl]₂₋₆—NHC(O)—. In some embodiments, L⁵ is —C(O)—CH₂CH₃—[OCH₂CH_{3]3}—NHC(O)— or —C(O)CH₂—[N(CH₃)CH₂CH_{3]3}—NHC(O)—. In some embodiments, E¹ is hydrogen, —CH₂C(O)OH or —CH₂C(O)NH₂; and L⁵ is —C(O)—CH₂CH₃—[OCH₂CH_{3]3}—NHC(O)— or —C(O)CH₂—[N(CH₃)CH₂CH_{3]3}—NHC(O)—.

In some embodiments, provided herein is a compound or a pharmaceutically acceptable salt thereof, or a stereoisomer or mixture of stereoisomers thereof, having the structure of Formula (III-C) or Formula (IV-C), wherein:
E¹ is hydrogen, —CH₂C(O)NH₂ or —CH₂C(O)OH; and
L⁵ is —C(O)—C₂₋₄ alkyl-[OCH₂CH₂]₂₋₄—NHC(O)—, or
   —C(O)—C₁₋₄ alkyl-[N(CH₃)CH₂CH₂]₂₋₄—NHC(O)—.

In some embodiments E¹ is hydrogen; and L⁵ is —C(O)—C₁₋₆ alkyl-[N(CH₃)CH₂CH₂]₂₋₄—NHC(O)—. In some embodiments, E¹ is hydrogen; and L⁵ is —C(O)—CH₂—[N(CH₃)CH₂CH_{2]3}—NHC(O)—. In some embodiments E¹ is —CH₂C(O)NH₂; and L⁵ is —C(O)—C₁₋₆ alkyl-[N(CH₃)CH₂CH₂]₂₋₄—NHC(O)—. In some embodiments, E¹ is —CH₂C(O)NH₂; and L⁵ is —C(O)—CH₂—[N(CH₃)CH₂CH_{2]3}—NHC(O)—. In some embodiments, E¹ is —CH₂C(O)OH; and L⁵ is —C(O)—C₂₋₆ alkyl-[OCH₂CH₂]₂₋₄—NHC(O)—. In some embodiments, E¹ is —CH₂C(O)OH; and L⁵ is —C(O)—CH₂CH₂—[OCH₂CH_{2]3}—NHC(O)—.

In some embodiments, L⁵ is:
—C(O)—C₂₋₆ alkyl-[OCH₂CH₂]₂₋₄—NHC(O)—; or
—C(O)—C₁₋₆ alkyl-[N(CH₃)CH₂CH₂]₂₋₄—NHC(O)—.

In some embodiments, L⁵ is:
—C(O)—CH₂CH₂—[OCH₂CH_{2]3}—NHC(O)—; or
—C(O)—CH₂—[N(CH₃)CH₂CH_{2]3}—NHC(O)—.

In some embodiments, provided herein is a compound of Formula (III-C), Formula (IV-C), Formula (III-C′), or Formula (IV-C′) wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -(-L-Asn)-(N-Me-L-Ala)-(L-Ala-)-C(O)CH₂-(pyridine); and
R^{Y} is hydrogen or —C₁₋₆ alkyl.

In some embodiments, provided herein is a compound of Formula (III-C), Formula (IV-C), Formula (III-C′), or Formula (IV-C′) wherein:
R^{X} is hydrogen, —CH₂CH₃, —C(O)CH₃, —C(O)CF₃, —C(O)OBn or —C(O)CH₂(pyridine), -Asn-C(O)CH₂(pyridine), or -(-L-Asn)-(N-Me-L-Ala)-(L-Ala-)-C(O)CH₂-(pyridine); and
R^{Y} is hydrogen or —CH₂CH₃.

In some embodiments, provided herein is a compound of Formula (III-C), Formula (IV-C), Formula (III-C′), or Formula (IV-C′) wherein:
R^{X} is hydrogen, —C(O)CH₃, or —C(O)OBn;
R^{Y} is hydrogen;
E¹ is —CH₂C(O)OH; and
L⁵ is —C(O)—C_{2F-6} alkyl-[O—C₂₋₆ alkyl]₁₋₈-NHC(O)—.

In some embodiments, E¹ is —CH₂C(O)OH. In some embodiments, L⁵ is —C(O)—C₁₋₄ alkyl-[OC₂₋₆ alkyl]₁₋₈-NHC(O)—. In some embodiments, E¹ is —CH₂C(O)OH; and L⁵ is —C(O)—C₁₋₄ alkyl-[OC₂₋₆ alkyl]₁₋₈-NHC(O)—. In some embodiments, E¹ is —CH₂C(O)OH; and L⁵ is —C(O)—C₁₋₄ alkyl-[OCH₂CH₂]₂₋₄—NHC(O)—. In some embodiments, E¹ is —CH₂C(O)OH; and L⁵ is —C(O)—CH₂CH₂—[OCH₂CH_{2]3}—NHC(O)—.

In some embodiments:
R^{X} is hydrogen;
E¹ is —CH₂C(O)OH; and
L⁵ is —C(O)—CH₂CH₂—[OCH₂CH_{2]3}—NHC(O)—.

In some embodiments:
R^{X} is —C(O)CH₃;
E¹ is —CH₂C(O)OH; and
L⁵ is —C(O)—CH₂CH₂—[OCH₂CH_{2]3}—NHC(O)—.

In some embodiments:
R^{X} is —C(O)OBn;
E¹ is —CH₂C(O)OH; and
L⁵ is —C(O)—CH₂CH₂—[OCH₂CH_{2]3}—NHC(O)—.

In some embodiments, provided herein is a compound having the structure:

In some embodiments, provided herein is a compound of Formula (III-C) or Formula (IV-C), having the structure:

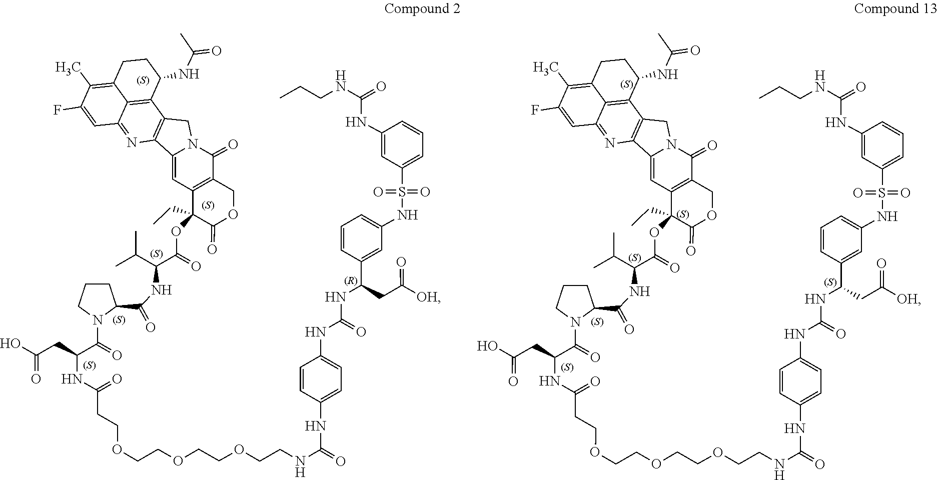

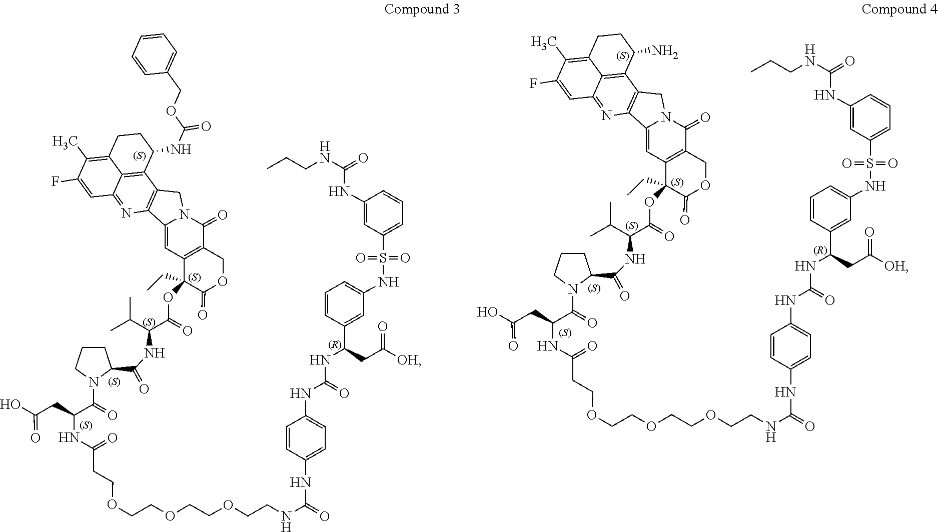

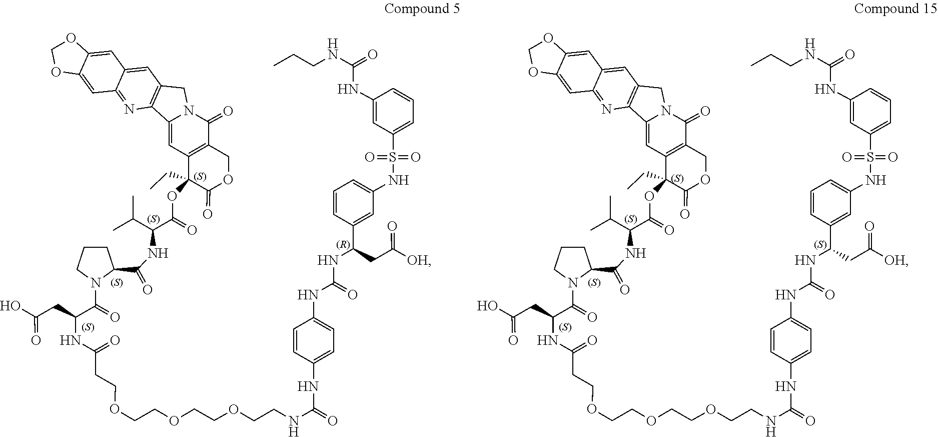

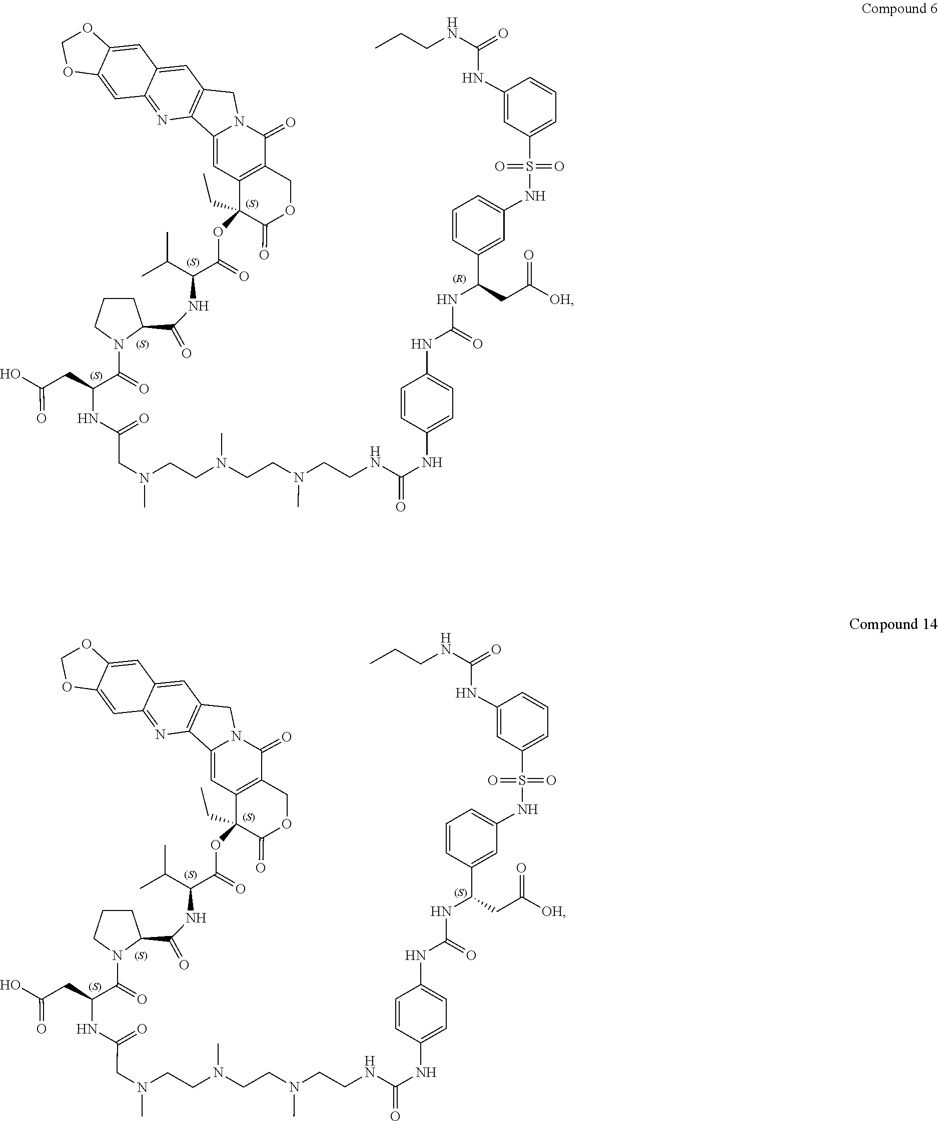

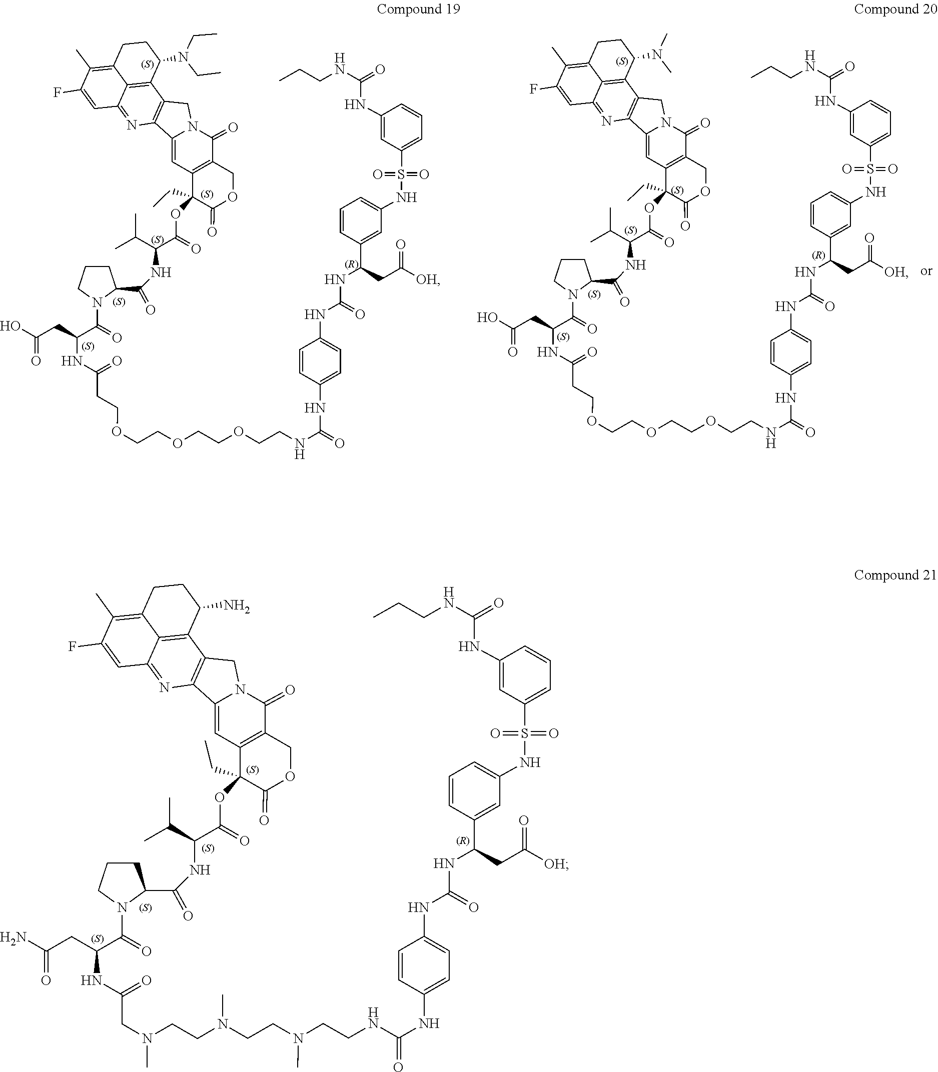

or a pharmaceutically acceptable salt thereof, or a stereoisomer or mixture of stereoisomers thereof.

In some embodiments, provided herein is a compound of Formula (III-C), Formula (IV-C), Formula (III-C′), or Formula (IV-C′) wherein:
R^{X} is —CH₂CH₃, —C(O)CF₃, —C(O)CH₂(pyridine), -Asn-C(O)CH₂(pyridine), or -(-L-Asn)-(N-Me-L-Ala)-(L-Ala-)-C(O)CH₂-(pyridine);
R^{Y} is hydrogen or CH₂CH₃;
E¹ is —CH₂C(O)OH; and
L⁵ is —C(O)—C₂₋₆ alkyl-[O—C₂₋₆ alkyl]₁₋₈-NHC(O)—.

In some embodiments, provided herein is a compound of Formula (III-C) or Formula (IV-C), having the structure:

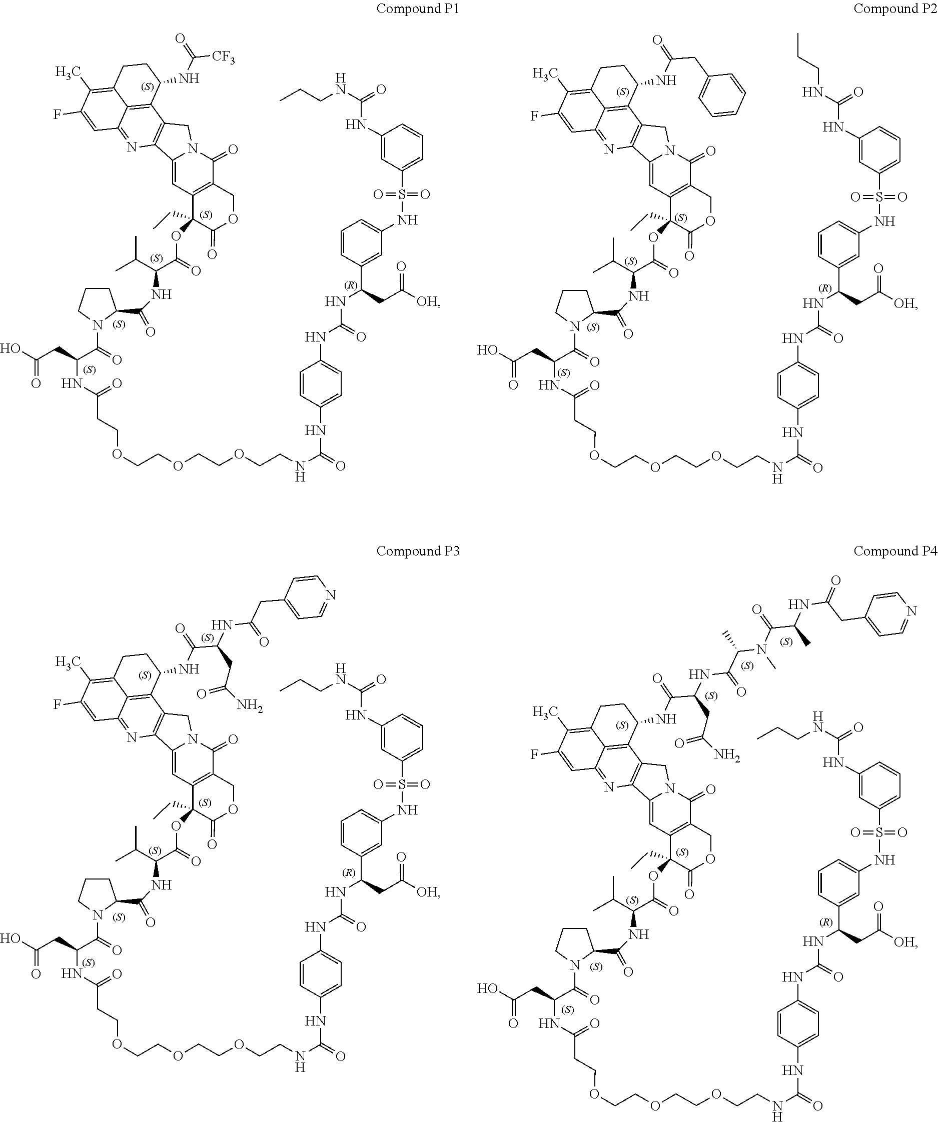

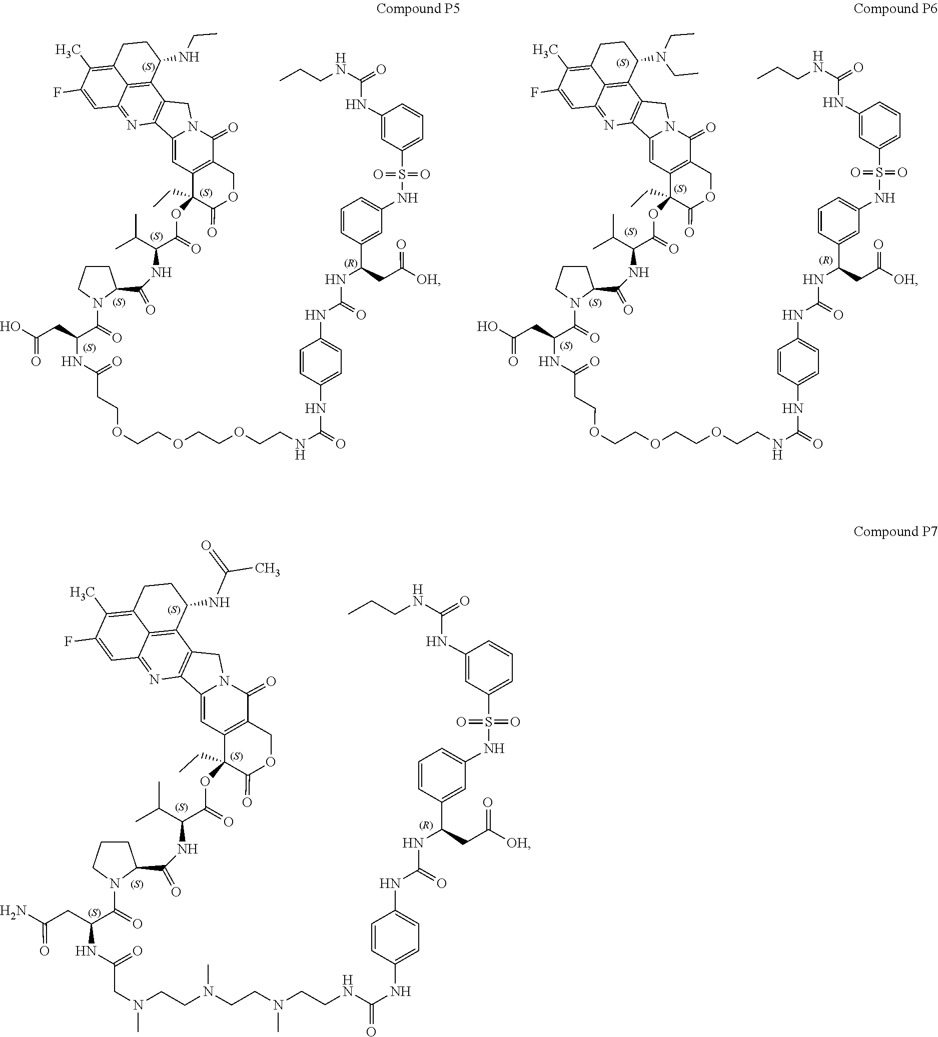

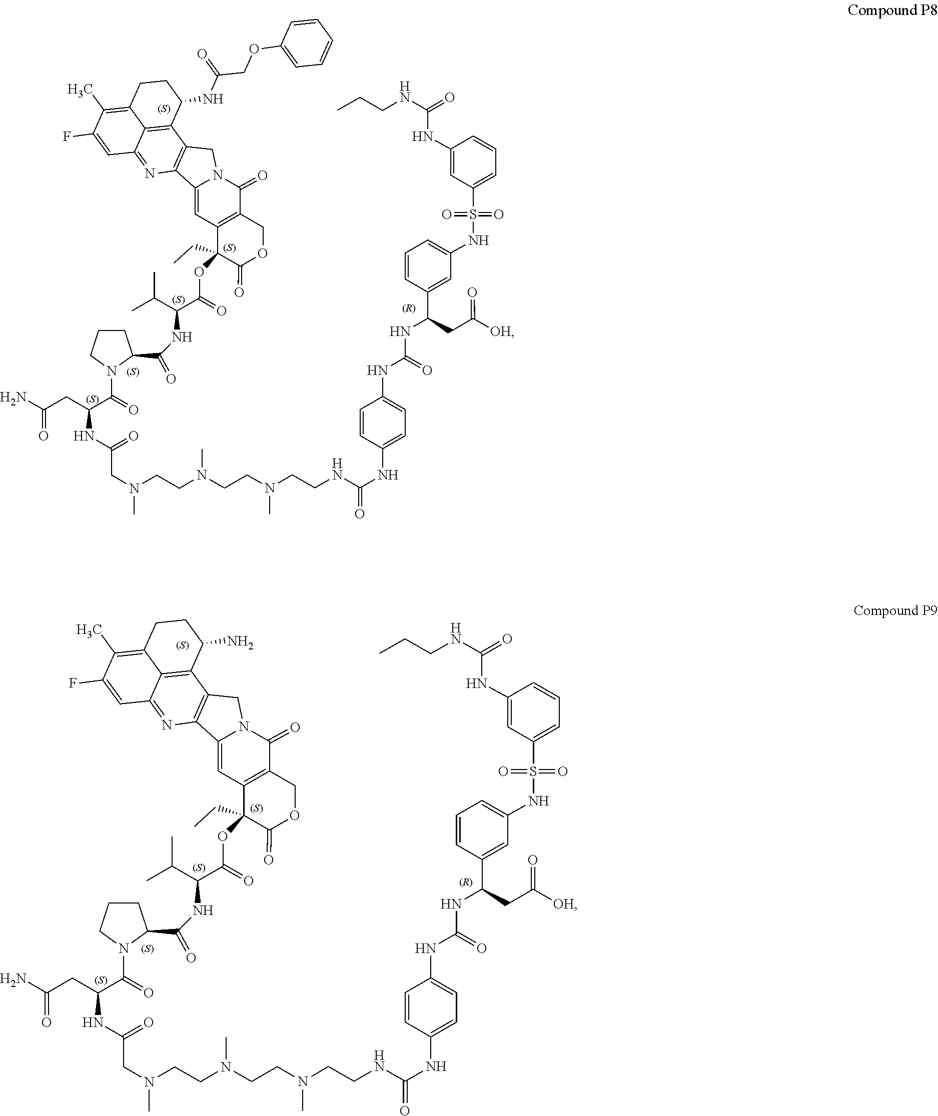

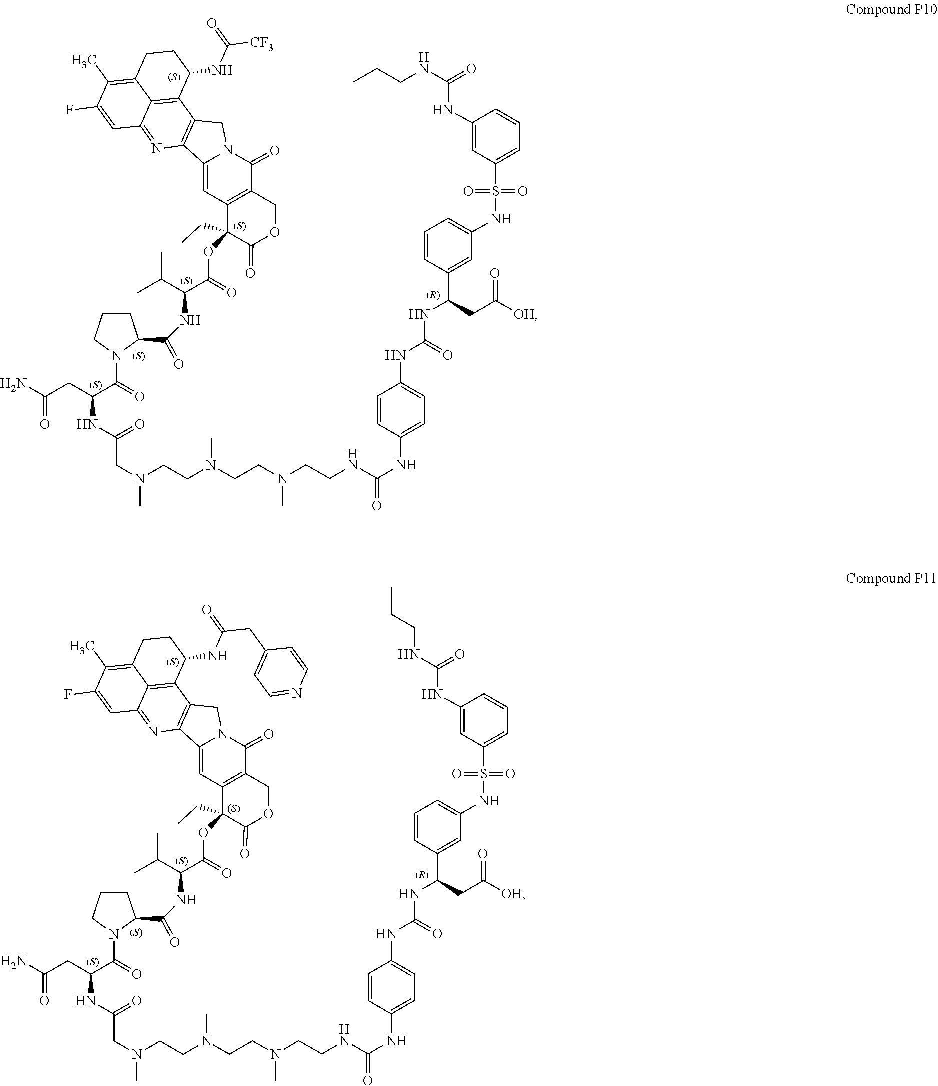

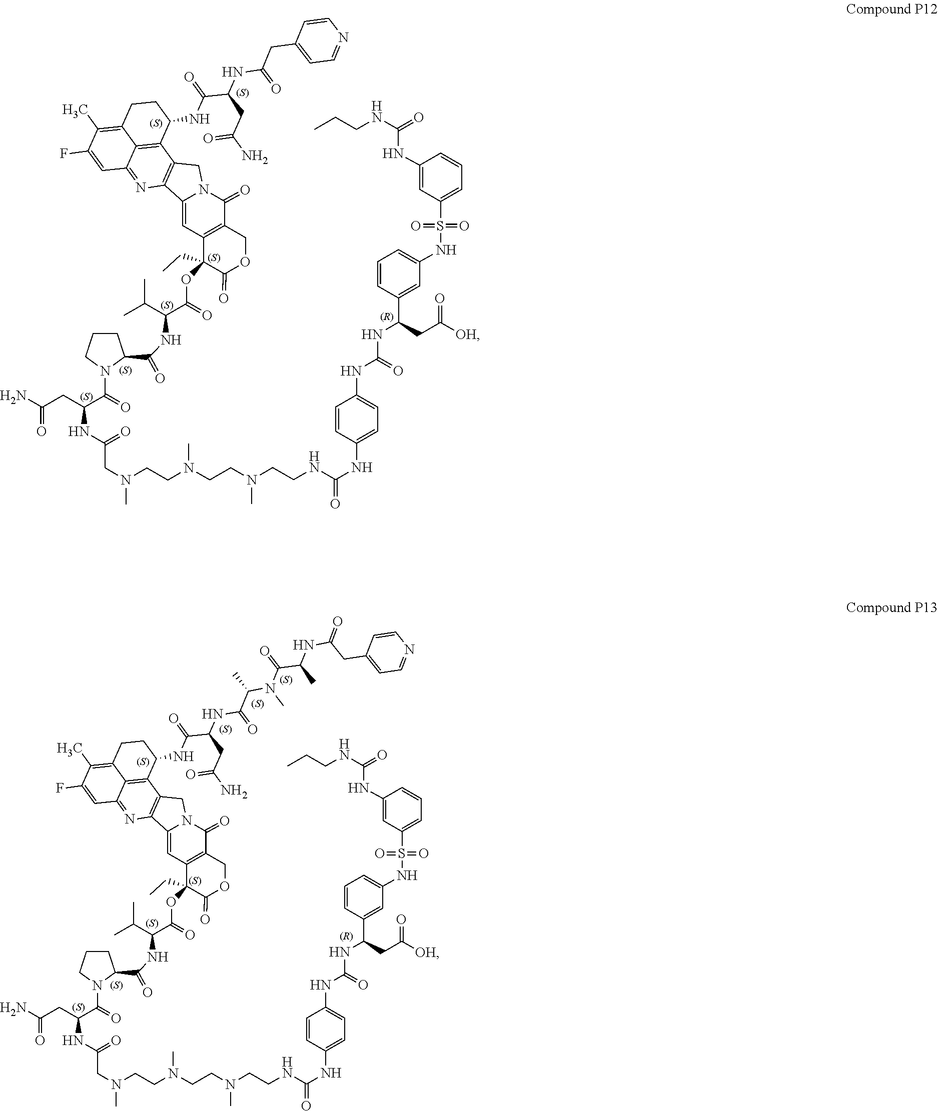

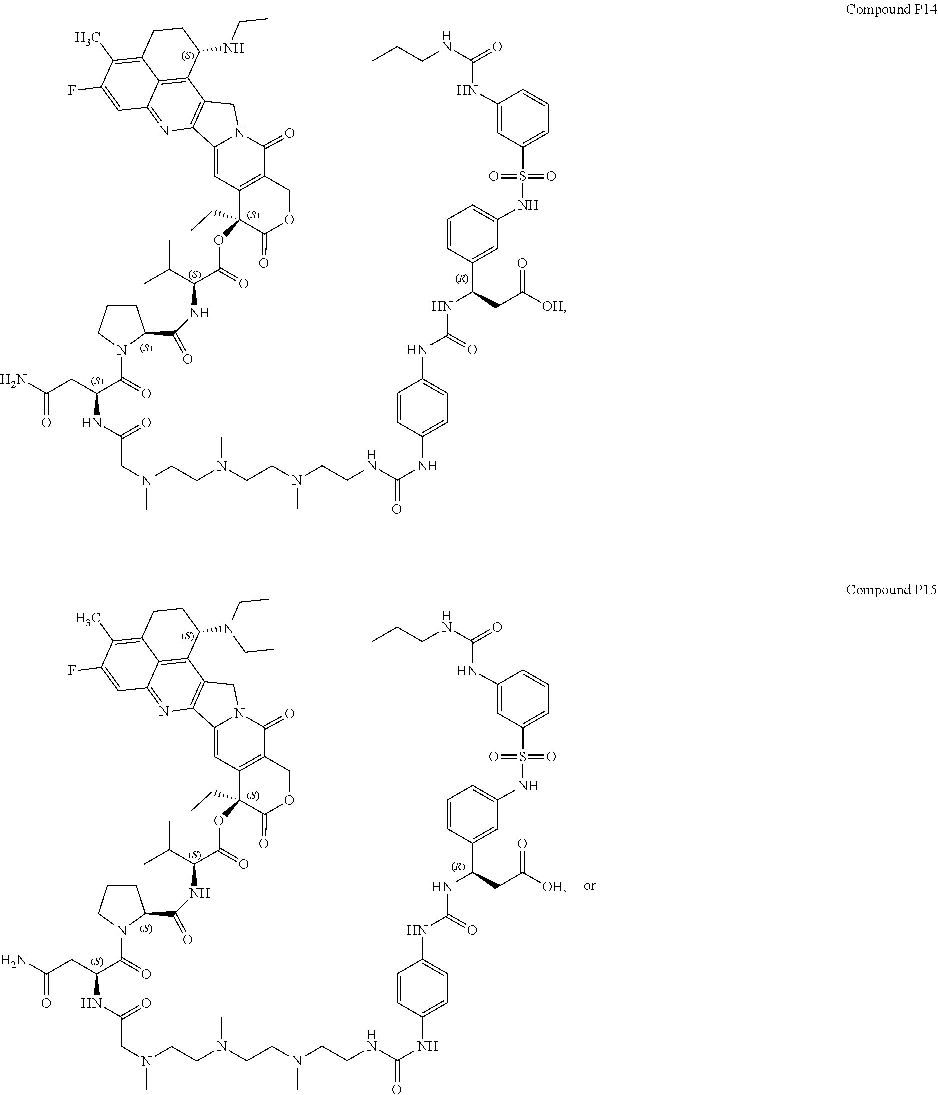

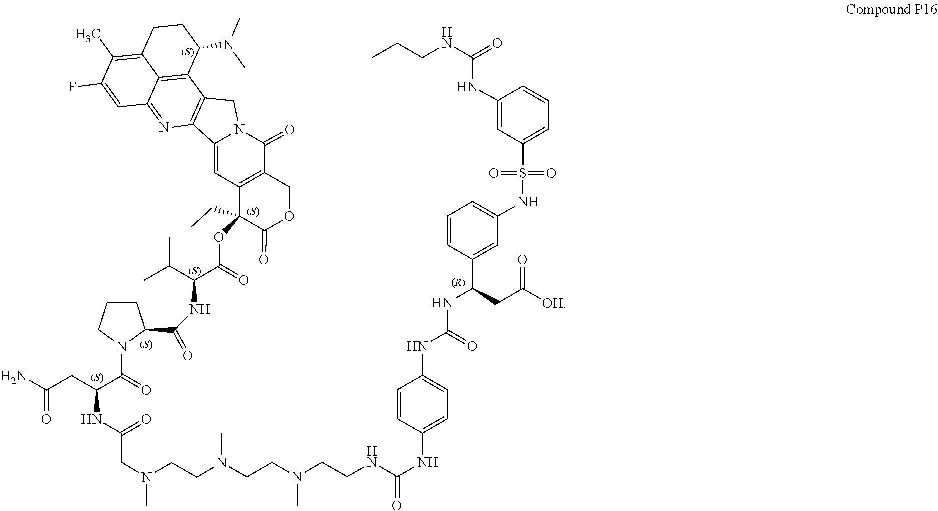

In another aspect, provided herein is a compound of Formula (I):

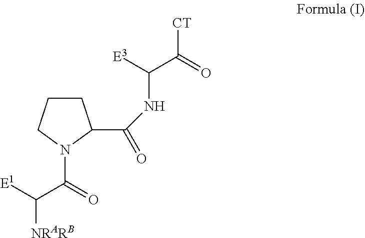

or a pharmaceutically acceptable salt thereof, or a stereoisomer or mixture of stereoisomers thereof; wherein CT is:

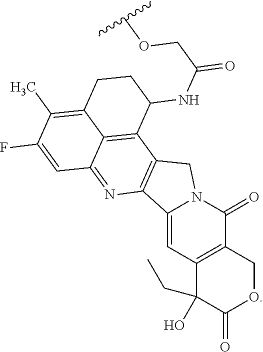 [SMILES: *OCC(=O)NC1CCc2c(C)c(F)cc3nc4c(c1c23)Cn1c-4cc2c(c1=O)COC(=O)C2(O)CC]

In another aspect, provided herein is a compound of Formula (V) or Formula (V′):

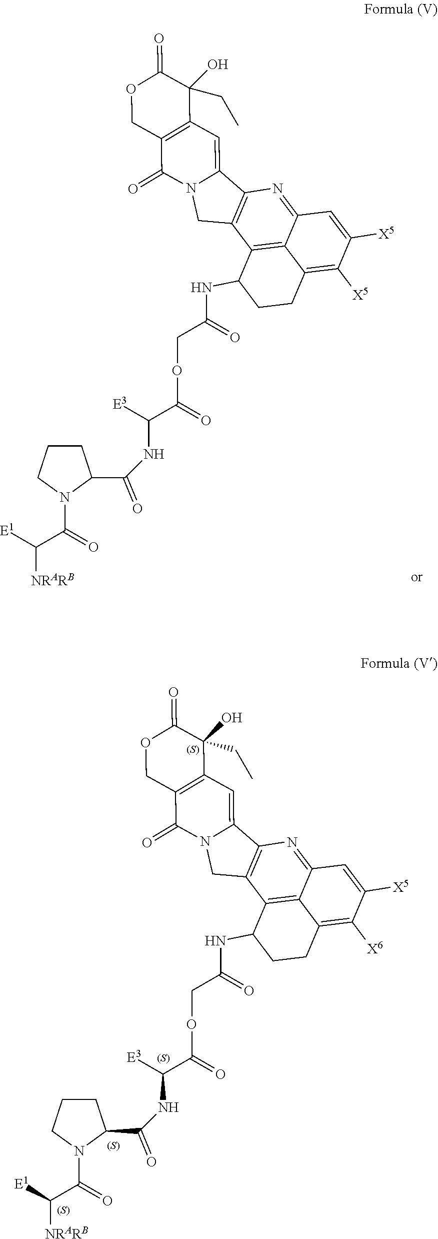

or a pharmaceutically acceptable salt thereof, or a stereoisomer or mixture of stereoisomers thereof.

In some embodiments, provided herein is a compound or a pharmaceutically acceptable salt thereof, or a stereoisomer or mixture of stereoisomers thereof, of Formula (V) or Formula (V′), wherein:
X⁵ and X⁶ are each independently hydrogen, halogen, C₁₋₄ alkyl, or C₁₋₄ haloalkyl;
E¹ is hydrogen, —CH₃, —CH₂CH₃, —CH₂CH₂CH₃, —CH₂C(O)NH₂, —CH₂C(O)OH, or —CH₂C(O)OR¹;
E³ is CH(CH₃)₂, CH₂CH(CH₃)₂, or CH(CH₃)CH₂CH₃;
R^{A} is hydrogen or C₁₋₆ alkyl;
R^{B} is -L¹-A¹(L²-(IN))(L³-(IN)), -L⁵-IN, or L⁷-IN;
   wherein:
   IN is a monovalent radical of an integrin binder;
   A¹ is a trivalent radical;
   each of L¹, L², L³, L⁵, and L⁷ is independently a substituted or unsubstituted C₁₋₃₀ alkyl, or substituted or unsubstituted heteroalkyl;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl.

In some embodiments, provided herein is a compound or a pharmaceutically acceptable salt thereof, or a stereoisomer or mixture of stereoisomers thereof, of Formula (V) or Formula (V′), wherein:
X⁵ is —F, —Cl, or —CH₃; and
X⁶ is —F, —Cl, or —CH₃.

In some embodiments, provided herein is a compound or a pharmaceutically acceptable salt thereof, or a stereoisomer or mixture of stereoisomers thereof, of Formula (V) or Formula (V′), wherein X⁵ is —F and X⁶ is —CH₃.

In some embodiments, provided herein is a compound or a pharmaceutically acceptable salt thereof, or a stereoisomer or mixture of stereoisomers thereof, of Formula (V) or Formula (V′), wherein E¹ is hydrogen. In some embodiments, E¹ is —CH₂C(O)NH₂. In some embodiments, E¹ is —CH₂C(O)OH.

In some embodiments, provided herein is a compound or a pharmaceutically acceptable salt thereof, or a stereoisomer or mixture of stereoisomers thereof, of Formula (V) or Formula (V′), wherein E³ is —CH(CH₃)₂. In some embodiments, E³ is CH₂CH(CH₃)₂. In some embodiments, E³ is —CH(CH₃)CH₂CH₃.

In some embodiments, provided herein is a compound or a pharmaceutically acceptable salt thereof, or a stereoisomer or mixture of stereoisomers thereof, of Formula (V) or Formula (V′), wherein R^{A} is hydrogen. In some embodiments, R^{A} is —CH₃.

In some embodiments, provided herein is a compound or a pharmaceutically acceptable salt thereof, or a stereoisomer or mixture of stereoisomers thereof, having the structure of Formula (V-A) or Formula (V-C):

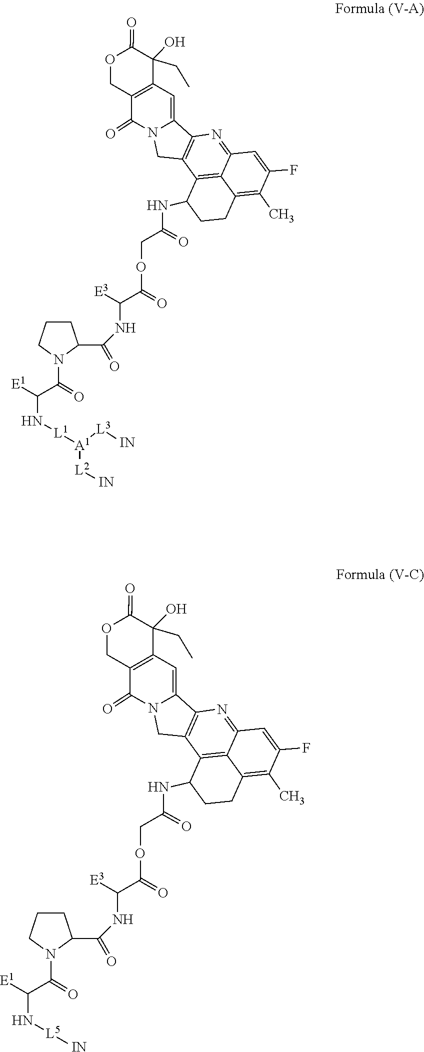

In some embodiments, provided herein is a compound or a pharmaceutically acceptable salt thereof, or a stereoisomer or mixture of stereoisomers thereof, having the structure of Formula (V-A) or Formula (V-C); wherein:
E¹ is hydrogen, —CH₂C(O)NH₂, —CH₂C(O)OH, or —CH₂C(O)OR¹;
E³ is —CH(CH₃)₂;
R¹ is a C₁₋₆ alkyl group that is substituted with -L⁶-IN, —NH₂, —NH(C₁₋₆alkyl), —NHL⁶-IN, —N(C₁₋₆alkyl)₂, or —N(C₁₋₆alkyl)₃⁺; and L⁶ is —C(O)—;
IN is, in each instance, independently an integrin binder;
A¹ is a trivalent heteroalkyl radical;
L¹, L², L³, and L⁵ are each independently:
   (i) —C(O)—,
   (ii) —(CO)ₘ(CH₂)ₙ(OC₂₋₆ alkyl)ₒ(NH)ₚ(CO)_{q}—,
   (iii) —(CO)ᵣ(CH₂)ₛ(NR¹⁰C₂₋₆ alkyl)ₜ(NR¹¹)ᵤ(CO)ᵥ—; or
   (iv) —(CO)ᵣ(CH₂)ₛ(NR¹⁰C(O)—C₁₋₆ alkyl)ₜ(NR¹¹)ᵤ(CO)ᵥ—;
   wherein:
   R¹⁰ is, in each instance, independently hydrogen or C₁₋₃ alkyl;
   R¹¹ is, in each instance, independently hydrogen or C₁₋₃ alkyl;
   m is, in each instance, independently 0 or 1;
   n is in each instance, independently an integer from 0 to 10;
   is in each instance, independently an integer from 0 to 10;
   p is in each instance, independently 0 or 1;
   q is in each instance, independently 0 or 1;
   r is in each instance, independently 0 or 1;
   s is in each instance, independently an integer from 0 to 10;
   t is in each instance, independently an integer from 0 to 10;
   u is in each instance, independently 0 or 1; and
   v is in each instance, independently 0 or 1.

In some embodiments, provided herein is a compound or a pharmaceutically acceptable salt thereof, or a stereoisomer or mixture of stereoisomers thereof, having the structure of Formula (V-C), wherein:
E¹ is hydrogen, —CH₂C(O)NH₂ or —CH₂C(O)OH;
L⁵ is —C(O)—C₁₋₆ alkyl-NHC(O)—,
   —C(O)—C₁₋₆ alkyl-N(CH₃)C(O)—,
   —C(O)—C₂₋₆ alkyl-[O—C₂₋₆ alkyl]₁₋₈-NHC(O)—,
   —C(O)—C₂₋₆ alkyl-[O—C₂₋₆ alkyl]₁₋₈-N(CH₃)C(O)—,
   —C(O)—C₁₋₆ alkyl-[NH—C₂₋₆ alkyl]₁₋₈-NHC(O)—,
   —C(O)—C₁₋₆ alkyl-[NH—C₂₋₆ alkyl]₁₋₈-N(CH₃)C(O)—,
   —C(O)—C₁₋₆ alkyl-[N(CH₃)—C₂₋₆ alkyl]₁₋₈-NHC(O)—, or
   —C(O)—C₁₋₆ alkyl-[N(CH₃)—C₂₋₆ alkyl]₁₋₈-N(CH₃)C(O)—; and
   IN is a monovalent radical of an integrin binder.

In some embodiments, provided herein is a compound or a pharmaceutically acceptable salt thereof, or a stereoisomer or mixture of stereoisomers thereof, having the structure of Formula (V-C), wherein:
E¹ is hydrogen, —CH₂C(O)NH₂ or —CH₂C(O)OH;
L⁵ is —C(O)—C₂₋₆ alkyl-[O—C₂₋₆ alkyl]₁₋₈-NHC(O)—,
   —C(O)—C₁₋₆ alkyl-[N(CH₃)—C₂₋₆ alkyl]₁₋₈-NHC(O)—, or
   —C(O)—C₁₋₆ alkyl-[N(CH₃)—C₂₋₆ alkyl]₁₋₈-N(CH₃)C(O)—; and
IN is a monovalent radical of an integrin binder.

In some embodiments, E¹ is —CH₂C(O)NH₂. In some embodiments, L⁵ is —C(O)—C₁₋₆ alkyl-[N(CH₃)—C₂₋₆ alkyl]₁₋₈-NHC(O)—. In some embodiments, E¹ is —CH₂C(O)NH₂ and L⁵ is —C(O)—C₁₋₆ alkyl-[N(CH₃)—C₂₋₆ alkyl]₁₋₈-NHC(O)—. In some embodiments, E¹ is —CH₂C(O)OH. In some embodiments, L⁵ is —C(O)—C₂₋₄ alkyl-[OC₂₋₆ alkyl]₁₋₈-NHC(O)—. In some embodiments, E¹ is —CH₂C(O)OH; and L⁵ is —C(O)—C₂₋₄ alkyl-[OC₂₋₆ alkyl]₁₋₈-NHC(O)—.

In some embodiments, provided herein is a compound or a pharmaceutically acceptable salt thereof, or a stereoisomer or mixture of stereoisomers thereof, having the structure of Formula (VII-A), wherein:
E¹ is —CH₂C(O)NH₂ or —CH₂C(O)OH; and
L⁵ is —C(O)—C₂₋₆ alkyl-[OCH₂CH₂]₂₋₄—NHC(O)—, or
   —C(O)—C₁₋₆ alkyl-[N(CH₃)CH₂CH₂]₂₋₄—NHC(O)—.

In some embodiments E¹ is —CH₂C(O)NH₂; and L⁵ is —C(O)—C₁₋₆ alkyl-[N(CH₃)CH₂CH₂]₂₋₄—NHC(O)—. In some embodiments, E¹ is —CH₂C(O)NH₂; and L⁵ is —C(O)—CH₂—[N(CH₃)CH₂CH_{2]3}—NHC(O)—. In some embodiments, E¹ is —CH₂C(O)OH; and L⁵ is —C(O)—C₂₋₆ alkyl-[OCH₂CH₂]₂₋₄—NHC(O)—. In some embodiments, E¹ is —CH₂C(O)OH; and L⁵ is —C(O)—CH₂CH₂—[OCH₂CH_{2]3}—NHC(O)—.

In some embodiments, provided herein is a compound or a pharmaceutically acceptable salt thereof, or a stereoisomer or mixture of stereoisomers thereof, having the structure of Formula (V-A′) or Formula (V-C′):

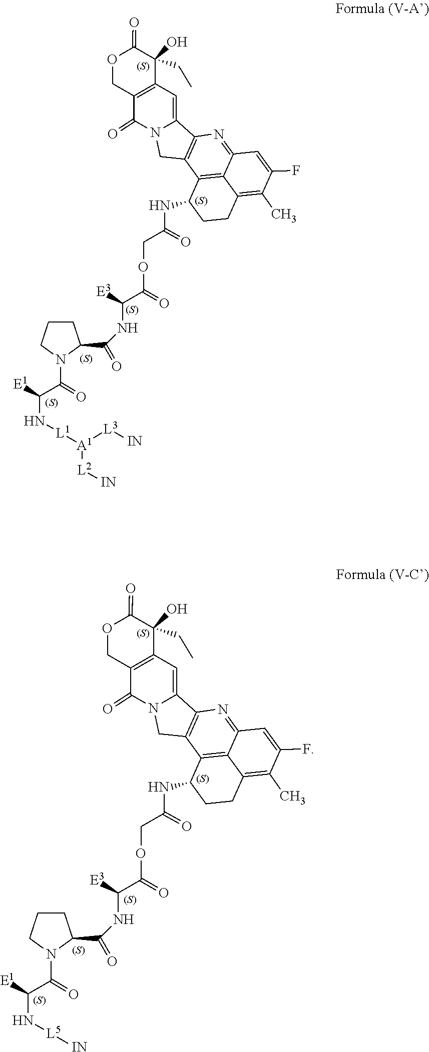

In some embodiments, provided herein is a compound having the structure:

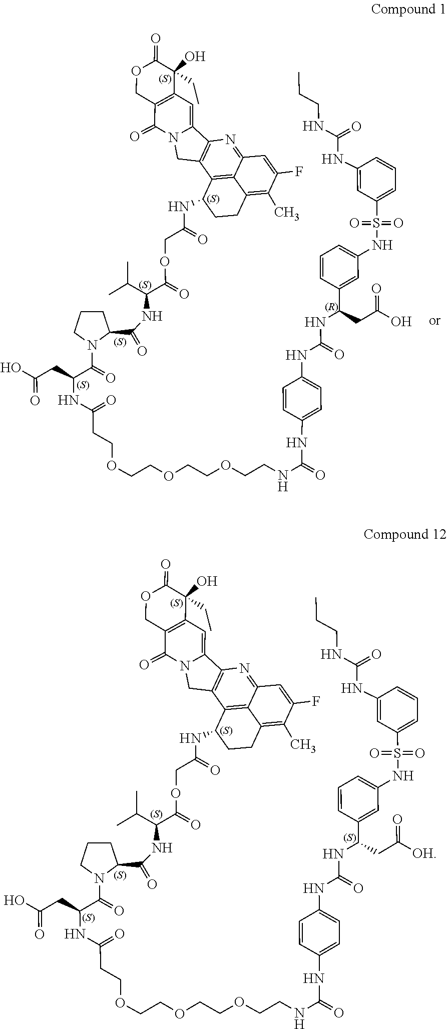

In still another aspect, provided herein is a compound or a pharmaceutically acceptable salt thereof, or a stereoisomer or mixture of stereoisomers thereof, of Formula (X):

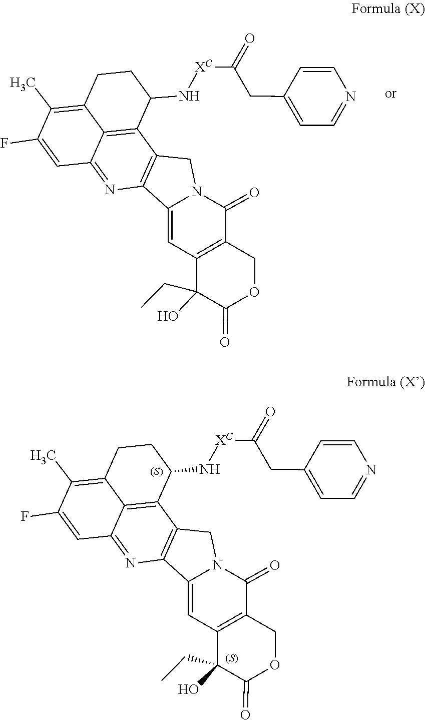

In some embodiments, provided herein is a compound or a pharmaceutically acceptable salt thereof, or a stereoisomer or mixture of stereoisomers thereof, of Formula (X) or Formula (X′), wherein:
X^{C} is a bond,
   (-L-Asn-)-,
   (-L-Asp)-(L-Pro)-(L-Val-)-,
   (-L-Asn)-(L-Pro)-(L-Val-)-,
   (-L-Ala)-(L-Ala)-(L-Asn-)-, or
   (-L-Ala)-(L-N-Me-Ala)-(L-Asn-)-.

In some embodiments, X^{C} is a bond, (-L-Asn-), or (-L-Ala)-(L-N-Me-Ala)-(L-Asn-). In some embodiments, X^{C} is a bond. In some embodiments, X^{C} is (-L-Asn). In some embodiments, X^{C} is (-L-Ala)-(L-N-Me-Ala)-(L-Asn-). In some embodiments, X^{C} is an enzymatically cleavable group (e.g., a legumain cleavable group). In some embodiments, X^{C} is a self-immolative or chemically labile group. In some embodiments, X^{C} contains a self-immolative group.

In some embodiments, X^{C} is a bond,

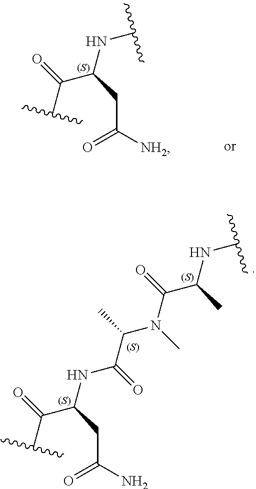

In some embodiments, provided herein is a compound having the structure:

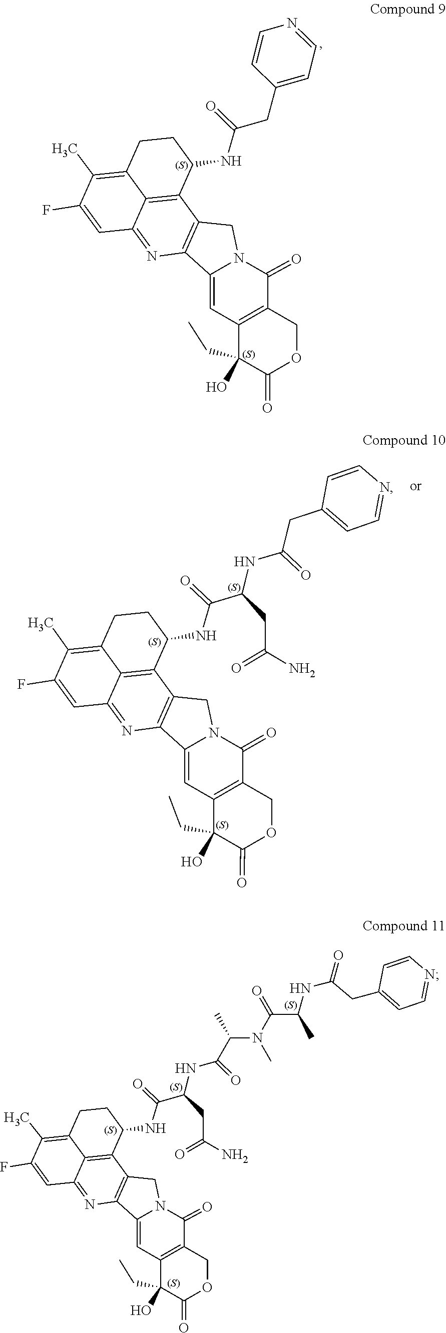

or a pharmaceutically acceptable salt thereof, or a stereoisomer or mixture of stereoisomers thereof.

In some embodiments, compound 10 and compound 11, and conjugates comprising compound 10 or 11 as a group “CT”, are prodrugs. In some embodiments, provided herein is a prodrug compound comprising a legumain-cleavable prodrug group (e.g., a group X^{C}) and a neutrophil elastase-cleavable linker (e.g., EL). In some embodiments, (e.g., wherein CT comprises a legumain-cleavable group X^{C} (e.g., compound 10 or compound 11)), the cytotoxic payload (CT) is activated by two (or more) tumor-associated enzymes (e.g., legumain and neutrophil elastase). In some embodiments, a compound comprising a legumain-cleavable prodrug group is less toxic (e.g., cytotoxic) compared to a compound without said legumain-cleavable group. In some embodiments, a compound comprising a legumain-cleavable prodrug as the payload provides a favorable modulation in pharmacokinetics, compared to a compound comprising an active payload (i.e., a non-prodrug payload, e.g., one having cytotoxic effects).

In some embodiments, provided herein is a compound that is:

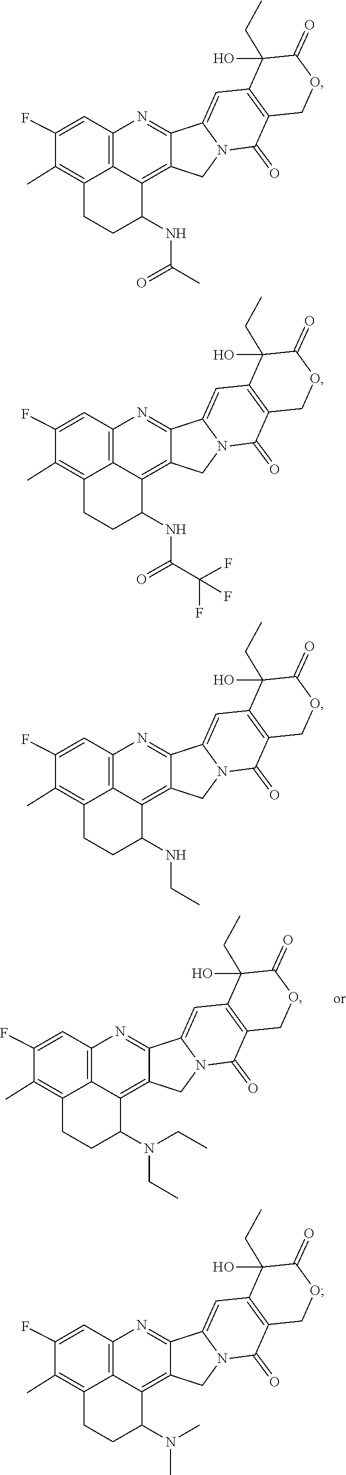

or a pharmaceutically acceptable salt thereof, or a stereoisomer or mixture of stereoisomers thereof. In some embodiments, the compound is for use in the manufacture of a medicament for treating a disease or disorder. In some embodiments, the compound is for use in the manufacture of a small molecule drug conjugate for treating a disease or disorder. In some embodiments, the compound is for use in treating a disease or disorder. In some embodiments, provided herein is a pharmaceutical composition comprising a compound disclosed herein and a pharmaceutically acceptable excipient.

In some embodiments, provided herein is a compound that is.

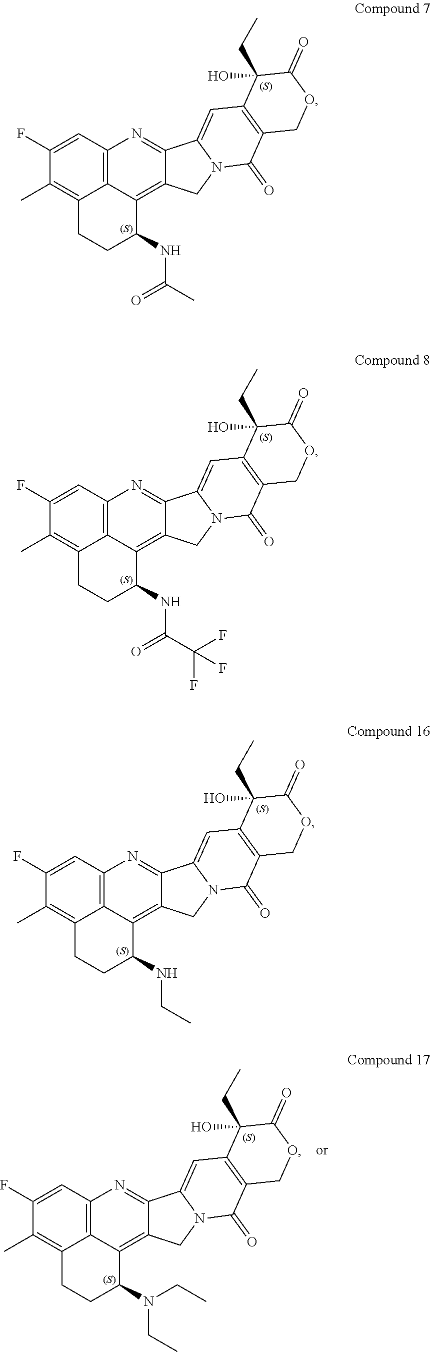

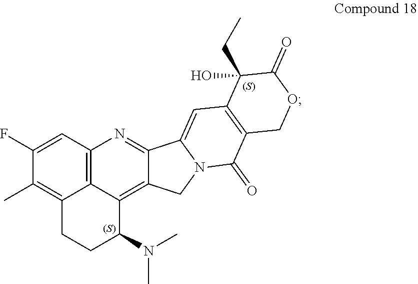

or a pharmaceutically acceptable salt thereof, or a stereoisomer or mixture of stereoisomers thereof. In some embodiments, the compound is for use in the manufacture of a medicament for treating a disease or disorder. In some embodiments, the compound is for use in the manufacture of a small molecule drug conjugate for treating a disease or disorder. In some embodiments, the compound is for use in treating a disease or disorder. In some embodiments, provided herein is a pharmaceutical composition comprising a compound disclosed herein and a pharmaceutically acceptable excipient.

In some embodiments, provided herein is a compound, or a pharmaceutically acceptable salt thereof, or a stereoisomer or mixture of stereoisomers thereof, having a structure according to Formula (II-AA) or Formula (II-CC):

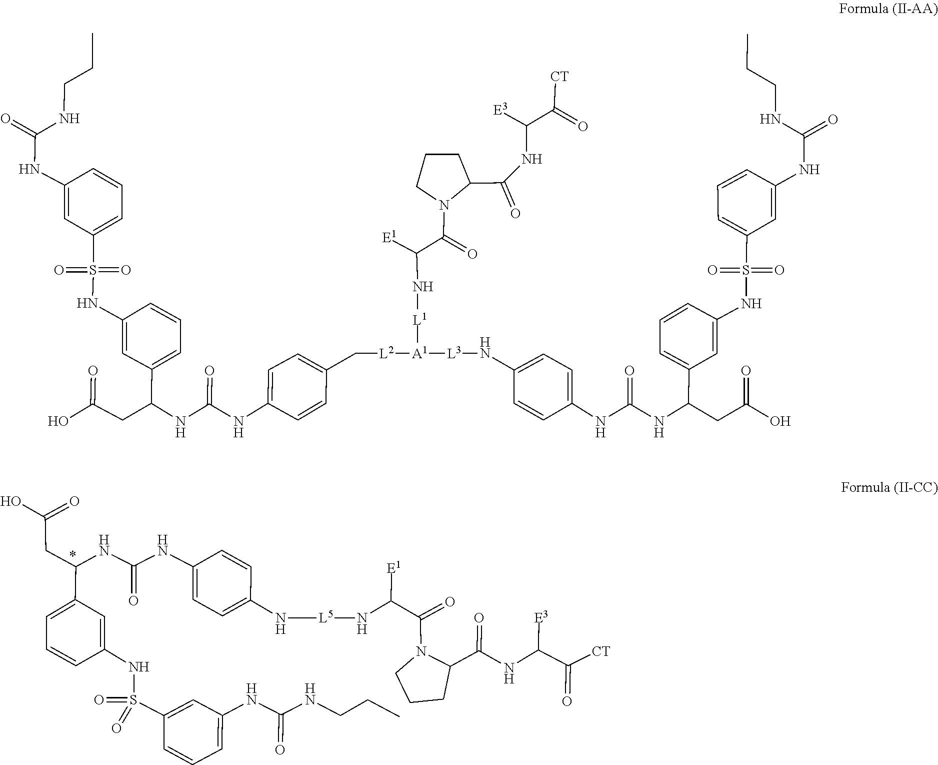

wherein:
E¹ is hydrogen, —CH₂C(O)NH₂, or —CH₂C(O)OH;
E³ is —CH₃ or —CH(CH₃)₂;
* denotes an (R) or (S) stereoisomer;
L¹ is a bivalent linker having the structure:

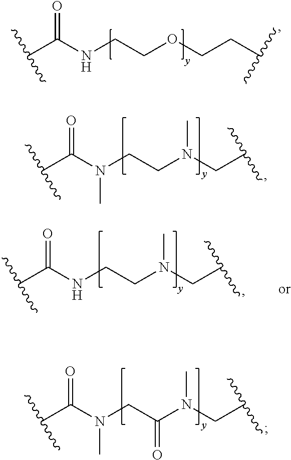

A¹ is a trivalent linker having the structure:

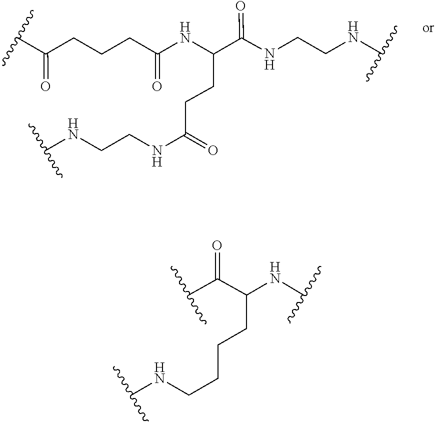 [SMILES: *NCCNC(=O)CCC(NC(=O)CCCC(*)=O)C(=O)NCCN* ; *NCCCCC(N*)C(*)=O]

each of L², L³, and L⁵ is a bivalent linker having the structure:

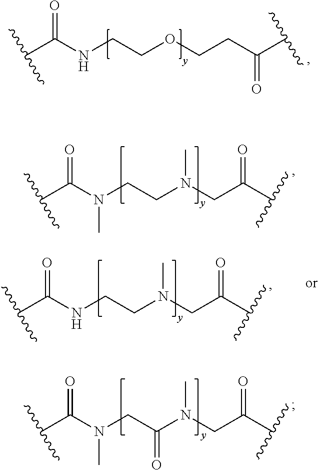

wherein y is 1, 2, 3, 4, 5, 6, 7, or 8 (preferably 2, 3, or 4); and
CT is a hexacyclic cytotoxic or cytostatic group having the structure:

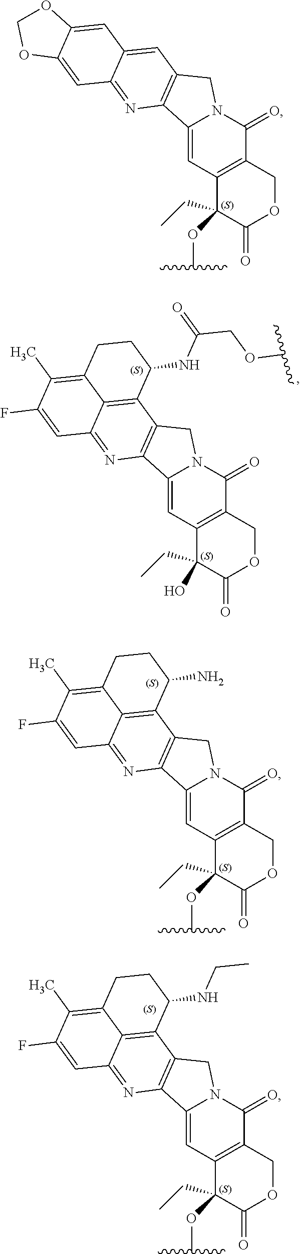

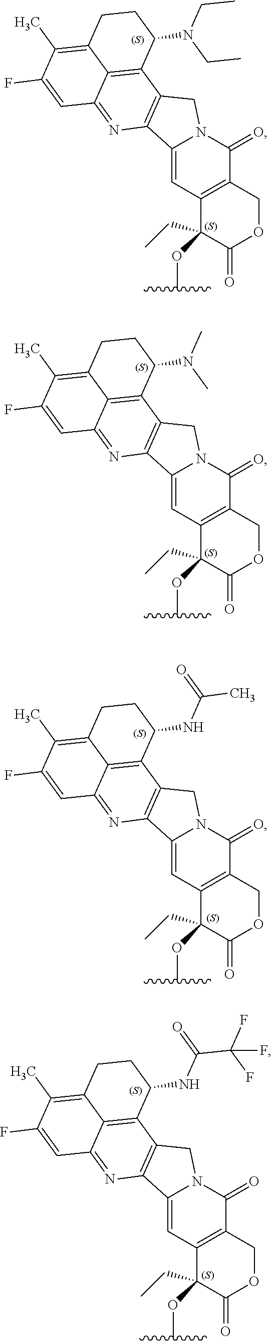

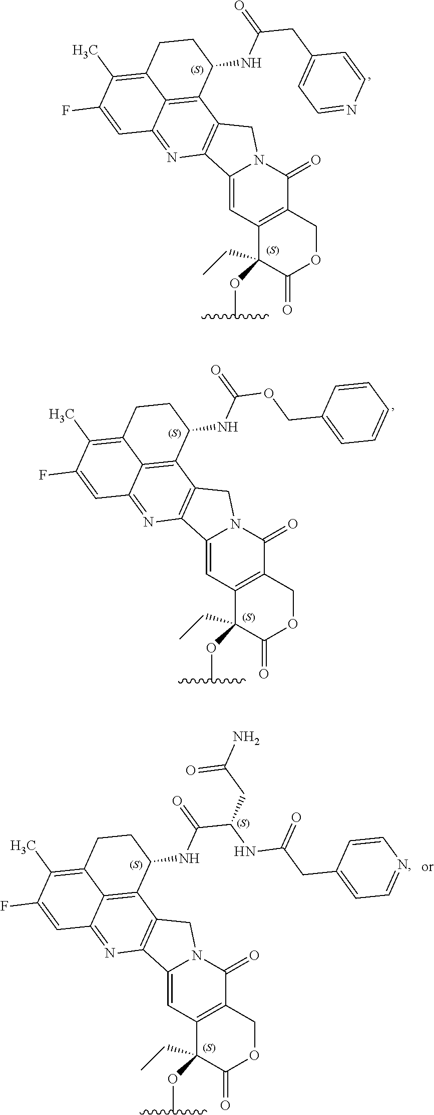

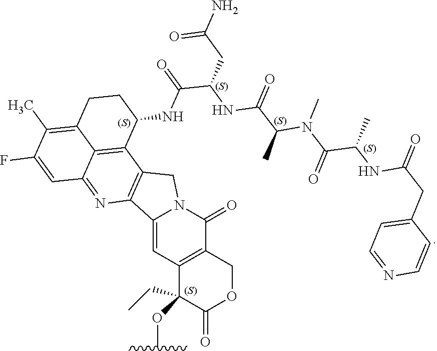

In some embodiments, provided herein is a compound, or a pharmaceutically acceptable salt thereof, or a stereoisomer or mixture of stereoisomers thereof, having a structure according to the formula:

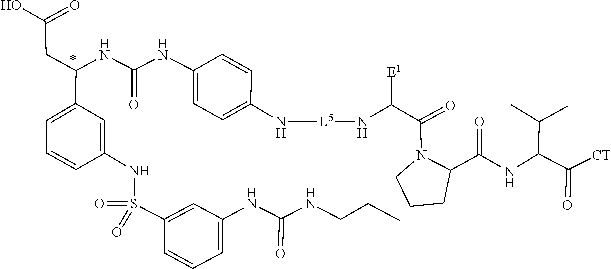

wherein E¹, L⁵, *, and CT are as shown in Table A.

TABLE A

| Com- | | | | |
|---|---|---|---|---|
| pound | E¹ | L⁵ | * | CT |
| 1 | —CH₂C(O)OH | 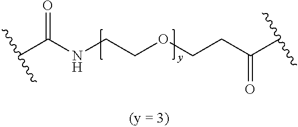 | (R) | 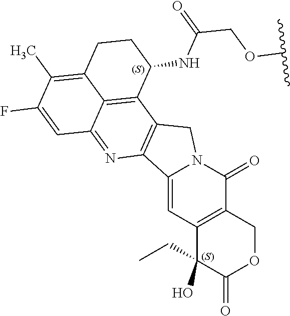 |
| 2 | —CH₂C(O)OH | 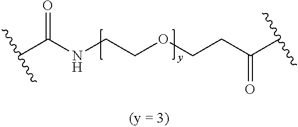 | (R) | 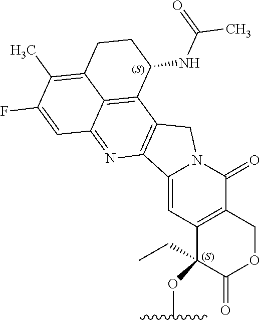 |
| 3 | —CH₂C(O)OH | 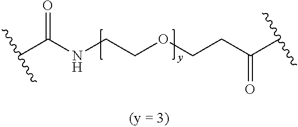 | (R) | 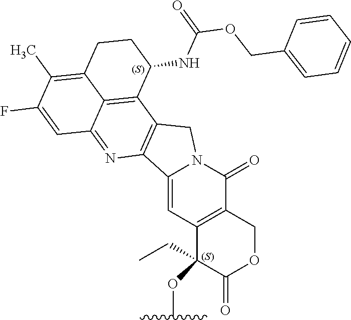 |
| 4 | —CH₂C(O)OH | 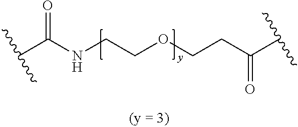 | (R) | 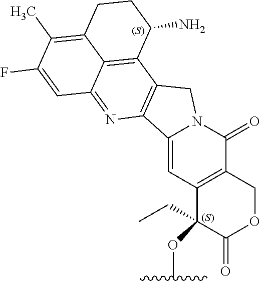 |
| 5 | —CH₂C(O)OH | 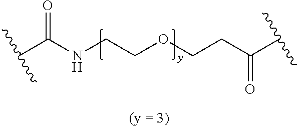 | (R) | 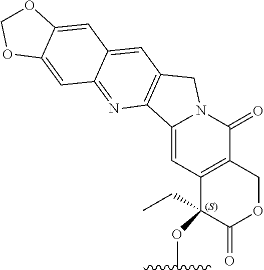 |
| 6 | —CH₂C(O)NH₂ | 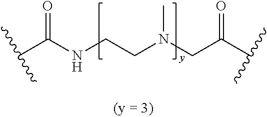 | (R) | 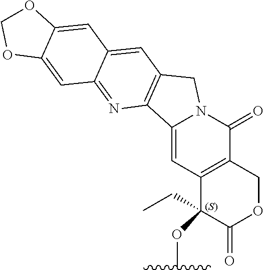 |
| 12 | —CH₂C(O)OH | 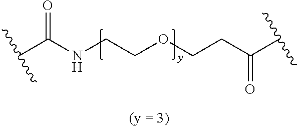 | (S) | 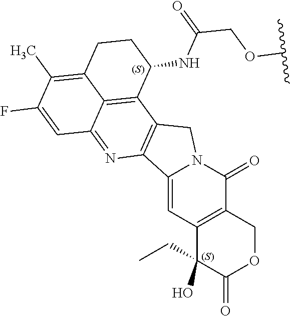 |
| 13 | —CH₂C(O)OH | 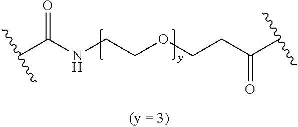 | (S) | 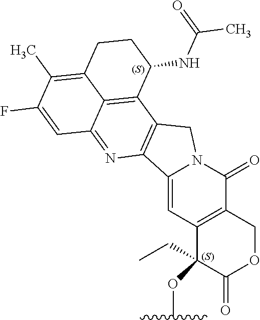 |
| 14 | —CH₂C(O)NH₂ | 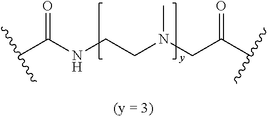 | (S) | 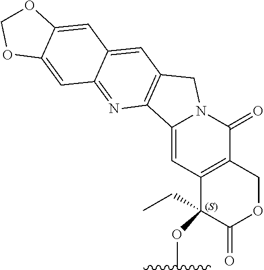 |
| 15 | —CH₂C(O)OH | 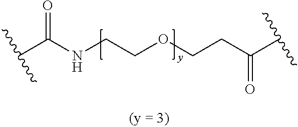 | (S) | 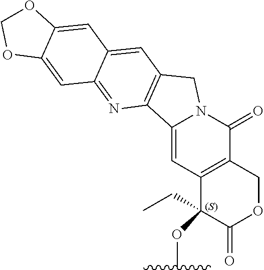 |
| 19 | —CH₂C(O)OH | 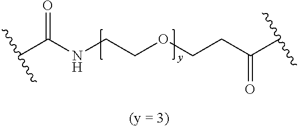 | (R) | 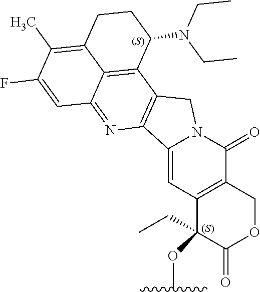 |
| 20 | —CH₂C(O)OH | 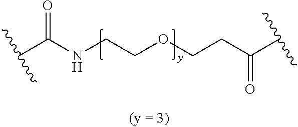 | (R) | 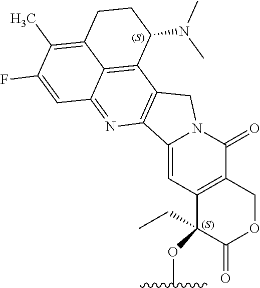 |
| 21 | —CH₂C(O)NH₂ | 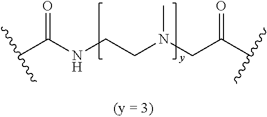 | (R) | 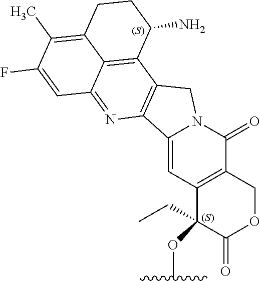 |

In some embodiments, provided herein is a compound, or a pharmaceutically acceptable salt thereof, or a stereoisomer or mixture of stereoisomers thereof, having a structure according to the formula:

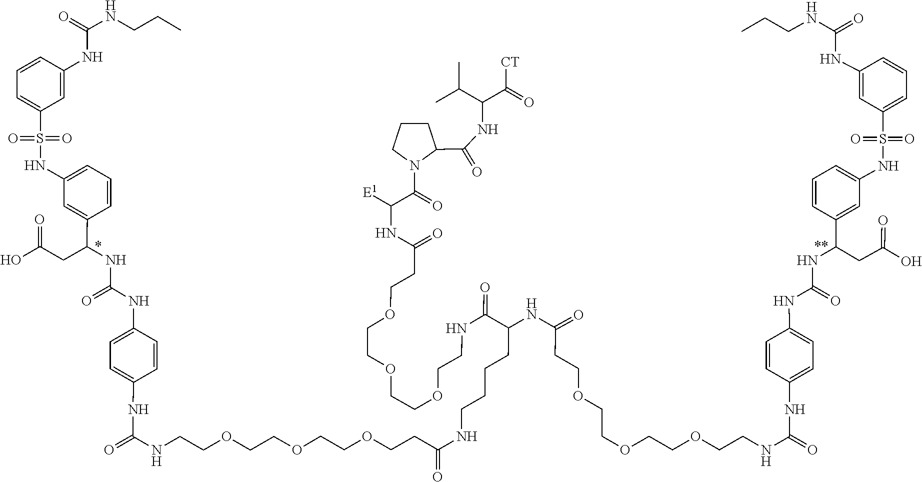

wherein E¹, *, **, and CT are as shown in Table B.

TABLE B

| Com- | | | | |
|---|---|---|---|---|
| pound | E¹ | * | ** | CT |
| 22 | —CH₂C(O)OH | (R) | (R) | 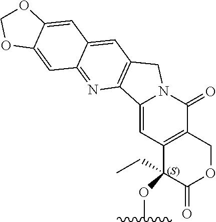 |
| 23 | —CH₂C(O)OH | (R) | (R) | 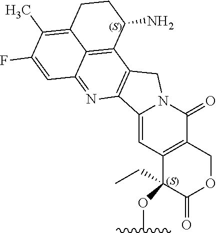 |
| 24 | —CH₂C(O)OH | (R) | (R) | 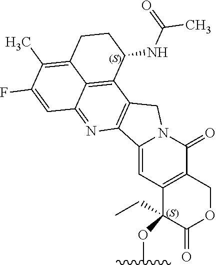 |

Spacers/Linkers (L¹, L², L³, L⁴, L⁵, L⁶, and L⁷)

Provided herein are compounds having linkers or spacers (used interchangeably) which serve to create physical space between one or more elements of the compound or conjugate. In some embodiments, the linkers or spacers provide additional utility (e.g., functional linkers). For example, in some embodiments, a linker is provided with a particular length that enables enhanced cleavage of an adjacent enzymatically cleavable moiety (e.g., EL). In some embodiments, a linker is provided with a particular length that reduces steric hinderance and/or enables greater target binding.

In some embodiments, provided herein is a compound comprising a linker (e.g., L¹, L², L³, L⁴, L⁵, L⁶, and/or L⁷), wherein the linker enhances retention of the compound within a tumor microenvironment. In some embodiments, a linker disclosed herein (e.g., a polyamine or polyamide linker) increases the tumor to plasma ratio of a compound, compared to a reference compound comprising an alkyl or PEG linker. In some embodiments, provided herein is a compound comprising a trivalent linker (A¹) and two linker-binder groups (L²IN, L³IN), or a linker-binder and linker-MOD group (L²IN)(L³MOD), wherein the compound comprising the L³IN or L³MOD group has an increased half-life and/or increased AUC, compared to a compound comprising a bivalent linker. In some embodiments, provided herein is a compound comprising a trivalent linker and two integrin binding groups, wherein the compound comprising the L³IN or L³MOD group has an increase in plasma protein binding, or a decrease in clearance, compared to a compound comprising a bivalent linker.

In some embodiments, a linker is a branched or linear chain of atoms selected from C, N, O, or S (each of which being substituted with hydrogens or bonds so as to fulfill standard valence). In some embodiments, a linker is a carbonyl (—C(O)—). In some embodiments, a linker contains six or less (non-hydrogen) atoms. In some embodiments, a linker contains about 10 to about 20 (non-hydrogen) atoms. In some embodiments, a linker contains about 10 to about 20 (non-hydrogen) atoms arranged in a linear chain. In some embodiments, a linker contains about 20 to about 30 (non-hydrogen) atoms. In some embodiments, a linker contains about 20 to about 30 (non-hydrogen) atoms arranged in a linear chain. In some embodiments, a linker contains about 30 to about 40 (non-hydrogen) atoms. In some embodiments, a linker contains about 30 to about 40 (non-hydrogen) atoms arranged in a linear chain. In some embodiments, multiple linkers are present, each of which having a different length. In some embodiments, a linker contains cyclic or branched moieties. In some embodiments, a linker is substituted with one or more alkyl, oxo, amino, or amide groups.

In some embodiments, each of L¹, L², L³, L⁵, L⁶, and L⁷ contains or is terminally substituted with one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—).

In some embodiments, each of L¹, L², L³, L⁵, L⁶, and L⁷ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —NHC(O)—, —N(CH₃)—, —N(CH₃)C(O)—, —NHC(O)NH—, —N(CH₃)C(O)NH—, —NHS(O)₂NH—, —NHS(O)₂NHC(O)—, —NHS(O)₂NHC(O)O—, —O—, —S—, —S(O)—, —S(O)₂—, or any combination thereof.

In some embodiments, L¹, L², L³, L⁴, L⁵, L⁶, and L⁷ is substituted with one or more side-chain groups (e.g., alkylamines, alkylacids, alkylamides, alkylalcohols, etc.). In some embodiments, L¹, L², L³, L⁴, L⁵, L⁶, and L⁷ is substituted with one or more side-chains comprising a carboxylic acid and/or an amine.

In some embodiments, a linker is ionized in a biological system (e.g., within an acidic tumor microenvironment). In some embodiments, an ionized or partially charged (e.g., partially positive or partially negative) moiety or moieties within a linker enhance localization of the compound in a preferred locale (e.g., extracellularly, within a tumor microenvironment).

In some embodiments, a linker (e.g., one or more of L¹, L², L³, L⁵, L⁶, and L⁷) is an optionally substituted polyamine or polyamide linker.

In some embodiments, a polyamine or polyamide linker is a functional linker.

In some embodiments, the polyamine or polyamide linker of one or more of L¹, L², L³, L⁵, L⁶, and L⁷ is substituted with one or more carbonyl groups.

In some embodiments, the polyamine or polyamide linker of one or more of L¹, L², L³, L⁵, L⁶, and L⁷ forms an aminium ion (or optionally multiple aminium ions) in an acidic tumor microenvironment.

In some embodiments, the polyamine or polyamide linker of one or more of L¹, L², L³, L⁵, L⁶, and L⁷ is selectively retained in a tumor microenvironment.

In some embodiments, the present invention provides compounds having a structure of formulae disclosed herein, or a pharmaceutically acceptable salt thereof; or a stereoisomer or mixture of stereoisomers thereof; wherein each of L¹, L², L³, and L⁶ is independently a bond, substituted or unsubstituted C₁₋₃₀ alkyl, or substituted or unsubstituted heteroalkyl. In some embodiments, each of L¹, L², L³, and L⁶ is independently —C(O)—, substituted or unsubstituted C₂₋₃₀ alkyl, or substituted or unsubstituted heteroalkyl.

In some embodiments, each of L¹, L², L³, L⁴, L⁵, L⁶ and L⁷ is a non-cleavable linker. In some embodiments, each of L¹, L², L³, L⁴, L⁵, L⁶ and L⁷ is a non-cleavable heteroalkyl linker, optionally comprising one or more (e.g., 1 to 10) polymeric subunits (e.g., PEG or PEI).

In some embodiments, each of L¹, L², L³, and L⁶ is a substituted or unsubstituted C₂₋₂₀ alkyl chain that is optionally interrupted one or more times by groups, each independently selected from —O—, —S—, —NH—, —N(CH₃)—, —C(O)—, —C(O)NH—, —C(O)N(CH₃)—, —C(O)O—, —NHC(O)—, N(CH₃)C(O)—, or —NHC(O)NH—, or any combination thereof.

In some embodiments, each of L¹, L², L³, and L⁶, contains (e.g., is terminally substituted with) one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—). In some embodiments, spacers such as L¹, L², L³, and L⁶ contain one or more polymeric units such as:

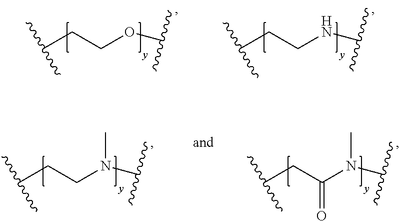

wherein y is an integer from 1 to 20, preferably between 1 and 10, more preferably between 2 and 8. In some embodiments, y is 2, 3, 4, 5, 6, 7, 8, 9, or 10. In some embodiments, y is 2, 3, 4, 5, or 6. In some embodiments, y is 6, 7, 8, 9, or 10. In some embodiments, y is 2, 3, or 4. In some embodiments, y is 6, 7, 8, or 9.

In some embodiments, one or more L¹, L², L³, L⁴, L⁵, L⁶, and/or L⁷ is an optionally substituted polyamine linker. In some embodiments, a polyamine linker of L¹, L², L³, L⁴, L⁵, L⁶, and/or L⁷ forms an aminium ion within an acidic tumor microenvironment. In some embodiments, a polyamine linker of L¹, L², L³, L⁴, L⁵, L⁶, and/or L⁷ is selectively retained in a tumor microenvironment. In some embodiments, a polyamine or polyamide linker of L¹, L², L³, L⁴, L⁵, L⁶, and/or L⁷ enhances retention of a conjugate in a tumor microenvironment. In some embodiments, a polyether linker of L¹, L², L³, L⁴, L⁵, L⁶, and/or L⁷ enhances the aqueous solubility of a conjugate and/or reduces aggregation. In some embodiments, an extended linker provides a preferred distance or reduces steric hinderance between an integrin binder and a cytotoxic or cytostatic radical. In some embodiments, a polyamide linker of L¹, L², L³, L⁴, L⁵, L⁶, and/or L⁷, (e.g., a polysarcosine linker) increases the tumor to plasma ratio of the compound comprising said linker, compared to a reference compound comprising an analogous alkyl or PEG linker. In some embodiments, a polyamide linker of L¹, L², L³, L⁴, L⁵, L⁶, and/or L⁷, (e.g., a polysarcosine linker) improves the pharmacokinetics of a compound comprising said linker, compared to a reference compound comprising an analogous alkyl or PEG linker.

In some embodiments, L¹, L², L³, L⁴, L⁵ and/or L⁶ is a simple spacer selected from the group consisting of:

| | |
|---|---|
| —O— | —S— |
| —S(O)₂— | —C(O)— |
| —C₁₋₃₀ alkyl-, | —C(O)—C₁₋₃₀ alkyl |
| —C₁₋₃₀ alkyl-C(O)—, | —C(O)—C₁₋₃₀ alkyl-C(O)—, |
| —C₁₋₃₀ alkyl-C(O)NH—, | —C(O)—C₁₋₃₀ alkyl-C(O)NH—, |
| —C₁₋₃₀ alkyl-C(O)N(CH₃)—, | —C(O)—C₁₋₃₀ alkyl-C(O)N(CH₃)—, |
| —C₁₋₃₀ alkyl-NH—, | —C(O)—C₁₋₃₀ alkyl-NH—, |
| —C₁₋₃₀ alkyl-NHC(O)—, | —C(O)—C₁₋₃₀ alkyl-NHC(O)—, |
| —C₁₋₃₀ alkyl-N(CH₃)—, | —C(O)—C₁₋₃₀ alkyl-N(CH₃)—, |
| —C₁₋₃₀ alkyl-N(CH₃)C(O)—, | —C(O)—C₁₋₃₀ alkyl-N(CH₃)C(O)—, |
| —C(O)NH—, | —C(O)N(CH₃)— |
| —C(O)NH—C₁₋₃₀ alkyl-, | —C(O)N(CH₃)—C₁₋₃₀ alkyl |
| —C(O)NH—C₁₋₃₀ alkyl-C(O)—, | —C(O)N(CH₃)—C₁₋₃₀ alkyl-C(O)—, |
| —C(O)NH—C₁₋₃₀ alkyl-C(O)NH—, | —C(O)N(CH₃)—C₁₋₃₀ alkyl-C(O)NH—, |
| —C(O)NH—C₁₋₃₀ alkyl-C(O)N(CH₃)—, | —C(O)N(CH₃)—C₁₋₃₀ alkyl-C(O)N(CH₃)—, |
| —C(O)NH—C₁₋₃₀ alkyl-NH—, | —C(O)N(CH₃)—C₁₋₃₀ alkyl-NH—, |
| —C(O)NH—C₁₋₃₀ alkyl-NHC(O)—, | —C(O)N(CH₃)—C₁₋₃₀ alkyl-NHC(O)—, |
| —C(O)NH—C₁₋₃₀ alkyl-N(CH₃)—, | —C(O)N(CH₃)—C₁₋₃₀ alkyl-N(CH₃)—, |
| —C(O)NH—C₁₋₃₀ alkyl-N(CH₃)C(O)—, | —C(O)N(CH₃)—C₁₋₃₀ alkyl-N(CH₃)C(O)—, |
| —NH—, | —NHC(O)—, |
| —NH—C₁₋₃₀ alkyl-, | —NHC(O)—C₁₋₃₀ alkyl-, |
| —NH—C₁₋₃₀ alkyl-C(O)—, | —NHC(O)—C₁₋₃₀ alkyl-C(O)—, |
| —NH—C₁₋₃₀ alkyl-C(O)NH—, | —NHC(O)—C₁₋₃₀ alkyl-C(O)NH—, |
| —NH—C₁₋₃₀ alkyl-C(O)N(CH₃)—, | —NHC(O)—C₁₋₃₀ alkyl-C(O)N(CH₃)—, |
| —NH—C₁₋₃₀ alkyl-NH—, | —NHC(O)—C₁₋₃₀ alkyl-NH—, |
| —NH—C₁₋₃₀ alkyl-NHC(O)—, | —NHC(O)—C₁₋₃₀ alkyl-NHC(O)—, |
| —NH—C₁₋₃₀ alkyl-N(CH₃)—, | —NHC(O)—C₁₋₃₀ alkyl-N(CH₃)—, |
| —NH—C₁₋₃₀ alkyl-N(CH₃)C(O)—, | —NHC(O)—C₁₋₃₀ alkyl-N(CH₃C(O)—, |
| —N(CH₃)—, | —N(CH₃)C(O)—, |
| —N(CH₃)—C₁₋₃₀ alkyl-, | —N(CH₃)C(O)—C₁₋₃₀ alkyl-, |
| —N(CH₃)—C₁₋₃₀ alkyl-C(O)—, | —N(CH₃)C(O)—C₁₋₃₀ alkyl-C(O)—, |
| —N(CH₃)—C₁₋₃₀ alkyl-C(O)NH—, | —N(CH₃)C(O)—C₁₋₃₀ alkyl-C(O)NH—, |
| —N(CH₃)—C₁₋₃₀ alkyl-C(O)N(CH₃)—, | —N(CH₃)C(O)—C₁₋₃₀ alkyl-C(O)N(CH₃)—, |
| —N(CH₃)—C₁₋₃₀ alkyl-NH—, | —N(CH₃)C(O)—C₁₋₃₀ alkyl-NH—, |
| —N(CH₃)—C₁₋₃₀ alkyl-NHC(O)—, | —N(CH₃)C(O)—C₁₋₃₀ alkyl-NHC(O)—, |
| —N(CH₃)—C₁₋₃₀ alkyl-N(CH₃)—, | —N(CH₃)C(O)—C₁₋₃₀ alkyl-N(CH₃)—, |
| —N(CH₃)—C₁₋₃₀ alkyl-N(CH₃)C(O)—, and | —N(CH₃)C(O)—C₁₋₃₀ alkyl-N(CH₃C(O)—. |

In some embodiments, L¹, L², L³, L⁴, L⁵, L⁶ and/or L⁷ is a combination of two or three simple spacers. In some embodiments, L¹, L², L³, L⁴, L⁵, L⁶ and/or L⁷ is a combination of two or three simple spacers, interrupted by (e.g., conjoined by) an amino acid, a dipeptide, or a tripeptide. In some embodiments, L¹, L², L³, L⁴, L⁵, L⁶ and/or L⁷ is a combination of two or three simple spacers, interrupted by (e.g., conjoined by) a cycloalkyl, heterocycloalkyl, aryl, or heteroaryl. In some embodiments, the interrupting group is a substituted triazole. In some embodiments, the interrupting group is an amino acid. In some embodiments, the interrupting group is: —S—, —S(O)—, —S(O)₂⁻¹,

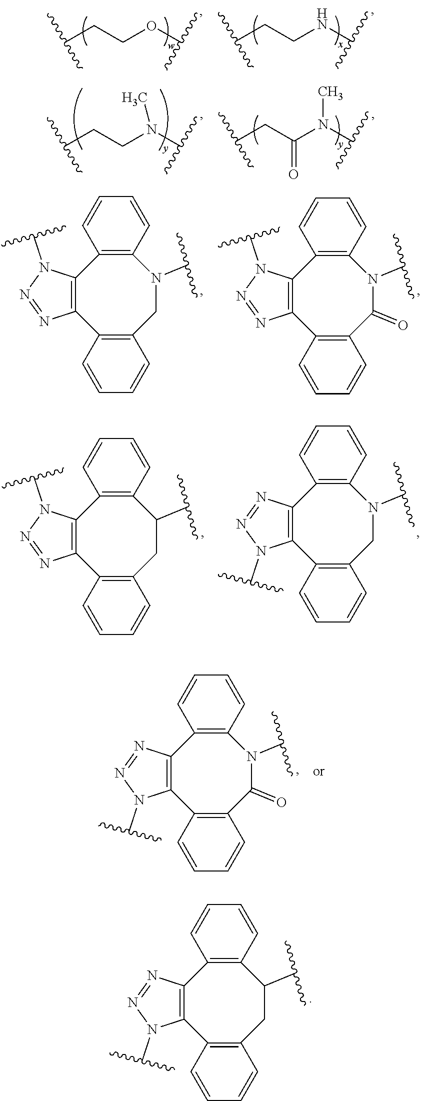

In some embodiments, L¹, L², L³, L⁴, L⁵, L⁶, and/or L⁷ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —O—, —S—, —S(O)—, —S(O)₂—, carbocyclyl, heterocyclyl, aralkyl, heteroaralkyl, or any combination thereof. In some embodiments, L¹, L², L³, L⁴, L⁵, L⁶, and/or L⁷ is a linker disclosed in WO2016207089, which is incorporated by reference in its entirety. In some embodiments, a linker comprises two simple spacers which are conjoined via an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

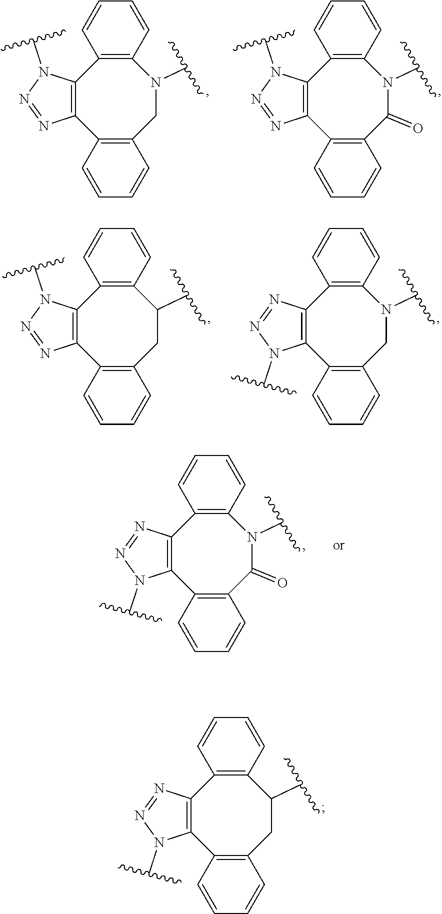

a derivative thereof, wherein the interrupting group is substituted on each end to form a linker (e.g., L¹, L², L³, L⁴, L⁵, L⁶, and/or L⁷).

In some embodiments, each of L¹, L², L³, L⁴, L⁵, and/or L⁶ is a polymeric spacer having a structure represented by formula (i), (ii), or (iii) below:
(i) —(CO)ₘ(CH₂)ₙ(OC₂₋₆ alkyl)ₒ(NH)ₚ(CO)_{q}—;
(ii) —(CO)ᵣ(CH₂)ₛ(NR¹⁰C₂₋₆ alkyl)ₜ(NR¹¹)ᵤ(CO)ᵥ—; or
(iii) —(CO)ᵣ(CH₂)ₛ(NR¹⁰C(O)C₁₋₆ alkyl)ₜ(NR¹¹)ᵤ(CO)ᵥ—;
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1;
v is 0 or 1.

In some embodiments, L¹, L², L³, L⁵ and/or L⁶ is a polymeric spacer selected from:
—C(O)—C₂₋₆ alkyl-[O—C₂₋₆ alkyl]₁₋₈-NHC(O)—,
—C(O)—C₂₋₆ alkyl-[O—C₂₋₆ alkyl]₁₋₈-N(CH₃)C(O)—,
—C(O)—C₁₋₆ alkyl-[NH—C₂₋₆ alkyl]₁₋₈-NHC(O)—,
—C(O)—C₁₋₆ alkyl-[NH—C₂₋₆ alkyl]₁₋₈-N(CH₃)C(O)—,
—C(O)—C₁₋₆ alkyl-[NHC(O)—C₁₋₆ alkyl]₁₋₈-NHC(O)—,
—C(O)—C₁₋₆ alkyl-[NHC(O)—C₁₋₆ alkyl]₁₋₈-N(CH₃)C(O)—,
—C(O)—C₁₋₆ alkyl-[N(CH₃)—C₂₋₆ alkyl]₁₋₈-NHC(O)—,
—C(O)—C₁₋₆ alkyl-[N(CH₃)—C₂₋₆ alkyl]₁₋₈-N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈-N(CH₃)C(O)—,
—C(O)—C₂₋₆ alkyl-[O—C₂₋₆ alkyl]₁₋₈-NH—,
—C(O)—C₂₋₆ alkyl-[O—C₂₋₆ alkyl]₁₋₈-N(CH₃)—,
—C(O)—C₁₋₆ alkyl-[NH—C₂₋₆ alkyl]₁₋₈-NH—,
—C(O)—C₁₋₆ alkyl-[NH—C₂₋₆ alkyl]₁₋₈-N(CH₃)—,
—C(O)—C₁₋₆ alkyl-[NHC(O)—C₁₋₆ alkyl]₁₋₈-NH—,
—C(O)—C₁₋₆ alkyl-[NHC(O)—C₁₋₆ alkyl]₁₋₈-N(CH₃)—,
—C(O)—C₁₋₆ alkyl-[N(CH₃)—C₂₋₆ alkyl]₁₋₈-NH—,
—C(O)—C₁₋₆ alkyl-[N(CH₃)—C₂₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₂₋₆ alkyl-[O—C₂₋₆ alkyl]₁₋₈-NHC(O)—,
—C₂₋₆ alkyl-[O—C₂₋₆ alkyl]₁₋₈-N(CH₃)C(O)—,
—C₁₋₆ alkyl-[NH—C₂₋₆ alkyl]₁₋₈-NHC(O)—,
—C₁₋₆ alkyl-[NH—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₂₋₆ alkyl-[O—C₂₋₆ alkyl]₁₋₈-NH—,
—C₂₋₆ alkyl-[O—C₂₋₆ alkyl]₁₋₈-N(CH₃)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, and
—C₁₋₆ alkyl-[N(CH₃)—C₂₋₆ alkyl]₁₋₈-N(CH₃)—.

In some embodiments, L⁴ is a polymeric spacer (i.e., a polyamine or polyamide linker) selected from:
—C(O)—C₁₋₆ alkyl-[NH—C₂₋₆ alkyl]₁₋₈-NHC(O)—,
—C(O)—C₁₋₆ alkyl-[NH—C₂₋₆ alkyl]₁₋₈-N(CH₃)C(O)—,
—C(O)—C₁₋₆ alkyl-[NHC(O)—C₁₋₆ alkyl]₁₋₈-NHC(O)—,
—C(O)—C₁₋₆ alkyl-[NHC(O)—C₁₋₆ alkyl]₁₋₈-N(CH₃)C(O)—,
—C(O)—C₁₋₆ alkyl-[N(CH₃)—C₂₋₆ alkyl]₁₋₈-NHC(O)—,
—C(O)—C₁₋₆ alkyl-[N(CH₃)—C₂₋₆ alkyl]₁₋₈-N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈-N(CH₃)C(O)—,
—C(O)—C₁₋₆ alkyl-[NH—C₂₋₆ alkyl]₁₋₈-NH—,
—C(O)—C₁₋₆ alkyl-[NH—C₂₋₆ alkyl]₁₋₈-N(CH₃)—,
—C(O)—C₁₋₆ alkyl-[NHC(O)—C₁₋₆ alkyl]₁₋₈-NH—,
—C(O)—C₁₋₆ alkyl-[NHC(O)—C₁₋₆ alkyl]₁₋₈-N(CH₃)—,
—C(O)—C₁₋₆ alkyl-[N(CH₃)—C₂₋₆ alkyl]₁₋₈-NH—,
—C(O)—C₁₋₆ alkyl-[N(CH₃)—C₂₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₁₋₆ alkyl-[NH—C₂₋₆ alkyl]₁₋₈-NHC(O)—,
—C₁₋₆ alkyl-[NH—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, and
—C₁₋₆ alkyl-[N(CH₃)—C₂₋₆ alkyl]₁₋₈-N(CH₃)—.

In some embodiments, L⁵ is a polymeric spacer (i.e., a PEG linker) selected from:
—C(O)—C₂₋₆ alkyl-[O—C₂₋₆ alkyl]₁₋₈-NHC(O)—,
—C(O)—C₂₋₆ alkyl-[O—C₂₋₆ alkyl]₁₋₈-N(CH₃)C(O)—,
—C(O)—C₂₋₆ alkyl-[O—C₂₋₆ alkyl]₁₋₈-NH—,
—C(O)—C₂₋₆ alkyl-[O—C₂₋₆ alkyl]₁₋₈-N(CH₃)—,
—C₂₋₆ alkyl-[O—C₂₋₆ alkyl]₁₋₈-NH—,
—C₂₋₆ alkyl-[O—C₂₋₆ alkyl]₁₋₈-NHC(O)—,
—C₂₋₆ alkyl-[O—C₂₋₆ alkyl]₁₋₈-N(CH₃)—, and
—C₂₋₆ alkyl-[O—C₂₋₆ alkyl]₁₋₈-N(CH₃)C(O)—.

In some embodiments, L⁷ is substituted C₁₂₋₃₀ alkyl, or a substituted or unsubstituted heteroalkyl having 15 to 60 (preferably 18 to 36) non-hydrogen atoms (preferably selected from C, N, O, and S). In some embodiments, L⁷ is a heteroalkyl having 15 to 60 atoms. In some embodiments, L⁷ is a heteroalkyl group that is conjoins IN to the rest of the molecule through 15 to 60 covalent bonds. In some embodiments, L⁷ is a heteroalkyl group. In some embodiments, L⁷ is a substituted or unsubstituted C₁₋₃₀ alkyl, or substituted or unsubstituted heteroalkyl. In some embodiments, L⁷ is a substituted or unsubstituted C₁₋₃₀ alkyl, or substituted or unsubstituted heteroalkyl containing 4-12 Z groups; wherein each Z group is independently —(O—C₁₋₆ alkyl)-, —(NH—C₁₋₆ alkyl)-, or —(N(C₁₋₃ alkyl)-C₁₋₆ alkyl. In some embodiments, L⁷ is a substituted or unsubstituted heteroalkyl containing 6 to 12 Z groups. In some embodiments, L⁷ is a substituted or unsubstituted heteroalkyl containing 6 to 10 Z groups. In some embodiments, each Z group is independently selected from —(OCH₂CH₂)—, —(OCH₂CH₂CH₂)—, —(OCH₂CH₂CH₂CH₂)—, —(OCH(CH₃)CH₂)—, —(OCH₂CH(CH₃))—, —(OC(CH₃)₂CH₂)—, —(OCH₂C(CH₃)₂)—, —(NHCH₂CH₂)—, —(NHCH₂CH₂CH₂)—, —(NHCH₂CH₂CH₂CH₂)—, —(NHCH(CH₃)CH₂)—, —(NHCH₂CH(CH₃))—, —(NHC(CH₃)₂CH₂)—, —(NHCH₂C(CH₃)₂)—, —(NHC(O)CH₂)—, —(NHC(O)CH₂CH₂)—, —(NHC(O)CHCH₃)—, —(NHC(O)C(CH₃)₂)—, —(NHC(O)C(CH₃)₂CH₂)—, —(N(CH₃)CH₂CH₂)—, —(N(CH₃)CH₂CH₂CH₂)—, —(N(CH₃)CH₂CH₂CH₂CH₂)—, —(N(CH₃)CH(CH₃)CH₂)—, —(N(CH₃)CH₂CH(CH₃))—, —(N(CH₃)C(CH₃)₂CH₂)—, —(N(CH₃)C(O)CH₂)—, —(N(CH₃)C(O)CH₂CH₂)—, —(N(CH₃)C(O)CHCH₃)—, —(N(CH₃)C(O)C(CH₃)₂)—, —(N(CH₃)C(O)C(CH₃)₂CH₂)—, and —(N(CH₃)CH₂C(CH₃)₂)—. In some embodiments, each Z group is independently selected from —(OCH₂CH₂)—, —(NHCH₂CH₂)—, and —(N(CH₃)CH₂CH₂)—, and —(N(CH₃)C(O)CH₂)—. In some embodiments, Z groups are separated by an alkyl group, an amide, an alkylamide, an alkylamine, a polyaminoalkyl group, or an amino acid (including modified or synthetic amino acids).

In some embodiments, a spacer (e.g., L⁷) is an elongated linker having a structure represented by formula (i) or (ii) below:
(i) —(CO)ₐ(C₀₋₆ alkyl)(Z¹C₂₋₆ alkyl)_{b}-Z²—(C₀₋₆ alkyl)(Z³C₂₋₆ alkyl)_{c}(Z⁴)_{d}(CO)ₑ—; or
(ii) —(CO)ₐ(C₀₋₆ alkyl)(Z¹C(O)C₁₋₆ alkyl)_{b}-Z²—;
wherein:
Z¹ is in each occurrence independently —O—, —NH—, or —N(C₁₋₃ alkyl)-;
Z² is a bond, —C(O)—, —C(O)NH—, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O—, —NH—, or —N(C₁₋₃ alkyl)-;
Z⁴ is —O—, —NH—, or —N(C₁₋₃ alkyl)-;
a is 0 or 1;
b is 1 to 10;
c is 1 to 10;
d is 0 or 1; and
e is 0 or 1.

In some embodiments, a spacer (e.g., L⁷) is an elongated linker having a structure represented by formula (iii) below:
(iii) —(CO)ₐ(C₀₋₆ alkyl)(Z¹C₂₋₆ alkyl)_{b}-Z²—(C₀₋₆ alkyl)(Z³C₂₋₆ alkyl)_{c}(Z⁴)_{d}(CO)ₑ—
wherein:
Z¹ is in each occurrence independently —O—, —NH—, or —N(C₁₋₃ alkyl)-;
Z² is a bond, —C(O)—, —C(O)NH—, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O— or —N(CH₃)—;
Z⁴ is —NH—, or —N(CH₃)—;
a is 0 or 1;
b is 1 to 6;
c is 1 to 6;
d is 0 or 1; and
e is 0 or 1.

In some embodiments, a spacer SP (e.g., L⁷) is an elongated spacer selected from the group consisting of:
—C(O)—C₂₋₄ alkyl-[O—C₂₋₄ alkyl]₂₋₄—NHC(O)—C₂₋₄ alkyl-[O—C₂₋₄ alkyl]₂₋₄—NHC(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄—NHC(O)—,
—C(O)—C₁₋₄ alkyl-[NH—C₂₋₄ alkyl]₂₋₄—NHC(O)—C₁₋₄ alkyl-[NH—C₂₋₄ alkyl]₂₋₄—NHC(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄—NHC(O)—,
—C(O)—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄—NHC(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—NHC(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄—NHC(O)—,
—C(O)—C₂₋₄ alkyl-[O—C₂₋₄ alkyl]₂₋₄—NHC(O)—C₁₋₄ alkyl-[N(CH₃)—C₂₋₄ alkyl]₂₋₄—NHC(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄—NHC(O)—,
—C(O)—C₁₋₄ alkyl-[N(CH₃)—C₂₋₄ alkyl]₂₋₄—NHC(O)—C₂₋₄ alkyl-[O—C₂₋₄ alkyl]₂₋₄—NHC(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄—NHC(O)—,
—C(O)—C₂₋₄ alkyl-[O—C₂₋₄ alkyl]₂₋₄—NHC(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₁₋₄ alkyl-[NH—C₂₋₄ alkyl]₂₋₄—NHC(O)—C₁₋₄ alkyl-[NH—C₂₋₄ alkyl]₂₋₄-N(CH₃)C(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C(O)—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—N(CH₃)C(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄—N(CH₃)C(O)—,
—C(O)—C₂₋₄ alkyl-[O—C₂₋₄ alkyl]₂₋₄—NHC(O)—C₁₋₄ alkyl-[N(CH₃)—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄—NHC(O)—C₂₋₄ alkyl-[O—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄—NHC(O)—C₂₋₄ alkyl-[O—C₂₋₄ alkyl]₂₋₄—NHC(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄—NHC(O)—,
—C₁₋₄ alkyl-[NH—C₂₋₄ alkyl]₂₋₄—NHC(O)—C₁₋₄ alkyl-[NH—C₂₋₄ alkyl]₂₋₄—NHC(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄—NHC(O)—,
—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄—NHC(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—NHC(O)—,
—C₂₋₄ alkyl-[O—C₂₋₄ alkyl]₂₋₄—NHC(O)—C₁₋₄ alkyl-[N(CH₃)—C₂₋₄ alkyl]₂₋₄—NHC(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄—NHC(O)—,
—C₁₋₄ alkyl-[N(CH₃)—C₂₋₄ alkyl]₂₋₄—NHC(O)—C₂₋₄ alkyl-[O—C₂₋₄ alkyl]₂₋₄—NHC(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄—NHC(O)—,
—C₂₋₄ alkyl-[O—C₂₋₄ alkyl]₂₋₄—NHC(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C₁₋₄ alkyl-[NH—C₂₋₄ alkyl]₂₋₄—NHC(O)—C₁₋₄ alkyl-[NH—C₂₋₄ alkyl]₂₋₄-N(CH₃)C(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄—NHC(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄—NHC(O)—C₂₋₄ alkyl-[O—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄—NHC(O)—C₂₋₄ alkyl-[O—C₂₋₄ alkyl]₂₋₄—NH—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄—NH—,
—C(O)—C₁₋₄ alkyl-[NH—C₂₋₄ alkyl]₂₋₄—NHC(O)—C₁₋₄ alkyl-[NH—C₂₋₄ alkyl]₂₋₄—NH—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄—NH—,
—C(O)—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄—NH—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—NH—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄—NH—,
—C(O)—C₂₋₄ alkyl-[O—C₂₋₄ alkyl]₂₋₄—NHC(O)—C₁₋₄ alkyl-[N(CH₃)—C₂₋₄ alkyl]₂₋₄—NH—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄—NH—,
—C(O)—C₁₋₄ alkyl-[N(CH₃)—C₂₋₄ alkyl]₂₋₄—NHC(O)—C₂₋₄ alkyl-[O—C₂₋₄ alkyl]₂₋₄—NH—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄—NH—,
—C(O)—C₂₋₄ alkyl-[O—C₂₋₄ alkyl]₂₋₄—NHC(O)—C₂₋₄ alkyl-[O—C₂₋₄ alkyl]₂₋₄-N(CH₃)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C(O)—C₁₋₄ alkyl-[NH—C₂₋₄ alkyl]₂₋₄—NHC(O)—C₁₋₄ alkyl-[NH—C₂₋₄ alkyl]₂₋₄-N(CH₃)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C(O)—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄—N(CH₃)—,
—C(O)—C₂₋₄ alkyl-[O—C₂₋₄ alkyl]₂₋₄—NHC(O)—C₁₋₄ alkyl-[N(CH₃)—C₂₋₄ alkyl]₂₋₄-N(CH₃)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄—NHC(O)—C₂₋₄ alkyl-[O—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—,
—C₂₋₄ alkyl-[O—C₂₋₄ alkyl]₂₋₄—NHC(O)—C₂₋₄ alkyl-[O—C₂₋₄ alkyl]₂₋₄—NH—,
—C₂₋₄ alkyl-[O—C₂₋₄ alkyl]₂₋₄-N(CH₃)C(O)—C₂₋₄ alkyl-[O—C₂₋₄ alkyl]₂₋₄—NH—,
—C₁₋₄ alkyl-[NH—C₂₋₄ alkyl]₂₋₄—NHC(O)—C₁₋₄ alkyl-[NH—C₂₋₄ alkyl]₂₋₄—NH—,
—C₁₋₄ alkyl-[NH—C₂₋₄ alkyl]₂₋₄-N(CH₃)C(O)—C₁₋₄ alkyl-[NH—C₂₋₄ alkyl]₂₋₄—NH—,
—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄—NH—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄—NH—,
—C₂₋₄ alkyl-[O—C₂₋₄ alkyl]₂₋₄—NHC(O)—C₁₋₄ alkyl-[N(CH₃)—C₂₋₄ alkyl]₂₋₄—NH—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄—NH—,
—C₁₋₄ alkyl-[N(CH₃)—C₂₋₄ alkyl]₂₋₄—NHC(O)—C₂₋₄ alkyl-[O—C₂₋₄ alkyl]₂₋₄—NH—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄—NH—,
—C₂₋₄ alkyl-[O—C₂₋₄ alkyl]₂₋₄—NHC(O)—C₂₋₄ alkyl-[O—C₂₋₄ alkyl]₂₋₄-N(CH₃)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C₁₋₄ alkyl-[NH—C₂₋₄ alkyl]₂₋₄—NHC(O)—C₁₋₄ alkyl-[NH—C₂₋₄ alkyl]₂₋₄-N(CH₃)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C₁₋₄ alkyl-[N(CH₃)—C₂₋₄ alkyl]₂₋₄—NHC(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄—NHC(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄—NHC(O)—C₂₋₄ alkyl-[O—C₂₋₄ alkyl]₂₋₄-N(CH₃)—, and
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—.

In some embodiments, a spacer (e.g., L¹, L², L³, L⁴, L⁵, L⁶, or L⁷) further comprises a spanner, wherein the spanner is a linker that conjoins the spacer to an adjacent group (e.g., EL, A¹, IN, MOD). A spanner can be a substituted or unsubstituted alkyl, or a substituted or unsubstituted heteroalkyl linker, typically comprising about 1 to about 20 atoms selected from carbon, nitrogen, and oxygen. Preferably, a spanner is a short alkyl or heteroalkl group having functional groups at its ends that are configured to form a stable bond (e.g., a peptide bond) with an adjacent group. Examples of spanners include groups such as:

| | |
|---|---|
| #C(O)—C₁₋₆ alkyl-C(O)—, | #C(O)NH—C₁₋₆ alkyl-C(O)—, |
| #C(O)N(CH₃)—C₁₋₆ alkyl-C(O)—, | #NH—C₁₋₆ alkyl-C(O)—, |
| #NHC(O)—C₁₋₆ alkyl-C(O)—, | #N(CH₃)—C₁₋₆ alkyl-C(O)—, |
| #N(CH₃)C(O)—C₂₋₆ alkyl-C(O)—, | #C(O)—C₁₋₆ alkyl-C(O)NH—, |
| #C(O)NH—C₁₋₆ alkyl-C(O)NH—, | #C(O)N(CH₃)—C₁₋₆ alkyl-C(O)NH—, |
| #NH—C₁₋₆ alkyl-C(O)NH—, | #NHC(O)—C₁₋₆ alkyl-C(O)NH—, |
| #N(CH₃)—C₁₋₆ alkyl-C(O)NH—, | #N(CH₃)C(O)—C₁₋₆ alkyl-C(O)NH—, |
| #C(O)—C₁₋₆ alkyl-C(O)N(CH₃)—, | #C(O)NH—C₁₋₆ alkyl-C(O)N(CH₃)—, |
| #C(O)N(CH₃)—C₁₋₆ alkyl-C(O)N(CH₃)—, | #NH—C₁₋₆ alkyl-C(O)N(CH₃)—, |
| #NHC(O)—C₁₋₆ alkyl-C(O)N(CH₃)—, | #N(CH₃)—C₁₋₆ alkyl-C(O)N(CH₃)—, |
| #N(CH₃)C(O)—C₁₋₆ alkyl-C(O)N(CH₃)—, | #C(O)—C₁₋₆ alkyl-NH—, |
| #C(O)NH—C₁₋₆ alkyl-NH—, | #C(O)N(CH₃)—C₁₋₆ alkyl-NH—, |
| #NH—C₁₋₆ alkyl-NH—, | #NHC(O)—C₁₋₆ alkyl-NH—, |
| #N(CH₃)—C₁₋₆ alkyl-NH—, | #N(CH₃)C(O)—C₁₋₆ alkyl-NH—, |
| #C(O)—C₁₋₆ alkyl-NHC(O)—, | #C(O)NH—C₁₋₆ alkyl-NHC(O)—, |
| #C(O)N(CH₃)—C₁₋₆ alkyl-NHC(O)—, | #NH—C₁₋₆ alkyl-NHC(O)—, |
| #NHC(O)—C₁₋₆ alkyl-NHC(O)—, | #N(CH₃)—C₁₋₆ alkyl-NHC(O)—, |
| #N(CH₃)C(O)—C₁₋₆ alkyl-NHC(O)—, | #C(O)—C₁₋₆ alkyl-N(CH₃)—, |
| #C(O)NH—C₁₋₆ alkyl-N(CH₃)—, | #C(O)N(CH₃)—C₁₋₆ alkyl-N(CH₃)—, |
| #NH—C₁₋₆ alkyl-N(CH₃)—, | #NHC(O)—C₁₋₆ alkyl-N(CH₃)—, |
| #N(CH₃)—C₁₋₆ alkyl-N(CH₃)—, | #N(CH₃)C(O)—C₁₋₆ alkyl-N(CH₃)—, |
| #C(O)—C₁₋₆ alkyl-N(CH₃)C(O)—, | #C(O)NH—C₁₋₆ alkyl-N(CH₃)C(O)—, |
| #C(O)N(CH₃)—C₁₋₆ alkyl-N(CH₃)C(O)—, | #NH—C₁₋₆ alkyl-N(CH₃)C(O)—, |
| #NHC(O)—C₁₋₆ alkyl-N(CH₃)C(O)—, | #N(CH₃)—C₁₋₆ alkyl-N(CH₃)C(O)—, |
| #N(CH₃)C(O)—C₁₋₆ alkyl-N(CH₃)C(O)—, | #C(O)—C₁₋₆ alkyl-, |
| #C(O)NH—C₁₋₆ alkyl-, | #C(O)N(CH₃)—C₁₋₆ alkyl-, |
| #NH—C₁₋₆ alkyl-, | #NHC(O)—C₁₋₆ alkyl-, |
| #N(CH₃)—C₁₋₆ alkyl-, | #N(CH₃)C(O)—C₁₋₆ alkyl-; |
| #C₁₋₆ alkyl-C(O)—, | #C₁₋₆ alkyl-C(O)NH—, |
| #C₁₋₆ alkyl-C(O)N(CH₃)—, | #C₁₋₆ alkyl-NH—, |
| #C₁₋₆ alkyl-NHC(O)—, | #C₁₋₆ alkyl-N(CH₃)—, or |
| #C₁₋₆ alkyl-N(CH₃)C(O)—, | |

wherein # denotes a bond to the spacer, and -denotes a bond to the adjacent group.

In some embodiments, a spanner is #C(O)—C₂₋₆ alkyl-C(O)— or #NH—C₂₋₆ alkyl-NH—. In some embodiments, a spanner is #C(O)(CH₂CH₂CH₂)C(O)— or #NH(CH₂CH₂)NH—. As used herein spanners are denoted S¹, S², S³, etc. corresponding to the linker to which they are attached. Alternatively, a spanner may be described as a member of A¹, in which case the S¹, S², S³, numbering still pertains to the linker to which A¹ is bonded. In some embodiments, a spanner is a bond. In some embodiments, a spanner is absent.

In some embodiments, L¹ is selected from:

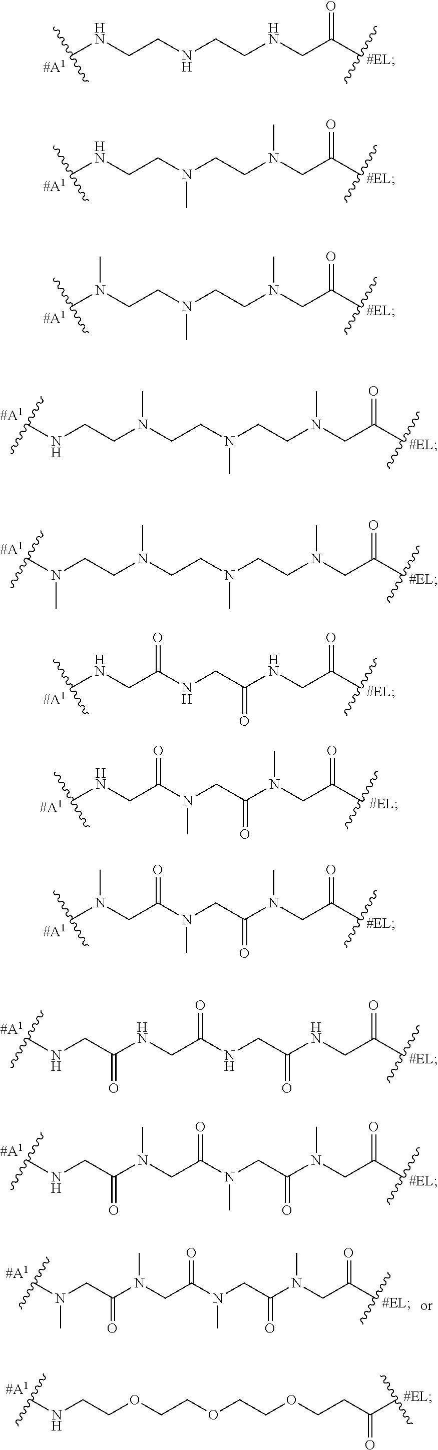

wherein #EL denotes a bond to EL (or SIL); and #A¹ denotes a bond to A¹.

In some embodiments, L² and L³ are each independently selected from.

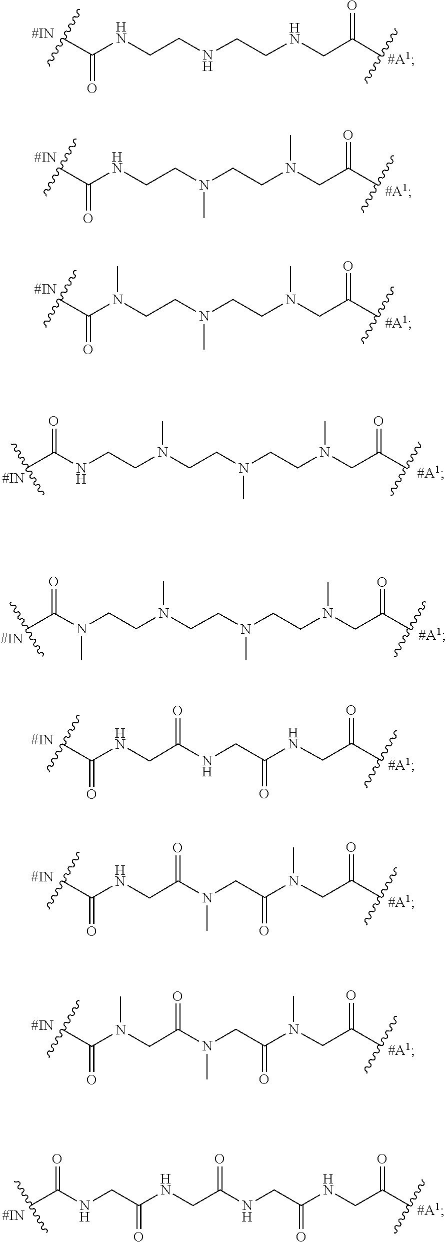

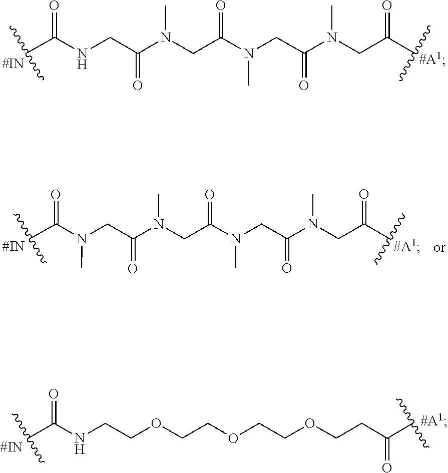 [SMILES: *C(=O)CN(C)C(=O)CN(C)C(=O)CN(C)C(=O)CNC(*)=O ; *C(=O)CN(C)C(=O)CN(C)C(=O)CN(C)C(=O)CN(C)C(*)=O ; *C(=O)CCOCCOCCOCCNC(*)=O]

wherein #IN denotes a bond to IN or MOD; and #A¹ denotes a bond to A¹.

In some embodiments, L⁴ is:

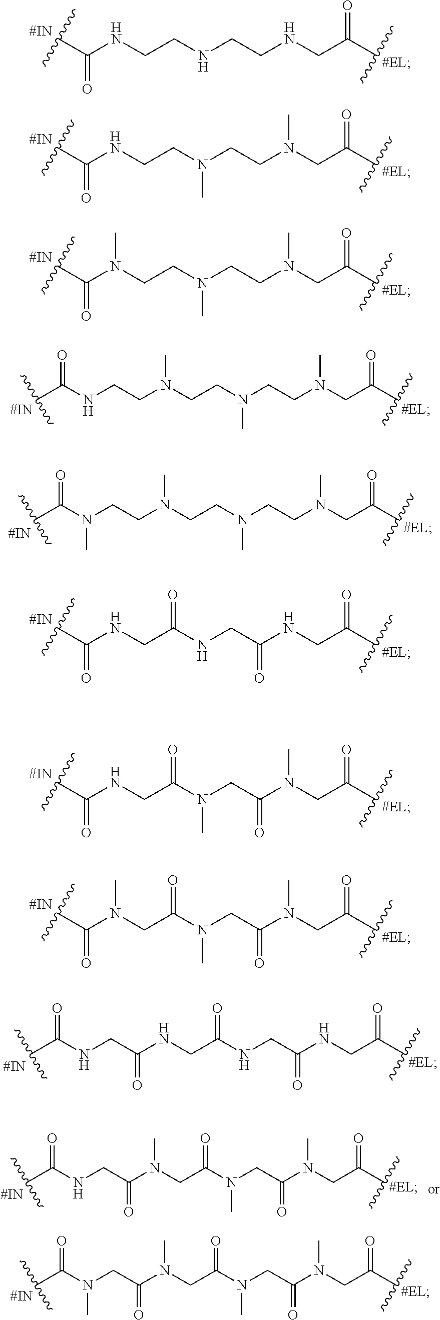

wherein #EL denotes a bond to EL (or SIL); and #IN denotes a bond to IN.

In some embodiments, L⁴ is:

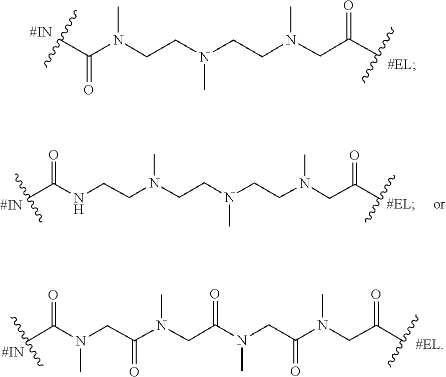 [SMILES: *C(=O)CN(C)CCN(C)CCN(C)C(*)=O ; *C(=O)CN(C)CCN(C)CCN(C)CCNC(*)=O ; *C(=O)CN(C)C(=O)CN(C)C(=O)CN(C)C(=O)CN(C)C(*)=O]

In some embodiments, L⁵ is:

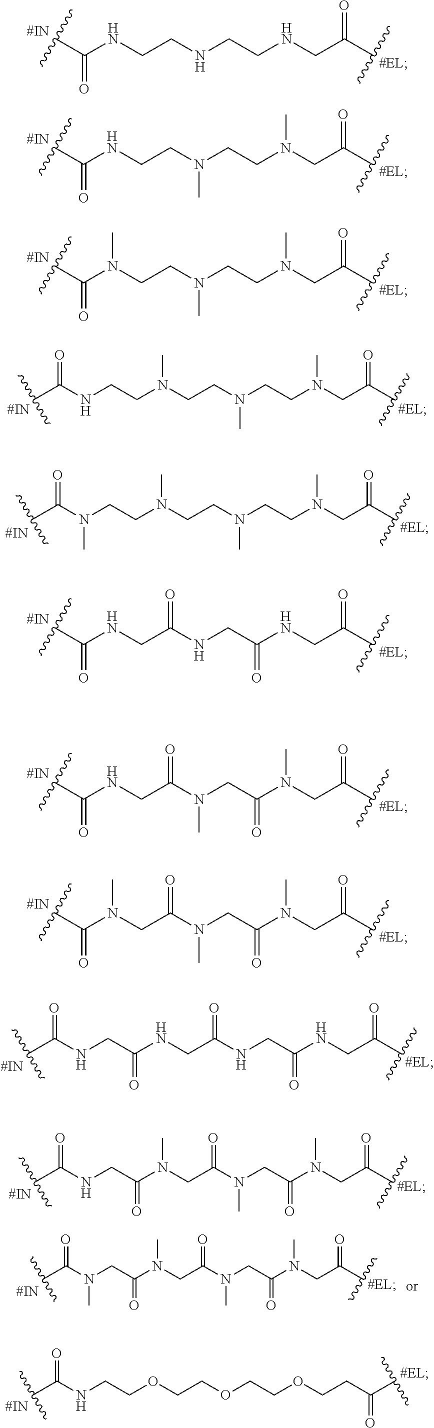

wherein #EL denotes a bond to EL (or SIL); and #IN denotes a bond to IN.

In some embodiments, L⁵ is:

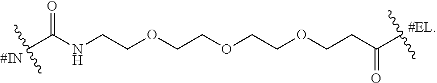 [SMILES: *C(=O)CCOCCOCCOCCNC(*)=O]

In some embodiments, L⁶ is: —C₀₋₁₂ alkyl-C(O)—; —C₀₋₁₂ alkyl-C(O)NH—;
—C₀₋₁₂ alkyl-C(O)N(CH₃)—; —C₀₋₁₂ alkyl-NH—; —C₀₋₁₂ alkyl-NHC(O)—;
—C₀₋₁₂ alkyl-N(CH₃)—; or —C₀₋₁₂ alkyl-N(CH₃)C(O)—;

In some embodiments, L⁶ is: —C(O)—.

In some embodiments, L⁷ is:

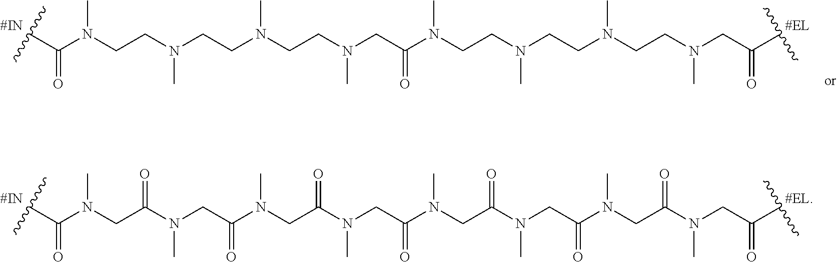 [SMILES: *C(=O)CN(C)CCN(C)CCN(C)CCN(C)C(=O)CN(C)CCN(C)CCN(C)CCN(C)C(*)=O ; *C(=O)CN(C)C(=O)CN(C)C(=O)CN(C)C(=O)CN(C)C(=O)CN(C)C(=O)CN(C)C(=O)CN(C)C(=O)CN(C)C(*)=O]

### Trivalent Radicals (A¹)

In some embodiments, a trivalent radical A¹ is a moiety containing 1 to 100 atoms selected from H, C, N, O, and S, configured to bond to linkers L¹, L², and L³. In some embodiments, A¹ contains a central atom or group (Y) that is N or CH. In some embodiments, the central atom or group (Y) has one or more arms (e.g., alkyl or heteroalkyl chains, optionally substituted with, or interrupted by, one or more groups independently selected from carbonyls (—C(O)—), ethers (—O—), amines (e.g., —NH— or —N(CH₃)—), or amides (e.g., —C(O)NH—, —C(O)N(CH₃)—, —NHC(O)—, or —N(CH₃)C(O)—, alternatively referred to as amide linkers). In some embodiments, A¹ is trivalent heteroalkyl radical. In some embodiments, A¹ is trivalent radical with amide linker arms (e.g., B¹, B², B³). In some embodiments, the trivalent radical is referred to as a branching unit, a branching group. In some embodiments, A of Formula (01) or Formula (05) is A¹. In some embodiments, A¹ is a trivalent heteroaryl, heteroaralkyl, aryl, aralkyl, heteroalkyl-aryl, heteroalkyl-heteroaryl radical. In some embodiments, A¹ contains a central atom or group (Y) that is a carbocycle (e.g., cycloalkyl or aryl) or heterocycle (e.g., heterocycloalkyl or heteroaryl). In some embodiments, (Y) is phenyl, triazinyl, or triazolyl, each of which is optionally substituted by 1-3 heteroalkyl arms.

In some embodiments, A or A¹ is a trivalent linker, optionally comprising arms or spanners, that is configured to conjoin a payload to two integrin binders, or to an integrin binder and a MOD group (e.g., via a spacer L and/or an enzymatic linker EL). In some embodiments, A or A¹ is a trivalent linker. In some embodiments, A or A¹ is a trivalent linker comprising arms B¹, B², and B³, and optionally further comprising spanners S¹, S², and S³, wherein the trivalent linker is configured to form an amide bond with an adjacent group (e.g., L¹, L², L³, IN, MOD, or EL). In some embodiments, A or A¹ is a trivalent amide linker. In this context, a trivalent amide linker can be a chemical group having three valencies (or capable of forming bonds to 3 agents), and generally composed of alkyl, carbonyl, and amine groups, and preferably capable of forming an amide bond with an adjacent group (e.g., L¹, L², L³, IN, MOD, or EL). In some embodiments, the trivalent radical is a trivalent amide linker. In some embodiments, the trivalent amide linker comprises a central amino acid (e.g., lysine, glutamine, glutamate, etc.), wherein the amino acid forms a bond with an adjacent group (e.g., L¹, L², L³, IN, MOD, or EL), optionally bridged via a linking arm (e.g., B¹, B², B³) and/or a spanner (i.e., S¹, S², or S³). Preferably, a trivalent radical as described herein conjoins a payload (e.g., an enzymatically-cleavable payload (CP)) with two integrin binders, or with an integrin binder and a group MOD, and wherein the payload, integrin binder(s) and MOD may be conjoined via a linker/spacer (L¹, L², or L³).

In some embodiments, A¹ has a structure represented by the formula:

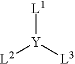

or wherein (Y) is N, CH, phenyl, or heteroaryl, and each of B¹, B², and B³ represents an arm that connects to a corresponding spacer (e.g., L¹, L², and L³ respectively) and wherein * denotes a bond to L¹, ** denotes a bond to L², *** denotes a bond to L³.

In some embodiments, A¹ is a trivalent radical of the formula:

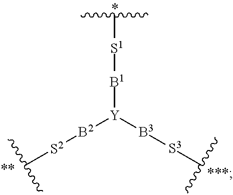

wherein:
* denotes a bond to L¹,
** denotes a bond to L²
*** denotes a bond to L³,
each of S¹, S², S³, is spanner; and
each of B¹, B², B³, is a linking arm.
and Y is CH or N.

In some embodiments, one or more of S¹, S², and S³ is a bond. In some embodiments, one or more of S¹, S², and S³ is —C(O)—C₂₋₆ alkyl-C(O)—, —N(CH₃)—C₁₋₆ alkyl-N(CH₃)—, —NH—C₁₋₆ alkyl-NH—, or —NH—C₁₋₆ alkyl-N(CH₃)—. In some embodiments, S¹, S², and/or S³ is —C(O)—C₂₋₆ alkyl-C(O)— or —NH—C₁₋₆ alkyl-NH—.

In some embodiments, each B¹, B², and B³, is an amide linker (e.g., an alkyl amide and/or a polyamide linker). In some embodiments, each B¹, B², and B³, is an amide linker comprising 1 to about 32 atoms. In some embodiments, the 1 to 32 atoms are selected from carbon, nitrogen, and oxygen.

In some embodiments, each B¹, B², and B³, is selected from:
—C(O)—;
—C(O)NH—;
—C(O)N(CH₃)—;
—C(O)NH—C₀₋₆ alkyl-C(O)—;
—C(O)NH—C₀₋₆ alkyl-C(O)NH—;
—C(O)NH—C₀₋₆ alkyl-C(O)N(CH₃)—;
—C(O)NH—C₀₋₆ alkyl-NH—;
—C(O)NH—C₀₋₆ alkyl-N(CH₃)—;
—C(O)NH—C₀₋₆ alkyl-NHC(O)—;
—C(O)NH—C₀₋₆ alkyl-N(CH₃)C(O)—;
—C(O)N(CH₃)—C₀₋₆ alkyl-C(O)—;
—C(O)N(CH₃)—C₀₋₆ alkyl-C(O)NH—;
—C(O)N(CH₃)—C₀₋₆ alkyl-C(O)N(CH₃)—;
—C(O)NH—C₀₋₆ alkyl-NH—;
—C(O)NH—C₀₋₆ alkyl-N(CH₃)—;
—C(O)N(CH₃)—C₀₋₆ alkyl-NHC(O)—;
—C(O)N(CH₃)—C₀₋₆ alkyl-N(CH₃)C(O)—;
—C(O)—C₁₋₆ alkyl-;
—C(O)—C₀₋₆ alkyl-C(O)—;
—C(O)—C₀₋₆ alkyl-C(O)NH—;
—C(O)—C₀₋₆ alkyl-C(O)N(CH₃)—;
—C(O)—C₀₋₆ alkyl-NH—;
—C(O)—C₀₋₆ alkyl-N(CH₃)—;
—C(O)—C₀₋₆ alkyl-NHC(O)—;
—C(O)—C₀₋₆ alkyl-N(CH₃)C(O)—;
—C₀₋₆ alkyl-C(O)—;
—C₀₋₆ alkyl-C(O)NH—;
—C₀₋₆ alkyl-C(O)N(CH₃)—;
—C₀₋₆ alkyl-C(O)NH—C₀₋₆ alkyl-C(O)—;
—C₀₋₆ alkyl-C(O)NH—C₀₋₆ alkyl-C(O)NH—;
—C₀₋₆ alkyl-C(O)NH—C₀₋₆ alkyl-C(O)N(CH₃)—;
—C₀₋₆ alkyl-C(O)NH—C₀₋₆ alkyl-NH—;
—C₀₋₆ alkyl-C(O)NH—C₀₋₆ alkyl-N(CH₃)—;
—C₀₋₆ alkyl-C(O)NH—C₀₋₆ alkyl-NHC(O)—;
—C₀₋₆ alkyl-C(O)NH—C₀₋₆ alkyl-N(CH₃)C(O)—;
—C₀₋₆ alkyl-C(O)N(CH₃)—C₀₋₆ alkyl-C(O)—;
—C₀₋₆ alkyl-C(O)N(CH₃)—C₀₋₆ alkyl-C(O)NH—;
—C₀₋₆ alkyl-C(O)N(CH₃)—C₀₋₆ alkyl-C(O)N(CH₃)—;
—C₀₋₆ alkyl-C(O)NH—C₀₋₆ alkyl-NH—;
—C₀₋₆ alkyl-C(O)NH—C₀₋₆ alkyl-N(CH₃)—;
—C₀₋₆ alkyl-C(O)N(CH₃)—C₀₋₆ alkyl-NHC(O)—;
—C₀₋₆ alkyl-C(O)N(CH₃)—C₀₋₆ alkyl-N(CH₃)C(O)—;
—C₀₋₆ alkyl-C(O)—C₀₋₆ alkyl-C(O)—;
—C₀₋₆ alkyl-C(O)—C₀₋₆ alkyl-C(O)NH—;
—C₀₋₆ alkyl-C(O)—C₀₋₆ alkyl-C(O)N(CH₃)—;
—C₀₋₆ alkyl-C(O)—C₀₋₆ alkyl-NH—;
—C₀₋₆ alkyl-C(O)—C₀₋₆ alkyl-N(CH₃)—;
—C₀₋₆ alkyl-C(O)—C₀₋₆ alkyl-NHC(O)—;
—C₀₋₆ alkyl-C(O)—C₀₋₆ alkyl-N(CH₃)C(O)—,
—C₀₋₆ alkyl-NH—;
—C₀₋₆ alkyl-NHC(O)—;
—C₀₋₆ alkyl-NHC(O)—C₁₋₆ alkyl-C(O)—;
—C₀₋₆ alkyl-NHC(O)—C₁₋₆ alkyl-C(O)NH—;
—C₀₋₆ alkyl-NHC(O)—C₁₋₆ alkyl-C(O)N(CH₃)—;
—C₀₋₆ alkyl-NHC(O)—C₁₋₆ alkyl-NH—;
—C₀₋₆ alkyl-NHC(O)—C₁₋₆ alkyl-N(CH₃)—;
—C₀₋₆ alkyl-NHC(O)—C₁₋₆ alkyl-NHC(O)—;
—C₀₋₆ alkyl-NHC(O)—C₁₋₆ alkyl-N(CH₃)C(O)—;
—C₀₋₆ alkyl-N(CH₃)—;
—C₀₋₆ alkyl-N(CH₃)C(O)—;
—C₀₋₆ alkyl-N(CH₃)C(O)—C₁₋₆ alkyl-C(O)—;
—C₀₋₆ alkyl-N(CH₃)C(O)—C₁₋₆ alkyl-C(O)NH—;
—C₀₋₆ alkyl-N(CH₃)C(O)—C₁₋₆ alkyl-C(O)N(CH₃)—;
—C₀₋₆ alkyl-N(CH₃)C(O)—C₁₋₆ alkyl-NH—;
—C₀₋₆ alkyl-N(CH₃)C(O)—C₁₋₆ alkyl-N(CH₃)—;
—C₀₋₆ alkyl-N(CH₃)C(O)—C₁₋₆ alkyl-NHC(O)—; and
—C₀₋₆ alkyl-N(CH₃)C(O)—C₁₋₆ alkyl-N(CH₃)C(O)—.

In some embodiments, A¹ is an amino acid or a derivative thereof. In some embodiments, A¹ is an amino acid derivative comprising an amino acid, wherein the carboxylate group(s) is/are substituted with C₁₋₆ aminoalkyl groups. In some embodiments, A¹ is an amino acid derivative comprising an amino acid (e.g., Glu or Asp), wherein the carboxylate groups are substituted with C₁₋₆ aminoalkyl groups (e.g., beta-alanine groups). In some embodiments, A¹ is an amino acid. In some embodiments, A¹ is lysine. In some embodiments, A¹ is L-Lys. In some embodiments, A¹ is D-Lys. In some embodiments, A¹ is Glu, or an aminoalkyl derivative thereof. In some embodiments, A¹ is L-Glu or a beta-alanine-substituted derivative thereof. In some embodiments, A¹ is D-Glu or a beta-alanine-substituted derivative thereof.

In some embodiments, A¹ is an amino acid or a derivative thereof. In some embodiments, A¹ is a Lys (e.g., L-Lys or D-Lys), Glu (L-Glu or D-Glu), or Asp (L-Asp or D-Asp), or a derivative thereof (e.g., substituted with one or more arms (e.g., B¹, B², or B³)). In some embodiments, A¹ has one of the following structures:
*C(O)—C₀₋₆ alkyl-Y(C₀₋₆ alkyl-NH**)(C₀₋₆ alkyl-NH**),
*C(O)—C₀₋₆ alkyl-Y(C₀₋₆ alkyl-NH**)(C₀₋₆ alkyl-C(O)***),
*C(O)—C₀₋₆ alkyl-Y(C₀₋₆ alkyl-C(O)**)(C₀₋₆ alkyl-C(O)***),
*C(O)—C₀₋₆ alkyl-C(O)NH—Y(C₀₋₆ alkyl-NH**)(C₀₋₆ alkyl-NH**),
*C(O)—C₀₋₆ alkyl-C(O)NH—Y(C₀₋₆ alkyl-NH**)(C₀₋₆ alkyl-C(O)***),
*C(O)—C₀₋₆ alkyl-C(O)NH—Y(C₀₋₆ alkyl-C(O)**)(C₀₋₆ alkyl-C(O)***),
*C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH**);
   wherein each of *, **, and *** denotes a bond to a spacer L¹, L², or L³; and Y is N or CH. In some embodiments, Y is lysine or glutamine.

In some embodiments, A¹ has one of the following structures:

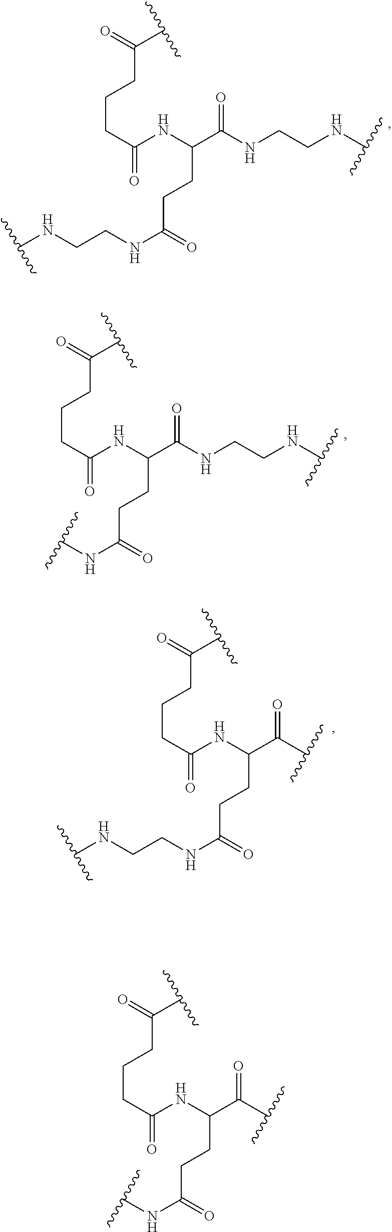 [SMILES: *NCCNC(=O)CCC(NC(=O)CCCC(*)=O)C(=O)NCCN* ; *NCCNC(=O)C(CCC(=O)N*)NC(=O)CCCC(*)=O ; *NCCNC(=O)CCC(NC(=O)CCCC(*)=O)C(*)=O ; *NC(=O)CCC(NC(=O)CCCC(*)=O)C(*)=O]

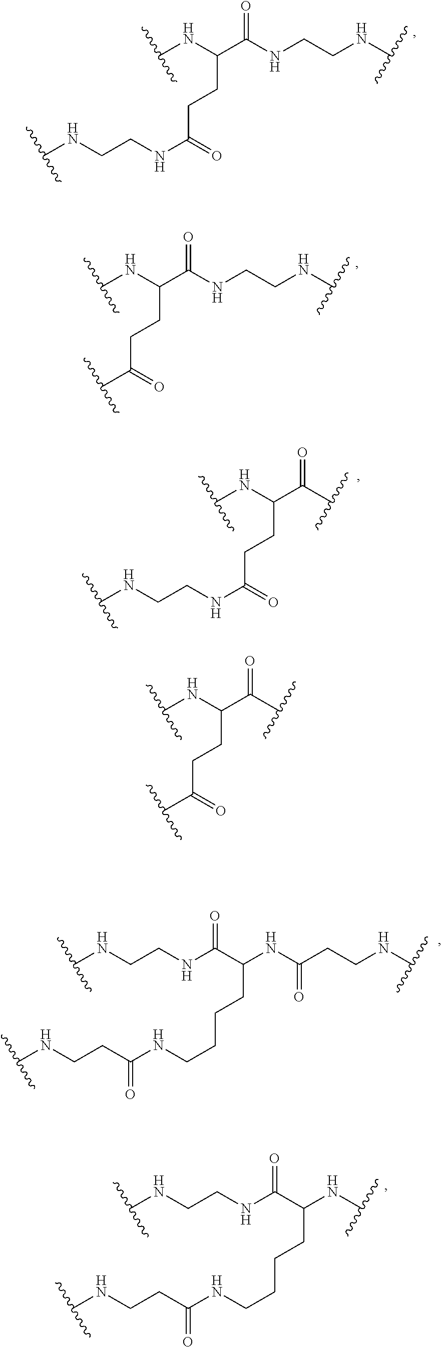 [SMILES: *NCCNC(=O)CCC(N*)C(=O)NCCN* ; *NCCNC(=O)C(CCC(*)=O)N* ; *NCCNC(=O)CCC(N*)C(*)=O ; *NC(CCC(*)=O)C(*)=O ; *NCCNC(=O)C(CCCCNC(=O)CCN*)NC(=O)CCN* ; *NCCNC(=O)C(CCCCNC(=O)CCN*)N*]

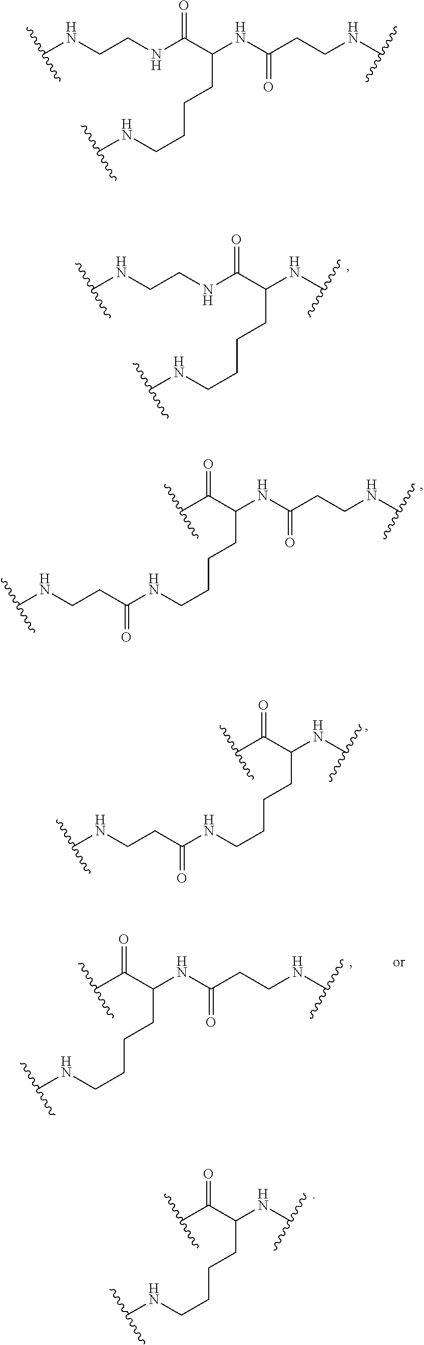 [SMILES: *NCCCCC(NC(=O)CCN*)C(=O)NCCN* ; *NCCCCC(N*)C(=O)NCCN* ; *NCCC(=O)NCCCCC(NC(=O)CCN*)C(*)=O ; *NCCC(=O)NCCCCC(N*)C(*)=O ; *NCCCCC(NC(=O)CCN*)C(*)=O ; *NCCCCC(N*)C(*)=O]

In some embodiments, A¹ is of Formula (A¹-1a):

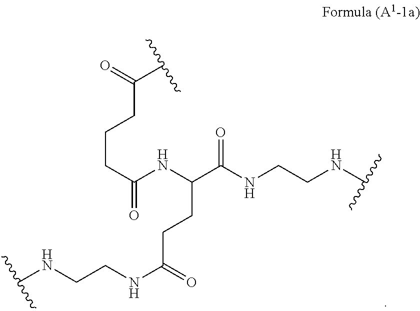 [SMILES: *NCCNC(=O)CCC(NC(=O)CCCC(*)=O)C(=O)NCCN*]

In some embodiments, A¹ is of Formula (A¹-1b):

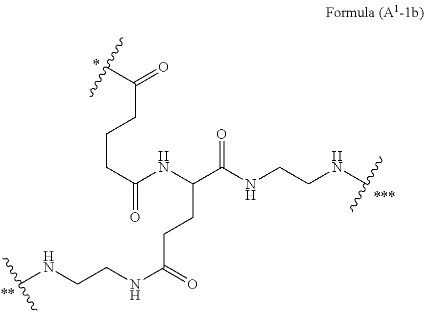 [SMILES: *NCCNC(=O)CCC(NC(=O)CCCC(*)=O)C(=O)NCCN*]

wherein each of *, **, and *** denotes a bond to a spacer L¹, L², or L³.

In some embodiments, A¹ is of Formula (A¹-1c) or Formula (A¹-1d):

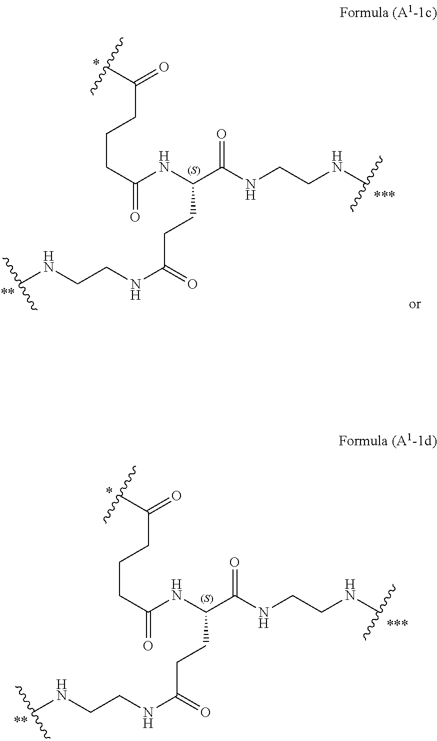

wherein each of *, **, and *** denotes a bond to a spacer L¹, L², or L³.

In some embodiments, A¹ is of Formula (A¹-2a):

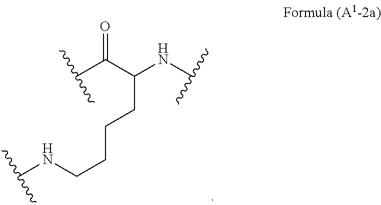 [SMILES: *NCCCCC(N*)C(*)=O]

In some embodiments, A¹ is of Formula (A¹-2b):

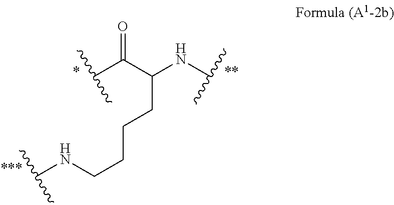 [SMILES: *NCCCCC(N*)C(*)=O]

wherein each of *, **, and *** denotes a bond to a spacer L¹, L², or L³.

In some embodiments, A¹ is of Formula (A¹-2c) or Formula (A¹-2d):

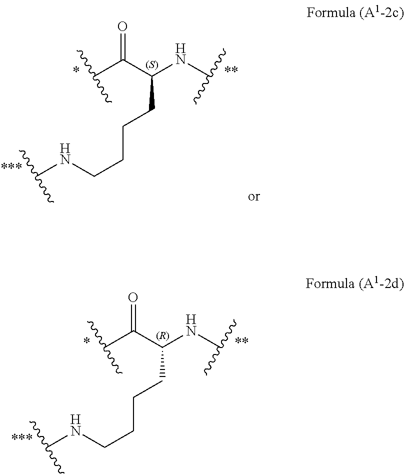

wherein each of *, **, and *** denotes a bond to a spacer L¹, L², or L³.

### Physicochemical Modulators (MOD)

In some embodiments, the present disclosure provides conjugates comprising a physicochemical modulator (“MOD”). A group MOD may be connected to the rest of the conjugate via a linker (e.g., a stable liner). In some embodiments, MOD is connected to EL via a linker (e.g., L⁶). In some embodiments, MOD is bonded to A¹ via a linker (e.g., L³). In some embodiments, MOD is a charged or a polar group. Examples of charged or polar groups include hydroxides and alcohols, carboxylates and carboxylic acids, aminiums and amines, guanidiniums and guanidines, and the like. In some embodiments, MOD is or comprises an amine. In some embodiments, MOD is a polyamine group. In some embodiments, MOD is —NR₂ or —NR₃⁺. In some embodiments, MOD is —C₁₋₆ alkyl-NR₂ or —C₁₋₆ alkyl-NR₃⁺ (e.g., —C₁₋₆ alkyl-NH₂, —C₁₋₆ alkyl-NHCH₃, —C₁₋₆ alkyl-N(CH₃)₂, or —C₁₋₆ alkyl-N(CH₃)₃⁺). In some embodiments, MOD is —C₁₋₆ alkyl-NHC(═NH⁺)NH₂. In some embodiments, MOD is a polyacid group. In some embodiments, MOD is —COOH or —COO⁻. In some embodiments, MOD is —C₁₋₆ alkyl-COOR (e.g., —C₁₋₆ alkyl-COOH, —C₁₋₆ alkyl-COO⁻, —C₁₋₆ alkyl-COO⁻Na⁺ or —C₁₋₆ alkyl-COOCH₃). In some embodiments, MOD is a group that is converted to a polar or charged group in vivo (e.g., MOD is an alkyl ester, which is cleaved in vivo to an alkyl acid, thereby reducing membrane permeability). In some embodiments, MOD is —OH or —O—. In some embodiments, MOD is an amino acid. In some embodiments, MOD is a basic amino acid. In some embodiments, MOD is Arg, His, or Lys. In some embodiments, MOD is an acidic amino acid. In some embodiments, MOD is Glu or Asp. In some embodiments, MOD is an unnatural amino acid (e.g., D-Glu or D-Asp). In some embodiments, MOD is a polar amino acid (e.g., Ser, Thr, Asn, Gln). In some embodiments, provided herein is a compound of the formula:

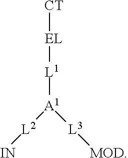

In some embodiments, L³ is a heteroalkyl linker. In some embodiments, L³ is a simple spacer. In some embodiments, L³ is a carbonyl, an acyl linker, an amide linker, or an alkyl linker. In some embodiments, L³ is an optionally substituted alkyl linker (e.g., an optionally substituted alkyl amide linker). In some embodiments, MOD is —OH, —COOH, —NH₂, NHCH₃, —N(CH₃)₂, —(CH₃)₃⁺, or a salt thereof. In some embodiments, L³ is C₁₋₁₂ alkyl. In some embodiments, L³ is NHC₁₋₁₂ alkyl. In some embodiments, L³ is NHC(O)—C₁₋₁₂ alkyl. In some embodiments, L³ is C(O)—C₁₋₁₂ alkyl. In some embodiments, L³ is a linker (L) or spacer (SP) as defined herein.

In some embodiments, L³ is —(CO)ᵣ(CH₂)ₛ(NR¹⁰C(O)C₁₋₆ alkyl)ₜ(NR¹¹)ᵤ(CO)ᵥ—. In some embodiments, L³ is —[N(CH₃)CH₂C(O)]₁₋₁₂ and MOD is —OH or —O—. Examples of L³-MOD include: —[N(CH₃)CH₂C(O)]₄O⁻, —[N(CH₃)CH₂C(O)]₆O⁻, and —[N(CH₃)CH₂C(O)]₈O⁻. In some embodiments, MOD is —OH, —COOH, —NH₂, NHCH₃, —N(CH₃)₂, —(CH₃)₃⁺, or a salt thereof. In some embodiments, L³ is C₁₋₁₂ alkyl. In some embodiments, L³ is NHC₁₋₁₂ alkyl. In some embodiments, L³ is NHC(O)—C₁₋₁₂ alkyl. In some embodiments, L³ is C(O)—C₁₋₁₂ alkyl. In some embodiments, L³ is as described herein.

Enzymatically-Cleavable linker (EL)

In an aspect, provided herein is a compound, or a pharmaceutically acceptable salt thereof; or a stereoisomer or mixture of stereoisomers thereof, having a structure represented by any one of Formulae (01)-(05):

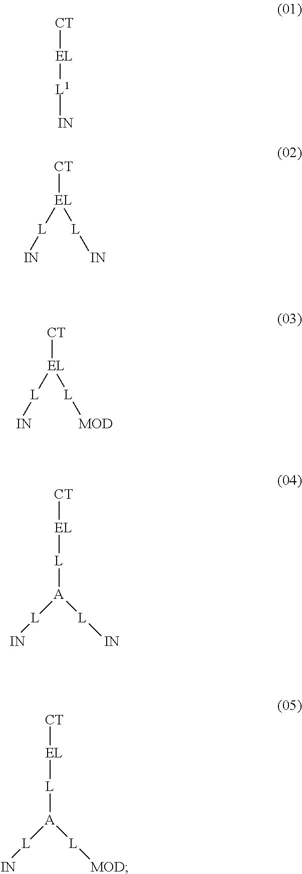

wherein:
CT is a hexacyclic cytotoxic or cytostatic ligand;
EL is an enzymatically-cleavable linker;
L is a stable linker;
A is a branching group;
IN is an integrin binding group; and
MOD is a physicochemical modulator group.

In some embodiments, EL is a dipeptide having the formula -AA¹-AA²-, or a tripeptide having the formula AA¹-AA²-AA³-, wherein each AA¹, AA², and AA³ is independently an amino acid, or a derivative thereof. In some embodiments, EL further comprises a self-immolative linker (SIL).

In some embodiments, provided herein is a compound of any one of Formulae (01)-(05), wherein EL has the formula:
*-AA¹-AA²-(AA³)₀₋₁-(SIL)₀₋₁-**;
wherein:
each AA¹, AA², and AA³ is independently an amino acid, or a derivative thereof,
SIL is a self-immolative linker,
* is a bond to L (e.g., L¹, L⁴, L⁵, L⁶, or L⁷); and
** is a bond to CT.

In some embodiments, SIL is a PAB (p-aminobenzyl carbamate) group.

In some embodiments, EL is a dipeptide, having the formula -AA¹-AA²-, wherein each AA¹ and AA² is independently an amino acid.

In some embodiments, EL is a tripeptide having the formula -AA¹-AA²-AA³-, wherein each AA¹, AA², and AA³ is independently an amino acid.

In some embodiments, the present invention provides compounds having a structure of formulae disclosed herein, or a pharmaceutically acceptable salt thereof, or a stereoisomer or mixture of stereoisomers thereof; wherein EL is cleaved by an enzyme from the class of proteases.

In some embodiments, the present invention provides compounds having a structure of formulae disclosed herein, or a pharmaceutically acceptable salt thereof, or a stereoisomer or mixture of stereoisomers thereof; wherein EL is cleaved by neutrophil elastase.

In some embodiments, the present invention provides compounds having a structure of formulae disclosed herein, or a pharmaceutically acceptable salt thereof, or a stereoisomer or mixture of stereoisomers thereof, wherein EL is cleaved by neutrophil elastase.

In some embodiments, the present invention provides compounds having a structure of formulae disclosed herein, or a pharmaceutically acceptable salt thereof, or a stereoisomer or mixture of stereoisomers thereof, wherein EL has the formula: L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-. In some embodiments, EL having the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-is cleavable by neutrophil elastase.

In some embodiments, EL has the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, or L-His-L-Pro-L-Val-. In some embodiments, EL has the formula L-Asp-L-Pro-L-Val, L-Asn-L-Pro-L-Val, or -Gly-L-Pro-L-Val-.

In some embodiments, EL is of Formula (EL-1a):

L-Asp-L-Pro-L-Val Formula (EL-1a).

In some embodiments, EL is of Formula (EL-1b):

L-Asp*-L-Pro-L-Val Formula (EL-1b)
wherein Asp* represents a prodrug of aspartate, such as an alkyl ester of Asp, wherein the alkyl group is substituted with an amine (e.g., NH₂, N(CH₃)₂, N(CH₃)₃⁺) or a linker to an integrin binder (e.g., -L⁶-IN, NHL⁶-IN, or NHCO—IN).

In some embodiments, EL is of Formula (EL-2):

L-Asn-L-Pro-L-Val Formula (EL-2).

In some embodiments, EL is of Formula (EL-3):

Gly-L-Pro-L-Val Formula (EL-3).

In some embodiments, EL is represented by Formula (I):

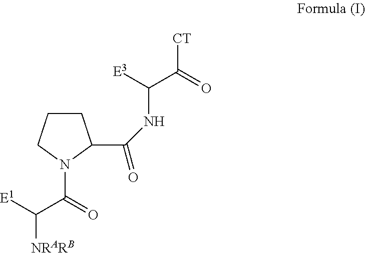

wherein CP, E³, and E¹ are as defined herein;
R^{A} is hydrogen or C₁₋₆ alkyl; and
R^{B} is a bond to a spacer (SP) as described herein, or linker (L¹, L⁵, or L⁷).

### Integrin Binders (IN)

In some embodiments, the present invention provides compounds having a structure of formulae disclosed herein, or a pharmaceutically acceptable salt thereof, or a stereoisomer or mixture of stereoisomers thereof; wherein IN is an integrin binder comprising peptides, proteins, antibodies, or small molecules. In some embodiments, molecular weight of such peptides, proteins, antibodies, or small molecules is <1 kD.

In some embodiments, the present invention provides compounds having a structure of formulae disclosed herein, or a pharmaceutically acceptable salt thereof, or a stereoisomer or mixture of stereoisomers thereof, wherein IN is a peptidic or peptidomimetic integrin binder.

In some embodiments, the present invention provides compounds having a structure of formulae disclosed herein, or a pharmaceutically acceptable salt thereof, or a stereoisomer or mixture of stereoisomers thereof, wherein IN is an integrin binder comprising non-peptides, non-antibodies, or small molecules. In some embodiments, molecular weight of such non-peptides, non-proteins, non-antimobodies, or small molecules (e.g., <1 kD). In some embodiments, IN is a small molecule integrin binder. In some embodiments, IN is a small molecule integrin binder having two or more urea groups (—NHC(O)NH—). In some embodiments, IN is a small molecule αᵥβ₃ integrin binder.

In some embodiments, the present invention provides compounds having a structure of formulae disclosed herein, or a pharmaceutically acceptable salt thereof, or a stereoisomer or mixture of stereoisomers thereof, wherein IN is a linear integrin binding peptide.

In some embodiments, IN is a macrocyclic integrin binding peptide.

In some embodiments, the linear integrin binding peptide is a constrained macrocyclic integrin binding peptide.

In some embodiments, IN is a non-peptidic or non-peptidomimetic integrin binder.

In some embodiments, IN is a tumor-binding moiety. In some embodiments, IN binds to both tumor and non-tumor cells. In some embodiments, IN binds rapidly dividing cells (e.g., tumor cells or other hyperproliferating cells).

In some embodiments, IN is an αᵥβ₃-binding moiety.

In some embodiments, IN is IN-1a or IN-1b, wherein

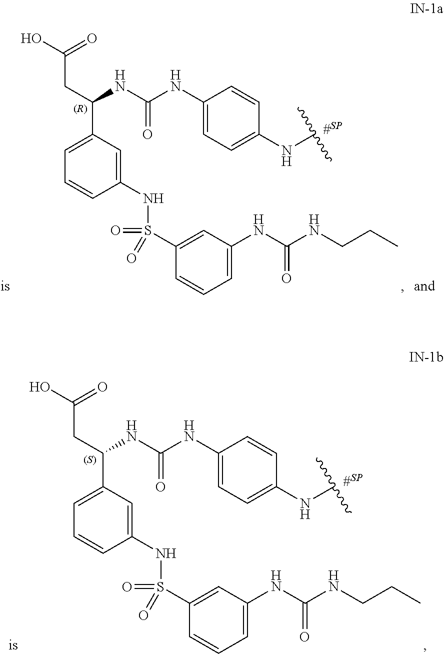

or a pharmaceutically acceptable salt thereof; or a stereoisomer or mixture of stereoisomers thereof; wherein #SP denotes a bond to a spacer, e.g., one of L², L³, L⁴, L⁵, L⁶, and/or L⁷.

As denoted in Tables A and B, IN-1a is indicated when *═(R), and IN-1b corresponds to *═(S). The (R) isomer is preferred for binding to an integrin receptor (e.g., an αᵥβ₃ integrin receptor), as this isomer has superior binding (lower IC₅₀) to the (S) isomer.

In some embodiments, IN has a structure:

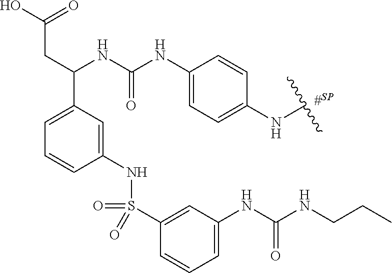 [SMILES: *Nc1ccc(NC(=O)NC(CC(=O)O)c2cccc(NS(=O)(=O)c3cccc(NC(=O)NCCC)c3)c2)cc1]

or a pharmaceutically acceptable salt thereof; or a stereoisomer or mixture of stereoisomers thereof; wherein #^{SP} denotes a bond to a spacer, e.g., one of L², L³, L⁴, L⁵, L⁶, and/or L⁷.

### Cytotoxic or Cytostatic Radicals (CT)

Disclosed herein are compounds having a hexacyclic cytotoxic or cytostatic radical, CT. In some embodiments, CT is a (hexacyclic) small molecule radical (e.g., <1 kD in molecular weight). In some embodiments, CT is a macrocyclic radical. In some embodiments, CT is a polycyclic radical. In some embodiments, CT is a polycyclic radical having six rings. In some embodiments, CT is a polycyclic radical having six fused rings. In some embodiments, CT is a hexacyclic radical. In some embodiments, CT is a hexacyclic radical having an alpha-hydroxy lactone moiety in one of the rings.

In some embodiments, CT is a C₂₀ hydroxy radical of

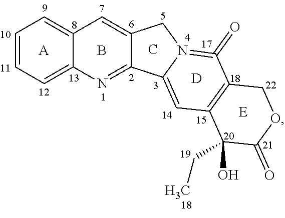

wherein a fused ring is formed between C₇ and C₉, wherein said fused ring is substituted with —NR^{X}R^{Y}. In some embodiments, the fused ring between C₇ and C₉ (i.e., X³ and X⁴ respectively in Formula (I)) is a cycloalkyl ring. In some embodiments, the fused ring between C₇ and C₉ (i.e., X³ and X⁴ respectively in Formula (I)) is a cycloalkyl ring substituted with —NR^{X}R^{Y}. In some embodiments, the fused ring between C₇ and C₉ (i.e., X³ and X⁴ respectively in Formula (I)) is a cyclohexyl ring substituted with —NR^{X}R^{Y}. In some embodiments, CT is a hexacyclic camptothecin derivative. In some embodiments, R^{X} is hydrogen, C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂(pyridine); and R^{Y} is hydrogen or C₁₋₆ alkyl.

In some embodiments, CT is:

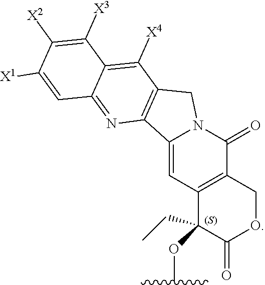

In some embodiments:
X¹ and X² taken together form an ethylenedioxy or methylenedioxy heterocycle; and
X³ and X⁴ are each independently hydrogen, halogen, C₁₋₆ haloalkyl, C₁₋₆ alkyl, or C₁₋₆ alkylamine.

In some embodiments:
X¹ and X² taken together form a methylenedioxy heterocycle; and
X³ and X⁴ are each independently hydrogen, C₁₋₆ alkyl, or C₁₋₆ alkylamine.

In some embodiments:
X¹ and X² taken together form a methylenedioxy heterocycle;
X³ is hydrogen; and
X⁴ is hydrogen, C₁₋₆ alkyl, or C₁₋₆ alkylamine.

In some embodiments:
X¹ and X² taken together form a methylenedioxy heterocycle; and
X³ is hydrogen; and
X⁴ is hydrogen, —CH₂CH₃, —CH₂NH₂, or —CH₂N(CH₃)₂.

In some embodiments:
X¹ and X² taken together form a methylenedioxy heterocycle;
X³ is hydrogen; and
X⁴ is hydrogen or —CH₂NH₂.

In some embodiments:
X¹ and X² are each independently hydrogen, halogen, C₁₋₄ alkyl, or C₁₋₄ haloalkyl; and
X³ and X⁴ taken together form a carbocyclic ring A substituted with —NR^{X}R^{Y};
wherein:
R^{X} is hydrogen, C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂(pyridine);
   wherein:
   EL² is (-L-Asp)-(L-Pro)-(L-Val-), (-L-Asn)-(L-Pro)-(L-Val-), (-L-Ala)-(L-Ala)-(L-Asn-), or (-L-Ala)-(L-N-Me-Ala)-(L-Asn-); and
R^{Y} is hydrogen or C₁₋₆alkyl.

In some embodiments:
X¹ and X² are each independently hydrogen, halogen, C₁₋₄ alkyl, or C₁₋₄ haloalkyl; and
X³ and X⁴ taken together form a carbocyclic ring A substituted with —NR^{X}R^{Y}; wherein R^{X} is hydrogen, —C(O)CH₃, or —C(O)OBn; and R^{Y} is hydrogen.

In some embodiments:
X¹ and X² are each independently halogen or —CH₃; and
X³ and X⁴ taken together form a cyclohexyl ring substituted with —NR^{X}R^{Y}; wherein:
   R^{X} is hydrogen, C₁₋₆ alkyl, —C(O)CH₃, —C(O)CF₃, —C(O)OBn, or —C(O)CH₂(pyridine), -Asn-C(O)CH₂(pyridine), or
   R^{Y} is hydrogen or C₁₋₆alkyl.

In some embodiments:
X¹ and X² are each independently hydrogen, halogen, or —CH₃; and
X³ and X⁴ taken together form a cyclohexyl ring substituted with —NR^{X}R^{Y}; wherein R^{X} is hydrogen, —C(O)CH₃, or —C(O)OBn; and R^{Y} is hydrogen.

In some embodiments:
X¹ is —F;
X² is —CH₃; and
X³ and X⁴ taken together form a cyclohexyl ring substituted with —NH₂, —NHC(O)CH₃, or —NHC(O)OBn.

In some embodiments:
(i) X¹ and X² taken together form a heterocycle, and X³ and X⁴ are each hydrogen; or
(ii) X¹ and X² are each independently hydrogen, halogen, C₁₋₄ alkyl, or C₁₋₄ haloalkyl, and X³ and X⁴ taken together form a carbocyclic ring A substituted with —NR^{X}R^{Y}; wherein:
R^{X} is hydrogen, C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂(pyridine);
   wherein:
   EL² is (-L-Asp)-(L-Pro)-(L-Val-), (-L-Asn)-(L-Pro)-(L-Val-), (-L-Ala)-(L-Ala)-(L-Asn-), or (-L-Ala)-(L-N-Me-Ala)-(L-Asn-);
R^{Y} is hydrogen, substituted or unsubstituted alkyl, substituted or unsubstituted heteroalkyl, substituted or unsubstituted carbocycle, or a substituted or unsubstituted heterocycle; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo (═O), —SO₃H, —SO₂(C₁₋₆alkyl), —SO₂NH₂, —SO₂NH(C₁₋₆alkyl), and —SO₂N(C₁₋₆alkyl)₂.

In some embodiments, R^{X} is hydrogen, C₁₋₆ alkyl, —C(O)CH₃, —C(O)CF₃, or —C(O)OBn. In some embodiments, R^{X} is hydrogen, C₁₋₆ alkyl, —C(O)CH₃, or —C(O)CF₃. In some embodiments, R^{X} is hydrogen, C₁₋₆ alkyl, or —C(O)CH₃. In some embodiments, R^{X} is hydrogen. In some embodiments, R^{X} is C₁₋₆ alkyl, or —C(O)CH₃. In some embodiments, R^{X} is —CH₂CH₃ or —C(O)CH₃. In some embodiments, R^{X} is —CH₃. In some embodiments, R^{X} is —CH₃ or —CH₂CH₃, and R is hydrogen or —C₁₋₆ alkyl. In some embodiments, R^{X} and R^{Y} are each independently hydrogen, —CH₃ or —CH₂CH₃, or —C(O)CH₃.

In some embodiments, R^{Y} is hydrogen or substituted alkyl, wherein the alkyl is substituted with one or more groups selected from deuterium, halogen, —CONH₂, —CONH(C₁₋₆alkyl), —CON(C₁₋₆alkyl)₂, —COOH, —COO(C₁₋₆alkyl), —NH₂, —NH(C₁₋₆alkyl), —N(C₁₋₆alkyl)₂, —OH, —O(C₁₋₆alkyl), and oxo (═O). In some embodiments, R^{Y} is hydrogen or substituted C₁₋₆ alkyl, wherein the alkyl is substituted with one or more groups selected from deuterium, halogen, —CONH₂, —CONH(CH₃), —CONH(CH₂CH₃), —CON(CH₃)₂, —CON(CH₂CH₃)₂, —NH₂, —NH(CH₃), —NH(CH₂CH₃), —N(CH₃)₂, —N(CH₂CH₃)₂, —OH, or oxo (═O). In some embodiments, R is hydrogen or substituted C₁₋₆ alkyl, wherein the alkyl is substituted with one or more groups selected from deuterium, halogen, and oxo (═O). In some embodiments, R^{Y} is substituted or unsubstituted alkyl; wherein if R is substituted, it is substituted with one or more groups selected from deuterium, fluorine, chlorine, and oxo. In some embodiments, R^{Y} is hydrogen, C₁₋₆alkyl, C₁₋₆deuteroalkyl, C₁₋₆haloalkyl, —C(O)C₁₋₆alkyl, —C(O)C₁₋₆deuteroalkyl, or —C(O)C₁₋₆haloalkyl. In some embodiments, R^{Y} is hydrogen, C₁₋₆alkyl, C₁₋₆deuteroalkyl, —C(O)C₁₋₆alkyl, or —C(O)C₁₋₆haloalkyl. In some embodiments, R^{Y} is hydrogen, —CH₃, —CD₃, —CH₂CH₃, —CH₂CD₃, —CD₂CD₃, —CH₂CH₂CH₃, —CH(CH₃)₂, —C(O)CH₃, —C(O)CH₂CH₃, —C(O)CF₃, —C(O)CHF₂, —C(O)CH₂F, or —C(O)CH₂CF₃. In some embodiments, R^{Y} is hydrogen, —CH₃, —CD₃, —CH₂CH₃, —CD₂CD₃, —C(O)CH₃, —C(O)CD₃, —C(O)CH₂CH₃, —C(O)CF₃, —C(O)CHF₂, —C(O)CH₂F, or —C(O)CH₂CF₃. In some embodiments, R^{Y} is hydrogen, —CH₃, —CH₂CH₃, —C(O)CH₃, —C(O)CH₂CH₃, or —C(O)CF₃. In some embodiments, R^{Y} is hydrogen, —CH₂CH₃, —C(O)CH₃, or —C(O)CF₃. In some embodiments, R^{Y} is hydrogen or C₁₋₆alkyl. In some embodiments, R^{Y} is hydrogen, —CH₃, or —CH₂CH₃. In some embodiments, R^{Y} is hydrogen or —CH₂CH₃. In some embodiments, R^{Y} is hydrogen. In some embodiments, R^{Y} is —C₁₋₆alkyl. In some embodiments, R^{Y} is —CH₂CH₃. In some embodiments, R^{Y} is hydrogen, C₁₋₆alkyl, —C(O)C₁₋₆alkyl, or —C(O)C₁₋₆haloalkyl. In some embodiments, R^{Y} is aralkyl, heteroaralkyl, heteroalkyl-aryl, heteroalkyl-heteroaryl, each alkyl, heteroalkyl, aryl, or heteroaryl group being optionally substituted with one or more halo, oxo, hydroxy, amino, amide, and/or acid groups.

In some embodiments, CT is a radical of a cytotoxic or cytostatic moiety. In some embodiments, CT is a radical of a hexacyclic cytotoxic or cytostatic moiety. In some embodiments, CT is a radical of a hexacyclic topoisomerase inhibitor. In some embodiments, CT is an alpha-hydroxy lactone radical of a hexacyclic topoisomerase inhibitor. In some embodiments, CT is a hydroxy radical of a hexacyclic topoisomerase inhibitor. In some embodiments, CT is a C₂₀ hydroxy radical of a hexacyclic topoisomerase inhibitor. In some embodiments, CT is a hydroxy radical of exatecan, DXd, 10,11-methylenedioxy-camptothecin (FL118), or a derivative thereof. In some embodiments, an alpha-hydroxy lactone radical of a hexacyclic topoisomerase inhibitor is more stable (i.e., undergoes less ring-opening isomerism of the lactone ring) than an N-linked or N-glycyl linked analog thereof. In some embodiments, provided herein are compounds and conjugates containing a hexacyclic topoisomerase inhibitor linked via an ester bond via the hydroxy radical at the lactone ring), wherein lactone ring hydrolysis and ring-opening is suppressed compared to N-linked compounds or conjugates with an unmodified hydroxy lactone ring (e.g., Compound 1 or trastuzumab deruxtecan).

In some embodiments, CT is a radical of exatecan or DXd, or a derivative thereof. In some embodiments, CT is selected from:

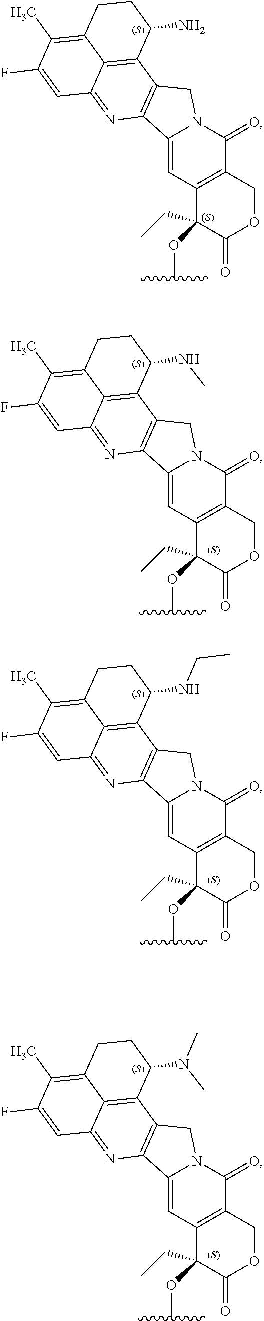

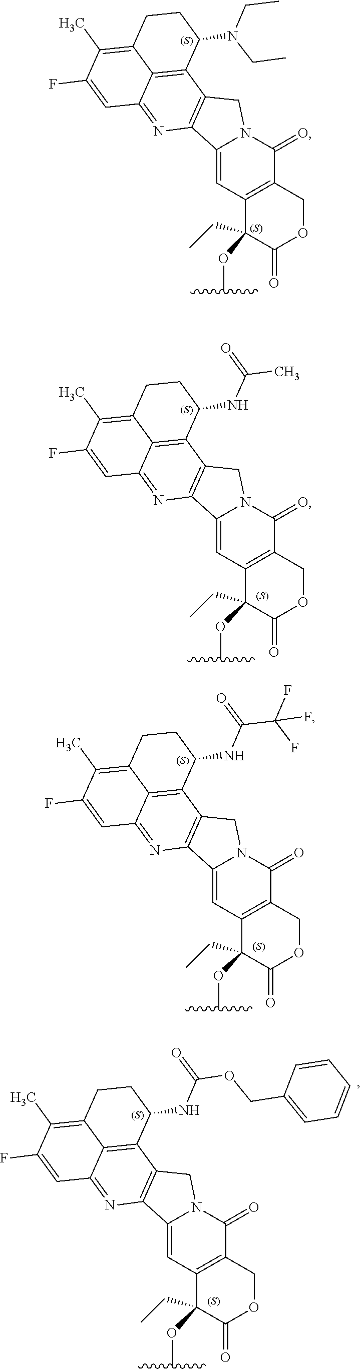

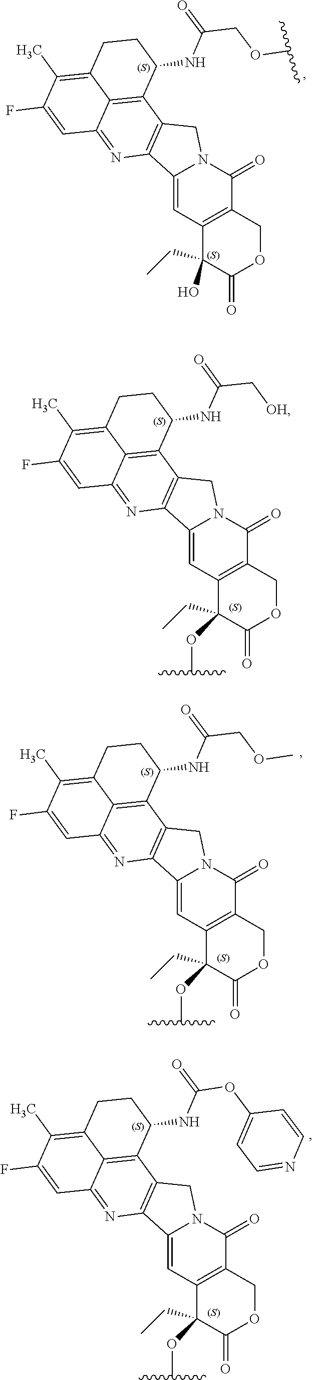

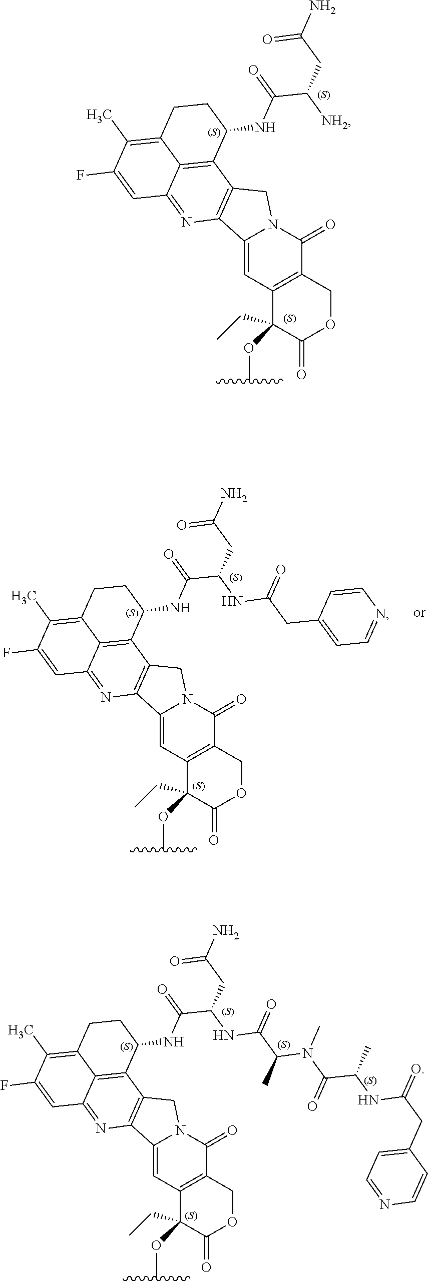

In some embodiments, CT is a radical of exatecan, or a derivative thereof. In some embodiments, CT is selected from:

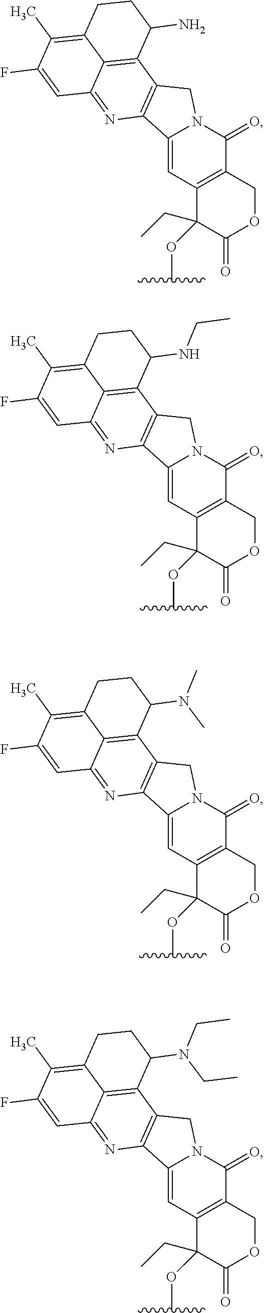

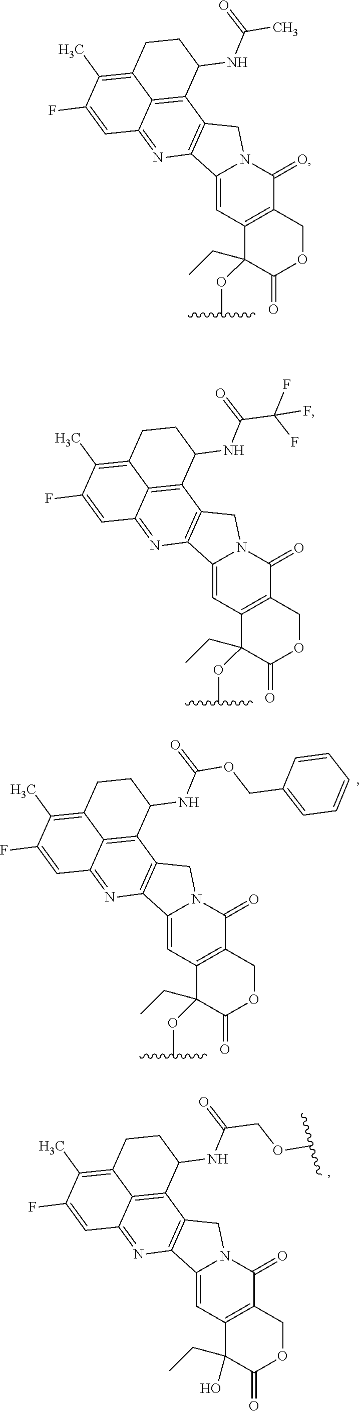

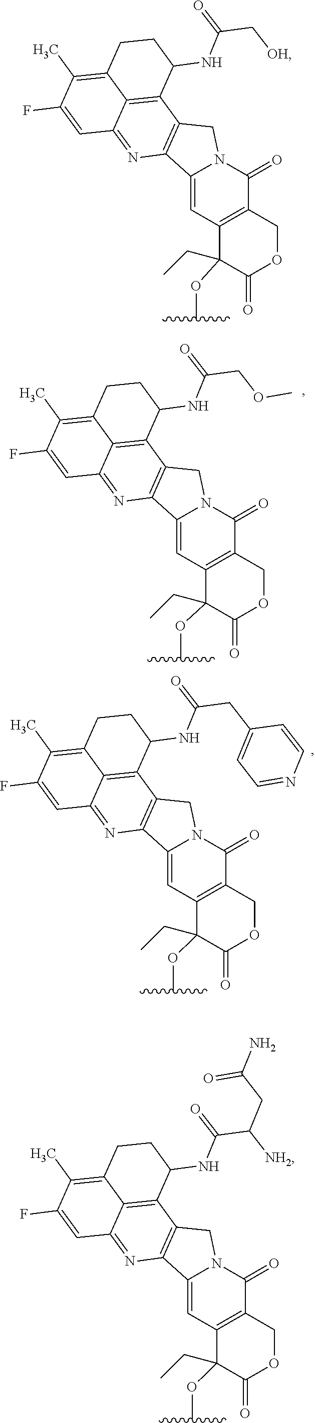

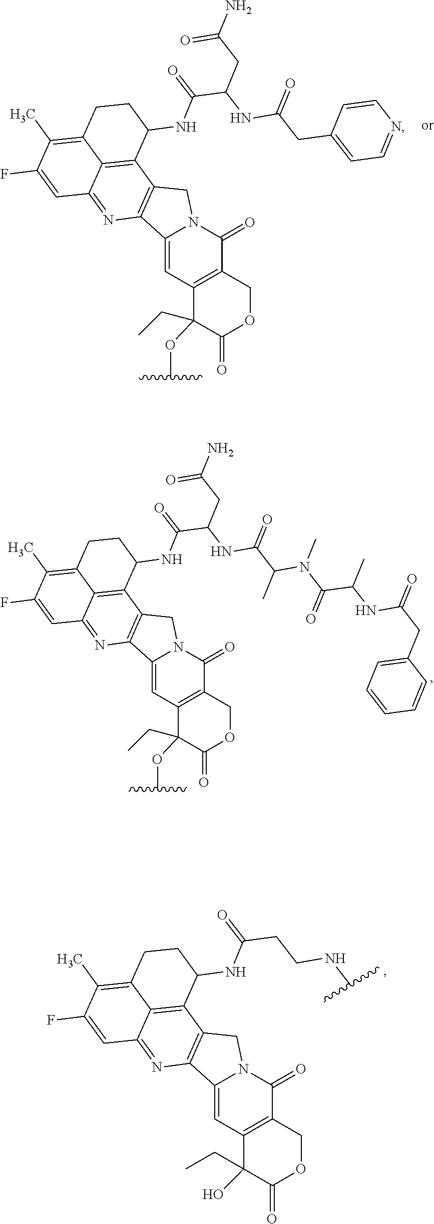

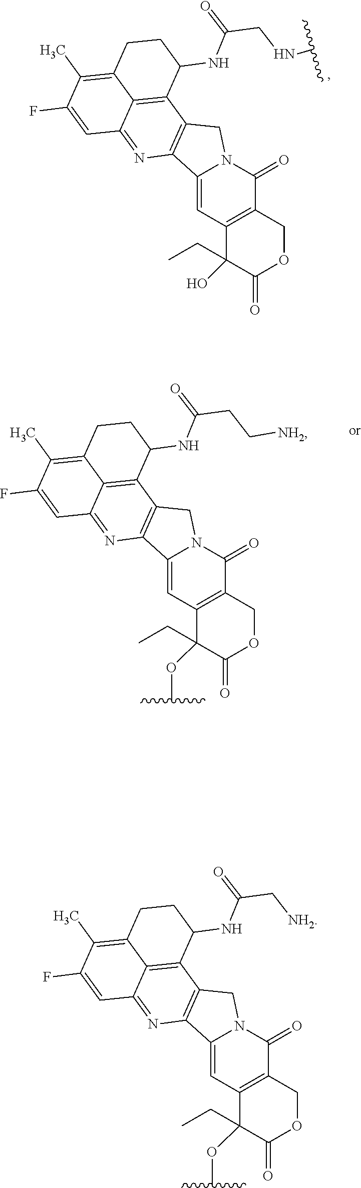

In some embodiments, CT is a 10,11-ethylenedioxy-camptothecin radical. In some embodiments, CT is a radical of lurtotecan, or a derivative thereof. In some embodiments, CT is a radical of 10,11-methylenedioxy-camptothecin (FL118), or a derivative thereof. In some embodiments, CT is a radical of 7-methanamine-10,11-methylenedioxy-camptothecin, or a derivative thereof.

In some embodiments, CT is selected from:

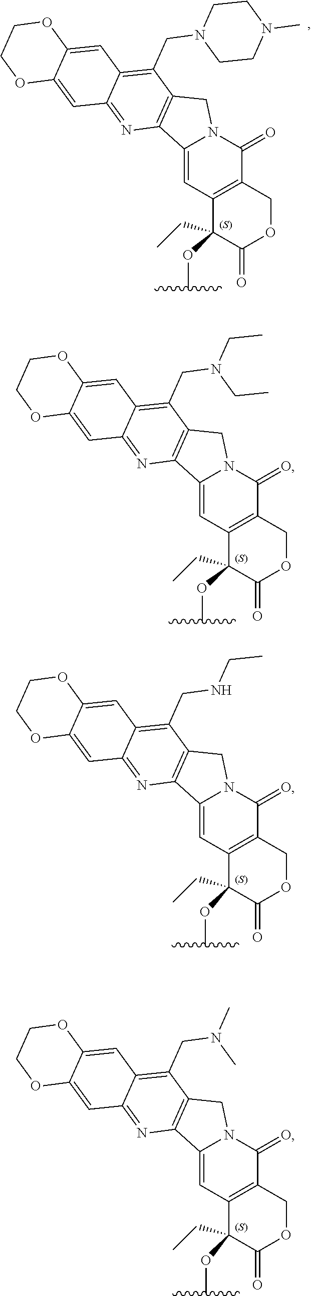

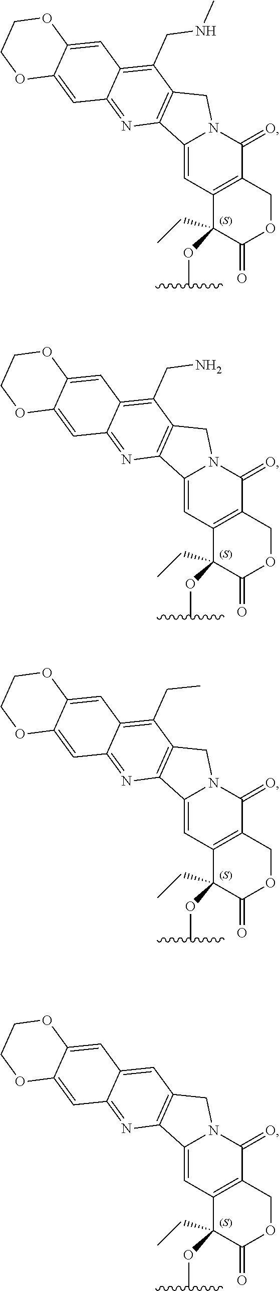

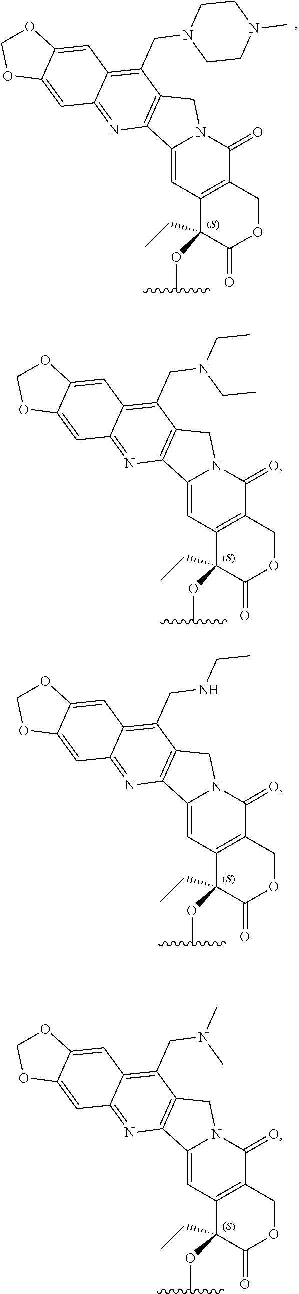

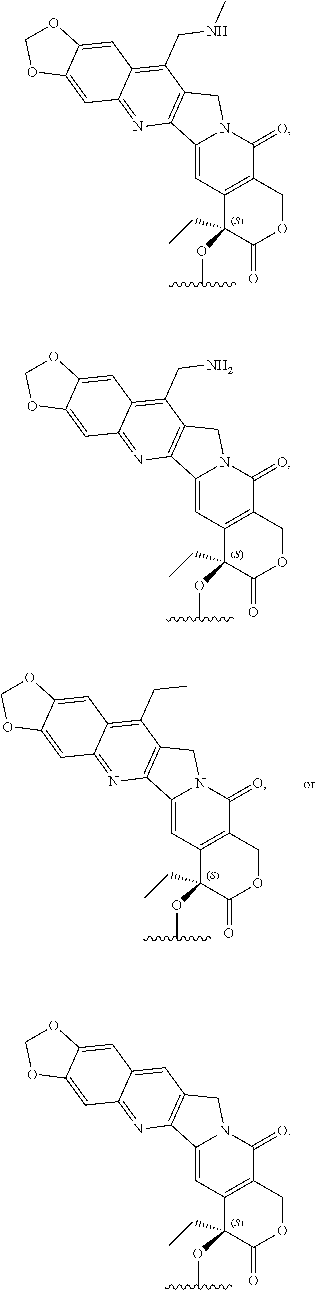

In some embodiments, CT is selected from:

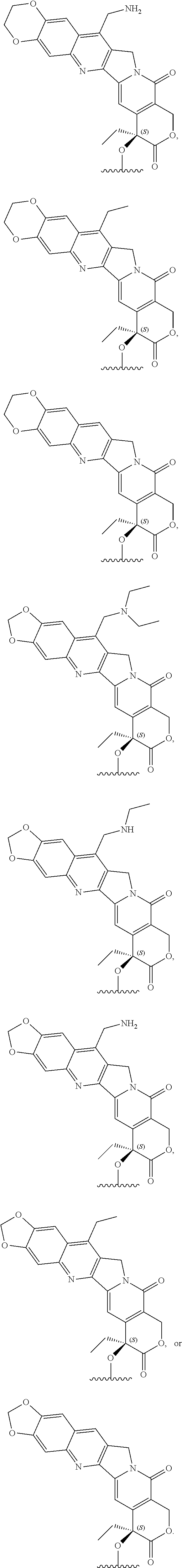

In some embodiments, CT is a 10,11-methylenedioxy-camptothecin radical, which forms an ester linkage at the C₂₀ hydroxyl group to an amino acid (e.g., an amino acid of EL or Formula (I)). In some embodiments, CT is a 7-methanamine-10,11-methylenedioxy-camptothecin radical, which forms an ester linkage at the hydroxyl group at the lactone ring to an amino acid (e.g., an amino acid of EL or Formula (I)). In some embodiments, CT is a 10,11-methylenedioxy-camptothecin or 7-methanamine-10,11-methylenedioxy-camptothecin radical bonded via an ester linkage to the carbonyl of a valine residue.

In some embodiments, CT is:

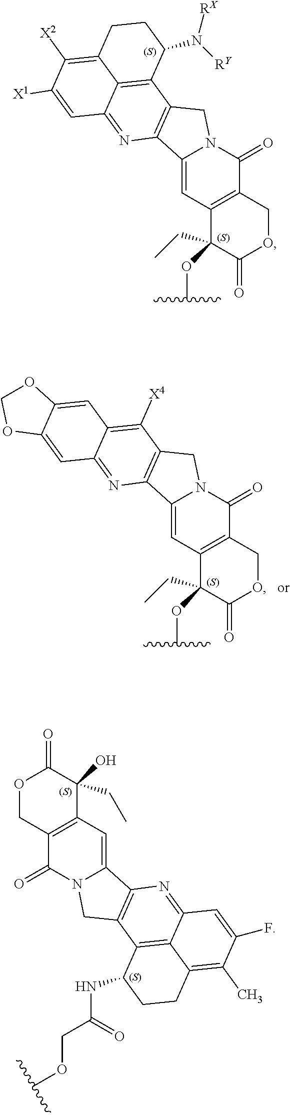

In some embodiments, X¹ is F, X² is —CH₃, and X⁴ is hydrogen or —CH₂NH₂. In some embodiments, R^{X} is hydrogen, —CH₂CH₃, —C(O)CH₃, —C(O)CF₃, —C(O)OBn, —C(O)CH₂(pyridine), -Asn-C(O)CH₂(pyridine), or -(-L-Asn)-(N-Me-L-Ala)-(L-Ala-)-C(O)CH₂-(pyridine) and R^{Y} is hydrogen or —CH₂CH₃.

Any combination of the groups described above for the various variables is contemplated herein. Throughout the specification, groups and substituents thereof are chosen by one skilled in the field to provide stable moieties and compounds.

### Pharmaceutically Acceptable Salts

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, pharmaceutically acceptable salts are obtained by reacting a conjugate described herein with an acid. In some embodiments, the conjugate described herein (e.g., free base form) is basic and is reacted with an organic acid or an inorganic acid. Inorganic acids include, but not limited to, hydrochloric acid, hydrobromic acid, sulfuric acid, phosphoric acid, nitric acid, and metaphosphoric acid. Organic acids include, but not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts of the inventive compounds especially include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid, and embonic acid.

In some embodiments, a conjugate described herein is prepared as a chloride salt, sulfate salt, bromide salt, mesylate salt, maleate salt, citrate salt or phosphate salt.

In some embodiments, pharmaceutically acceptable salts are obtained by reacting a conjugate described herein with a base. In some embodiments, the conjugate described herein is acidic and is reacted with a base. In such situations, an acidic proton of the conjugate described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt, e.g., a monosodium salt, a disodium salt, a trisodium salt, or a tetrasodium salt. In some embodiments, the salt is a disodium or trisodium salt. In some embodiments, the salt is a trifluoroacetate (TFA) salt, e.g., a mono-TFA, di-TFA, tri-TFA, or tetra-TFA salt.

In some embodiments, pharmaceutically acceptable salts of the inventive compounds also include salts derived from conventional bases, by way of example alkali metal salts (e.g. sodium and potassium salts), alkaline earth metal salts (e.g. calcium and magnesium salts), zinc salts and ammonium salts derived from ammonia or organic amines having 1 to 20 carbon atoms, by way of example ethylamine, diethylamine, triethylamine, N,N-ethyldiisopropylamine, monoethanolamine, diethanolamine, triethanolamine, dimethylaminoethanol, diethylaminoethanol, tris(hydroxymethyl)aminomethane, choline, benzalkonium, procaine, dibenzylamine, dicyclohexylamine, N-methylmorpholine, N-methylpiperidine, arginine, lysine, and 1,2-ethylenediamine.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, the context of the invention is described as those forms of the inventive compounds which form a complex in the solid or liquid state by coordination with solvent molecules. Hydrates are a specific form of the solvates in which the coordination is with water. Solvates preferred in the context of the present invention are hydrates.

The methods and formulations described herein include the use of N-oxides (if appropriate), or pharmaceutically acceptable salts of compounds having the structure of any one of the formulae disclosed herein, as well as active metabolites of these compounds having the same type of activity.

In some embodiments, sites on the organic radicals (e.g. alkyl groups, aromatic rings) of compounds of formulae disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, particularly compounds labelled with ³H, ¹⁴C, or ¹⁸F isotopes are suitable for the purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore possibly also constitute a preferred embodiment of the present invention. Isotopic variants of the inventive compounds can be prepared by commonly used processes known to those skilled in the art, for example by the methods described further down and the procedures described in the working examples, by using corresponding isotopic modifications of the respective reagents or starting compounds.

In some embodiments, the compounds disclosed herein possess one or more stereocenters and each stereocenter exists independently in either the R or S configuration. In some embodiments, the conjugate described herein exists in the R configuration. In some embodiments, the conjugate described herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In some embodiments, a cytotoxic or cytostatic prodrug (e.g., a topoisomerase inhibitor prodrug (e.g., an exatecan or methylenedioxy-camptothecin prodrug) disclosed herein is to increase the therapeutic window of cytotoxic or cytostatic moiety and achieve a tumor targeting of this class of anti-tumor compounds. The inventive prodrug conjugates consist of an αᵥß₃ integrin binding moiety which is linked to camptothecin moiety via a peptide linker, which is cleavable by proteases present in the tumor microenvironment to release the parent camptothecin compound at the site of action. Enzymes present in the tumor microenvironment include, but not limited to, neutrophil elastase. A prodrug may be converted into the parent drug in vivo via an enzymatic or a chemical process.

In some embodiments, a prodrug is activated upon enzymatic cleavage (e.g., a tumor-associated enzyme such as neutrophil elastase). In some embodiments, the prodrug contains a peptide sequence that is recognized by a given enzyme. In some embodiments, a prodrug is selectively cleaved by a specific enzyme. In some embodiments, N-methylation of a peptide moiety enhances selectivity for a specific enzyme (e.g., legumain) over other enzymes (e.g., proteases such as cathespsins).

In some embodiments, a prodrug further comprises a non-peptidic prodrug moiety (e.g., an ester) which is released in vivo. Non-peptidic prodrug moiety indicates that the bond being metablized or broken to release the active agent is not a peptide bond (—C(O)—NH—). In some embodiments, the non-peptidic prodrug moiety is an alkyl ester (including substitutions such as alkyl substitutions on the alkyl group). In some embodiments, the non-peptidic prodrug moiety is an ester of an amino acid, such as an aspartate or glutamate residue. In some embodiments, the alkyl ester prodrug is indicated by Asp* or Glu*, indicating an alkyl (e.g., substituted alkyl) group is masking the carboxylic acid moiety of the side-chain. In some embodiments, provided herein are prodrug compounds wherein an alkylamine (—C₁₋₆alkyl-NR₂) or alkylaminium (—C₁₋₆alkyl-NR₃^{m}) group is released in vivo (where “R” as used here is hydrogen or C₁₋₆alkyl) from an amino acid ester (e.g., Asp* or Glu*) to liberate the acid moiety. In some embodiments, a non-peptidic prodrug moiety (e.g., an ester (e.g., an alkylamine or alkylaminium ester)) is released (i.e., cleaved) in plasma, while an enzymatically cleavable moiety remains intact (e.g., when the cleaving enzyme is not present or abundant). In some embodiments, the prodrug ester is slowly cleaved in plasma with no detectable release of the enzymatically cleavable moiety or the payload (e.g., the cytotoxic or cytostatic ligand) attached thereto. In some embodiments, the non-peptidic (i.e., ester) prodrug moiety is cleaved before the peptidic prodrug moiety. In some embodiments, the non-peptidic (i.e., ester) prodrug moiety is cleaved independently of protease activity (e.g., non-proteolytic cleavage). In some embodiments, the non-peptidic (i.e., ester) prodrug moiety is cleaved to a degree of about 50% (±5%) slowly. As used here, “slowly” means the prodrug is cleaved to a degree of about 50% after at least two hours in plasma. In some embodiments, slow cleavage constitutes about 50% prodrug release after about 2, about 3, about 4, about 5, about 6, about 8, about 10, or about 12 hours in plasma. In some embodiments, slow cleavage constitutes about 50% prodrug release after about 2 hours to about 12 hours in plasma. In some embodiments, an ester prodrug is cleaved to a degree of about 50% after about 2 hours to about 6 hours in plasma. In some embodiments, an ester prodrug is cleaved to a degree of about 50% after about 2 hours to about 4 hours in plasma. As described previously, the non-peptidic (i.e., ester) prodrug moiety is cleaved independently (e.g., in the absence or presence) of proteolytic enzymes such as neutrophil elastase. In some embodiments, the ester prodrug moiety is cleaved to a degree of about 50% after about 3 or 4 hours (and within about 6 hours) of administration in vivo. The degradation product of such a prodrug is, in some embodiments, the parent conjugate sans alkyl ester, meaning the enzymatically cleavable portion, cytotoxic or cytostatic moiety, linker/spacer, and integrin binder remain intact. In some embodiments, enzymatic cleavage takes place after release of the ester prodrug moiety. In some embodiments, the protease responsible for cleaving the enzymatically cleavable moiety recognizes the free Asp or Glu residue, but not the Asp* or Glu* prodrug ester.

Prodrugs of the compounds described herein include, but not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (e.g., the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a conjugate described herein as set forth herein are included within the scope of the claims. In some cases, some of the herein-described compounds is a prodrug for another derivative or active compound.

In some embodiments, any one of the hydroxyl group(s), amino group(s), or carboxylic acid group(s) are functionalized in a suitable manner to provide a prodrug moiety. In some embodiments, the prodrug moiety is as described above.

### Pharmaceutical Compositions

The present invention provides a pharmaceutical composition comprising a compound of formulae disclosed herein, or a pharmaceutically acceptable salt thereof; or a stereoisomer or mixture of stereoisomers thereof, and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated to provide slow or controlled release of the active ingredient therein. All formulations for oral administration should be in dosages suitable for such administration. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

### Compounds for Use in Treating Diseases or Disorders

In some embodiments, the present invention provides compounds of formulae disclosed herein, or a pharmaceutically acceptable salt thereof; or a stereoisomer or mixture of stereoisomers thereof, for the treatment of a disease or disorder.

In some embodiments, the present invention provides compounds of formulae disclosed herein, or a pharmaceutically acceptable salt thereof; or a stereoisomer or mixture of stereoisomers thereof, for use in the manufacture of a medicament for treating a disease or disorder described herein.

In some embodiments, the present invention provides compounds of formulae disclosed herein, or a pharmaceutically acceptable salt thereof; or a stereoisomer or mixture of stereoisomers thereof; for the treatment of a hyperproliferative disorder.

In some embodiments, the present invention provides compounds of formulae disclosed herein, or a pharmaceutically acceptable salt thereof; or a stereoisomer or mixture of stereoisomers thereof, wherein for the treatment of an autoimmune disorder.

In some embodiments, the present invention provides compounds of formulae disclosed herein, or a pharmaceutically acceptable salt thereof; or a stereoisomer or mixture of stereoisomers thereof, for the treatment of an ophthalmological disease or disorder. In some embodiments, the ophthalmological disease or disorder is macular degeneration. In some embodiments, the ophthalmological disease or disorder is uveal melanoma.

In some embodiments, the present invention provides compounds of formulae disclosed herein, or a pharmaceutically acceptable salt thereof; or a stereoisomer or mixture of stereoisomers thereof, wherein for the treatment of a skin disorder. In some embodiments, the skin disorder is psoriasis.

In some embodiments, the present invention provides compounds of formulae disclosed herein, or a pharmaceutically acceptable salt thereof; or a stereoisomer or mixture of stereoisomers thereof, for the treatment of a cancer. In some embodiments, the cancer is an invasive cancer. In some embodiments, the cancer is a metastatic cancer. In some embodiments, the cancer is of the breast, respiratory tract, brain, reproductive organs, digestive tract, urinary tract, eye, liver, skin, head and neck, thyroid, parathyroid or a metastasis thereof.

In some embodiments, the cancer of the breast is invasive ductal carcinoma, invasive lobular carcinoma, ductal carcinoma in situ, lobular carcinoma in situ, inflammatory breast cancer, basal breast cancer, or triple negative breast cancer.

In some embodiments, the cancer of the respiratory tract is small-cell, non-small-cell lung carcinoma, bronchial adenoma, or pleuropulmonary blastoma.

In some embodiments, the cancer of the brain is a brain stem glioma, hypothalmic glioma, glioblastoma, cerebellar astrocytoma, cerebral astrocytoma, medulloblastoma, ependymoma, neuroectodermal tumor, or pineal tumor.

In some embodiments, the cancer of the reproductive organs is a prostate cancer, testicular cancer, endometrial cancer, cervical cancer, ovarian cancer, vaginal cancer, vulvar cancer, or a sarcoma of the uterus.

In some embodiments, the cancer of the digestive tract is anal cancer, colon cancer, colorectal cancer, esophageal cancer, gallbladder cancer, gastric cancer, pancreatic cancer, rectal cancer, small intestinal cancer, or salivary gland cancer.

In some embodiments, the cancer of the urinary tract is bladder cancer, penile cancer, kidney cancer, renal pelvis cancer, ureter cancer, or urethral cancer.

In some embodiments, the cancer of the eye is intraocular melanoma or retinoblastoma.

In some embodiments, the cancer of the liver is to hepatocellular carcinoma, liver cell carcinoma with or without fibrolamellar variant, cholangiocarcinoma, intrahepatic bile duct carcinoma, or mixed hepatocellular cholangiocarcinoma.

In some embodiments, the cancer of the skin is squamous cell carcinoma, Kaposi's sarcoma, malignant melanoma, Merkel cell skin cancer, or non-melanoma skin cancer.

In some embodiments, the cancer of the head-and-neck is laryngeal cancer, hypopharyngeal cancer, nasopharyngeal cancer, oropharyngeal cancer, or a lip and oral cavity cancer.

In some embodiments, the cancer is a sarcoma. In some embodiments, the sarcoma is Ewing sarcoma, osteosarcoma, or fibrosarcoma. In some embodiments, the sarcoma is a sarcoma of the soft tissue, osteosarcoma, malignant fibrous histiocytoma, lymphosarcoma, or rhabdomyosarcoma

In some embodiments, the cancer is a lymphoma. In some embodiments, the lymphoma is AIDS-related lymphoma, non-Hodgkin's lymphoma, cutaneous T-cell lymphoma, Hodgkin's disease, or lymphoma of the central nervous system.

In some embodiments, the cancer is a leukemia. In some embodiments, the leukemia is acute myeloid leukemia, acute lymphoblastic leukemia, chronic lymphocytic leukemia, chronic myelogenous leukemia, or hairy cell leukemia.

In some embodiments, the cancer is breast cancer, colon cancer, renal cancer, or lung cancer.

### Methods of Dosing and Treatment Regimens

In some embodiments, the present invention provides a method of treating a disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the compound used in the method of treating a disease or disorder is a compound of Formula (A), Formula (B), Formula (C), Formula (D), Formula (I), Formula (I-A), Formula (I-B), Formula (I-C), Formula (I-D), Formula (II), Formula (II-A), Formula (II-B), Formula (II-C), Formula (II-D), Formula (III-A), Formula (III-B), Formula (III-C), or Formula (III-D), or a pharmaceutically acceptable salt thereof, or a stereoisomer or mixture of stereoisomers thereof.

In some embodiments, the present invention provides a method of treating a hyperproliferative disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating an autoimmune disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating an ophthalmological disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating macular degeneration in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a skin disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating psoriasis in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer in a subject, comprising administering a therapeutically effective amount of a compound of formulae disclosed herein, or a pharmaceutically acceptable salt thereof, or a stereoisomer or mixture of stereoisomers thereof; or a pharmaceutical composition thereof, to an individual in need thereof. In some embodiments, the present invention provides a method of treating an invasive cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a metastatic cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the breast, respiratory tract, brain, reproductive organs, digestive tract, urinary tract, eye, liver, skin, head and neck, thyroid, parathyroid or a metastasis thereof in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the breast in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating an invasive ductal carcinoma, invasive lobular carcinoma, ductal carcinoma in situ, lobular carcinoma in situ, inflammatory breast cancer, basal breast cancer, or triple negative breast cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the respiratory tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a small-cell lung carcinoma, non-small-cell lung carcinoma, bronchial adenoma, or pleuropulmonary blastoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the brain in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a brain stem glioma, hypothalmic glioma, glioblastoma, cerebellar astrocytoma, cerebral astrocytoma, medulloblastoma, ependymoma, neuroectodermal tumor, or pineal tumor in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the reproductive organs in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating prostate cancer, testicular cancer, endometrial cancer, cervical cancer, ovarian cancer, vaginal cancer, vulvar cancer, or a sarcoma of the uterus in a subject, comprising administering a therapeutically effective amount of a compound of formulae disclosed herein, or a pharmaceutically acceptable salt thereof; or a stereoisomer or mixture of stereoisomers thereof, or a pharmaceutical composition thereof, to an individual in need thereof.

In some embodiments, the present invention provides a method of treating a cancer of the digestive tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating anal cancer, colon cancer, colorectal cancer, esophageal cancer, gallbladder cancer, gastric cancer, pancreatic cancer, rectal cancer, small intestinal cancer, or salivary gland cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the urinary tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating bladder cancer, penile cancer, kidney cancer, renal pelvis cancer, ureter cancer, or urethral cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the eye in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating intraocular melanoma or retinoblastoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the liver in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating hepatocellular carcinoma, liver cell carcinoma with or without fibrolamellar variant, cholangiocarcinoma, intrahepatic bile duct carcinoma, or mixed hepatocellular cholangiocarcinoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the skin in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a squamous cell carcinoma, Kaposi's sarcoma, malignant melanoma, Merkel cell skin cancer, or non-melanoma skin cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the head-and-neck in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating laryngeal cancer, hypopharyngeal cancer, nasopharyngeal cancer, oropharyngeal cancer, or a lip and oral cavity cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a sarcoma in a subject, comprising administering a therapeutically effective amount of a compound of formulae disclosed herein, or a pharmaceutically acceptable salt thereof, or a stereoisomer or mixture of stereoisomers thereof; or a pharmaceutical composition thereof, to an individual in need thereof. In some embodiments, the present invention provides a method of treating Ewing sarcoma, osteosarcoma, fibrosarcoma, sarcoma of the soft tissue, malignant fibrous histiocytoma, lymphosarcoma, or rhabdomyosarcoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating breast cancer, colon cancer, renal cancer, or lung cancer in a subject, comprising administering a therapeutically effective amount of a compound of formulae disclosed herein, or a pharmaceutically acceptable salt thereof; or a stereoisomer or mixture of stereoisomers thereof, or a pharmaceutical composition thereof, to an individual in need thereof.

In another aspect, provided herein is a method of treating a disease or disorder, the method comprising administering to a subject in need thereof a therapeutically effective amount of a pharmaceutical composition comprising any one of the compounds of the formulae disclosed herein, or a pharmaceutically acceptable salt thereof, or a stereoisomer or mixture of stereoisomers thereof; or a pharmaceutical composition thereof, to an individual in need thereof.

The total amount of the active compound to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The present invention further provides the use of the compound of the invention for the preparation of a pharmaceutical compositions for the treatment of the aforesaid disorders.

### Administration

It is possible for the compounds according to the invention to have systemic or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly or in a modified manner, such as, for example, tablets (e.g., uncoated or coated tablets, for example with enteric or controlled release coatings that dissolve with a delay or are insoluble), orally-disintegrating tablets, films/wafers, films/lyophylisates, capsules (e.g., hard or soft gelatin capsules), sugar-coated tablets, granules, pellets, powders, emulsions, suspensions, aerosols or solutions. It is possible to incorporate the compounds according to the invention in crystalline or amorphised or dissolved form into said dosage forms.

Parenteral administration can be affected with avoidance of an absorption step (for example intravenous, intraarterial, intracardial, intraspinal or intralumbal) or with inclusion of absorption (for example, intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders. In some embodiments, a compound disclosed herein is administered via injection (e.g., parenteral injection). In some embodiments, a compound disclosed herein is formulated for injection. In some embodiments, a compound disclosed herein is formulated with a pharmaceutically acceptable excipient (e.g., a carrier or vehicle, e.g., an aqueous vehicle)) for injection. In some embodiments, the injection is intravenous. In some embodiments, the intravenous injection is an intravenous infusion.

Examples which are suitable for other administration routes are pharmaceutical forms for inhalation (e.g., powder inhalers, nebulizers, etc.), nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (e.g., lotions, mixturae agitandae, etc.), lipophilic suspensions, emulsions, ointments, creams, transdermal therapeutic systems (e.g., patches, etc.), milk, pastes, foams, dusting powders, implants or stents.

The compounds according to the invention can be incorporated into the stated administration forms. This can be affected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colorants (for example inorganic pigments such as, for example, iron oxides, titanium dioxide),
flavorings, sweeteners, flavor- or odor-masking agents.

The present invention furthermore relates to a pharmaceutical composition which comprise at least one compound according to the invention, conventionally together with one or more pharmaceutically suitable excipient(s), and to their use according to the present invention.

### Combination Treatments

In certain instances, it is appropriate to administer at least one conjugate described herein, or a pharmaceutically acceptable salt thereof, in combination with one or more other therapeutic agents.

In accordance with another aspect, the present invention covers pharmaceutical compositions, in particular, medicines, comprising at least one compound disclosed herein in the present invention and at least one or more further active ingredients, in particular, for the treatment or prophylaxis of a hyperproliferative disorder.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known active ingredients for the treatment and/or prophylaxis of a hyperproliferative disorder.

Examples of active ingredients for the treatment and/or prophylaxis of a hyperproliferative disorder include, but not limited to, 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polatuzumab, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, venetoclax, vinblastine, vincristine, vindesine, vinflunine, vinorelbine, vismodegib, vorinostat, vorozole, yttrium-90 glass microspheres, zanubrutinib, zinostatin, zinostatin stimalamer, zoledronic acid, and zorubicin.

In one embodiment, the therapeutic effectiveness of one of the compounds described herein is enhanced by administration of an adjuvant (e.g., by itself the adjuvant has minimal therapeutic benefit, but in combination with another therapeutic agent, the overall therapeutic benefit to the patient is enhanced). In some embodiments, the benefit experienced by a patient is increased by administering one of the compounds described herein with another agent (which also includes a therapeutic regimen) that also has therapeutic benefit.

In one specific embodiment, a conjugate described herein, or a pharmaceutically acceptable salt thereof, is co-administered with a second therapeutic agent, wherein the conjugate described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The conjugates described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Definitions

Unless otherwise defined, all of the technical terms used herein have the same meaning as commonly understood by one of ordinary skill in the art in the field to which this disclosure belongs.

Unless otherwise stated, the following terms used in this application have the definitions given below.

The section headings used herein are for organizational purposes only and are not to be construed as limiting the subject matter described.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the term “about” a number refers to that number plus or minus 10% of that number. The term ‘about’ when used in the context of a range refers to that range minus 10% of its lowest value and plus 10% of its greatest value.

Whenever the term “at least”, “greater than”, or “greater than or equal to” precedes the first numerical value in a series of two or more numerical values, the term “at least”, “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than”, “less than”, or “less than or equal to” precedes the first numerical value in a series of two or more numerical values, the term “no more than”, “less than”, or “less than or equal to” applies to each of the numerical values in that series of numerical values. For example, less than or equal to 3, 2, or 1 is equivalent to less than or equal to 3, less than or equal to 2, or less than or equal to 1.

The use of the term “including” as well as other forms, such as “include”, “includes,” and “included,” is not limiting.

The phrase “one or more pharmaceutically acceptable excipients” is used herein to refer that one pharmaceutically acceptable excipient or more than one pharmaceutically acceptable excipient may be used in any combination. The number of pharmaceutically acceptable excipients to be used may be at the discretion of a person skilled in the art, and they may be of different types.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “specific pH” herein refers to a desired pH value of a solvent or a solution comprising CocE obtained by adding a pharmaceutically acceptable excipient.

The term “% wt” is used to describe the weight percentage of one component in a mixture of components.

The term “a trace” herein refers more than, but close to about 0%.

The term “about” herein refers to ±10%, ±20%, ±30%, ±40%, or ±50%, or to the nearest significant figure.

An “amino acid” refers to a natural amino acid or a non-natural amino acid. The natural amino acid is a naturally-occurring amino acid, including, but not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. The non-natural amino acid may be incorporated into peptides for many different reasons, for instance, to increase the activity or selectivity and plasma stability of peptides or to induce or stabilize secondary structure (e.g., helices, sheets, or turns). The non-natural amino acid includes, but not limited to, N-alkyl (e.g., N-methyl) amino acids, stereoisomers (D-amino acids), homo amino acids, alpha-methyl amino acids, beta¹ amino acids, beta² amino acids, beta³ amino acids, peptides, aminocyclohexanecarboxylate (ACHC), and non-protein amino acids, such as, citrulline, ornithine, homoalanine, norvaline, norleucine, etc. In some embodiment, the non-natural amino acid includes N-methyl alanine and citrulline.

As used herein, C₁-Cₓ includes C₁-C₂, C₁-C₃ . . . C₁-Cₓ. By way of example only, a group designated as “C₁-C₆” indicates that there are one to six carbon atoms in the moiety, e.g., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, e.g., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, e.g. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (or vinyl), propenyl (or allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term, “heteroalkyl” generally refers to a straight-chain and/or branched hydrocarbon chain which has 1 to 30 carbon atoms and may be interrupted once or more than once by one or more of the groups —O—, —S—, —C(═O)—, —S(═O)—, —S(═O)₂—, —NR^{y}—, —NR^{Y}C(═O)—, —C(═O)—NR^{y}—, —NR^{Y}NR^{Y}—, —S(═O)₂—NR^{y}NR^{Y}—, —C(═O)—NR^{y}NR^{Y}—, —CR^{X}═N—O—, and where the hydrocarbon chain including the side chains, if present, may be substituted by —NH—C(═O)—NH₂, —C(═O)—OH, —OH, —NH₂, —NH—C(═NNH₂)—, sulfonamide, sulfone, sulfoxide, sulfonic acid, sulfamide, or a combination thereof. In this context, R^{y} in each case is —H, phenyl, C₁-C₁₀-alkyl, C₂-C₁₀-alkenyl or C₂-C₁₀-alkynyl, which may in turn be substituted in each case by —NHC(O)NH₂, —COOH, —OH, —NH₂, —NH—C(═NNH₂)—, sulfonamide, sulfone, sulfoxide, sulfonic acid, sulfamide, or a combination thereof. In this context, R^{X} is —H, C₁-C₃-alkyl or phenyl. The terms “heteroalkyl-aryl” and “heteroalkyl-heteroaryl” as used herein generally refer to a heteroalkyl group (as defined above) substituted with an aromatic carbocycle or an aromatic heterocycle respectively; and wherein each is optionally substituted.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (or rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (or an arylene group).

As used herein, the term “aralkyl” generally refers to a monocyclic aromatic carbocycle (e.g., phenyl), to which a C₁₋₄-alkyl group is bonded. Illustrative aralkyl groups include benzyl and ethylphenyl.

The term “cycloalkyl” refers to a monocyclic or polycyclic aliphatic, non-aromatic radical, wherein each of the atoms forming the ring (or skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl. The term “heteroaralkyl” as used herein generally refers to a lower alkyl group (e.g., C₁₋₆ alkyl) that is substituted with a heteraryl group. Illustrative examples include —CH₂-pyridine, —CH₂CH₂-triazole, etc.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R^{s} groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O) C₁-C₆alkyl, —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

“Pharmaceutically acceptable,” as used herein, refers a material, such as a carrier or diluent, which does not abrogate the biological activity or properties of the compound, and is relatively nontoxic at the concentration or amount used, e.g., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a conjugate described herein, or a pharmaceutically acceptable salt thereof, and a co-agent, are both administered to a patient simultaneously in the form of a single entity or dosage. The term “non-fixed combination” means that the active ingredients, e.g. a conjugate described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating”, or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically or therapeutically.

The term “tumor microenvironment,” or “TME,” as used herein, includes the environment around a tumor, including the surrounding blood vessels, immune cells, fibroblasts, signaling molecules and the extracellular matrix. A tumor microenvironment includes tumor cells, necrotic cells, and cells surrounding the tumor which may be influenced (e.g., by signaling factors, growth factors, growth factor receptors, hormones, cytokines, and the like) by a tumor. A tumor microenvironment includes the area immediately surrounding a tumor (e.g., within about 1 cm of the tumor). In some instances, a tumor microenvironment includes cells, tissues, blood vessels, and other biological materials wherein the pH is lower than analogous tissues that are not within the vicinity or under the influence of a tumor. A tumor microenvironment may be defined by having a low (i.e., acidic) pH.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds disclosed herein, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

### Examples

As used above, and throughout the description of the invention, the following abbreviations, unless otherwise indicated, shall be understood to have the following meanings:

### Abbreviations

786-0 human tumour cell line
A431NS human tumour cell line
A549 human tumour cell line
ABCBl ATP-binding cassette sub-family B member I (synonym for P-gp and MDRl)
abs. absolute
Abu α(alpha)-amino butyric acid
Ac acetyl
ACN acetonitrile
ADC antibody drug conjugate
A2DC antibody 2 drug conjugate
aq. aqueous, aqueous solution
ATP adenosine triphosphate
BCRP breast cancer resistance protein, an efflux transporter
BEP 2-bromo-1-ethylpyridinium tetrafluoroborate
Boc tert-butoxycarbonyl
br. broad (in NMR)
Ex. Example
BxPC3 human tumour cell line
ca. circa, about
C-DAR cysteine drug to antibody ratio (linker attached to cystein residues)
C₁ chemical ionization (in MS)
D doublet (in NMR)
D day(s)
TLC thin-layer chromatography
DCI direct chemical ionization (in MS)
DCM dichloromethane
Dd doublet of doublets (in NMR)
DIEA N, N diisopropyl ethyl amine (Hünig's base)
DMAP 4-N,N-dimethylaminopyridine
DME 1,2-dimethoxyethane
DMEM Dulbecco's modified eagle medium (standardized nutrient medium for cell culture)
DMF N,N-dimethylformamide
DMSO dimethyl sulphoxide
DAR drug to antibody ratio
DPBS Dulbecco's phosphate-buffered salt solution
Dt doublet of triplets (in NMR)
DTT DL-dithiothreitol
d. Th. of theory (in chemical yield)
EDC N′-(3-dimethylaminopropyl)-N-ethylcarbodi-imide hydrochloride
EGFR epidermal growth factor receptor
ee: enantiomeric excess
EI electron impact ionization (in MS)
ELISA enzyme-linked immunosorbent assay
eq. equivalent(s)
ESI electrospray ionization (in MS)
FCS foetal calf serum
Fmoc (9H-fluoren-9-ylmethoxy)carbonyl
sat. saturated
GTP guanosine-5′-triphosphate
h hour(s)
HATU O-(7-azabenzotriazol-1-yl)-N,N,N′,N′-tetramethyluronium hexafluoro phosphate
HCT-116 human tumour cell line
HEPES 4-(2-hydroxyethyl)piperazine-1-ethane-sulphonic acid
HOAc acetic acid
HOAt 1-hydroxy-7-azabenzotriazole
HOBt 1-hydroxy-1H-benzotriazole hydrate
HOSu N-hydroxysuccinimide
HPLC high-pressure, high-performance liquid chromatography
HT29 human tumour cell line
IC50 half-maximal inhibitory concentration
i.m. intramuscularly, administration into the muscle
i.v. intravenously, administration into the vein
K-DAR lysine drug to antibody ratio (linker attached to lysine residues)
KPL-4 human tumour cell lines
KU-19-19 human tumour cell line
LC-MS liquid chromatography-coupled mass spectrometry
LLC-PKl cells Lewis lung carcinoma pork kidney cell line
L-MDR human MDRl transfected LLC-PKl cells
LoVo human tumour cell line
m multiplet (in NMR)
Me methyl
MDRl Multidrug resistance protein I
MeCN acetonitrile
min minute(s)
MS mass spectrometry
MTBE methyl tert.-butyl ether
MTT 3-(4,5-dimethylthiazol-2-yl)-2,5-diphenyl-2H-tetrazolium bromide
NCI-H292 human tumour cell line
NCI-H520 human tumour cell line
NMM N-methylmorpholine
NMP N-methyl-2-pyrrolidinone
NMR nuclear magnetic resonance spectrometry
NMRI mouse strain originating from the Naval Medical Research Institute
Nude mice experimental animals
NSCLC non small cell lung cancer
PBS phosphate-buffered salt solution
Pd/C palladium on activated carbon
P-gp P-glycoprotein, a transporter protein
PNGaseF enzyme for cleaving sugar
Quant quantitative (in yield)
quart quartet (in NMR)
quint quintet (in NMR)
RT room temperature
Rt retention time (in HPLC)
s singlet (in NMR)
s.c. subcutaneously, administration under the skin
SCC-4 human tumour cell line
SCC-9 human tumour cell line
SCID mice test mice with severe combined immunodeficiency
SK-HEP-I human tumour cell line
t triplet (in NMR)
T3P® 2,4,6-tripropyl-1,3,5,2,4,6-trioxatriphosphinane 2,4,6-trioxide
TBAF tetra-n-butylammonium fluoride
TCEP tris(2-carboxyethyl)phosphine
TEMPO (2,2,6,6-tetramethylpiperidin-1-yl)oxyl
tert tertiary
TFA trifluoroacetic acid
THF tetrahydrofuran
TLC thin-layer chromatography
UV ultraviolet spectrometry
v/v volume to volume ratio (of a solution)
Z benzyloxycarbonyl

The following examples are provided for illustrative purposes only and not to limit the scope of the claims provided herein.

### Analytical Methods (LC-MS)

The following examples are provided for illustrative purposes only and not to limit the scope of the claims provided herein.

### Analytical Methods (LC-MS)

General method 1 (LC-MS): Instrument: Waters ACQUITY SQD UPLC System; Column: Waters Acquity UPLC HSS T3 1.8μ 50×1 mm; Eluent A: 1 l Water+0.25 mL 99% ige formic acid, Eluent B: 1 l acetonitrile+0.25 mL 99% formic acid; Gradient: 0.0 min 90% A→1.2 min 5% A→2.0 min 5% A Stove: 50° C.; Flow: 0.40 mL/min; UV-Detection: 208-400 nm.

Method 2 (LC-MS): System MS: Thermo Scientific FT-MS; System UHPLC+: Thermo Scientific UltiMate 3000; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm

Method 3 (LC-MS): System MS: Waters TOF instrument; System UPLC: Waters Acquity I-CLASS; Column: Waters, HSST3, 2.1×50 mm, C18 1.8 μm; Eluent A: 1 l Water+0.01% Formic acid; Eluent B: 1 l Acetonitrile+0.01% Formic acid; Gradient: 0.0 min 2% B→0.5 min 2% B→7.5 min 95% B→10.0 min 95% B; Oven: 50° C.; Flow: 1.00 ml/min; UV-Detection: 210 nm

Method 4 (LC-MS): System MS: Thermo Scientific FT-MS; System UHPLC+: Thermo Scientific Vanquish; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm

Method 5 (LC-MS): System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 90% A→1.2 min 5% A→2.0 min 5% A Oven: 50° C.; Flow: 0.40 ml/min; UV-Detection: 210 nm.

Method 6 (LC-MS): System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 95% A→6.0 min 5% A→7.5 min 5% A Oven: 50° C.; Flow: 0.35 ml/min; UV-Detection: 210 nm.

### SYNTHESIS EXAMPLES

### Building Blocks

### Example B1: Preparation of (3R)-3-{[(4-aminophenyl)carbamoyl]amino}-3-{3-[({3-[(propyl carbamoyl)amino] phenyl} sulfonyl)amino]phenyl}propanoic acid (Building Block 1)

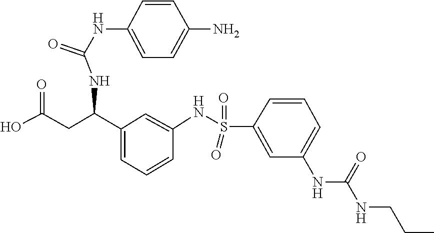 [SMILES: CCCNC(=O)Nc1cccc(S(=O)(=O)Nc2cccc([C@@H](CC(=O)O)NC(=O)Nc3ccc(N)cc3)c2)c1]

The synthesis of Building block 1 has been described in WO2020/094471

¹H-NMR (500 MHz, D₄-methanol): δ=0.93 (t, 3H), 1.5 (m, 2H), 2.74 (d, 2H), 3.1 (dt, 2H), 5.15 (t, 1H), 6.68 (d, 2H), 6.85 (d, 1H), 7.05 (d, 1H), 7.1 (d, 1H), 7.13 (t, 1H), 7.28-7.4 (m, 3H), 7.6 (s, 1H), 7.66 (d, 1H).

### Example B2: Preparation of (3R)-3-{[(4-{[(4-nitrophenoxy)carbonyl]amino} phenyl)carbamoyl]amino}-3-{3-[({3-[(propylcarbamoyl)amino]phenyl} sulfonyl)amino]phenyl}propanoic acid (Building Block 2)

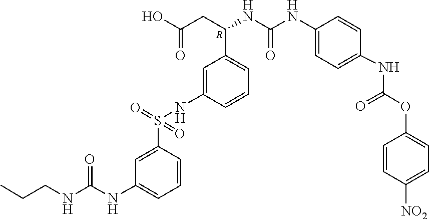

The synthesis of building block 2 has been described in WO2020/094471.

### Example B3: Preparation of (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building Block 3)

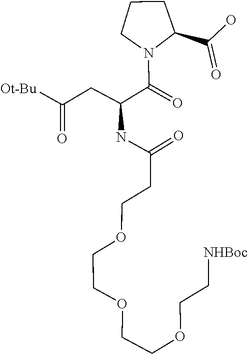

Building block 3 was synthesized using classical methods of peptide synthesis starting with the coupling of Z-Asp(OtBu)-OH with benzyl L-prolinate hydrochloride (1:1) in THE in the presence of T3P and DIPEA and subsequent removal of the Z-protecting group as well as the benzyl ester by hydrogenolysis over Pd/C to give (2S)-1-[(2S)-2-amino-4-tert-butoxy-4-oxobutanoyl] pyrrolidine-2-carboxylic acid. This partially protected dipeptide was acylated with tert-butyl{2-[2-(2-{3-[(2,5-dioxopyrrolidin-1-yl)oxy]-3-oxopropoxy}ethoxy)ethoxy]ethyl} carbamate to give the title compound. Tert-butyl{2-[2-(2-{3-[(2,5-dioxopyrrolidin-1-yl)oxy]-3-oxopropoxy}ethoxy) ethoxy]ethyl}carbamate was previously prepared by reacting 2,2-dimethyl-4-oxo-3,8,11,14-tetraoxa-5-azaheptadecan-17-oic acid with N-Hydroxysuccinimide in dioxane in the presence of EDCI. LC-MS: Rₜ=0.81 min; MS (ESIpos): m/z=590 [M+H]⁺.

### Example B4: Preparation of N-{2-[{2-[(tert-butoxycarbonyl)(methyl)amino]ethyl} (methyl)amino]ethyl}-N-methylglycine (Building Block 4)

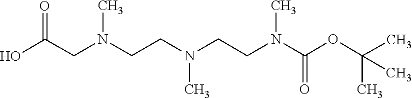 [SMILES: CN(CCN(C)CC(=O)O)CCN(C)C(=O)OC(C)(C)C]

Initially, tert-butyl methyl(2-{methyl[2-(methylamino)ethyl] amino}ethyl)carbamate was obtained starting from commercially available N,N′-dimethyl-N-[2-(methylamino)ethyl]ethane-1,2-diamine.

Benzyl bromoacetate (609 mg, 2.66 mmol) was initially dissolved under argon in acetonitrile (25 mL) and potassium carbonate (734 mg, 5.31 mmol) was added. The solution was cooled to 0° C. and a solution of tert-butyl methyl(2-{methyl[2-(methylamino)ethyl] amino}ethyl)carbamate (652 mg, 2.66 mmol) in acetonitrile was added. The batch was stirred at rt for 1 h and subsequently filtered. The filtrate was evaporated in vacuo and the remaining residue was purified by prep. HPLC. Relevant fractions were collected and evaporated to dryness. 665 mg (97% purity, 62% yield) of the protected intermediate benzyl N-{2-[{2-[(tert-butoxycarbonyl)(methyl)amino]ethyl}(methyl)amino]ethyl}-N-methylglycinate were obtained as a colorless oil. This intermediate (687 mg, 1.75 mmol) was dissolved in DCM/methanol and hydrogenated over 10% Pd on charcoal at rt for 2 h. The catalyst was filtered off and the filtrate was concentrated in vacuo to give Building block 4 (555 mg, 61% purity, 64% yield) as a colorless oil. LC-MS: Rₜ=0.74 min; MS (ESIpos): m/z=304 [M+H]⁺

### Example B5: Preparation of N-{2-[{2-[(tert-butoxycarbonyl)(methyl)amino]ethyl}(methyl)amino]ethyl}-N-methylglycyl-L-asparaginyl-L-proline (Building Block 5)

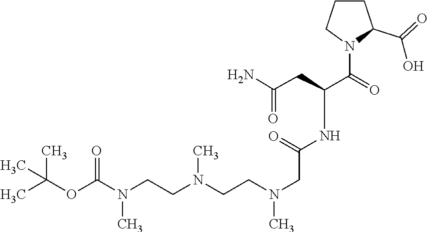 [SMILES: CN(CCN(C)CC(=O)N[C@@H](CC(N)=O)C(=O)N1CCC[C@H]1C(=O)O)CCN(C)C(=O)OC(C)(C)C]

Building block 5 was synthesized using classical peptide synthesis methods starting with the coupling of Boc-Asn with benzyl L-prolinate hydrochloride (1:1) in DMF in the presence of HATU and N,N-Diisopropylethylamine and subsequent removal of the Boc-protecting group with TFA in DCM. This partially protected dipeptide was acylated with N-{2-[{2-[(tert-butoxycarbonyl)(methyl)amino]ethyl}(methyl)amino]ethyl}-N-methylglycine (Building block 4) in DMF in the presence of HATU and N, N-Diisopropylethylamine. In the final step the benzylester was removed by hydrogenolysis over 10% Pd/charcoal. LC-MS: Rₜ=0.81 min; MS (ESIpos): m/z=515 [M+H]⁺

### Example B6: Preparation of N-(tert-butoxycarbonyl)-L-alanyl-N-methyl-L-alanine (Building Block 6)

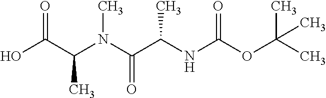 [SMILES: C[C@H](NC(=O)OC(C)(C)C)C(=O)N(C)[C@@H](C)C(=O)O]

Building block 6 was synthesized by coupling 2,5-dioxopyrrolidin-1-yl N-(tert-butoxycarbonyl)-L-alaninate to N-methyl-L-alanine in DMF in the presence of N,N-diisopropylethylamine.

### Example B7: Preparation of N-methyl-N-(2,2,8,11-tetramethyl-4-oxo-3-oxa-5,8,11-triazatridecan-13-yl)glycine (Building Block 7)

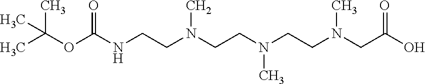 [SMILES: CN(CCNC(=O)OC(C)(C)C)CCN(C)CCN(C)CC(=O)O]

Commercially available N,N′-dimethyl-N-[2-(methylamino) ethyl]ethane-1,2-diamine (500 mg, 3.44 mmol) was dissolved under argon in acetonitrile (50 mL) and potassium carbonate (476 mg, 3.44 mmol) was added. The solution was cooled to 0° C. and a solution of benzyl bromoacetate (394 mg, 1.72 mmol)) in acetonitrile was added. The batch was stirred at rt for 20 h and was filtered subsequently. The filtrate was evaporated and the remaining residue was purified by prep. HPLC. Relevant fractions were collected and evaporated to dryness to yield 476 mg (99% purity, 34% yield) of the protected intermediate as a colorless oil. This intermediate was alkylated reductively in methanol and 2.5 equivalents of acetic acid with tert-butyl (2-oxoethyl)carbamate in the presence of trihydrido(pyridine)boron. Subsequently, the benzylester was removed by hydrogenolysis over 10% Pd/charcoal. LC-MS: Rₜ=0.47 min; MS (ESIpos): m/z=347 [M+H]⁺. ¹H NMR (600 MHz, DMSO-d₆) δ ppm 1.394 (s, 9H) 2.378 (s, 3H) 2.542 (s, 2H) 2.779 (s, 3H) 2.812 (s, 3H) 2.852-2.944 (m, 4H) 3.145 (br t, J=6.26 Hz, 2H) 3.229 (br t, J=5.87 Hz, 1H) 3.262-3.344 (m, 4H) 3.975 (s, 3H) 7.098 (br t, J=5.09 Hz, 1H).

### Example B8: Preparation of N-methyl-N-(2,2,8,11-tetramethyl-4-oxo-3-oxa-5,8,11-triazatridecan-13-yl)glycyl-L-asparaginyl-L-proline (Building Block 8)

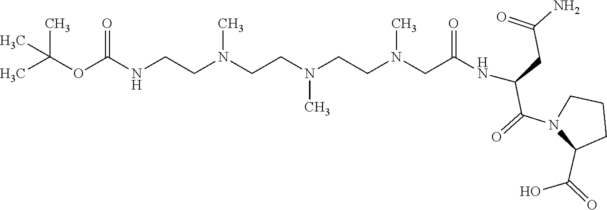 [SMILES: CN(CCNC(=O)OC(C)(C)C)CCN(C)CCN(C)CC(=O)N[C@@H](CC(N)=O)C(=O)N1CCC[C@H]1C(=O)O]

Building block 8 was synthesized using classical peptide synthesis methods starting with the coupling of Boc-Asn with benzyl L-prolinate hydrochloride (1:1) in DMF in the presence of HATU and N, N-diisopropylethylamine and subsequent removal of the Boc-protecting group with TFA in DCM to yield the dipeptide H-L-Asn-L-Pro-OBzl as the trifluoroacetate salt. This partially protected dipeptide was acylated with N-methyl-N-(2,2,8,11-tetramethyl-4-oxo-3-oxa-5,8,11-triazatridecan-13-yl)glycine (Building block 7) in DMF in the presence of HATU and N, N-diisopropylethylamine. In the final step, the benzylester was removed by hydrogenolysis over 10% Pd/charcoal. LC-MS: Rₜ=0.54 min; MS (ESIpos): m/z=558 [M+H]⁺. ¹H NMR (600 MHz, DMSO-d₆) δ ppm 1.393 (s) 1.851-1.956 (m) 2.074 (s) 2.129 (br dd, J=12.13, 8.22 Hz) 2.365-2.413 (m) 2.480-2.516 (m) 2.543 (s) 2.768 (s) 2.820 (s) 2.843-2.906 (m) 3.158 (br t, J=6.06 Hz) 3.217 (br t, J=5.87 Hz) 3.284-3.347 (m) 3.383 (dd, J=8.51, 5.77 Hz) 3.613-3.654 (m) 3.793 (br d, J=13.30 Hz) 4.227 (dd, J=8.61, 4.11 Hz) 4.712-4.752 (m) 4.848-4.902 (m) 4.915-4.955 (m) 6.902 (br s) 6.966 (s) 7.103 (br t, J=5.28 Hz) 7.452 (br s) 7.472 (br s) 8.769-8.817 (m) 8.941 (br d, J=7.63 Hz).

### Example B9: Preparation of (3S)-3-{[(4-aminophenyl)carbamoyl]amino}-3-{3-[({3-[(propyl carbamoyl)amino] phenyl} sulfonyl)amino]phenyl}propanoic acid (Building Block 9)

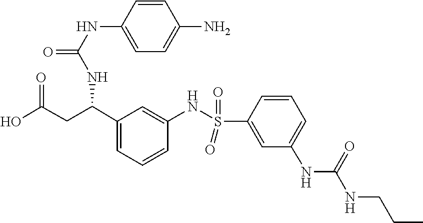 [SMILES: CCCNC(=O)Nc1cccc(S(=O)(=O)Nc2cccc([C@H](CC(=O)O)NC(=O)Nc3ccc(N)cc3)c2)c1]

The synthesis of building block 9 has been described in WO2020/094471.

### Example B10: Preparation of (3S)-3-{[(4-{[(4-nitrophenoxy)carbonyl]amino} phenyl)carbamoyl]amino}-3-{3-[({3-[(propylcarbamoyl)amino]phenyl}sulfonyl)amino]phenyl} propanoic acid (Building Block 10)

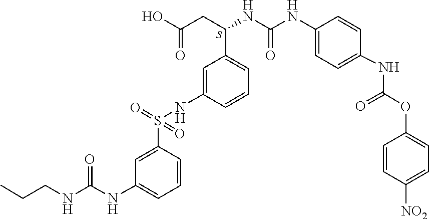

The synthesis of building block 10 has been described in WO2020/094471. LC-MS: Rₜ=0.89 min; MS (ESIpos): m/z=720 [M+H]⁺.

### Intermediates

### Example I1: Preparation of 2-(Benzyloxy)-2-oxoethyl N-(tert-butoxycarbonyl)-L-valinate (Intermediate 1)

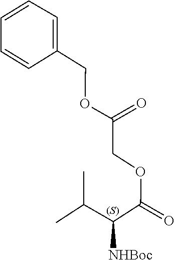

Benzyl hydroxyacetate (1.00 g, 6.02 mmol) was dissolved in DCM (50 mL). N-(tert-butoxycarbonyl)-L-valine (1.31 g, 6.02 mmol), 1-(3-dimethylaminopropyl)-3-ethylcarbodiimide hydrochloride (1.38 g, 7.22 mmol) and DMAP (36.8 mg, 301 μmol) were added. The reaction was stirred at rt overnight and then concentrated in vacuo. The residue was purified by prep. HPLC to give Intermediate 1 (1.27 g, 93% purity, 54% yield) as a colourless foam. LC-MS: Rₜ=2.20 min; MS (ESIpos): m/z=388 [M+Na]⁺

### Example I2: Preparation of {[N-(Tert-butoxycarbonyl)-L-valyl]oxy}acetic acid (Intermediate 2)

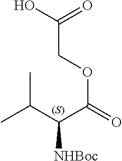

2-(Benzyloxy)-2-oxoethyl N-(tert-butoxycarbonyl)-L-valinate (Intermediate 1) (1.27 g, 3.48 mmol) was dissolved in DCM/methanol (1:1, 100 mL). Pd/C 10% (50.0 mg) was added. The reaction was hydrogenated at rt at normal pressure for 2 h. The catalyst was filtered off and the filtrate was concentrated in vacuo to give Intermediate 2 (822 mg, 80% purity, 69% yield) as a colourless oil. LC-MS: Rₜ=1.42 min; MS (ESIneg): m/z=274 [M−H]⁻.

### Example I3: Preparation of 2-{[(1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(tert-butoxycarbonyl)-L-valinate (Intermediate 3)

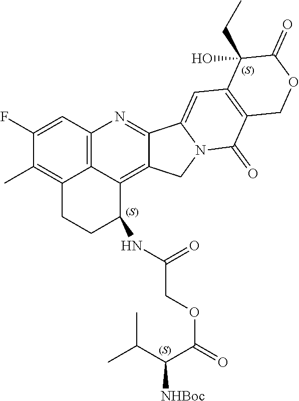

Methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (50.0 mg, 94.1 μmol) was dissolved in ethyl acetate. It was extracted with sat. NaHCO₃ solution. The cloudy org. phase contained undissolved starting material. The aqueous phase was separated off and it was confirmed by HPLC that it contained no starting material. The org. phase was concentrated in vacuo, dissolve in acetonitrile/water and freeze-dried. The residue was re-dissolved in DMF (5.0 mL). {[N-(Tert-butoxycarbonyl)-L-valyl]oxy}acetic acid (Intermediate 2) (51.8 mg, 188 μmol), HATU (46.5 mg, 122 μmol) and DIEA (66 μl, 380 μmol) were added. The reaction was stirred at rt for 1 h and the reaction was evaporated to dryness. The residue was purified by prep. HPLC to give Intermediate 3 (61.0 mg, 95% purity, 89% yield) as a yellow foam. LC-MS: Rₜ=1.95 min; MS (ESIneg): m/z=691 [M−H]⁻.

### Example I4: Preparation of Trifluoroacetic acid-2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl]amino}-2-oxoethyl L-valinate (1/1) (Intermediate 4)

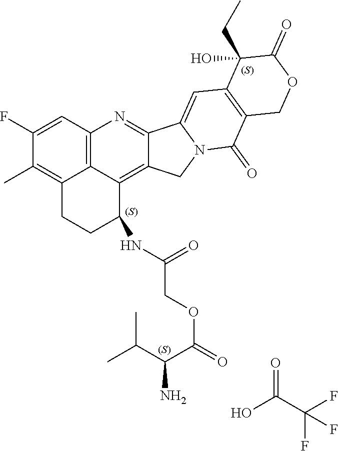

2-{[(1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(tert-butoxycarbonyl)-L-valinate (Intermediate 3) (61.0 mg, 88.1 μmol) was dissolved in DCM (5.0 mL), then TFA (1.0 mL) was added. The batch was stirred for 2 h at rt and then concentrated in vacuo. The residue was dissolved in ACN/water and freeze-dried to give Intermediate 4 (58.0 mg, 93% purity, 86% yield) as a colourless foam. LC-MS: Rₜ=1.13 min; MS (ESIpos): m/z=593 [M+H]⁺.

### Example I5: Preparation of Tert-butyl (19S)-19-[(2S)-2-{[(2S)-1-(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 5)

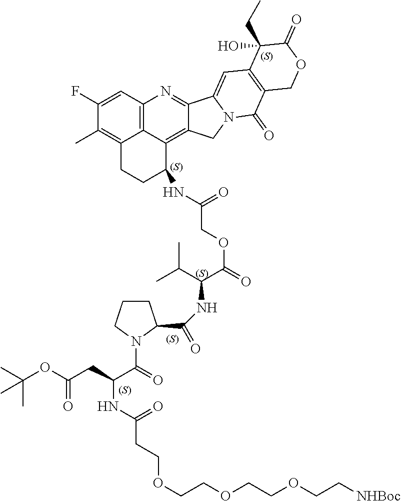

Trifluoroacetic acid-2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl L-valinate (1/1) (Intermediate 4) (58.0 mg, 82.1 μmol) was dissolved in DMF (5.0 mL). (2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) (58.1 mg, 98.5 μmol), HATU (40.6 mg, 107 μmol) and DIEA (57 μl, 330 μmol) were added. The reaction was stirred at rt for 1 h. The reaction was evaporated to dryness and the residue was separated by prep. HPLC to give Intermediate 5 (89.0 mg, 93% yield) as a yellow foam. LC-MS: Rₜ=3.52 min; MS (ESIpos): m/z=1165 [M+H]⁺.

### Example I6: Preparation of 2-{[(1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(3-{2-[2-(2-aminoethoxy)ethoxy]ethoxy}propanoyl)-L-alpha-aspartyl-L-prolyl-L-valinate-trifluoroacetic acid (1/1) (Intermediate 6)

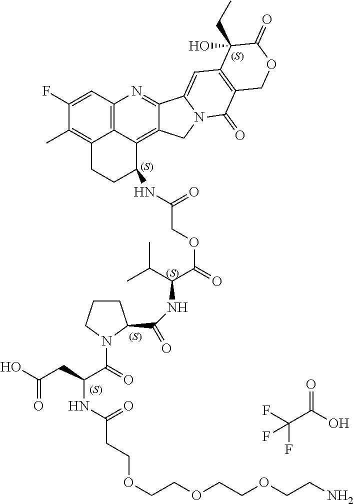

Tert-butyl (19S)-19-[(2S)-2-{[(2S)-1-(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 5) (89.0 mg, 76.4 μmol) was dissolved in DCM (4.3 mL), then TFA (870 μl) was added. The reaction was stirred for 2 h at rt and then concentrated in vacuo. The residue was dissolved in ACN/water and freeze-dried to give Intermediate 6 (85.0 mg, 85% purity, 84% yield) as a yellow foam. LC-MS: Rₜ=2.78 min; MS (ESIpos): m/z=1008 [M+H]⁺.

### Example I7: Preparation of (1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 7)

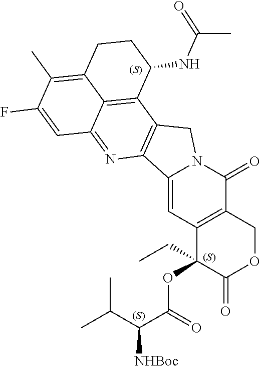

N-[(1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]acetamide (Compound 7, see Example C7) (24.0 mg, 50.3 μmol) was suspended in DCM (4.0 mL), then tert-butyl (4S)-2,5-dioxo-4-(propan-2-yl)-1,3-oxazolidine-3-carboxylate (24.5 mg, 101 μmol) and DMAP (6.14 mg, 50.3 μmol; CAS: 1122-58-3) were added and the reaction was stirred at rt for 2 days. The reaction was concentrated in vacuo and separated by prep. HPLC to give Intermediate 7 (31.9 mg, 100% purity, 94% yield) as a colourless foam. LC-MS: Rₜ=3.63 min; MS (ESIpos): m/z=677 [M+H]⁺.

### Example I8: Preparation of Trifluoroacetic acid-(1S,9S)-1-acetamido-9-ethyl-5-fluoro-4-methyl-10,13-dioxo-2,3,9,10,13,15-hexahydro-1H,12H-benzo[de]pyrano[3′,4′:6,7] indolizino[1,2-b]quinolin-9-yl L-valinate (1/1) (Intermediate 8)

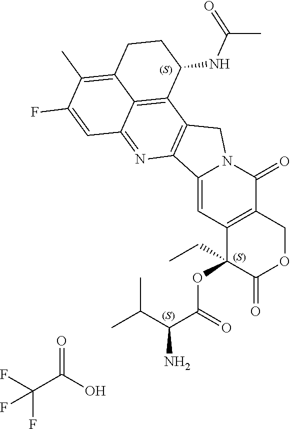

(1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 7) (31.5 mg, 46.5 μmol) was dissolved in DCM (2.6 mL), then TFA (530 μl) was added. The reaction was stirred for 1 h at rt and then concentrated in vacuo. The residue was dissolved in ACN/water and freeze-dried to give Intermediate 8 as a yellow foam (35.0 mg, 97% purity, 105% yield). LC-MS: Rₜ=1.11 min; MS (ESIpos): m/z=577 [M+H]⁺.

### Example I9: Preparation of Tert-butyl (19S)-19-[(2S)-2-{[(2S)-1-{[(1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 9)

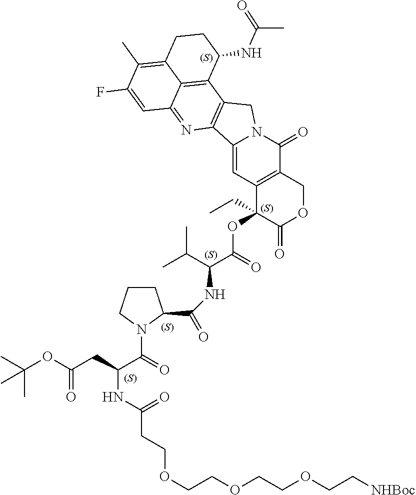

Trifluoroacetic acid-(1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (Intermediate 8) (35.0 mg, 50.7 μmol) was dissolved in DMF (5.0 mL). (2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) (35.9 mg, 60.8 μmol), HATU (25.0 mg, 65.9 μmol) and DIEA (35 μl, 200 μmol) were added. The reaction was stirred at rt for 1 h and then concentrated in vacuo. The residue was purified by prep. HPLC to give Intermediate 9 (45.0 mg, 84% purity, 65% yield) as a yellow foam. LC-MS: Rₜ=4.73 min; MS (ESIpos): m/z=1149 [M+H]⁺.

### Example I10: Preparation of (1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy) ethoxy] ethoxy}propanoyl)-L-alpha-aspartyl-L-prolyl-L-valinate-trifluoroacetic acid (1/1) (Intermediate 10)

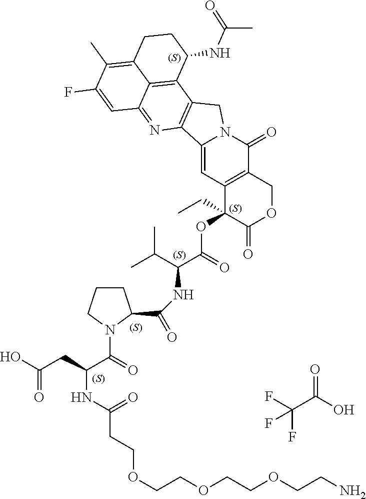

Tert-butyl (19S)-19-[(2S)-2-{[(2S)-1-{[(1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 9) (45.0 mg, 39.2 μmol) was dissolved in DCM (4.7 mL), then TFA (940 μl) was added. The reaction was stirred for 4 h at rt and then concentrated in vacuo. The residue was dissolved in ACN/water and freeze-dried to give a yellow oil, which was purified by prep. HPLC to give Intermediate 10 as two rotamers as a yellow foam (14.2 mg, 100% purity, 32% yield). LC-MS: Rₜ=1.98 min; MS (ESIpos): m/z=992 [M+H]⁺.

### Example I11: Preparation of Benzyl [(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]carbamate (Intermediate 11)

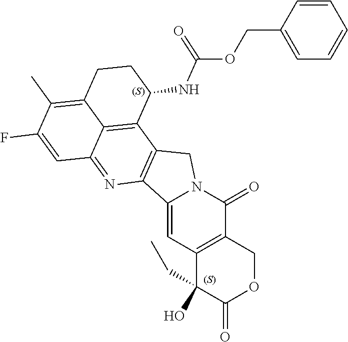

Methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (50.0 mg, 94.1 μmol) was dissolved in DMF (10.0 mL), then 1-{[(benzyloxy)carbonyl]oxy}pyrrolidine-2,5-dione (28.1 mg, 113 μmol) and DIEA (49 μl, 280 μmol) were added. The reaction was stirred overnight at rt. The reaction was concentrated in vacuo and the residue was mixed with ACN/H₂O/DMF. A solid precipitated, which was filtered off, dried and purified by prep. HPLC to give Intermediate 11 (50.0 mg, 92% purity, 87% yield) as a white foam. LC-MS: Rₜ=3.21 min; MS (ESIpos): m/z=570 [M+H]⁺.

### Example I12: Preparation of (1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7] indolizino[1,2-b]quinolin-9-yl N-(tert-butoxycarbonyl)-L-valinate (Intermediate 12)

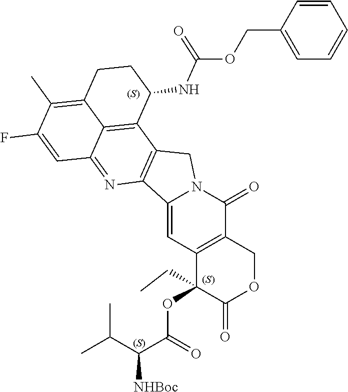

Benzyl [(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]carbamate (Intermediate 11) (50.0 mg, 87.8 μmol) was added to DCM (20.0 mL), then tert-butyl (4S)-2,5-dioxo-4-(propan-2-yl)-1,3-oxazolidine-3-carboxylate (51.2 mg, 211 μmol) and DMAP (19.3 mg, 158 μmol; CAS: 1122-58-3) were added. The mixture was refluxed while stirring for 7 h. The reaction was concentrated in vacuo. Water was added, upon which the product precipitated. The mixture was filtered, the filter residue was very poorly soluble, but was soluble in DMSO+ACN or MeOH/DCM. The compound was purified by prep. HPLC to give Intermediate 12 (54.0 g, 98% purity, 78% yield). LC-MS: Rₜ=2.48 min; MS (ESIpos): m/z=769 [M+H]⁺.

### Example I13: Preparation of Trifluoroacetic acid-(1S,9S)-1-{[(benzyloxy)carbonyl] amino}-9-ethyl-5-fluoro-4-methyl-10,13-dioxo-2,3,9,10,13,15-hexahydro-1H,12H-benzo[de] pyrano[3′,4′:6,7]indolizino[1,2-b]quinolin-9-yl L-valinate (1/1) (Intermediate 13)

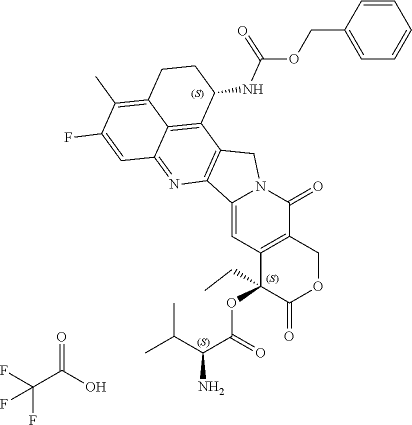

(1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 12) (44.0 mg, 98% purity, 55.9 μmol) was dissolved in DCM (6.3 mL), TFA (1.3 mL) was added and the reaction was stirred at rt for 30 min. The reaction was concentrated in vacuo, the residue was dissolved in ACN/H₂O and lyophilized to give Intermediate 13 (44.0 mg, 98% purity, 98% yield). LC-MS: Rₜ=2.56 min; MS (ESIpos): m/z=669 [M+H]⁺.

### Example I14: Preparation of Tert-butyl (19S)-19-[(2S)-2-{[(2S)-1-{[(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano [3′,4′:6,7] indolizino [1,2-b]quinolin-9-yl]oxy}-3-methyl-1-oxobutan-2-yl]carbamoyl} pyrrolidine-1-carbonyl]-2,2-dimethyl-4,17-dioxo-3, 8,11,14-tetraoxa-5,18-diazahenicosan-21-oate (Intermediate 14)

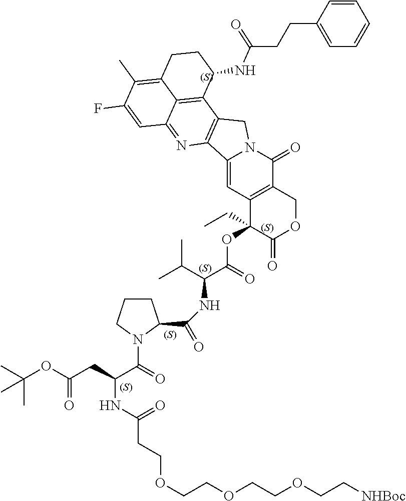

To a solution of trifluoroacetic acid-(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano [3′,4′:6,7]indolizino [1,2-b]quinolin-9-yl L-valinate (1/1) (Intermediate 13) (44.0 mg, 98% purity, 54.9 μmol) and (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) (38.9 mg, 65.9 μmol) in DMF (8.0 mL) were added HATU ((27.1 mg, 71.4 μmol) and DIEA (29 μl, 160 μmol). The solution was stirred at rt for 30 min and then concentrated in vacuo. The residue was purified by prep. HPLC, then lyophilized to give the title compound (50.0 mg, 100% purity, 73% yield) as a yellow foam. LC-MS: Rₜ=4.28 min; MS (ESIpos): m/z=1239 [M+H]⁺.

### Example I15: Preparation of (1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7] indolizino[1,2-b]quinolin-9-yl N-(3-{2-[2-(2-aminoethoxy)ethoxy]ethoxy}propanoyl)-L-alpha-aspartyl-L-prolyl-L-valinate-trifluoroacetic acid (1/1) (Intermediate 15)

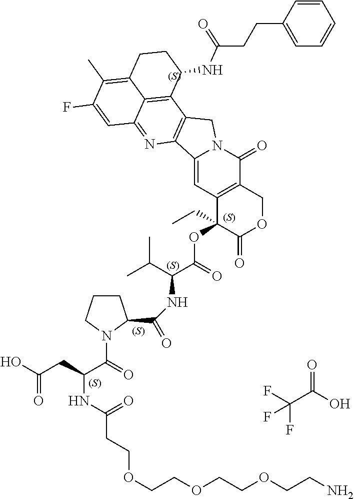

Tert-butyl (19S)-19-[(2S)-2-{[(2S)-1-{[(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano [3′,4′:6,7] indolizino [1,2-b]quinolin-9-yl]oxy}-3-methyl-1-oxobutan-2-yl]carbamoyl} pyrrolidine-1-carbonyl]-2,2-dimethyl-4,17-dioxo-3,8,11,14-tetraoxa-5,18-diazahenicosan-21-oate (Intermediate 14) (50.0 mg, 40.3 μmol) was dissolved in DCM (10 mL) and TFA (5.0 mL) was added. The reaction was stirred at rt for 4 h, before it was concentrated in vacuo. The residue was dissolved in ACN/H₂O and lyophilized to give Intermediate 15 (49.0 mg, 100%, 101% yield) as a yellow residue. LC-MS: Rₜ=2.63 min; MS (ESIpos): m/z=1084 [M+H]⁺.

### Example I16: Preparation of (7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-(tert-butoxycarbonyl)-L-valinate (Intermediate 16)

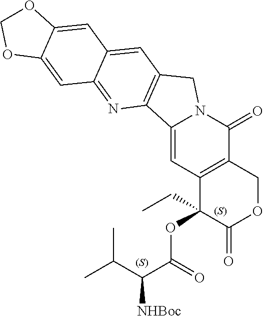

(7S)-7-ethyl-7-hydroxy-2H,10H-[1,3]dioxolo[4,5-g]pyrano[3′,4′:6,7]indolizino[1,2-b]quinoline-8,11(7H,13H)-dione (10,11-methylenedioxy-camptothecin) (20 mg, 0.051 mmol) was dissolved in DCM (5 mL), then tert-butyl (4S)-2,5-dioxo-4-(propan-2-yl)-1,3-oxazolidine-3-carboxylate (24.8 mg, 0.102 mmol) and DMAP (4.80 mg, 0.039 mmol) were added. The reaction was stirred for 12 h at rt. The same amounts of tert-butyl (4S)-2,5-dioxo-4-(propan-2-yl)-1,3-oxazolidine-3-carboxylate and DMAP were added again to the reaction and the reaction was stirred for a further 4 h. The reaction was concentrated in vacuo and purified by prep. HPLC to give Intermediate 16 (7.20 mg, 80% purity, 19% yield) as an almost colourless foam. LC-MS: Rₜ=3.47 min; MS (ESIpos): m/z=592 [M+H]⁺.

### Example I17: Preparation of Trifluoroacetic acid-(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl L-valinate (1/1) (Intermediate 17)

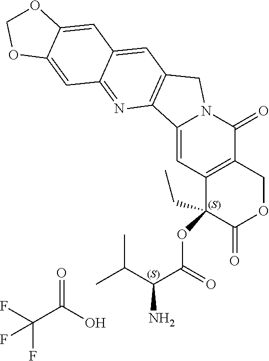

(7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-(tert-butoxycarbonyl)-L-valinate (Intermediate 16) (50.0 mg, 84.5 μmol) was dissolved in DCM (15.0 mL), then TFA (3.0 mL) was added. The reaction was stirred for 30 min at rt. The reaction was concentrated in vacuo and the residue was dissolved in ACN/H₂O and freeze-dried to give Intermediate 17 (10.2 mg, 82% purity, 16% yield) as a colourless foam. LC-MS: Rₜ=1.69 min; MS (ESIpos): m/z=492 [M+H]⁺. ¹H-NMR (500 MHz, DMSO-d₆) δ[ppm]: 0.935 (1.23), 0.949 (2.16), 0.964 (1.23), 1.029 (1.70), 1.043 (1.71), 1.085 (1.20), 1.097 (2.50), 1.111 (1.69), 1.141 (1.17), 1.155 (1.18), 1.232 (2.32), 1.248 (12.26), 1.262 (16.00), 1.276 (8.18), 2.217 (0.48), 2.232 (0.62), 2.248 (0.53), 2.731 (0.46), 2.891 (0.49), 3.125 (0.41), 3.131 (1.08), 3.140 (1.11), 3.146 (1.10), 3.154 (1.06), 3.594 (0.54), 3.602 (0.60), 3.607 (0.95), 3.615 (1.00), 3.620 (1.20), 3.628 (1.20), 3.633 (1.00), 3.641 (0.96), 3.646 (0.61), 3.654 (0.57), 5.243 (1.37), 5.260 (0.58), 5.520 (1.14), 5.527 (1.87), 6.293 (2.87), 7.126 (1.11), 7.231 (1.55), 7.431 (1.66), 7.531 (1.62), 7.537 (1.18), 8.387 (0.53), 8.481 (1.09), 8.492 (0.80), 8.574 (0.50).

### Example I18: Preparation of Tert-butyl (19S)-19-[(2S)-2-{[(2S)-1-{[(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 18)

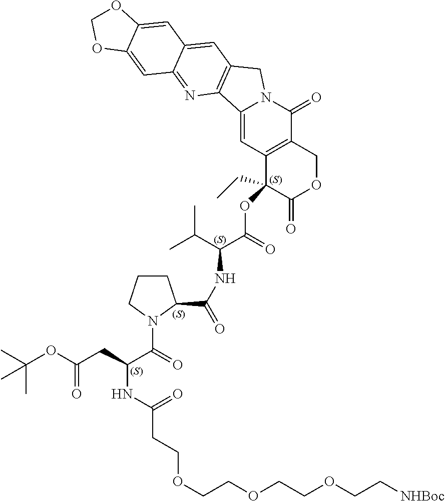

Trifluoroacetic acid-(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl L-valinate (1/1) (Intermediate 17) (25.0 mg, 41.3 μmol) was dissolved in DMF (5.0 mL). (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building block 3) (29.2 mg, 49.5 μmol), HATU (23.5 mg, 61.9 μmol) and DIEA (22 μl, 120 μmol) were added. The reaction was stirred at rt for 1 h and then concentrated in vacuo. The residue was purified by prep. HPLC to give Intermediate 18 (20.9 mg, 100% purity, 48% yield) as a light, yellow foam. LC-MS: Rₜ=1.98 min; MS (ESIpos): m/z=1063 [M+H]⁺.

### Example I19: Preparation of (7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-(3-{2-[2-(2-aminoethoxy) ethoxy] ethoxy}propanoyl)-L-alpha-aspartyl-L-prolyl-L-valinate-trifluoroacetic acid (1/1) (Intermediate 19)

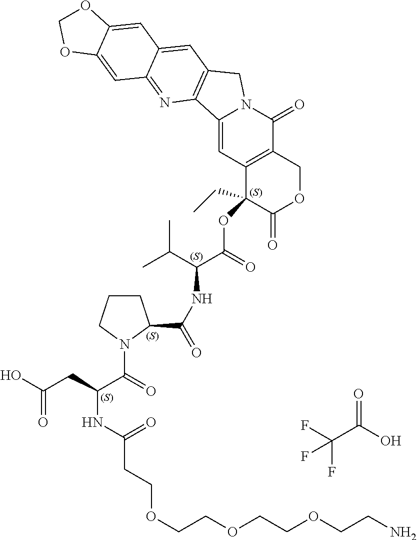

Tert-butyl (19S)-19-[(2S)-2-{[(2S)-1-{[(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 18) (37.0 mg, 34.8 μmol) was dissolved in DCM (5.0 mL) and TFA (1.0 mL) was added. The reaction was stirred for 30 min at rt and then concentrated in vacuo. The residue was dissolved in ACN/H₂O and freeze-dried to give Intermediate 19 (36.0 mg, 100% purity, 101% yield) as a colourless foam. LC-MS: Rₜ=2.66 min; MS (ESIpos): m/z=907 [M+H]⁺.

### Example I20: Preparation of Benzyl N²-(tert-butoxycarbonyl)-L-asparaginyl-L-prolinate (Intermediate 20)

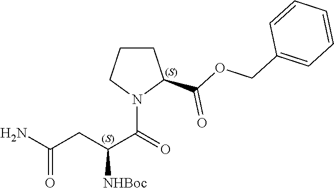

N²-(tert-butoxycarbonyl)-L-asparagine (500 mg, 2.15 mmol) was dissolved in DMF (40 mL). Benzyl L-prolinate-hydrogen chloride (1/1) (520 mg, 2.15 mmol), HATU (982 mg, 2.58 mmol) and DIEA (750 μl, 4.3 mmol) were added. The reaction was stirred at rt for 4 h and then concentrated in vacuo. The residue was purified by prep. HPLC to give Intermediate 20 (700 mg, 99% purity, 77% yield) as an almost colourless foam. LC-MS: Rₜ=1.52 min; MS (ESIpos): m/z=420 [M+H]⁺.

### Example I21: Preparation of Trifluoroacetic acid-benzyl L-asparaginyl-L-prolinate (2/1) (Intermediate 21)

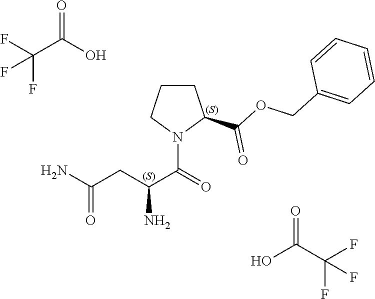

Benzyl N²-(tert-butoxycarbonyl)-L-asparaginyl-L-prolinate (Intermediate 20) (700 mg, 1.67 mmol) was dissolved in DCM (20 mL), then TFA (4.0 mL) was added. The reaction was stirred for 30 min at rt and the reaction was concentrated in vacuo. The residue was dissolved in ACN/H₂O and freeze-dried to give Intermediate 21 (831 mg, 98% purity, 89% yield) as a colourless foam. LC-MS: Rₜ=0.47 min; MS (ESIpos): m/z=320 [M+H]⁺.

### Example I22: Preparation of Trifluoroacetic acid-benzyl N-methyl-N-(2-{methyl[2-(methylamino) ethyl]amino}ethyl) glycinate (1/1) (Intermediate 22)

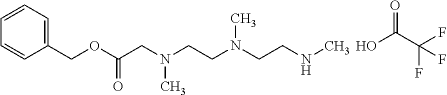 [SMILES: CNCCN(C)CCN(C)CC(=O)OCc1ccccc1 ; O=C(O)C(F)(F)F]

The reaction was conducted under argon. N¹,N²-dimethyl-N¹-[2-(methylamino)ethyl] ethane-1,2-diamine (500 mg, 3.44 mmol) was dissolved in ACN (50 mL) and potassium carbonate (476 mg, 3.44 mmol; CAS: 584-08-7) was added. At 0° C. a solution of benzyl bromoacetate (394 mg, 1.72 mmol) in ACN was added. The reaction was stirred at rt for 20 h. The reaction was filtered to separate off the solids and the filtrate was concentrated and purified by prep. HPLC to give Intermediate 22 (476 mg, 99% purity, 34% yield) as a colourless oil. LC-MS: Rₜ=0.49 min; MS (ESIpos): m/z=294 [M+H]⁺. ¹H-NMR (600 MHz, DMSO-d₆) δ[ppm]: 2.615 (16.00), 2.644 (12.71), 3.013 (2.29), 3.025 (2.19), 3.037 (2.39), 3.047 (1.67), 3.114 (1.55), 3.123 (2.48), 3.132 (1.26), 3.201 (1.72), 3.212 (2.98), 3.223 (1.38), 3.947 (6.33), 5.204 (10.48), 7.354 (0.62), 7.361 (1.24), 7.368 (1.39), 7.375 (1.02), 7.382 (0.73), 7.397 (11.06), 7.404 (8.52).

### Example I23: Preparation of Benzyl N-methyl-N-(2,2,8,11-tetramethyl-4-oxo-3-oxa-5,8,11-triazatridecan-13-yl)glycinate (Intermediate 23)

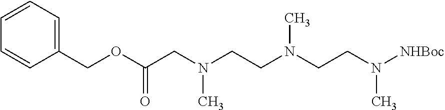 [SMILES: CN(CCN(C)CC(=O)OCc1ccccc1)CCN(C)NC(=O)OC(C)(C)C]

Trifluoroacetic acid-benzyl N-methyl-N-(2-{methyl[2-(methylamino) ethyl]amino} ethyl) glycinate (1/1) (Intermediate 22) (470 mg, 1.15 mmol), tert-butyl (2-oxoethyl)carbamate (367 mg, 2.31 mmol) and AcOH (170 μl) were dissolved in MeOH (50 mL) to give a clear solution. Then trihydrido(pyridine)boron (515 mg, 5.54 mmol) was added, and the reaction was stirred at rt for 5 h. The reaction was concentrated in vacuo and purified twice by prep. HPLC to give Intermediate 23 (366 mg, 99% purity, 72% yield) as a colourless oil. LC-MS: Rₜ=0.95 min; MS (ESIpos): m/z=437 [M+H]⁺.

### Example I24: Preparation of N-methyl-N-(2,2,8,11-tetramethyl-4-oxo-3-oxa-5,8,11-triazatridecan-13-yl)glycine (Intermediate 24)

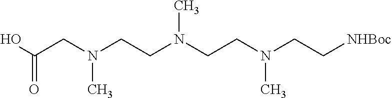 [SMILES: CN(CCNC(=O)OC(C)(C)C)CCN(C)CCN(C)CC(=O)O]

Benzyl N-methyl-N-(2,2,8,11-tetramethyl-4-oxo-3-oxa-5,8,11-triazatridecan-13-yl)glycinate (Intermediate 23) (366 mg, 838 μmol) was dissolved in DCM/MeOH (1:1, 30 mL). Pd/C 10% (12.0 mg) was added, and the reaction was hydrogenated at rt under normal pressure for 2 h. The catalyst was filtered off and the filtrate was concentrated to give Intermediate 24 (312 mg, 107% yield) as a colourless oil. LC-MS: Rₜ=0.47 min; MS (ESIpos): m/z=347 [M+H]⁺. ¹H-NMR (600 MHz, DMSO-d₆) δ[ppm]: 1.395 (16.00), 2.073 (1.10), 2.370 (5.09), 2.770 (6.00), 2.803 (5.43), 2.865 (0.82), 2.876 (1.83), 2.889 (1.69), 2.900 (0.82), 3.124 (0.70), 3.134 (1.29), 3.145 (0.78), 3.205 (0.89), 3.215 (1.56), 3.225 (0.80), 3.268 (0.85), 3.279 (1.40), 3.290 (1.15), 3.302 (1.20), 3.312 (1.06), 3.964 (2.49), 7.097 (0.57).

### Example I25: Preparation of Benzyl N-methyl-N-(2,2,8,11-tetramethyl-4-oxo-3-oxa-5,8,11-triazatridecan-13-yl)glycyl-L-asparaginyl-L-prolinate (Intermediate 25)

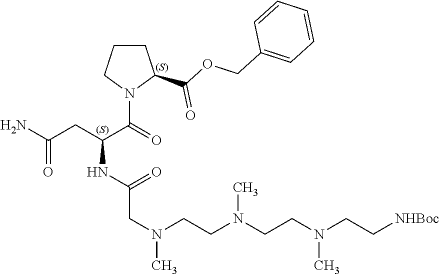

Benzyl L-asparaginyl-L-prolinate trifluoroacetate (1:2) (Intermediate 21) (244.9 mg, 0.447 mmol) was dissolved in DMF (20.0 mL). N-methyl-N-(2,2,8,11-tetramethyl-4-oxo-3-oxa-5,8,11-triazatridecan-13-yl)glycine (Building block 7) (155.0 mg, 0.447 mmol), HATU (255.2 mg, 671.0 μmol) and DIEA (233.8 μL, 1342 μmol) were added. The reaction was stirred at rt for 1 h and then concentrated in vacuo. The residue was separated by prep. HPLC to give Intermediate 25 (227 mg, 92% purity, 72% yield) as a colourless foam. LC-MS: Rₜ=1.49 min; MS (ESIpos): m/z=648 [M+H]⁺.

### Example I27: Preparation of (7S)-7-Ethyl-8,11-dioxo-7,8,11,13-tetrahydro-2H,10H-[1,3]dioxolo[4,5-g]pyrano[3′,4′:6,7] indolizino [1,2-b]quinolin-7-yl N-methyl-N-(2,2,8,11-tetramethyl-4-oxo-3-oxa-5,8,11-triazatridecan-13-yl) glycyl-L-asparaginyl-L-prolyl-L-valinate (Intermediate 27)

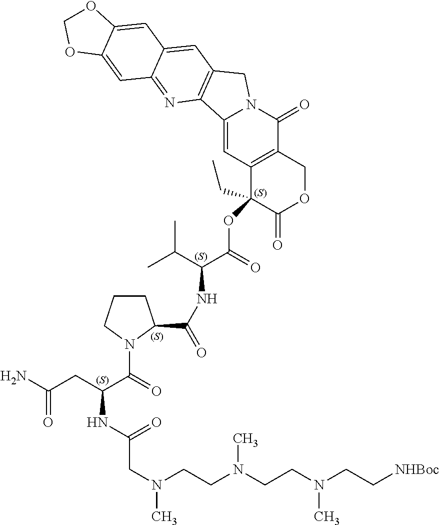

Trifluoroacetic acid-(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl L-valinate (1/1) (Intermediate 17) (28.0 mg, 46.2 μmol) was dissolved in DMF (5.0 mL). N-methyl-N-(2,2,8,11-tetramethyl-4-oxo-3-oxa-5,8,11-triazatridecan-13-yl)glycyl-L-asparaginyl-L-proline (Building block 8) (25.8 mg, 46.2 μmol), HATU (26.4 mg, 69.4 μmol) and DIEA (24 μl, 140 μmol) were added. The reaction was stirred at rt for 1 h. The reaction was concentrated in vacuo. The residue was separated by prep. HPLC to give Intermediate 27 (28.0 mg, 93% purity, 54% yield) as a light, yellow foam. LC-MS: Rₜ=2.60 min; MS (ESIpos): m/z=1031 [M+H]⁺.

### Example I28: Preparation of Trifluoroacetic acid-(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl N-{2-[{2-[(2-aminoethyl)(methyl)amino]ethyl} (methyl) amino]ethyl}-N-methylglycyl-L-asparaginyl-L-prolyl-L-valinate (1/1) (Intermediate 28)

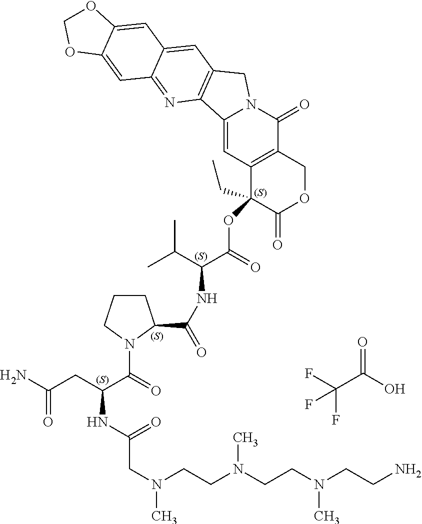

(7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-methyl-N-(2,2,8,11-tetramethyl-4-oxo-3-oxa-5,8,11-triazatridecan-13-yl)glycyl-L-asparaginyl-L-prolyl-L-valinate (Intermediate 27) (28.0 mg, 27.2 μmol) was dissolved in DCM (5.0 mL), then TFA (1.0 mL) was added. The reaction was stirred for 30 min at rt and then concentrated in vacuo. The residue was dissolved in ACN/H₂O and freeze-dried to give Intermediate 28 (28.0 mg, 100% purity, 99% yield) as a colourless foam. LC-MS: Rₜ=2.17 min; MS (ESIpos): m/z=931 [M+H]⁺.

### Example I29: Preparation of tert-butyl ((S)-4-amino-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl)amino)-1,4-dioxobutan-2-yl)carbamate (Intermediate 29)

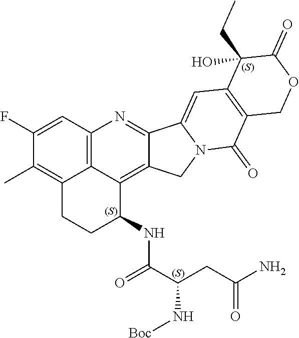

To a solution of methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (30.0 mg, 56.4 umol) in DMF (10 mL) were added 2,5-dioxopyrrolidin-1-yl N²-(tert-butoxycarbonyl)-L-asparaginate (37.2 mg, 112.9 umol) and DIEA (39.3 ul, 225.7 umol). The reaction was stirred at RT, for 3 h, then another 1 eq. Boc-Asn-OSu (18.6 mg, 56.4 umol) and 1 eq. DIEA (10.0 uL, 56.4 umol) were added and the reaction was stirred overnight. The reaction was concentrated in vacuo, purified by prep. HPLC and lyophilized to give the title compound (37.0 mg, 100% purity, 100% yield) as a yellow foam.

LC-MS: Rₜ=2.52 min; MS (ESIpos): m/z=650 [M+H]⁺.

### Example I30: Preparation of Trifluoroacetic acid-N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl]-L-aspartamide (1/1) (Intermediate 30)

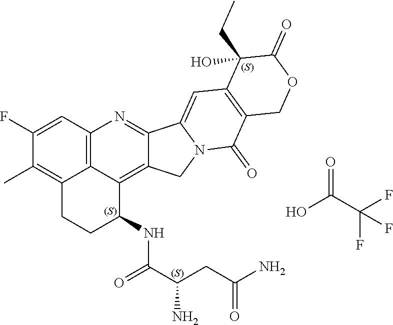

tert-butyl ((S)-4-amino-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl)amino)-1,4-dioxobutan-2-yl)carbamate (Intermediate 29) (37 mg, 57 μmol) was dissolved in DCM (6 mL), then TFA (1 mL) was added. The reaction was stirred for 30 min at rt and then concentrated in vacuo. The residue was dissolved in ACN/H₂O and lyophilized to give Intermediate 30 (38.0 mg, 98% purity, 99% yield) as a yellow foam. LC-MS: Rₜ=0.92 min; MS (ESIpos): m/z=550 [M+H]⁺.

### Example I31: Preparation of tert-butyl ((S)-1-(((S)-1-(((S)-4-amino-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-1,4-dioxobutan-2-yl)amino)-1-oxopropan-2-yl)(methyl)amino)-1-oxopropan-2-yl)carbamate (Intermediate 31)

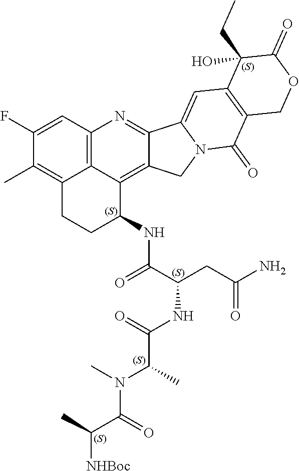

Trifluoroacetic acid-N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]-L-aspartamide (1/1) (Intermediate 30) (10 mg, 14.8 μmol), was dissolved in DMF (4 mL). N-(tert-butoxycarbonyl)-L-alanyl-N-methyl-L-alanine (Building block 6) (4.9 mg, 18 μmol), HATU (8.5 mg, 22 μmol) and DIEA (6 mg, 45 μmol) were added. The reaction was stirred for 10 min at rt and then concentrated in vacuo. The residue was purified by prep. HPLC and afterwards lyophilized to give Intermediate 131 (5.0 mg, 100% purity, 42%. yield) as a yellow foam. LC-MS: Rₜ=1.55 min; MS (ESIpos): m/z=806 [M+H]⁺.

### Example I32: Preparation of Trifluoroacetic acid L-alanyl-N-methyl-L-alanyl-N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino [1,2-b]quinolin-1-yl]-L-aspartamide (1/1) (Intermediate 32)

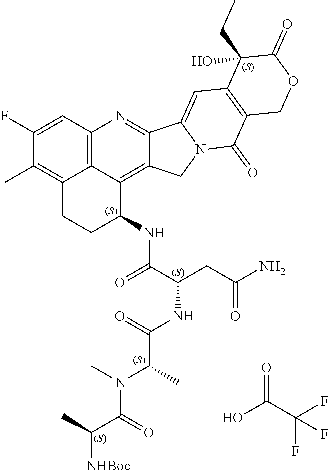

tert-butyl ((S)-4-amino-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl)amino)-1,4-dioxobutan-2-yl)carbamate (Intermediate 31) (11 mg, 14 μmol) was dissolved in DCM (4.6 mL), then TFA (0.9 mL) was added. The reaction was stirred for 30 min at rt and then concentrated in vacuo. The residue was redissolved in ACN/H2O and lyophilized to give Intermediate 32 (10.0 mg, 100% purity, 89% yield) as a yellow foam. LC-MS: Rₜ=0.98 min; MS (ESIpos): m/z=706 [M+H]⁺.

### Example I33: (1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate trifluoroacetic acid (1/1) (Intermediate 33)

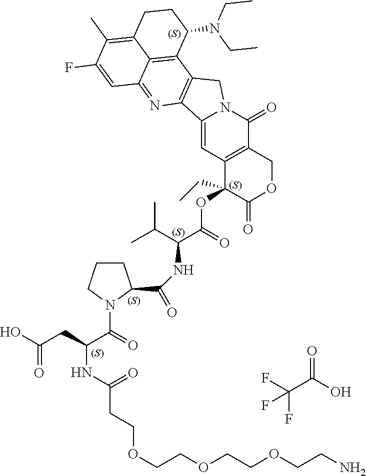

Tert-butyl (19S)-19-[(2S)-2-{[(2S)-1-{[(1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (18.1 mg, 15.6 μmol) (Intermediate 34) was dissolved in DCM (5.0 ml), then TFA (2.0 ml) was added. The reaction was stirred for 1 hour at RT and concentrated in vacuo. The residue was dissolved in ACN/H2O and freeze-dried to give Intermediate 33 (17.0 mg, 95% purity, 92% yield) as a light yellow foam. LC-MS (Method 3): Rₜ=2.65 min; MS (ESIpos): m/z=1006 [M+H]⁺.

### Example I34: Tert-butyl (19S)-19-[(2S)-2-{[(2S)-1-{[(1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 34)

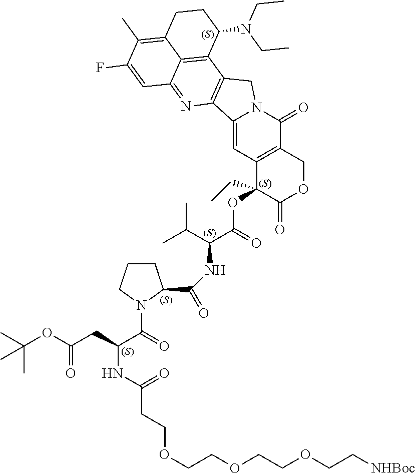

Trifluoroacetic acid (1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (14.0 mg, 19.9 μmol) (Intermediate 35) was dissolved in DMF (5.0 ml). (2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (14.1 mg, 23.8 μmol) (Building block 3), HATU (9.82 mg, 25.8 μmol; CAS-RN:[148893-10-1]) and N,N-diisopropylethylamine (10 μl, 60 μmol; CAS-RN:[7087-68-5]) were added. The reaction was stirred at RT for 1 hour and concentrated in vacuo. The residue was purified by prep. HPLC to give Intermediate 34 (18.1 mg, 91% purity, 71% yield) as a light yellow foam. LC-MS (Method 3): Rₜ=4.69 min; MS (ESIpos): m/z=1163 [M+H]⁺.

### Example I35: Trifluoroacetic acid (1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (Intermediate 35)

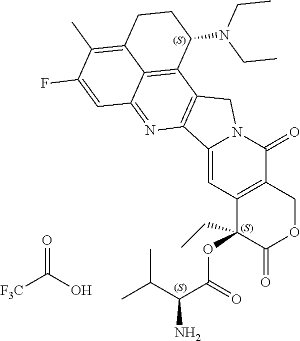

(1S,9S)-1-(Diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (15.0 mg, 21.7 μmol) (Intermediate 36) was dissolved in DCM (7.5 ml), then TFA (1.5 ml) was added. The reaction was stirred for 1 hour at RT and concentrated in vacuo. The residue was dissolved in ACN/H2O and freeze-dried to give Intermediate 35 (14.0 mg, 100% purity, 91% yield) as a beige foam. LC-MS (Method 3): Rₜ=2.54 min; MS (ESIpos): m/z=591 [M+H]⁺.

### Example I36: (1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 36)

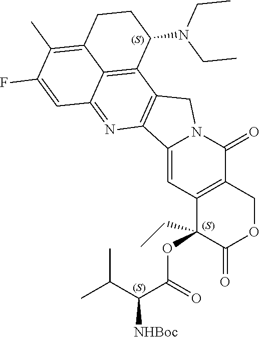

(1S,9S)-1-(Diethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (20.0 mg, 40.7 μmol) (Compound 17) was dissolved in DCM (5.0 ml). Boc-Val-NCA: tert-butyl (4S)-2,5-dioxo-4-(propan-2-yl)-1,3-oxazolidine-3-carboxylate (23.8 mg, 97.6 μmol) and DMAP (8.95 mg, 73.2 μmol; CAS-RN:[1122-58-3]) were added. The reaction was stirred for 22 hours at reflux and then concentrated in vacuo. The residue was separated by prep. HPLC to give Intermediate 36 (15.0 mg, 100% purity, 53% yield) as a beige foam. LC-MS (Method 3): Rₜ=4.74 min; MS (ESIpos): m/z=691 [M+H]⁺.

### Example I37: (1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate trifluoroacetic acid (Intermediate 37)

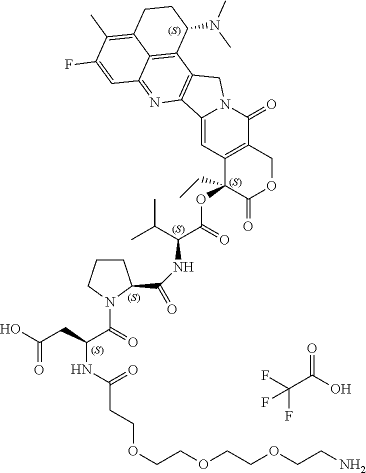

Tert-butyl (19S)-19-[(2S)-2-{[(2S)-1-{[(1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (36.0 mg, 31.7 μmol) (Intermediate 38) was dissolved in DCM (5.0 ml), then TFA (2.5 ml) was added. The reaction was stirred for 3 hour at RT and concentrated in vacuo. The residue was dissolved in ACN/H2O and freeze-dried to give Intermediate 37 (33.0 mg, 100% purity, 95% yield) as a light yellow foam. LC-MS (Method 2) Rₜ=0.91 min; MS (ESIneg): m/z=976 [M−H]⁻.

### Example I38: Tert-butyl (19S)-19-[(2S)-2-{[(2S)-1-{[(1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 38)

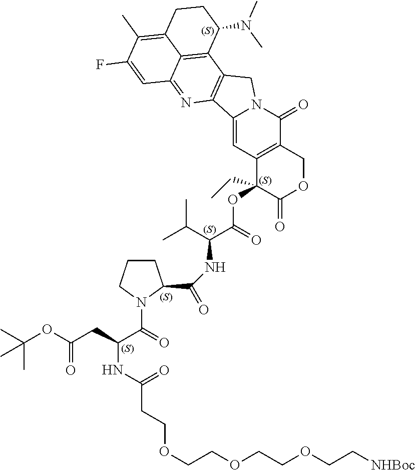

Trifluoroacetic acid (1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (23.0 mg, 34.0 μmol) (Intermediate 39) was dissolved in DMF (8.6 ml). (2S)-1-[(19S)-19-(2-Tert-butoxy-2-oxoethyl)-2,2-dimethyl-4,17,20-trioxo-3,8,11,14-tetraoxa-5,18-diazaicosan-20-yl]pyrrolidine-2-carboxylic acid (24.1 mg, 40.8 μmol) (Building block 3), HATU (16.8 mg, 44.2 μmol; CAS-RN:[148893-10-1]) and N,N-diisopropylethylamine (18 μl, 100 μmol; CAS-RN:[7087-68-5]) were added. The reaction was stirred at RT for 1 hour and then concentrated in vacuo. The residue was separated by prep. HPLC to give Intermediate 38 (36.0 mg, 99% purity, 92% yield) as a light yellow foam. LC-MS (Method 6): Rₜ=3.03 min; MS (ESIpos): m/z=1134 [M+H]⁺.

### Example I39: Trifluoroacetic acid (1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl L-valinate (1/1) (Intermediate 39)

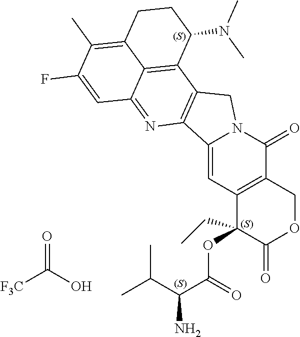

(1S,9S)-1-(Dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (23.0 mg, 34.7 μmol) (Intermediate 40) was dissolved in DCM (5.2 ml), then TFA (1.0 ml) was added. The reaction was stirred for 1 hour at RT and concentrated in vacuo. The residue was dissolved in ACN/H2O and freeze-dried to give Intermediate 39 (23.0 mg, 97% purity, 95% yield) as a beige foam. LC-MS (Method 3): Rₜ=2.06 min; MS (ESIpos): m/z=563 [M+H]⁺.

### Example I40: (1S,9S)-1-(Dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(tert-butoxycarbonyl)-L-valinate (Intermediate 40)

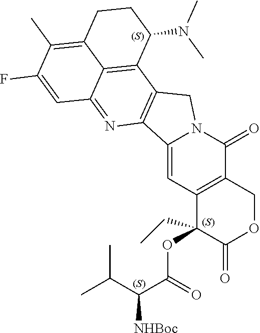

(1S,9S)-1-(Dimethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (40.0 mg, 86.3 μmol) (Intermediate 41) was dissolved in DCM (33 ml). Boc-Val-NCA: tert-butyl (4S)-2,5-dioxo-4-(propan-2-yl)-1,3-oxazolidine-3-carboxylate (50.4 mg, 207 μmol) and 4-(Dimethylamino) pyridine (DMAP): (19.0 mg, 155 μmol; CAS-RN:[1122-58-3]) were added. The reaction was stirred for 22 hours at reflux and then concentrated in vacuo. The residue was separated by prep. HPLC to give Intermediate 40 (23.0 mg, 100% purity, 40% yield)) as a beige foam. LC-MS (Method 3): Rₜ=3.96 min; MS (ESIpos): m/z=663 [M+H]⁺.

### Example I41: (1S,9S)-1-(Dimethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Intermediate 41)

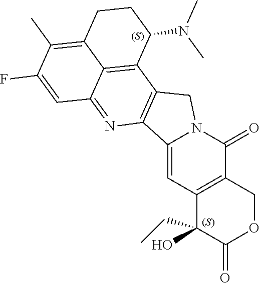

Methanesulfonic acid (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (Exatecan mesylate) (50.0 mg, 94.1 μmol) was dissolved in methanol (10 ml). Sodium hydrogen carbonate (15.8 mg, 188 μmol; CAS-RN:[144-55-8]) was added. Formaldehyde (61.1 mg, 37% purity, 752 μmol) was added and the reaction was stirred for 1 hour at 50° C. 2× more formaldehyde was added and stirring was continued for 1 h at 50° C. The reaction was then cooled to 0° C. and sodium triacetoxyborohydride (79.7 mg, 376 μmol; CAS-RN:[56553-60-7]) was added. The reaction was stirred at RT for 2 h and then concentrated in vacuo. The residue was dissolved in ACN/H2O, the precipitate was filtered off with suction and the filtrate was purified by prep. HPLC. The precipitate was dissolved in ACN/H2O and freeze-dried to give Intermediate 41 (15.0 mg, 87% purity, 30% yield) as a colourless foam. LC-MS (Method 2): Rₜ=0.94 min; MS (ESIpos): m/z=464 [M+H]⁺.

### Example I42: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{2-[{2-[(2-aminoethyl)(methyl)amino]ethyl}(methyl)amino]ethyl}-N-methylglycyl-L-asparaginyl-L-prolyl-L-valinate trifluoroacetate (1:3) (Intermediate 42)

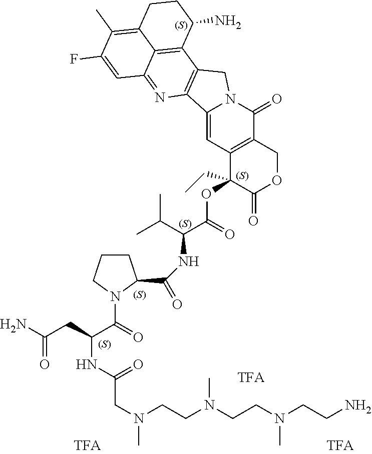

(1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{2-[{2-[(2-aminoethyl)(methyl)amino]ethyl}(methyl)amino]ethyl}-N-methylglycyl-L-asparaginyl-L-prolyl-L-valinate trifluoroacetate (1:3) (44.0 mg, 30.3 μmol) (Intermediate 43) was dissolved in ethanol (10.0 ml). Pd/C 10% was added, and the reaction was hydrogenated at RT at atmospheric pressure for 2 hours. The catalyst was filtered off and the filtrate was concentrated. The residue was dissolved in ACN/H2O and freeze-dried to give Intermediate 42 (37.0 mg, 72% purity, 67% yield) as a colorless foam. LC-MS (Method 3): Rₜ=1.76 min; MS (ESIpos): m/z=973 [M+H]⁺⁻.

### Example I43: Trifluoroacetic acid (1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{2-[{2-[(2-aminoethyl)(methyl)amino]ethyl}(methyl)amino]ethyl}-N-methylglycyl-L-asparaginyl-L-prolyl-L-valinate (Intermediate 43)

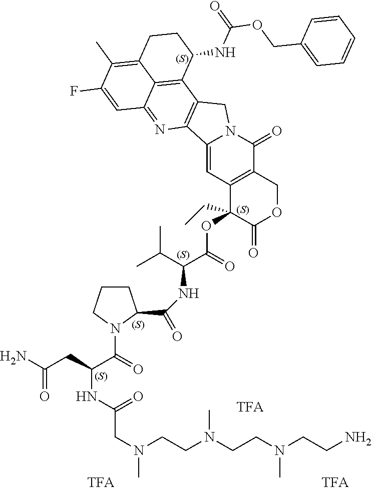

(1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-methyl-N-(2,2,8,11-tetramethyl-4-oxo-3-oxa-5,8,11-triazatridecan-13-yl)glycyl-L-asparaginyl-L-prolyl-L-valinate (58.0 mg, 48.0 μmol) (Intermediate 44) was dissolved in DCM (10 ml), then TFA (2.0 ml, 26 mmol; CAS-RN:[76-05-1]) was added. The reaction was stirred for 1 hour at RT and then concentrated in vacuo. The residue was dissolved in ACN/H2O and freeze-dried to give Intermediate 43 (59.0 mg, 95% purity, 80% yield) as a light yellow foam. LC-MS (Method 3): Rₜ=2.99 min; MS (ESIpos): m/z=1108 [M+H]⁺.

### Example I44: (1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-methyl-N-(2,2,8,11-tetramethyl-4-oxo-3-oxa-5,8,11-triazatridecan-13-yl)glycyl-L-asparaginyl-L-prolyl-L-valinate (Intermediate 44)

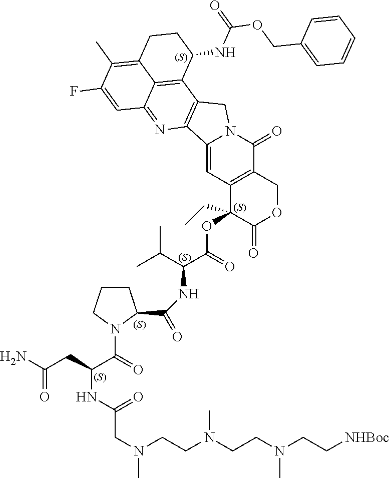

Trifluoroacetic acid (1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinoline-9-yl L-valinate (1/1) (98.0 mg, 125 μmol) (Intermediate 13) was dissolved in DMF (9.1 ml). N-methyl-N-(2,2,8,11-tetramethyl-4-oxo-3-oxa-5,8,11-triazatridecan-13-yl)glycyl-L-asparaginyl-L-proline (69.8 mg, 125 μmol) (Building block 8), HATU (71.4 mg, 188 μmol; CAS-RN:[148893-10-1]) and N,N-diisopropylethylamine (44 μl, 250 μmol; CAS-RN:[7087-68-5]) were added. The reaction was stirred at RT for 3 hour and then concentrated in vacuo. The residue was separated by prep. HPLC to give Intermediate 44 (58.0 mg, 93% purity, 36% yield) as a light yellow foam. LC-MS (Method 3): Rₜ=3.36 min; MS (ESIpos): m/z=1208 [M+H]⁺.

### Example I45: (7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-(3-{2-[2-(2-{[N2,N6-bis(14-{4-[({(1R)-2-carboxy-1-[3-({3-[(propylcarbamoyl)amino]benzene-1-sulfonyl}amino)phenyl]ethyl}carbamoyl)amino] anilino}-14-oxo-4,7,10-trioxa-13-azatetradecanan-1-oyl)-L-lysyl]amino}ethoxy)ethoxy]ethoxy} propanoyl)-L-alpha-aspartyl-L-prolyl-L-valinate (Intermediate 45)

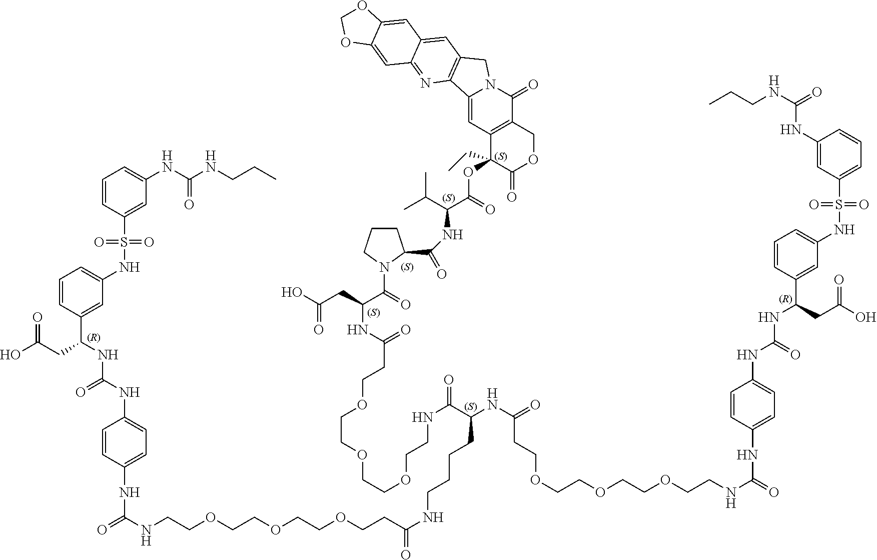

(7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate trifluoroacetic acid (1/2) (135 mg, 80.9 μmol) (Intermediate 46) was dissolved in DMF (40 ml). (3R)-3-{[(4-{[(4-nitrophenoxy)carbonyl]amino}phenyl) carbamoyl]amino}-3-[3-({3-[(propylcarbamoyl)amino] benzene-1-sulfonyl}amino)phenyl] propanoic acid (116 mg, 162 μmol) (Building block 2) and N,N-diisopropylethylamine (280 μl, 1.6 mmol; CAS-RN:[7087-68-5]) were added. The reaction was stirred for 30 minutes at RT and then concentrated in vacuo. The residue was separated twice by prep. HPLC to give Intermediate 45 (89.0 mg (94% purity, 40% yield) as a yellow foam. LC-MS (Method 3): Rₜ=4.00 min; MS (ESIpos): m/z=2602 [M+H]⁺.

### Example I46: (7S)-7-ethyl-8,11-dioxo-7,8,11,13-tetrahydro-2H,10H-[1,3]dioxolo[4,5-g]pyrano [3′,4′:6,7] indolizino [1,2-b]quinolin-7-yl N-(3-{2-[2-(2-{[N2,N6-bis(3-{2-[2-(2-aminoethoxy) ethoxy] ethoxy}propanoyl)-L-lysyl]amino}ethoxy)ethoxy]ethoxy}propanoyl)-L-alpha-aspartyl-L-prolyl-L-valinate trifluoroacetic acid (1/2) (Intermediate 46)

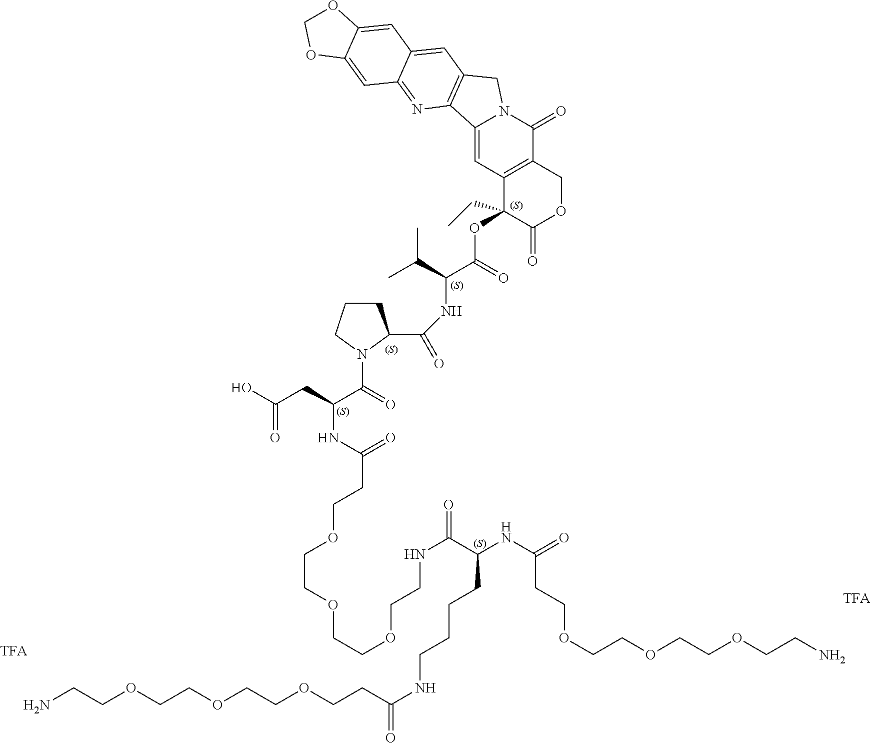

(7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy]ethoxy} propanoyl)-L-alpha-aspartyl-L-prolyl-L-valinate (113 mg, 68.8 μmol) (Intermediate 47) was dissolved in DCM (20 ml), then TFA (4.0 ml) was added. The reaction was stirred for 1 hour at RT and the concentrated in vacuo. The residue was dissolved in ACN/H2O and freeze-dried to give Intermediate 46 (135 mg, 100% purity, 117% yield) as a yellow foam. LC-MS (Method 3): Rₜ=2.34 min; MS (ESIpos): m/z=1441 [M+H]⁺.

### Example I47: (7S)-7-Ethyl-8,11-dioxo-7,8,11,13-tetrahydro-2H,10H-[1,3]dioxolo[4,5-g]pyrano [3′,4′:6,7]indolizino[1,2-b]quinolin-7-yl N-(3-{2-[2-(2-{[N2,N6-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-L-valinate (Intermediate 47)

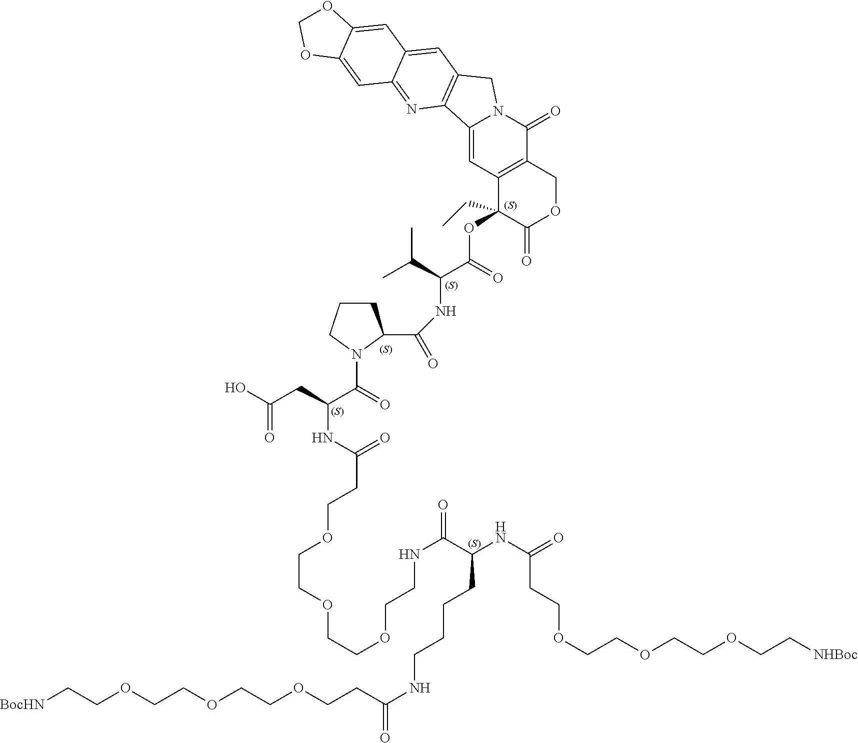

(7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-[3-(2-{2-[2-(L-lysylamino)ethoxy]ethoxy}ethoxy)propanoyl]-L-alpha-aspartyl-L-prolyl-L-valinate trifluoroacetic acid (1/2) (145 mg, 115 μmol) (Intermediate 48) was dissolved in DMF (32 ml). Tert-butyl {2-[2-(2-{3-[(2,5-dioxopyrrolidin-1-yl)oxy]-3-oxopropoxy}ethoxy)ethoxy]ethyl} carbamate (115 mg, 275 μmol) (Intermediate 49) and N,N-diisopropyl ethyl amine (160 μl, 920 μmol; CAS-RN:[7087-68-5]) were added. The reaction was stirred at RT for 3 h and the concentrated in vacuo. The residue was purified by prep. HPLC to give Intermediate 47 (113 mg, 100% purity, 60% yield) as a colorless foam. LC-MS (Method 3): Rₜ=4.14 min; MS (ESIpos): m/z=1641 [M+H]⁺.

### Example I48: (7S)-7-ethyl-8,11-dioxo-7,8,11,13-tetrahydro-2H,10H-[1,3]dioxolo[4,5-g]pyrano [3′,4′:6,7]indolizino[1,2-b]quinolin-7-yl N-[3-(2-{2-[2-(L-lysylamino)ethoxy]ethoxy}ethoxy) propanoyl]-L-alpha-aspartyl-L-prolyl-L-valinate trifluoroacetic acid (1/2) (Intermediate 48)

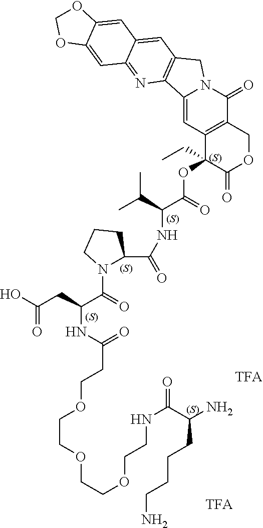

(7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-(3-{2-[2-(2-{[N²,N⁶-bis(tert-butoxycarbonyl)-L-lysyl]amino} ethoxy)ethoxy]ethoxy}propanoyl)-L-alpha-aspartyl-L-prolyl-L-valinate (103 mg, 83.4 μmol) (Intermediate 50) was dissolved in DCM (15 ml), then TFA (5.0 ml) was added. The reaction was stirred for 1 hour at RT and concentrated in vacuo. The residue was dissolved in ACN/H2O and freeze-dried to give Intermediate 48 (108 mg, 100% purity, quantitative yield) as a yellow foam. LC-MS (Method 3): Rₜ=2.27 min; MS (ESIpos): m/z=1035 [M+H]⁺.

### Example I49: tert-butyl {2-[2-(2-{3-[(2,5-dioxopyrrolidin-1-yl)oxy]-3-oxopropoxy}ethoxy)ethoxy] ethyl}carbamate (Intermediate 49)

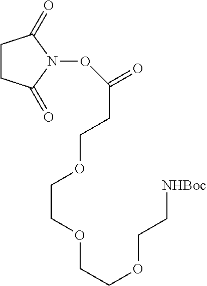 [SMILES: CC(C)(C)OC(=O)NCCOCCOCCOCCC(=O)ON1C(=O)CCC1=O]

t-Boc-N-amido-PEG3-acid: 2,2-dimethyl-4-oxo-3,8,11,14-tetraoxa-5-azaheptadecan-17-oic acid (3.00 g, 9.33 mmol) was suspended in DCM (50 ml). HO-Su: 1-hydroxypyrrolidine-2,5-dione (1.61 g, 14.0 mmol), EDCI (2.15 g, 11.2 mmol) and DMAP (5.00 mg, 40.9 μmol) were added. The reaction was stirred for 1 hour at RT and concentrated in vacuo. The residue was purified by prep. HPLC to give Intermediate 49 (3.15 g, 90% purity, 72% yield) as a colorless oil. LC-MS (Method 2): Rₜ=1.38 min; MS (ESIpos): m/z=419 [M+H]⁺.

### Example I50: (7S)-7-Ethyl-8,11-dioxo-7,8,11,13-tetrahydro-2H,10H-[1,3]dioxolo[4,5-g]pyrano [3′,4′:6,7]indolizino[1,2-b]quinolin-7-yl N-(3-{2-[2-(2-{[N2,N6-bis(tert-butoxycarbonyl)-L-lysyl]amino}ethoxy)ethoxy]ethoxy}propanoyl)-L-alpha-aspartyl-L-prolyl-L-valinate (Intermediate 50)

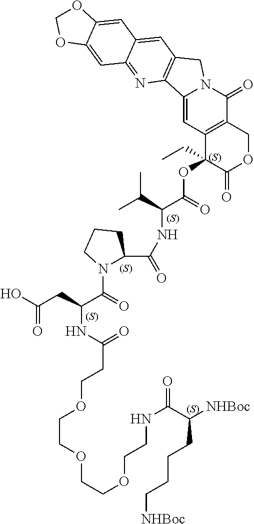

(7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-(3-{2-[2-(2-aminoethoxy)ethoxy]ethoxy} propanoyl)-L-alpha-aspartyl-L-prolyl-L-valinate trifluoroacetic acid (1/1) (109 mg, 107 μmol) (Intermediate 19) was dissolved in DMF (21 ml). Boc-Lys(Boc)-OSu: 2,5-dioxopyrrolidin-1-yl N²,N⁶-bis(tert-butoxycarbonyl)-L-lysinate (61.6 mg, 139 μmol) and N,N-diisopropylethylamine (56 μl, 320 μmol; CAS-RN:[7087-68-5]) were added. The reaction was stirred at RT for 20 hour and concentrated in vacuo. The residue was separated by prep. HPLC to give Intermediate 50 (103 mg, 100% purity, 78% yield) as a light yellow foam. LC-MS (Method 3): Rₜ=4.24 min; MS (ESIpos): m/z=1236 [M+H]⁺.

### Example I51: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N2,N6-bis(14-{4-[({(1R)-2-carboxy-1-[3-({3-[(propylcarbamoyl)amino]benzene-1-sulfonyl}amino)phenyl] ethyl} carbamoyl)amino]anilino}-14-oxo-4,7,10-trioxa-13-azatetradecanan-1-oyl)-L-lysyl]amino}ethoxy) ethoxy]ethoxy}propanoyl)-L-alpha-aspartyl-L-prolyl-L-valinate (Intermediate 51)

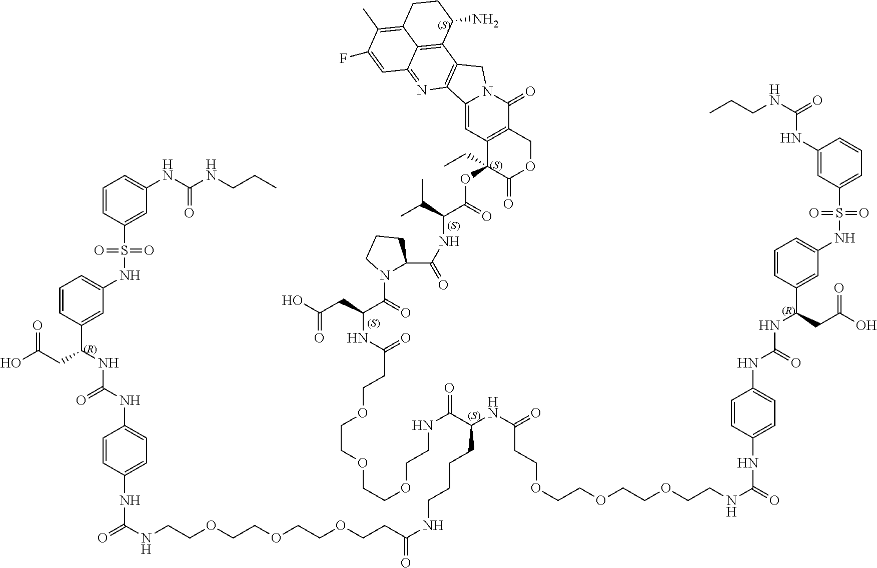

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate trifluoroacetic acid (1/2) (86.0 mg, 50.2 μmol) (Intermediate 52) was dissolved in DMF (25 ml). (3R)-3-{[(4-{[(4-Nitrophenoxy)carbonyl]amino}phenyl) carbamoyl]amino}-3-[3-({3-[(propylcarbamoyl)amino]benzene-1-sulfonyl}amino)phenyl] propanoic acid (72.3 mg, 100 μmol) (Building block 2) and N,N-diisopropylethylamine (170 μl, 1.0 mmol; CAS-RN:[7087-68-5]) were added. The reaction was stirred for 30 minutes at RT and concentrated in vacuo. The residue was separated by prep. HPLC. The purified substance could only be dissolved in DMSO, which is why it had to be separated three times to give Intermediate 51 (5.50 mg, 100% purity, 4% yield) as a yellow foam. LC-MS (Method 3): Rₜ=3.41 min; MS (ESIpos): m/z=2645 [M+H]⁺.

### Example I52: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N2,N6-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate trifluoroacetic acid (1/2) (Intermediate 52)

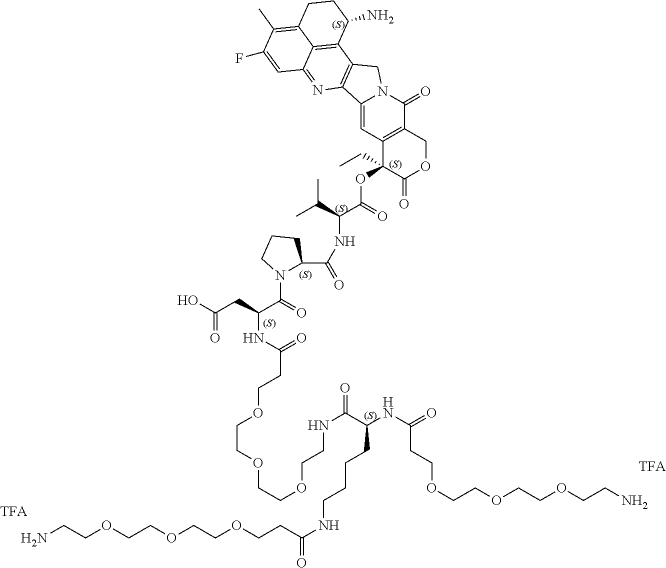

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(2,2-dimethyl-4,17-dioxo-3,8,11,14-tetraoxa-5-azaheptadecan-17-yl)-L-lysyl]amino}ethoxy)ethoxy] ethoxy}propanoyl)-L-alpha-aspartyl-L-prolyl-L-valinate (82.0 mg, 48.7 μmol) (Intermediate 53) was dissolved in DCM (14 ml), then TFA (2.8 ml) was added. The reaction was stirred for 1 hour at RT and then concentrated in vacuo. The residue was dissolved in ACN/H2O and freeze-dried to give Intermediate 52 (86.0 mg, 95% purity, 98% yield) as a yellow foam.

LC-MS (Method 3): Rₜ=1.96 min; MS (ESIpos): m/z=1484 [M+H]⁺.

### Example I53: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N2,N6-bis(2,2-dimethyl-4,17-dioxo-3,8,11,14-tetraoxa-5-azaheptadecan-17-yl)-L-lysyl]amino}ethoxy)ethoxy] ethoxy}propanoyl)-L-alpha-aspartyl-L-prolyl-L-valinate (Intermediate 53)

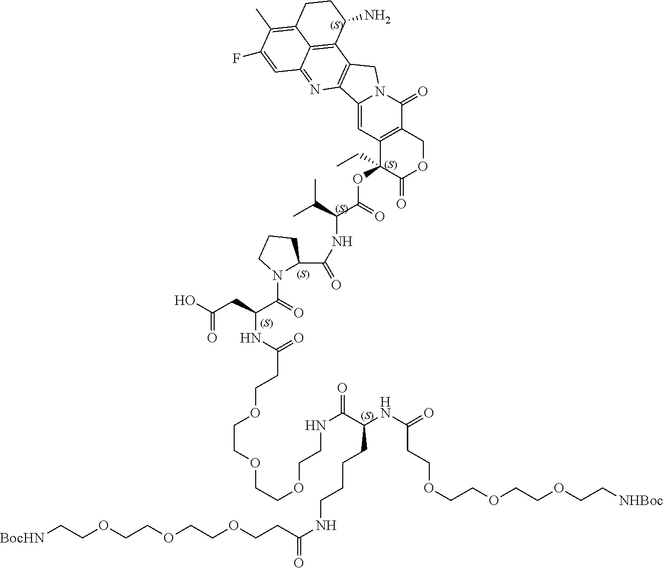

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-[3-(2-{2-[2-(L-lysylamino)ethoxy] ethoxy}ethoxy)propanoyl]-L-alpha-aspartyl-L-prolyl-L-valinate trifluoroacetic acid (1/2) (114 mg, 90% purity, 78.5 μmol) (Intermediate 54) was dissolved in DMF (25 ml). Tert-butyl {2-[2-(2-{3-[(2,5-dioxopyrrolidin-1-yl)oxy]-3-oxopropoxy}ethoxy)ethoxy]ethyl}carbamate (69.0 mg, 165 μmol) (Intermediate 49) and N,N-diisopropylethylamine (110 μl, 630 μmol; CAS-RN:[7087-68-5]) were added. The reaction was stirred at RT for 3 h and then concentrated in vacuo. The residue was purified by prep. HPLC to give Intermediate 53 (82.0 mg, 90% purity, 56% yield) as a colorless foam. LC-MS (Method 3): Rₜ=3.35 min; MS (ESIpos): m/z=1684 [M+H]⁺.

### Example I54: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-[3-(2-{2-[2-(L-lysylamino) ethoxy]ethoxy}ethoxy)propanoyl]-L-alpha-aspartyl-L-prolyl-L-valinate trifluoroacetic acid (1/2) (Intermediate 54)

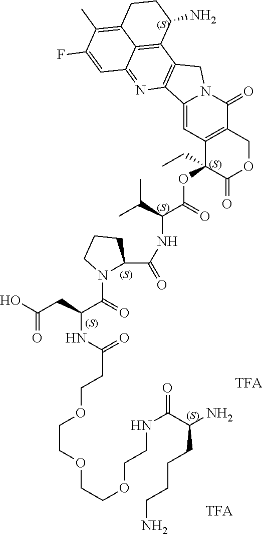

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(tert-butoxycarbonyl)-L-lysyl]amino}ethoxy)ethoxy]ethoxy}propanoyl)-L-alpha-aspartyl-L-prolyl-L-valinate (101 mg, 79.0 μmol) (Intermediate 55) was dissolved in DCM (15 ml), then TFA (5.0 ml) was added. The reaction was stirred for 1 hour at RT and concentrated in vacuo. The residue was dissolved in ACN/H2O and freeze-dried to give Intermediate 54 (114 mg, 100% purity, 110% yield) as a yellow foam. LC-MS (Method): Rₜ=1.17 min; MS (ESIpos): m/z=1078 [M+H]⁺.

### Example I55: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N2,N6-bis(tert-butoxycarbonyl)-L-lysyl]amino}ethoxy)ethoxy]ethoxy}propanoyl)-L-alpha-aspartyl-L-prolyl-L-valinate (Intermediate 55)

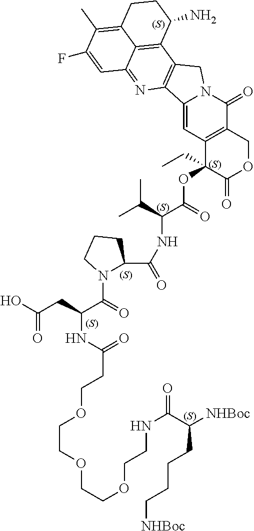

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate trifluoroacetic acid (1/1) (105 mg, 98.7 μmol) (Intermediate 56) was dissolved in DMF (20 ml). Boc-Lys(Boc)-OSu: 2,5-dioxopyrrolidin-1-yl N²,N⁶-bis(tert-butoxycarbonyl)-L-lysinate (48.1 mg, 109 μmol) and N,N-diisopropylethylamine (52 μl, 300 μmol; CAS-RN:[7087-68-5]) were added. The reaction was stirred at RT for 20 hour and concentrated in vacuo. The residue was separated by prep. HPLC to give Intermediate 55 (101 mg, 90% purity, 72% yield) as a colorless foam. LC-MS (Method 3): Rₜ=3.34 min; MS (ESIpos): m/z=1278 [M+H]⁺.

### Example I56: (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy) ethoxy]ethoxy}propanoyl)-L-alpha-aspartyl-L-prolyl-L-valinate trifluoroacetic acid (1/1) (Intermediate 56)

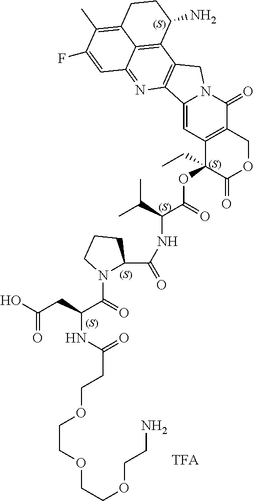

Tert-butyl (19S)-19-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (287 mg, 86% purity, 223 μmol) (Intermediate 57) was dissolved in DCM (30 ml), then TFA (5.0 ml) was added. The reaction was stirred for 1 hour at RT, then concentrated in vacuo. The residue was purified by prep. HPLC to give Intermediate 56 (216 mg, 95% purity, 87% yield) as a yellow foam. LC-MS (Method 3): Rₜ=2.04 min; MS (ESIpos): m/z=950 [M+H]⁺.

### Example I57: Tert-butyl (19S)-19-[(2S)-2-{[(2S)-1-{[(1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Intermediate 57)

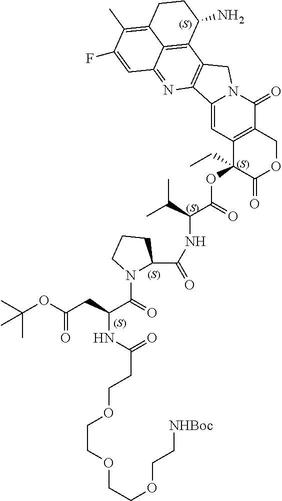

Tert-butyl (19S)-19-[(2S)-2-{[(2S)-1-{[(1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7] indolizino[1,2-b]quinolin-9-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (350 mg, 282 μmol) (Intermediate 14) was dissolved in ethanol (50 ml). The catalyst Pd/C 10% (50.0 mg) was added. The reaction was hydrogenated for 2 hour at standard pressure. The catalyst was filtered off and the filtrate was concentrated on a rotary evaporator. The residue was dissolved in ACN/H2O and freeze-dried to give Intermediate 57 (287 mg, 86% purity, 79% yield) as a colorless foam. LC-MS (Method 3): Rₜ=3.59 min; MS (ESIpos): m/z=1107 [M+H]⁺.

### Example I58: Preparation of (1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N2,N6-bis(14-{4-[({(1R)-2-carboxy-1-[3-({3-[(propylcarbamoyl)amino]benzene-1-sulfonyl}amino)phenyl] ethyl}carbamoyl)amino]anilino}-14-oxo-4,7,10-trioxa-13-azatetradecanan-1-oyl)-L-lysyl]amino} ethoxy)ethoxy]ethoxy}propanoyl)-L-alpha-aspartyl-L-prolyl-L-valinate (Intermediate 58)

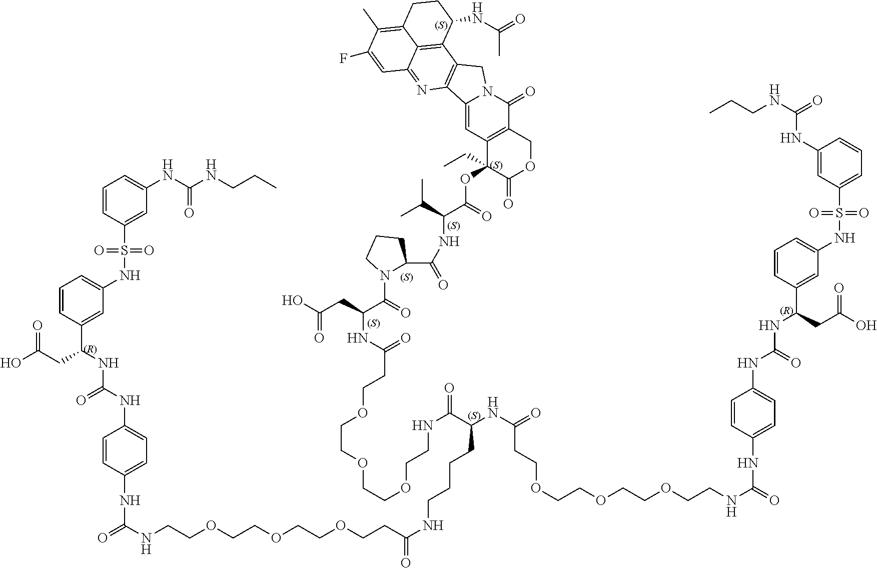

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl] ethyl}carbamoyl)amino]anilino}-14-oxo-4,7,10-trioxa-13-azatetradecanan-1-oyl)-L-lysyl] amino}ethoxy)ethoxy]ethoxy}propanoyl)-L-alpha-aspartyl-L-prolyl-L-valinate (20.0 mg, 7.56 μmol) (Intermediate 51) was dissolved in DMF (4.0 ml). Ac-OSu: 1-(acetyloxy)pyrrolidine-2,5-dione (2.38 mg, 15.1 μmol) and N,N-diisopropylethylamine (5.3 μl, 30 μmol; CAS-RN:[7087-68-5]) were added. The reaction was stirred at RT for 20 h and concentrated in vacuo. The residue was separated by prep. HPLC to give Intermediate 58 (3.40 mg, 92% purity, 15% yield) as a colorless foam. LC-MS (Method): Rₜ=7.65 min; MS (ESIpos): m/z=2687 [M+H]⁺.

### Compounds

### Example C1: Preparation of 2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(14-{4-[({(1R)-2-carboxy-1-[3-({3-[(propylcarbamoyl)amino]benzene-1-sulfonyl}amino)phenyl] ethyl}carbamoyl)amino]anilino}-14-oxo-4,7,10-trioxa-13-azatetradecanan-1-oyl)-L-alpha-aspartyl-L-prolyl-L-valinate (Compound 1)

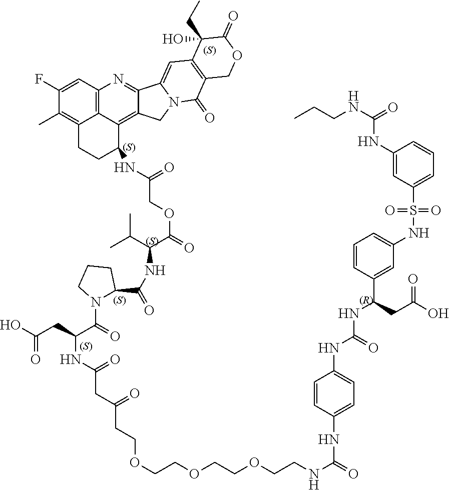

2-{[(1S,9S)-9-Ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(3-{2-[2-(2-aminoethoxy)ethoxy]ethoxy}propanoyl)-L-alpha-aspartyl-L-prolyl-L-valinate trifluoroacetic acid (1/1) (Intermediate 6) (15.0 mg, 13.4 μmol) was dissolved in DMF (5 mL). (3R)-3-{[(4-{[(4-nitrophenoxy) carbonyl]amino}phenyl)carbamoyl]amino}-3-[3-({3-[(propylcarbamoyl)amino]benzene-1-sulfonyl}amino)phenyl]propanoic acid (Building block 2) (9.62 mg, 13.4 μmol) and DIEA (47 μL, 270 μmol) were added. The reaction was stirred for 15 min at rt and then concentrated in vacuo. The residue was separated by prep. HPLC to give Compound 1 (1.93 mg. 94% purity, 8% yield) as a yellow foam. LC-MS (Method 3): Rₜ=3.81 min; MS (ESIpos): m/z=1588 [M+H]⁺.

### Example C2: Preparation of disodium (1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-valinate (Compound 2)

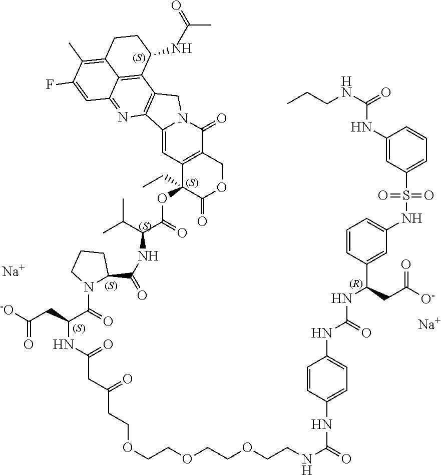

(1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy) ethoxy]ethoxy}propanoyl)-L-alpha-aspartyl-L-prolyl-L-valinate-trifluoroacetic acid (1/1) (Intermediate 10) (7.50 mg, 6.78 μmol) was dissolved in DMF (6 mL). (3R)-3-{[(4-{[(4-nitrophenoxy) carbonyl] amino} phenyl)carbamoyl]amino}-3-[3-({3-[(propylcarbamoyl) amino] benzene-1-sulfonyl}amino) phenyl]propanoic acid (Building block 2) (4.88 mg, 6.78 μmol) and DIEA (24 μL, 140 μmol) were added. The reaction was stirred for 15 min at rt. The reaction was evaporated to dryness and the residue was separated by prep. HPLC to give a yellow foam (9.4 mg, 5.98 μmol), which was dissolved in dioxane/H₂O (1:1, 3 mL). A sodium hydroxide solution (120 μl, 0.10 M, 12 μmol) was added. The solution was freeze-dried and Compound 2 (8.70 mg, 99% purity, 75% yield) was obtained as a colourless foam. The double peaks in the HPLC and LC-MS spectra were assigned to be rotamers based on previous structure-based verifications. LC-MS (Method 6): Rₜ=2.86 and 2.91 min; MS (ESIpos): m/z=1572 [M+H]⁺. ¹H-NMR (600 MHz, DMSO-d₆) δ[ppm]: 0.066 (0.44), 0.817 (7.99), 0.830 (16.00), 0.842 (8.80), 0.877 (7.47), 0.911 (13.20), 0.921 (8.84), 1.005 (1.16), 1.093 (0.50), 1.235 (2.54), 1.338 (0.80), 1.350 (3.00), 1.362 (5.39), 1.374 (5.26), 1.386 (2.72), 1.398 (0.68), 1.899 (14.94), 1.904 (12.97), 1.976 (1.51), 2.121 (3.08), 2.200 (1.94), 2.211 (2.72), 2.224 (2.83), 2.236 (2.42), 2.248 (1.96), 2.259 (2.12), 2.269 (2.36), 2.281 (1.91), 2.295 (1.93), 2.356 (1.93), 2.367 (2.25), 2.392 (11.38), 2.425 (0.84), 2.587 (1.95), 2.614 (1.52), 2.654 (0.55), 2.687 (0.96), 2.920 (2.21), 2.930 (4.59), 2.941 (4.48), 2.952 (1.95), 3.164 (4.08), 3.195 (4.12), 3.401 (5.37), 3.410 (6.59), 3.416 (6.36), 3.463 (5.92), 3.486 (8.50), 3.498 (14.62), 3.508 (12.45), 3.554 (2.04), 3.570 (2.44), 3.604 (2.40), 3.794 (1.95), 3.937 (0.86), 3.982 (0.95), 4.849 (1.55), 4.860 (1.55), 4.958 (2.17), 5.141 (1.08), 5.172 (1.68), 5.194 (1.95), 5.207 (1.80), 5.250 (1.79), 5.282 (1.09), 5.425 (0.64), 5.461 (5.77), 5.482 (0.94), 5.494 (0.65), 5.534 (1.75), 6.404 (0.51), 6.663 (2.41), 6.676 (2.49), 6.931 (2.52), 6.944 (3.04), 7.054 (2.33), 7.067 (4.00), 7.080 (2.34), 7.175 (3.23), 7.188 (3.92), 7.202 (2.87), 7.217 (7.67), 7.227 (7.02), 7.243 (2.60), 7.273 (3.82), 7.286 (4.97), 7.300 (2.44), 7.344 (1.51), 7.398 (1.25), 7.552 (1.83), 7.569 (5.55), 7.588 (1.68), 7.792 (2.47), 8.111 (1.56), 8.271 (1.37), 8.389 (0.71), 8.460 (2.72), 8.475 (2.67), 8.535 (0.55), 8.778 (2.62), 9.918 (0.79), 11.029 (0.51).

### Example C3: Preparation of (1S,9S)-1-{[(benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7] indolizino[1,2-b]quinolin-9-yl N-(14-{4-[({(1R)-2-carboxy-1-[3-({3-[(propylcarbamoyl)amino] benzene-1-sulfonyl}amino) phenyl]ethyl}carbamoyl)amino]anilino}-14-oxo-4,7,10-trioxa-13-azatetradecanan-1-oyl)-L-alpha-aspartyl-L-prolyl-L-valinate (Compound 3)

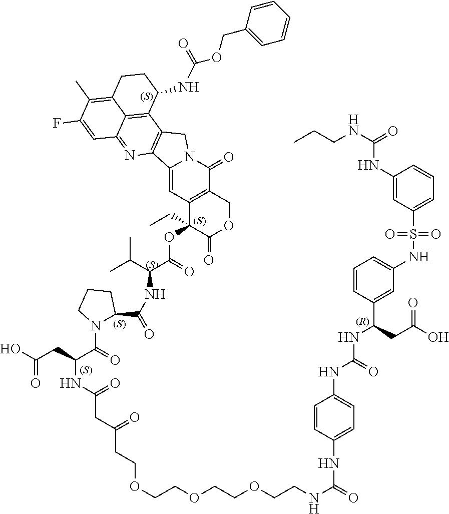

To a solution of (1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy) ethoxy]ethoxy}propanoyl)-L-alpha-aspartyl-L-prolyl-L-valinate-trifluoroacetic acid (1/1) (Intermediate 15) (48.0 mg, 40.1 μmol) in DMF (8 mL) were added (3R)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl]propanoic acid (Building block 2) (31.7 mg, 44.1 μmol) and DIEA (28 μl, 160 μmol). The reaction was then stirred for 2 h at rt and then concentrated in vacuo, purified by prep. HPLC and then lyophilized to give the title compound (44.5 mg, 100% purity, 67% yield) as a yellow foam. LC-MS (Method 3): Rₜ=4.56 min; MS (ESIpos): m/z=1664 [M+H]⁺.

### Example C4: Preparation of (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (Compound 4)

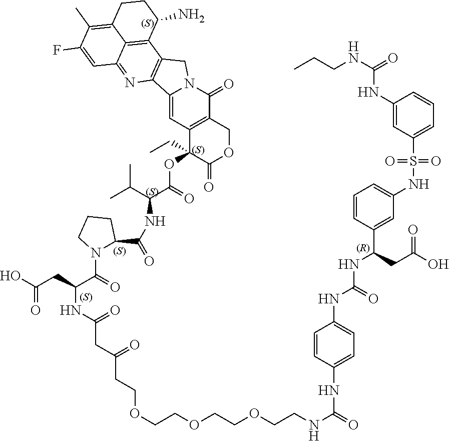

(1S,9S)-1-{[(Benzyloxy)carbonyl]amino}-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(14-{4-[({(1R)-2-carboxy-1-[3-({3-[(propylcarbamoyl)amino]benzene-1-sulfonyl}amino) phenyl]ethyl}carbamoyl)amino]anilino}-14-oxo-4,7,10-trioxa-13-azatetradecanan-1-oyl)-L-alpha-aspartyl-L-prolyl-L-valinate (Compound 3) (10.0 mg, 6.01 μmol) was hydrogenated with Pd/C 10% (2.0 mg) in EtOH (10 mL) at rt for 2 h and filtered. The filtrate was concentrated in vacuo, then solved in ACN/H₂O and lyophilized to give Compound 4 (5.50 mg, 92% purity, 55% yield) as a white foam. LC-MS (Method 3): Rₜ=3.15 min; MS (ESIpos): m/z=1530 [M+H]⁺. ¹H-NMR (600 MHz, DMSO-d₆): δ [ppm]=9.16-8.94 (m, 1H), 8.42-8.38 (m, 1H), 8.38-8.34 (m, 1H), 8.33 (s, 1H), 8.16-8.10 (m, 1H), 8.00 (br s, 1H), 7.61 (br d, 1H), 7.52-7.45 (m, 1H), 7.30-7.25 (m, 1H), 7.24 (s, 1H), 7.24-7.22 (m, 1H), 7.22-7.21 (m, 1H), 7.21 (br s, 1H), 7.20-7.18 (m, 4H), 7.14 (t, 1H), 6.98 (d, 1H), 6.88 (br d, 1H), 6.73-6.64 (m, 1H), 6.10-6.04 (m, 1H), 5.64 (br d, 1H), 5.53 (s, 1H), 5.49 (d, 2H), 5.46 (s, 1H), 5.41 (d, 1H), 4.99 (br d, 1H), 4.89 (br d, 1H), 4.78 (br dd, 1H), 4.74-4.66 (m, 1H), 4.09 (t, 1H), 3.75 (br d, 1H), 3.70-3.63 (m, 1H), 3.59 (br t, 2H), 3.51-3.50 (m, 1H), 3.54-3.50 (m, 4H), 3.50-3.45 (m, 6H), 3.45-3.43 (m, 1H), 3.43-3.39 (m, 4H), 3.39-3.38 (m, 3H), 3.27-3.18 (m, 7H), 3.18-3.13 (m, 3H), 3.09-2.95 (m, 4H), 2.72 (br dd, 1H), 2.69-2.59 (m, 3H), 2.54 (s, 16H), 2.53-2.52 (m, 1H), 2.46-2.41 (m, 2H), 2.41-2.36 (m, 4H), 2.36-2.31 (m, 2H), 2.26-2.10 (m, 6H), 2.03-1.95 (m, 2H), 1.95-1.87 (m, 2H), 1.51-1.33 (m, 4H), 1.33-1.16 (m, 5H), 1.01 (s, 1H), 0.94 (dd, 5H), 0.92-0.87 (m, 3H), 0.87-0.83 (m, 5H).

### Example C4b: Preparation of disodium (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-{(2S)-2-(carboxylatomethyl)-17-[4-({[(1R)-2-carboxylato-1-{3-[({3-[(propyl carbamoyl)amino]phenyl}sulfonyl)amino]phenyl}ethyl] carbamoyl}amino)anilino]-4,17-dioxo-7,10,13-trioxa-3,16-diazaheptadecan-1-oyl}-L-prolyl-L-valinate (Compound 4b)

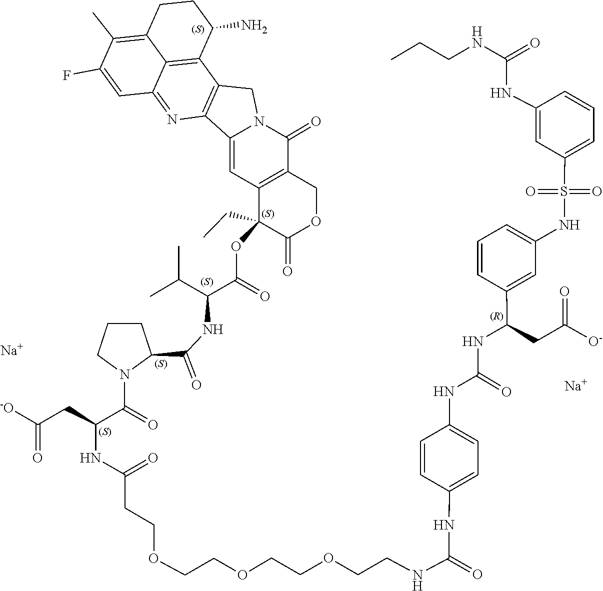

3 mg (2 μmol) of the Compound 4 was dissolved in dioxane/water (1:1, 2 mL). A sodium hydroxide solution (4 μmol, 0.1 M) was added. The solution was freeze-dried to give an almost colorless foam as Compound 4b (2 mg, 92% purity, 63% yield). LC-MS: (Method 6) Rₜ=2.36 min; MS (ESIpos): m/z=1530 [M+H]⁺.

### Example C5: Preparation of (7S)-7-ethyl-8,11-dioxo-7,8,11,13-tetrahydro-2H,10H-[1,3]dioxolo[4,5-g]pyrano [3′,4′:6,7]indolizino[1,2-b]quinolin-7-yl N-(14-{4-[({(1R)-2-carboxy-1-[3-({3-[(propylcarbamoyl) amino]benzene-1-sulfonyl}amino)phenyl]ethyl}carbamoyl) amino]anilino}-14-oxo-4,7,10-trioxa-13-azatetradecanan-1-oyl)-L-alpha-aspartyl-L-prolyl-L-valinate-bis sodium salt (Compound 5)

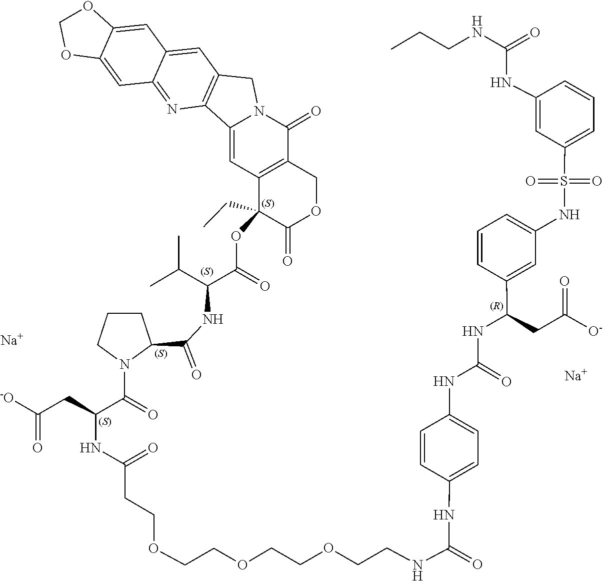

(7S)-7-Ethyl-8,11-dioxo-7,8,11,13-tetrahydro-2H,10H-[1,3]dioxolo[4,5-g]pyrano[3′,4′:6,7] indolizino[1,2-b]quinolin-7-yl N-(3-{2-[2-(2-aminoethoxy)ethoxy]ethoxy} propanoyl)-L-alpha-aspartyl-L-prolyl-L-valinate-trifluoroacetic acid (1/1) (Intermediate 18) (18.0 mg, 17.6 μmol) was dissolved in DMF (5 mL). (3R)-3-{[(4-{[(4-Nitrophenoxy)carbonyl] amino}phenyl)carbamoyl]amino}-3-[3-({3-[(propylcarbamoyl)amino]benzene-1-sulfonyl} amino)phenyl]propanoic acid (Building block 2) (14.0 mg, 19.4 μmol) and DIEA (61 μl, 350 μmol) were added. The batch was stirred for 30 min at rt. The reaction was evaporated to dryness and the residue was separated by prep. HPLC to give a yellow foam (18.5 mg, 12.44 μmol), which was dissolved in dioxane/water (1:1, 5 mL). A sodium hydroxide solution (24.87 μmol, 0.1 M) was added. The solution was freeze-dried to give an almost colorless foam as Compound 5 (19 mg, 100% purity, 67% yield). LC-MS (Method 3): Rₜ=3.81 min; MS (ESIpos): m/z=1487 [M+H]⁺. ¹H-NMR (600 MHz, DMSO-d₆): δ [ppm]=11.16-10.61 (m, 1H), 9.99-9.90 (m, 1H), 8.77-8.71 (m, 1H), 8.54-8.46 (m, 1H), 8.46-8.43 (m, 1H), 8.43-8.40 (m, 1H), 8.35-8.29 (m, 1H), 8.29-8.22 (m, 1H), 8.09-8.01 (m, 1H), 7.78-7.69 (m, 1H), 7.65-7.59 (m, 1H), 7.49 (s, 1H), 7.48 (br d, 1H), 7.30-7.29 (m, 1H), 7.29-7.27 (m, 1H), 7.25-7.23 (m, 1H), 7.24-7.22 (m, 1H), 7.21 (s, 2H), 7.19 (s, 2H), 7.19-7.17 (m, 1H), 7.08 (t, 1H), 6.94 (d, 1H), 6.69 (br d, 1H), 6.28 (d, 2H), 6.30-6.21 (m, 1H), 5.45 (s, 2H), 5.23-5.16 (m, 2H), 4.99-4.95 (m, 1H), 4.90-4.82 (m, 2H), 4.05 (br t, 1H), 3.80-3.75 (m, 1H), 3.75-3.69 (m, 1H), 3.62-3.58 (m, 1H), 3.58-3.54 (m, 1H), 3.50 (s, 4H), 3.50-3.47 (m, 3H), 3.46 (br d, 3H), 3.42 (br t, 3H), 3.20 (br dd, 3H), 2.98-2.91 (m, 2H), 2.74-2.67 (m, 1H), 2.64-2.58 (m, 1H), 2.54 (s, 9H), 2.53-2.52 (m, 1H), 2.43-2.33 (m, 3H), 2.33-2.27 (m, 1H), 2.26-2.21 (m, 1H), 2.21-2.15 (m, 2H), 2.37-2.12 (m, 1H), 2.14-2.07 (m, 1H), 2.04-1.98 (m, 1H), 1.97-1.85 (m, 2H), 1.41-1.34 (m, 2H), 1.27-1.22 (m, 1H), 1.03-1.00 (m, 1H), 0.94 (d, 3H), 0.93 (br d, 3H), 0.91-0.85 (m, 3H), 0.83 (t, 3H), 0.07 (d, 1H).

### Example C6: Preparation of (7S)-7-ethyl-8,11-dioxo-7,8,11,13-tetrahydro-2H,10H-[1,3]dioxolo[4,5-g]pyrano[3′,4′:6,7] indolizino[1,2-b]quinolin-7-yl N-{2-[{2-[{2-[({4-[({(1R)-2-carboxy-1-[3-({3-[(propylcarbamoyl) amino]benzene-1-sulfonyl}amino)phenyl]ethyl} carbamoyl)amino]phenyl}carbamoyl)amino] ethyl}(methyl)amino]ethyl}(methyl)amino]ethyl}-N-methylglycyl-L-asparaginyl-L-prolyl-L-valinate (Compound 6)

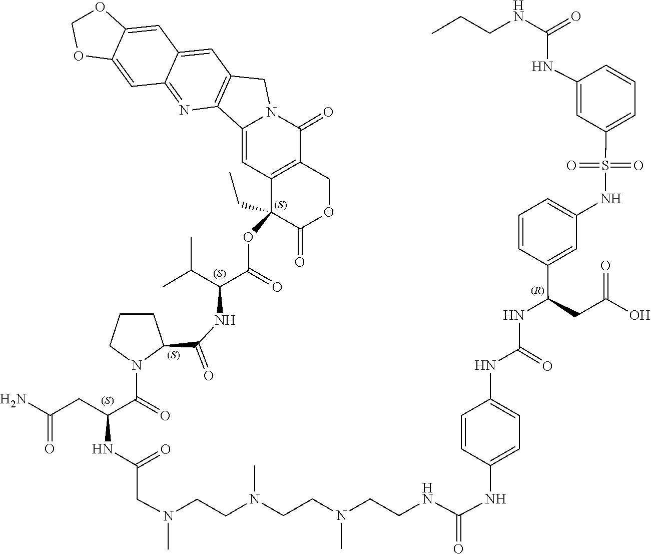

Trifluoroacetic acid-(7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl N-{2-[{2-[(2-aminoethyl)(methyl)amino] ethyl}(methyl)amino]ethyl}-N-methylglycyl-L-asparaginyl-L-prolyl-L-valinate (1/1) (Intermediate 28) (14.0 mg, 13.4 μmol) was dissolved in DMF (5 mL). (3R)-3-{[(4-{[(4-Nitrophenoxy)carbonyl]amino} phenyl)carbamoyl]amino}-3-[3-({3-[(propylcarbamoyl)amino] benzene-1-sulfonyl}amino) phenyl]propanoic acid (Building block 2) (10.6 mg, 14.7 μmol) and DIEA (47 μl, 270 μmol) were added. The reaction was stirred for 30 min at rt. The reaction was evaporated to dryness and the residue was purified by prep. HPLC to give Compound 6 (12.4 mg, 93% purity, 57% yield) as a yellow foam. LC-MS (Method 3): Rₜ=2.77 min; MS (ESIpos): m/z=1511 [M+H]⁺.

### Example C7: Preparation of N-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]acetamide (Compound 7)

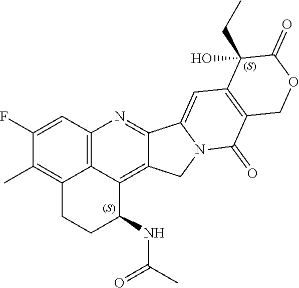

Methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (50.0 mg, 94.1 μmol) was dissolved in DMF (8.8 mL). 1-(Acetyloxy)pyrrolidine-2,5-dione (Ac-OSu) (29.6 mg, 188 μmol) and DIEA (66 μL, 380 μmol) were added. The batch was stirred at rt for 3 h. The reaction was evaporated to dryness. The residue was then purified by prep. HPLC to give Compound 7 (24.0 mg, 100% purity, 53% yield) as a light, yellow foam. LC-MS (Method 2): Rₜ=1.35 min; MS (ESIpos): m/z=478 [M+H]⁺.

### Example C8: Preparation of N-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]-2,2,2-trifluoroacetamide (Compound 8)

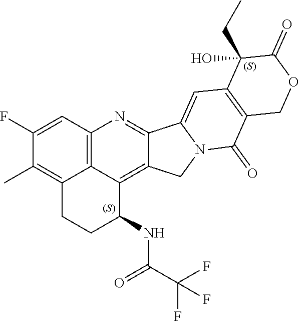

Methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (6 mg, 11.3 μmol) was added to DMF (2 mL). Trifluoroacetic anhydride (4.7 mg, 22.6 μmol) and DIEA (2.9 mg, 22.6 μmol) were added, and the reaction was stirred overnight at rt. More trifluoroacetic anhydride (20 μL) and DIEA (10 μL) were added and stirring was continued for an additional 48 h. The reaction was concentrated in vacuo, dissolved in ACN/H₂O and purified by prep. HPLC, then concentrated and lyophilized to give Compound 8 (3.20 mg, 100% purity, 53% yield) as a yellow residue. LC-MS (Method 2): Rₜ=1.67 min; MS (ESIpos): m/z=532 [M+H]⁺. ¹H NMR (500 MHz, DMSO-d₆) δ ppm 0.882 (t, J=7.32 Hz, 3H) 1.815-1.950 (m, 2H) 2.150-2.256 (m, 1H) 2.300 (s, 3H) 2.424 (s, 3H) 2.537 (br d, J=14.42 Hz, 1H) 3.052-3.171 (m, 1H) 3.242-3.337 (m, 2H) 5.105 (br s, 1H) 5.393-5.495 (m, 3H) 5.705 (d, J=19.15 Hz, 1H) 6.352-6.821 (m, 1H) 7.347 (s, 1H) 7.885 (d, J=10.83 Hz, 1H) 8.437 (br d, J=4.58 Hz, 3H)

### Example C9: Preparation of N-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]-2-(pyridin-4-yl)acetamide (Compound 9)

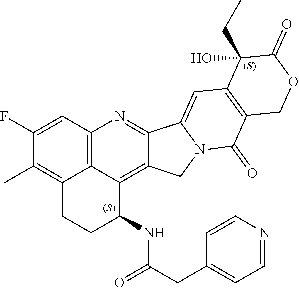

Methanesulfonic acid-(1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (6.00 mg, 11.3 μmol) was added to DMF (4 mL). Then (pyridin-4-yl)acetic acid-hydrogen chloride (1/1) (2.35 mg, 13.5 μmol), HATU (6.44 mg, 16.9 μmol) and DIEA (7.9 μl, 45 μmol) were added and the reaction was stirred for 30 min at rt. The reaction was concentrated in vacuo and the residue was purified by prep. HPLC and lyophilized to give Compound 9 (4.0 mg, 100% purity, 64% yield) as a yellow foam. LC-MS (Method 2): Rₜ=1.12 min; MS (ESIpos): m/z=555 [M+H]⁺.

### Example C10: Preparation of N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]-N²-[(pyridin-4-yl)acetyl]-L-aspartamide (Compound 10)

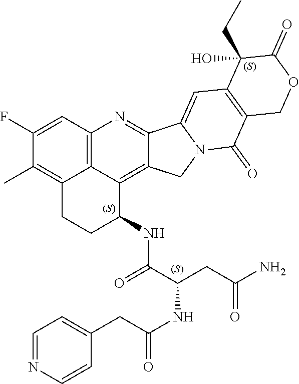

Trifluoroacetic acid-N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]-L-aspartamide (1/1) (Intermediate 30) (5.00 mg, 98% purity, 7.41 μmol) was added to DMF (2 mL), then (pyridin-4-yl)acetic acid-hydrogen chloride (1/1) (1.54 mg, 8.90 μmol), HATU (4.23 mg, 11.1 μmol) and DIEA (5.2 μl, 30 μmol) were added. The reaction was stirred for 10 min at rt. The reaction was concentrated in vacuo and the residue was purified by prep. HPLC and lyophilized to give Compound 10 (2.70 mg, 100% purity, 54% yield) as a yellow foam. LC-MS (Method 2): Rₜ=0.95 min; MS (ESIpos): m/z=669 [M+H]⁺.

### Example C11: Preparation of N-[(pyridin-4-yl)acetyl]-L-alanyl-N-methyl-L-alanyl-N¹-[(1S,9S)-9-ethyl-5-fluoro-9-hydroxy-4-methyl-10,13-dioxo-2,3,9,10,13,15-hexahydro-1H, 12H-benzo[de]pyrano [3′,4′:6,7] indolizino [1,2-b]quinolin-1-yl]-L-aspartamide (Compound 11)

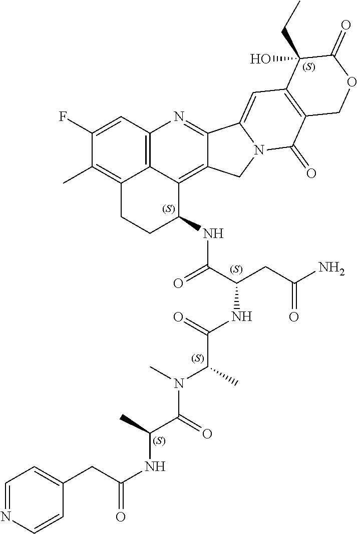

Trifluoroacetic acid-L-alanyl-N-methyl-L-alanyl-N¹-[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino [1,2-b]quinolin-1-yl]-L-aspartamide (Intermediate 32) (1/1) (5.00 mg, 6.10 μmol) was added to DMF (2 mL), then (pyridin-4-yl)acetic acid-hydrogen-chloride (1/1) (1.27 mg, 7.32 μmol), HATU (3.48 mg, 9.15 μmol) and DIEA (4.2 μl, 24 μmol) were added and the reaction was stirred for 10 min at rt. It was then concentrated in vacuo and the residue was purified by prep. HPLC and lyophilized to give Compound 11 (4.0 mg, 100% purity, 80% yield) as a yellow foam. LC-MS (Method 6): Rₜ=1.72 min; MS (ESIpos): m/z=825 [M+H]⁺.

### Example C12: Preparation of 2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethyl N-(14-{4-[({(1S)-2-carboxy-1-[3-({3-[(propylcarbamoyl) amino]benzene-1-sulfonyl}amino)phenyl] ethyl}carbamoyl)amino]anilino}-14-oxo-4,7,10-trioxa-13-azatetradecanan-1-oyl)-L-alpha-aspartyl-L-prolyl-L-valinate (Compound 12)

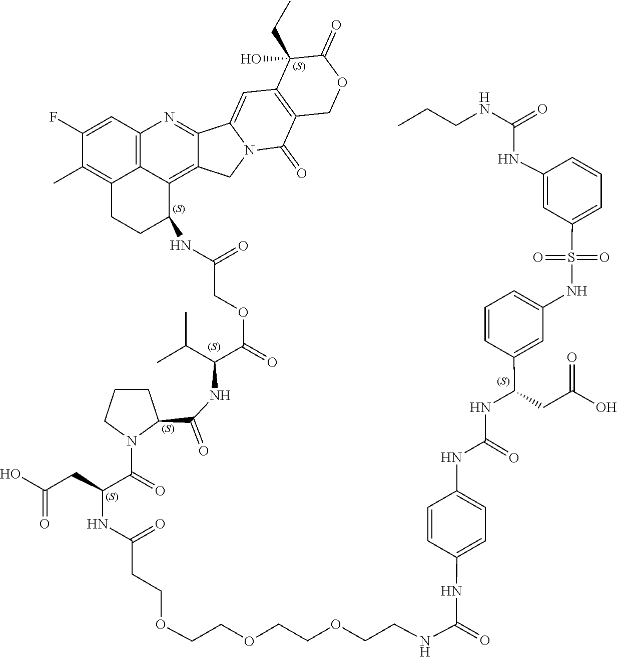

Compound 12 is the epimer of Compound 1 and was synthesized in analogy to Example C1 employing the enantiomeric Building block 10 instead of building block 2. Yield: 0.86 mg (100% purity, 4% yield). LC-MS (Method 6): Rₜ=2.8 min; MS (ESIpos): m/z=1588 [M+H]⁺.

### Example C13: Preparation of disodium (1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-[(2S)-2-(carboxylatomethyl)-17-{4-[({(1S)-2-carboxylato-1-[3-({3-[(propyl carbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-valinate (Compound 13)

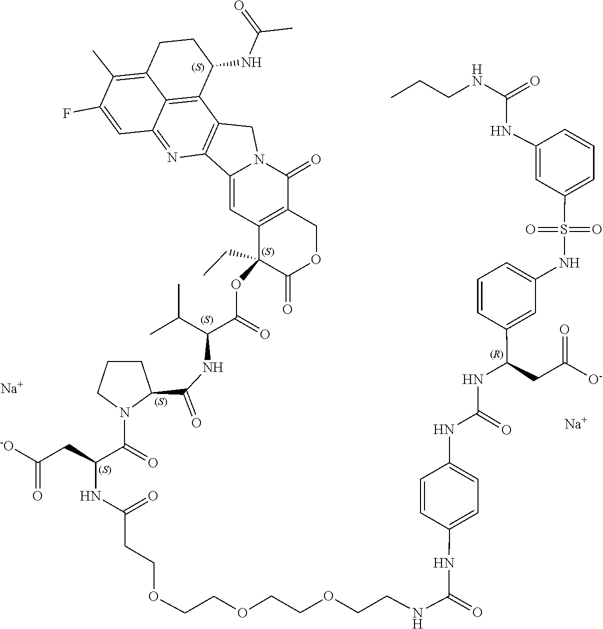

Compound 13 is the epimer of Compound 2 and was synthesized in analogy to Example C2 employing the enatiomeric Building block 10 instead of building block 2. Yield: 2.7 mg (97% purity, 57% yield over 2 steps). LC-MS (Method 6): Rₜ=2.89 min and 2.92 min; MS (ESIpos): m/z=1572 [M+H]⁺.

### Example C14: Preparation of (7S)-7-ethyl-8,11-dioxo-7,8,11,13-tetrahydro-2H,10H-[1,3]dioxolo[4,5-g]pyrano[3′,4′:6,7] indolizino[1,2-b]quinolin-7-yl N-{2-[{2-[{2-[({4-[({(1S)-2-carboxy-1-[3-({3-[(propylcarbamoyl) amino]benzene-1-sulfonyl}amino)phenyl]ethyl} carbamoyl)amino]phenyl}carbamoyl)amino] ethyl}(methyl)amino]ethyl}(methyl)amino]ethyl}-N-methylglycyl-L-asparaginyl-L-prolyl-L-valinate (Compound 14)

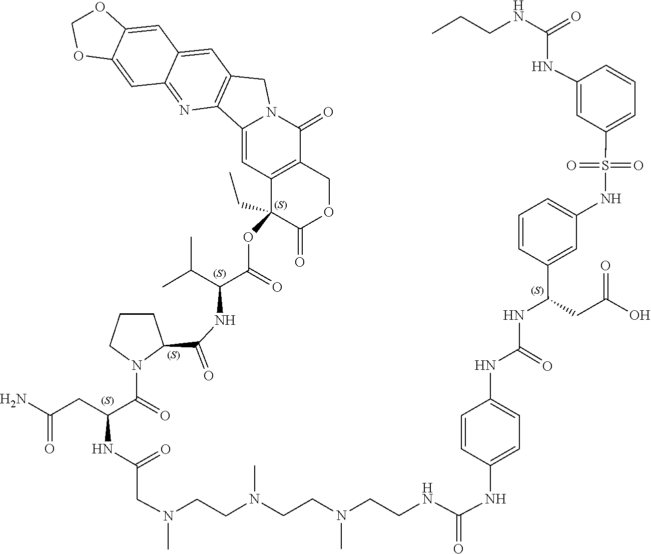

Compound 14 is the epimer of Compound 6 and was synthesized in analogy to Example C6 employing the enantiomeric Building block 10 instead of building block 2. Yield: 8.9 mg (93% purity, 38% yield). LC-MS (Method 6): Rₜ=2.07 min; MS (ESIpos): m/z=1511 [M+H]⁺.

### Example C15: Preparation of (7S)-7-ethyl-8,11-dioxo-7,8,11,13-tetrahydro-2H,10H-[1,3]dioxolo[4,5-g]pyrano [3′,4′:6,7]indolizino[1,2-b]quinolin-7-yl N-(14-{4-[({(1S)-2-carboxy-1-[3-({3-[(propylcarbamoyl) amino]benzene-1-sulfonyl}amino)phenyl]ethyl}carbamoyl) amino]anilino}-14-oxo-4,7,10-trioxa-13-azatetradecanan-1-oyl)-L-alpha-aspartyl-L-prolyl-L-valinate -bis sodium salt (Compound 15)

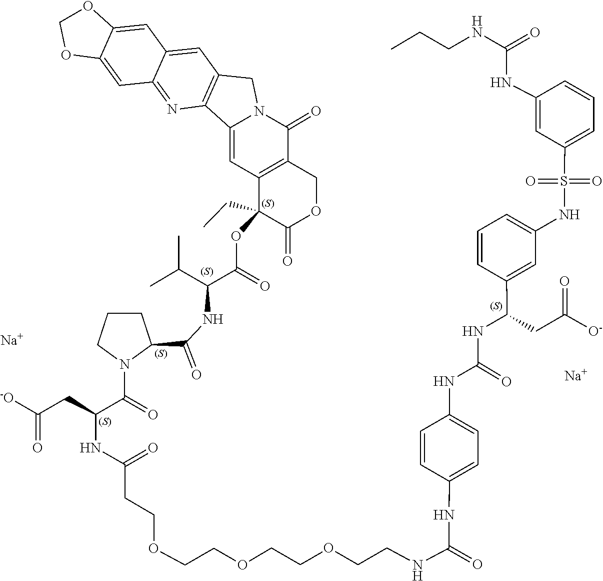

Compound 15 is the epimer of Compound 5 and was synthesized in analogy to Example C5 employing the enatiomeric Building block 10 instead of building block 2. Yield: 23 mg (100% purity, 83% yield). LC-MS (Method 3): Rₜ=3.80 min; MS (ESIpos): m/z=1487 [M+H]⁺. ¹H NMR (600 MHz, DMSO-d₆) δ ppm 0.831 (t, J=7.43 Hz, 3H) 0.859-0.916 (m, 3H) 0.928 (d, J=6.65 Hz, 5H) 1.202-1.298 (m, 1H) 1.313-1.428 (m, 2H) 1.862-1.956 (m, 2H) 1.956-2.034 (m, 1H) 2.063-2.140 (m, 1H) 2.171-2.220 (m, 2H) 2.220-2.285 (m, 1H) 2.285-2.321 (m, 1H) 2.332-2.370 (m, 1H) 2.370-2.392 (m, 1H) 2.525-2.562 (m, 8H) 2.562-2.577 (m, 1H) 2.577-2.655 (m, 1H) 2.665-2.732 (m, 1H) 2.903-3.029 (m, 2H) 3.143-3.285 (m, 4H) 3.376-3.531 (m, 13H) 3.539-3.608 (m, 3H) 3.663-3.818 (m, 2H) 3.951-4.079 (m, 1H) 4.837-4.930 (m, 2H) 4.930-5.002 (m, 1H) 5.184 (s, 1H) 5.450 (s, 1H) 6.251-6.295 (m, 2H) 6.686 (d, J=8.02 Hz, 1H) 6.943 (d, J=7.63 Hz, 1H) 7.073 (t, J=7.92 Hz, 1H) 7.172-7.195 (m, 1H) 7.195-7.222 (m, 2H) 7.222-7.270 (m, 3H) 7.270-7.308 (m, 2H) 7.445-7.476 (m, 1H) 7.481 (s, 1H) 7.561-7.629 (m, 1H) 7.712-7.832 (m, 1H) 8.023-8.097 (m, 1H) 8.259-8.307 (m, 1H) 8.307-8.396 (m, 1H) 8.420-8.455 (m, 1H) 8.520-8.620 (m, 1H) 8.713-8.899 (m, 1H) 9.872-10.008 (m, 1H) 10.875-11.069 (m, 1H).

### Example C16: Preparation of trifluoroacetic acid (1S,9S)-9-ethyl-1-(ethylamino)-5-fluoro-9-hydroxy-4-methyl-1,2,3,9,12,15-hexahydro-10H,13H-benzo[de]pyrano[3′,4′:6,7] indolizino[1,2-b]quinoline-10,13-dione (1/1) (Compound 16)

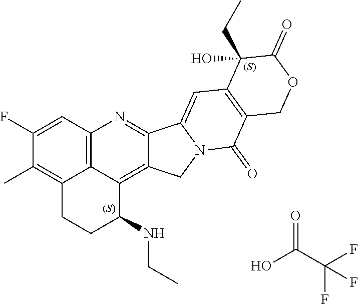

Methanesulfonic acid (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (25.0 mg, 47.0 μmol) was dissolved in MeOH (5.0 mL). Sodium hydrogen carbonate (15.8 mg, 188.1 μmol) was added. Then acetaldehyde (10.5 μl, 0.188 mmol) was added and the reaction was stirred for 2 h at reflux. The reaction was then cooled to 0° C., then sodium triacetoxyborohydride (39.9 mg, 188.1 μmol) was added. The reaction was stirred at RT for 1 h while monitoring by HPLC and LC-MS. The reaction was concentrated in vacuo and the residue was purified by prep. HPLC to give Compound 16 (10.80 mg, 100% purity, 40% yield) as a yellow foam. LC-MS (Method 2): Rₜ=0.86 min; MS (ESIpos): m/z=464 [M+H]⁺. ¹H-NMR (600 MHz, DMSO-d₆): δ [ppm]=9.04-8.94 (m, 1H), 8.77-8.65 (m, 1H), 7.89 (d, 1H), 7.35 (s, 1H), 6.64-6.49 (m, 1H), 5.55-5.50 (m, 1H), 5.46 (s, 2H), 5.45-5.40 (m, 1H), 5.04 (br s, 1H), 3.35-3.28 (m, 2H), 3.28-3.22 (m, 1H), 3.19-3.11 (m, 2H), 2.77-2.64 (m, 1H), 2.41 (s, 3H), 2.23-2.14 (m, 1H), 1.94-1.82 (m, 2H), 1.20 (t, 3H), 0.88 (t, 3H).

### Example C17: Preparation of (1S,9S)-1-(diethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Compound 17)

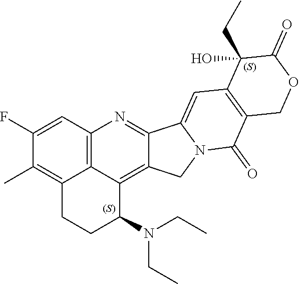

Methanesulfonic acid (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (1/1) (exatecan mesylate) (25.0 mg, 47.0 μmol) was dissolved in DCM (8.0 mL). Sodium hydrogen carbonate (7.90 mg, 94.1 μmol) was added. Acetaldehyde (21 μl, 380 μmol) was added and the reaction was stirred for 1 h at reflux. Acetaldehyde (21 μl, 380 μmol) was added twice more and the reaction was stirred for 1 h at reflux. The reaction was cooled to 0° C., then sodium triacetoxyborohydride (39.9 mg, 188 μmol) was added. The reaction was stirred at RT for 20 h. The reaction was concentrated in vacuo and purified by prep. HPLC to give Compound 17 (20.5 mg, 100% purity, 89% yield) as a colorless foam. LC-MS (Method 2): Rₜ=1.22 min; MS (ESIpos): m/z=492 [M+H]⁺. ¹H-NMR (600 MHz, DMSO-d₆): δ [ppm]=9.35-9.28 (m, 2H), 7.89 (s, 1H), 7.88 (s, 1H), 7.77-7.67 (m, 1H), 7.35 (s, 2H), 7.31-7.28 (m, 1H), 6.62-6.49 (m, 2H), 5.54-5.48 (m, 2H), 5.46-5.44 (m, 1H), 5.48-5.39 (m, 5H), 5.31 (br d, 2H), 5.36-5.23 (m, 1H), 5.07 (br d, 2H), 4.52-4.46 (m, 1H), 3.43-3.27 (m, 7H), 3.24-3.11 (m, 5H), 3.07-2.98 (m, 2H), 2.98-2.89 (m, 1H), 2.83-2.79 (m, 1H), 2.65-2.56 (m, 2H), 2.40 (br s, 5H), 2.44-2.28 (m, 5H), 2.23-2.15 (m, 2H), 2.07-1.95 (m, 1H), 1.93-1.81 (m, 6H), 1.34 (br t, 6H), 1.18-1.03 (m, 11H), 0.89 (br t, 8H).

### Example C18: Preparation of (1S,9S)-1-(dimethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Compound 18)

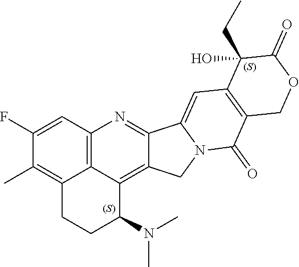

(1S,9S)-1-(dimethylamino)-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (Compound 18) was prepared in analogy to example compound C17 from exatecan mesylate LC-MS (Method 4): Rₜ=1.01 min; MS (ESIpos): m/z=464 [M+H]⁺.

### Example C19: Preparation of (1S,9S)-1-(diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(14-{4-[({(1R)-2-carboxy-1-[3-({3-[(propylcarbamoyl)amino]benzene-1-sulfonyl}amino)phenyl]ethyl} carbamoyl)amino]anilino}-14-oxo-4,7,10-trioxa-13-azatetradecanan-1-oyl)-L-alpha-aspartyl-L-prolyl-L-valinate (Compound 19)

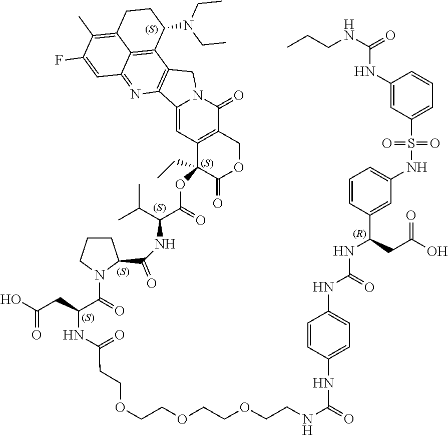

(1S,9S)-1-(Diethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate trifluoroacetic acid (1/1) (11.8 mg, 80% purity, 8.43 μmol) (Intermediate 33) was dissolved in DMF (4.6 ml). (3R)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl]propanoic acid (6.07 mg, 8.43 μmol) (Building block 2) and N,N-diisopropylethylamine (29 μl, 170 μmol; CAS-RN:[7087-68-5]) were added. The reaction was stirred for 30 minutes at RT and then concentrated in vacuo. The crude residue was separated twice by prep. HPLC to give Compound 19 (330 μg, 95% purity, 2% yield) as a colorless foam. LC-MS (Method 3): Rₜ=3.65 min; MS (ESIpos): m/z=1586 [M+H]⁺. ¹H NMR (600 MHz, DMSO-d₆) δ ppm 0.855 (t, J=7.43 Hz, 1H) 0.895-0.933 (m, 1H) 0.933-0.959 (m, 1H) 1.056-1.104 (m, 1H) 1.315-1.348 (m, 1H) 1.407-1.446 (m, 1H) 1.893-1.951 (m, 1H) 1.964-2.005 (m, 1H) 2.089-2.130 (m, 1H) 2.142-2.214 (m, 1H) 2.312-2.376 (m, 1H) 2.376-2.429 (m, 1H) 2.429-2.483 (m, 1H) 2.541 (s, 4H) 2.606-2.654 (m, 1H) 2.678-2.741 (m, 1H) 2.752-2.827 (m, 1H) 2.957-3.010 (m, 1H) 3.010-3.049 (m, 1H) 3.091-3.131 (m, 1H) 3.173-3.237 (m, 1H) 3.262-3.372 (m, 1H) 3.372-3.431 (m, 2H) 3.569-3.621 (m, 1H) 3.645-3.707 (m, 1H) 3.738-3.766 (m, 1H) 4.062-4.092 (m, 1H) 4.761-4.781 (m, 1H) 4.874-4.911 (m, 1H) 4.977-5.014 (m, 1H) 5.036-5.094 (m, 1H) 5.305 (br d, J=19.76 Hz, 1H) 5.494-5.523 (m, 1H) 5.514-5.577 (m, 1H) 6.016-6.086 (m, 1H) 6.215-6.228 (m, 1H) 6.630 (br d, J=8.22 Hz, 1H) 6.899 (d, J=7.94 Hz, 1H) 6.986 (d, J=7.63 Hz, 1H) 7.130-7.187 (m, 1H) 7.197 (s, 1H) 7.232-7.258 (m, 1H) 7.258-7.290 (m, 1 H) 7.401 (br d, J=8.41 Hz, 1H) 7.693 (d, J=9.78 Hz, 1H) 8.056 (s, 1H) 8.114 (br d, J=8.61 Hz, 1H) 8.312-8.341 (m, 1H) 8.351 (s, 1H) 8.753 (br d, J=0.98 Hz, 1H) 8.762 (s, 1H) 9.016-9.138 (m, 1H) 10.265 (s, 1H) 12.000-12.583 (m, 1H).

### Example C20: Preparation of (1S,9S)-1-(dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (Compound 20)

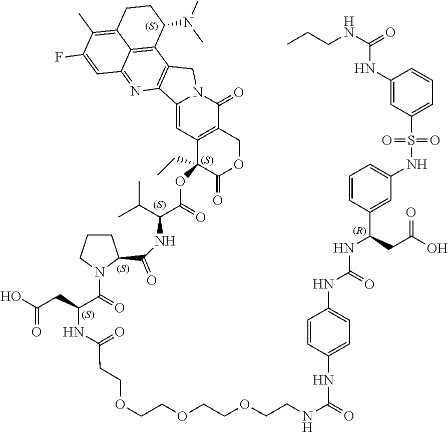

(1S,9S)-1-(Dimethylamino)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-aminoethoxy)ethoxy]ethoxy}propanoyl)-L-alpha-aspartyl-L-prolyl-L-valinate trifluoroacetic acid (1/1) (10.0 mg, 9.16 μmol) (Intermediate 37) was dissolved in DMF (5.1 ml). (3R)-3-{[(4-{[(4-Nitrophenoxy)carbonyl]amino}phenyl)carbamoyl]amino}-3-[3-({3-[(propylcarbamoyl)amino] benzene-1-sulfonyl}amino)phenyl]propanoic acid (6.59 mg, 9.16 μmol) (Building block 2) and N,N-diisopropylethylamine (32 μl, 180 μmol; CAS-RN:[7087-68-5]) were added. The reaction was stirred for 30 minutes at RT and then concentrated in vacuo. The residue was separated by prep. HPLC to give Compound 20 (1.70 mg, 100% purity, 12% yield) as a colorless foam. LC-MS (Method 3): Rₜ=3.33 min; MS (ESIpos): m/z=1558 [M+H]⁺.

### Example C21: Preparation of (1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino] ethyl}(methyl)amino]ethyl}-N-methylglycyl-L-asparaginyl-L-prolyl-L-valinate (Compound 21)

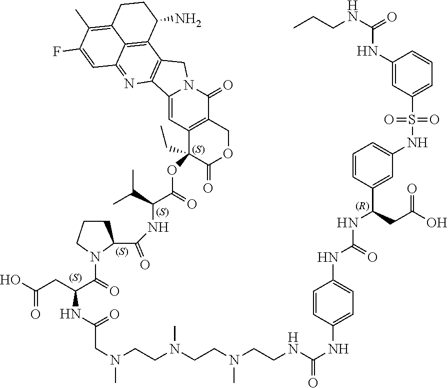

Trifluoroacetic acid (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-{2-[{2-[(2-aminoethyl)(methyl)amino]ethyl}(methyl)amino]ethyl}-N-methylglycyl-L-asparaginyl-L-prolyl-L-valinate (3/1) (16.0 mg, 12.2 μmol) (Intermediate 42) was dissolved in DMF (4.9 ml). (3R)-3-{[(4-{[(4-Nitrophenoxy)carbonyl]amino}phenyl)carbamoyl]amino}-3-[3-({3-[(propylcarbamoyl) amino]benzene-1-sulfonyl}amino)phenyl]propanoic acid (8.75 mg, 12.2 μmol) (Building block 2) and N,N-diisopropylethylamine (21 μl, 120 μmol; CAS-RN:[7087-68-5]) were added. The reaction was stirred for 30 minutes at RT and then concentrated in vacuo. The residue was separated by prep. HPLC to give Compound 21 (1.80 mg, 100% purity, 10% yield) as a yellow foam. LC-MS (Method 3): Rₜ=2.38 min; MS (ESIpos): m/z=1555 [M+H]⁺. ¹H NMR (600 MHz, DMSO-d₆) δ ppm 0.855 (t, J=7.43 Hz, 1H) 0.888-0.950 (m, 3H) 1.040 (dd, J=9.59, 6.65 Hz, 1H) 1.407-1.447 (m, 1H) 1.911-1.978 (m, 1H) 2.133-2.245 (m, 2H) 2.286-2.347 (m, 1H) 2.414 (s, 1H) 2.541 (s, 5H) 2.597-2.655 (m, 1H) 2.708-2.900 (m, 3H) 3.010-3.050 (m, 1H) 3.138-3.332 (m, 4H) 3.549-3.745 (m, 3H) 3.788-3.872 (m, 1H) 4.130 (br t, J=7.92 Hz, 1H) 4.714-4.737 (m, 1H) 4.937-5.013 (m, 1H) 5.089-5.110 (m, 1H) 5.443 (d, J=18.98 Hz, 1H) 5.471-5.560 (m, 1H) 5.698 (br d, J=18.58 Hz, 1H) 6.287-6.309 (m, 1H) 6.502-6.607 (m, 1H) 6.710 (br d, J=8.41 Hz, 1H) 6.880 (d, J=7.90 Hz, 1H) 6.971-6.989 (m, 1H) 7.135 (s, 1H) 7.147 (s, 1H) 7.161 (s, 1H) 7.201 (s, 1H) 7.225-7.254 (m, 1H) 7.237 (s, 1H) 7.258 (s, 1H) 7.271 (s, 1H) 7.284 (s, 1H) 7.406 (br d, J=8.80 Hz, 1H) 7.502 (br s, 1H) 7.663 (d, J=10.56 Hz, 1H) 8.060-8.071 (m, 1H) 8.280 (br d, J=9.00 Hz, 1H) 8.428 (br s, 1H) 8.491-8.542 (m, 1H) 8.637-8.743 (m, 1H) 8.831 (s, 1H) 8.912-9.018 (m, 1H) 10.258 (s, 1H) 12.188-12.440 (m, 1H).

### Example C22: Preparation of trisodium (7S)-7-ethyl-8,11-dioxo-7,8,11,13-tetrahydro-2H,10H-[1,3]dioxolo[4,5-g]pyrano[3′,4′:6,7]indolizino[1,2-b]quinolin-7-yl 1-[(2S)-15-{[N2,N6-bis(14-{4-[({(1R)-2-carboxylato-1-[3-({3-[(propylcarbamoyl)amino]benzene-1-sulfonyl}amino)phenyl]ethyl} carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-valinate (Compound 22)

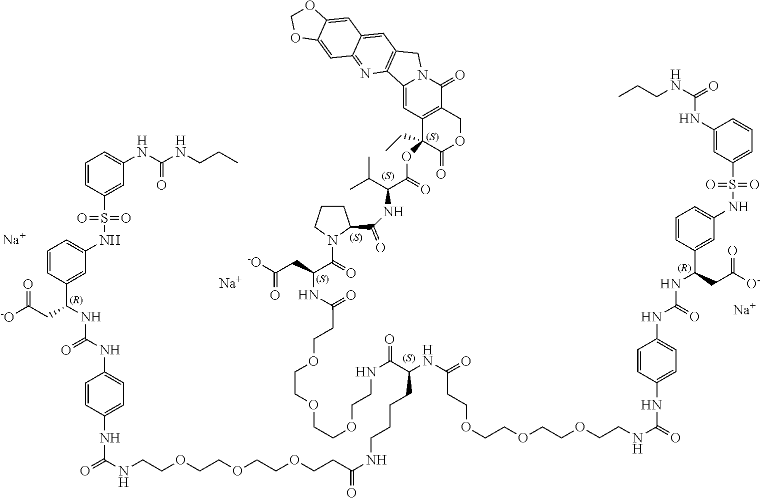

(7S)-7-Ethyl-8,11-dioxo-7,8,11,13-tetrahydro-2H,10H-[1,3]dioxolo[4,5-g]pyrano[3′,4′:6,7]indolizino[1,2-b]quinolin-7-yl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl]ethyl} carbamoyl)amino]anilino}-14-oxo-4,7,10-trioxa-13-azatetradecanan-1-oyl)-L-lysyl]amino} ethoxy)ethoxy]ethoxy}propanoyl)-L-alpha-aspartyl-L-prolyl-L-valinate (89.0 mg, 34.2 μmol) (Intermediate 45) was dissolved in dioxane/water (1:1, 30 ml). A sodium hydroxide solution (100 μl, 1.0 M, 100 μmol) was added and the solution was freeze-dried to give Compound 22 (90.0 mg, 100% purity, 99% yield) as a colorless foam. LC-MS (Method 6): Rₜ=3.00 min; MS (ESIpos): m/z=1302[M+2H]²⁺. ¹H-NMR (600 MHz, DMSO-d₆) δ[ppm]: 0.815 (5.47), 0.828 (11.48), 0.840 (5.74), 0.876 (2.07), 0.889 (4.05), 0.906 (5.47), 0.917 (5.15), 1.012 (0.45), 1.234 (1.02), 1.336 (1.28), 1.348 (2.86), 1.360 (4.23), 1.372 (3.77), 1.384 (1.92), 1.396 (0.49), 1.447 (0.47), 1.463 (0.47), 1.581 (0.44), 1.904 (0.67), 2.005 (0.48), 2.088 (0.40), 2.192 (1.28), 2.204 (1.24), 2.272 (1.59), 2.282 (2.71), 2.293 (1.51), 2.313 (1.05), 2.336 (0.84), 2.347 (1.24), 2.359 (1.17), 2.370 (1.72), 2.380 (1.79), 2.425 (0.42), 2.522 (1.50), 2.591 (1.35), 2.613 (1.30), 2.677 (0.42), 2.690 (0.46), 2.704 (0.42), 2.918 (1.35), 2.928 (2.84), 2.939 (2.81), 2.950 (1.30), 2.974 (0.95), 2.984 (1.14), 2.993 (0.92), 3.163 (0.92), 3.173 (1.08), 3.188 (1.16), 3.206 (3.39), 3.215 (3.44), 3.224 (1.68), 3.366 (4.67), 3.375 (2.62), 3.414 (3.54), 3.423 (5.47), 3.433 (3.58), 3.446 (6.10), 3.459 (12.22), 3.468 (4.35), 3.481 (5.03), 3.490 (4.80), 3.513 (16.00), 3.561 (2.46), 3.568 (8.86), 3.571 (4.99), 3.582 (4.63), 3.593 (1.93), 3.798 (0.81), 3.987 (0.58), 4.182 (0.63), 4.191 (0.63), 4.856 (0.60), 4.868 (0.59), 4.926 (0.41), 4.968 (1.16), 5.199 (1.61), 5.446 (3.03), 6.223 (0.63), 6.269 (2.29), 6.279 (2.17), 6.657 (1.56), 6.669 (1.58), 6.934 (1.88), 6.947 (2.08), 7.052 (1.85), 7.065 (2.95), 7.078 (1.32), 7.175 (2.07), 7.191 (4.17), 7.207 (4.60), 7.244 (5.48), 7.258 (3.06), 7.279 (3.00), 7.285 (3.52), 7.293 (3.90), 7.306 (2.38), 7.491 (2.80), 7.552 (2.02), 7.808 (2.49), 7.929 (0.90), 7.981 (0.70), 7.995 (0.63), 8.120 (1.00), 8.133 (0.95), 8.258 (0.51), 8.440 (2.20), 8.463 (0.92), 8.804 (1.74), 9.911 (0.70).

### Example C23: Trisodium (1S,9S)-1-amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-[(2S)-15-{[N2,N6-bis(14-{4-[({(1R)-2-carboxylato-1-[3-({3-[(propylcarbamoyl)amino]benzene-1-sulfonyl}amino) phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-valinate (Compound 23)

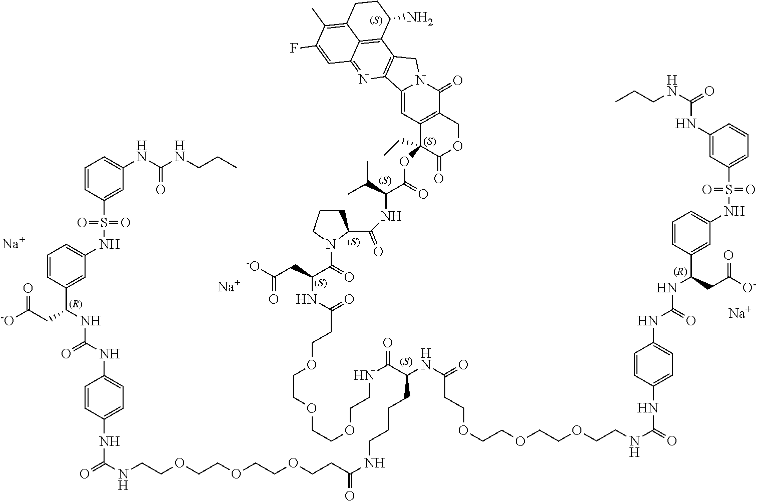

(1S,9S)-1-Amino-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl] ethyl}carbamoyl)amino]anilino}-14-oxo-4,7,10-trioxa-13-azatetradecanan-1-oyl)-L-lysyl] amino}ethoxy)ethoxy]ethoxy}propanoyl)-L-alpha-aspartyl-L-prolyl-L-valinate (5.50 mg, 2.08 μmol) (Intermediate 51) was dissolved in dioxane/water (1:1, 2.0 ml). A sodium hydroxide solution (62 μl, 0.10 M, 6.2 μmol) was added. The solution was freeze-dried to give Compound 23 (4.40 mg, 100% purity, 78% yield) as a colorless foam. LC-MS (Method 3): Rₜ=3.41 min; MS (ESIpos): m/z=1323 [M+2H]²⁺. [M+H]⁺. ¹H-NMR (600 MHz, DMSO-d₆) δ[ppm]: 0.815 (3.66), 0.828 (7.76), 0.840 (4.04), 0.877 (1.35), 0.889 (2.55), 0.907 (2.55), 0.918 (2.95), 0.925 (2.07), 1.005 (0.52), 1.234 (0.89), 1.339 (0.95), 1.351 (1.92), 1.363 (2.75), 1.374 (2.54), 1.387 (1.34), 1.399 (0.40), 1.897 (0.60), 2.211 (0.77), 2.222 (0.63), 2.268 (0.89), 2.279 (1.69), 2.290 (0.94), 2.324 (0.58), 2.335 (0.68), 2.369 (3.63), 2.518 (1.75), 2.521 (1.73), 2.587 (1.06), 2.612 (0.97), 2.652 (0.41), 2.699 (0.41), 2.924 (0.92), 2.934 (1.91), 2.945 (1.92), 2.956 (0.95), 2.981 (0.81), 3.204 (2.70), 3.214 (2.68), 3.363 (3.45), 3.412 (2.53), 3.421 (3.80), 3.430 (2.32), 3.449 (5.16), 3.460 (8.63), 3.479 (3.55), 3.488 (3.45), 3.511 (10.47), 3.566 (16.00), 3.580 (3.11), 3.590 (1.24), 3.769 (0.42), 4.180 (0.47), 4.190 (0.46), 4.375 (0.50), 4.867 (0.61), 4.879 (0.47), 4.962 (0.78), 5.366 (0.50), 5.398 (0.58), 5.472 (1.19), 5.479 (1.05), 5.583 (0.51), 6.153 (0.56), 6.675 (0.94), 6.687 (0.94), 6.934 (1.28), 6.947 (1.38), 7.057 (1.21), 7.070 (1.86), 7.083 (0.78), 7.178 (1.81), 7.186 (2.78), 7.189 (2.57), 7.200 (3.21), 7.237 (3.94), 7.252 (2.13), 7.275 (1.67), 7.288 (2.13), 7.301 (1.02), 7.526 (0.66), 7.545 (0.60), 7.594 (0.88), 7.747 (0.59), 7.794 (0.65), 7.911 (0.64), 7.952 (0.55), 7.965 (0.46), 8.053 (0.44), 8.300 (0.46), 8.398 (0.88), 8.758 (0.85).

### Example C24: Preparation of trisodium (1S,9S)-1-acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 1-[(2S)-15-{[N2,N6-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-valinate (Compound 24)

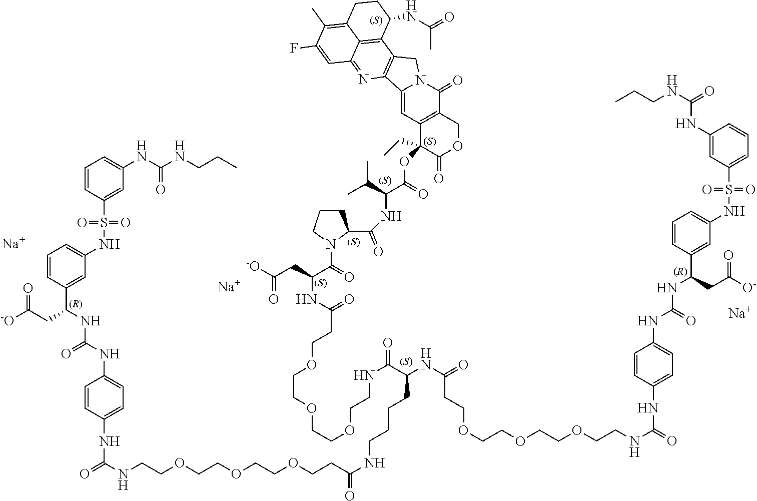

(1S,9S)-1-Acetamido-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N-(3-{2-[2-(2-{[N²,N⁶-bis(14-{4-[({(1R)-2-carboxy-1-[3-({3-[(propylcarbamoyl)amino]benzene-1-sulfonyl}amino)phenyl] ethyl}carbamoyl)amino]anilino}-14-oxo-4,7,10-trioxa-13-azatetradecanan-1-oyl)-L-lysyl]amino}ethoxy)ethoxy]ethoxy}propanoyl)-L-alpha-aspartyl-L-prolyl-L-valinate (3.40 mg, 1.26 μmol) (Intermediate 58) was dissolved in dioxane/H2O (1:1, 2.0 ml). A sodium hydroxide solution (38 μl, 0.10 M, 3.8 μmol) was added. The solution was freeze-dried to give Compound 24 (2.14 mg, 92% purity, 57% yield) as a colorless foam. LC-MS (Method 6): Rₜ=3.01 min; MS (ESIpos): m/z=1344 [M+2H]²⁺.

### Biological Evaluation:

### Example B1: Cytotoxicity In Vitro in the Presence and Absence of Elastase

Cultivation of cells was performed according to standard procedures with the media recommended by the provider. The cells, in a total volume of 100 μL, were seeded in a 96-well plate with a white bottom (#3610). After a 24 h incubation period at 37° C. and 500 CO₂, the medium was exchanged by adding 90 μL fresh medium. The treatment started by adding the test compound in 10 μL of culture medium to the cells in triplicates. Concentrations ranging from 10⁻⁵ M to 10⁻¹³ M were chosen. Two identically treated sets of samples were prepared. One set was treated with the test compound alone, whereas to the second set the compound and 10 nM elastase was added followed by a 72 h incubation period at 37° C. and 5% CO₂. The proliferation was detected using the MTT assay (ATCC). At the end of the incubation period the MTT reagent was added to all samples for 4 h. Lysis of the cells followed by addition of the detergent was done overnight. The formed dye was detected at 570 nm. The proliferation of untreated but otherwise identically handled cells was define as the 10000 value. The dose response curves allowed the determination of the respective IC₅₀ values, which are summarized in Table 1.

TABLE 1
Determination of the IC₅₀ values with or without elastase

| | 786-O | | HT29 | | NCI H292 | | SUM149 | |
|---|---|---|---|---|---|---|---|---|
| | IC₅₀ | | IC₅₀ | | IC ₅₀ | | IC₅₀ | |
| Compound | w/o | w elastase | w/o | w elastase | w/o | w elastase | w/o | w elastase |
| 1 | B | A | C | B | B | A | A | AA |
| 2 | D | A | DD | B | C | B | C | A |
| 3 | D | B | DD | B | C | A | D | A |
| 4 | C | A | D | A | C | A | C | AA |
| 4b | D | A | D | B | C | A | C | AA |
| 5 | C | AA | D | AA | C | AA | C | AA |
| 6 | C | A | C | A | C | A | B | AA |
| 7 | A | A | A | A | A | A | AA | AA |
| 8 | A | A | A | A | A | A | AA | AA |
| 9 | B | B | C | B | B | B | A | A |
| 12 | C | B | D | B | B | B | B | A |
| 13 | D | A | DD | B | C | A | C | A |
| 14 | C | A | C | A | C | AA | C | AA |
| 15 | D | A | D | A | C | A | C | AA |
| 16 | B | B | B | B | B | B | A | A |
| 17 | C | C | C | C | C | C | B | B |

AA = <0.01 nM;
A = 0.01-0.1 nM;
B = 0.1-1 nM;
C = 1.1-10 nM;
D = 10-100 nM;
DD = >100 nM

The Cytotoxicity assay (Version 2) was performed as above described. Some changes were applied as the assays were performed in an automated fashion and the IC₅₀ values determination was changed. Here the change in metabolic activity of the cells, in percent, was calculated by normalization of the measured values to the absorbance values of medium containing wells without cells (=−100%) and the absorbance of the DMSO-treated cells (=0%). AC₅₀ values (activity concentration at −50%) were determined by means of a 4 parameter Hill-fit using GeneData Screener software. Values are summarized in Table 1b.

TABLE 1b
Determination of the AC₅₀ values with or without elastase

| | 786-O | | HT29 | | NCI H292 | | SUM149 | |
|---|---|---|---|---|---|---|---|---|
| | AC₅₀ | | AC₅₀ | | AC₅₀ | | AC₅₀ | |
| Compound | −elastase | +elastase | −elastase | +elastase | −elastase | +elastase | −elastase | +elastase |
| C4b | D | B | D | B | D | B | C | A |
| C5 | D | B | D | B | D | A | C | A |
| C17 | B | B | C | C | B | B | B | B |
| C18 | C | C | C | B | B | B | B | B |
| C19 | D | B | D | C | D | C | D | B |
| C20 | D | B | D | C | D | B | D | B |
| C21 | D | B | D | B | B | A | C | A |
| C22 | D | A | D | B | B | A | C | A |
| C23 | D | B | D | B | D | B | C | A |
| C24 | D | B | D | C | D | B | C | B |

A < 1 nM
B = 1.1-10 nM
C = 11-100 nM
D > 100 nM
n.t. = not tested

### Example B2: Cytotoxicity In Vitro in the Presence and Absence of Legumain

Using the same assay design for examples with legumain cleavable residues reveals that addition of the respective cleavage enzyme allows no optimal generation of the payload due to cell culture conditions. The pH value for the cytotoxicity assay differs significantly from the pH optimum of the protease which is reflected in the minor improvement of the measured cytotoxic potency in Table 2.

TABLE 2
Determination of the IC₅₀ values with or without legumain

| | 786-O | | HT29 | | NCI H292 | | SUM149 | |
|---|---|---|---|---|---|---|---|---|
| | IC₅₀ | | IC₅₀ | | IC₅₀ | | IC₅₀ | |
| Compound | w/o | w legumain | w/o | w legumain | w/o | w legumain | w/o | w legumain |
| 10 | n.t. | n.t. | D | D | D | D | D | D |
| 11 | n.t. | n.t. | D | D | C | C | B | B |

A < 1 nM
B = 1.1-10 nM
C = 11-100 nM
D > 100 nM
n.t. = not tested

### Example B3: αᵥβ₃ Binding Assay

Binding affinity of the respective αᵥβ₃ inhibitor is tested in an ELISA-like manner. 96-well plates are coated with vitronectin (1 μg/ml) or fibronectin 0.5 μg/ml) and afterwards uncoated surface space is blocked with BSA. After a 2 h pre-incubation of a serial dilution of the test compound (starting at 20 μM in a 1:5 dilution) with the extracellular domain of the integrin (2 μg/ml, αvβ3 or α5β1) the solution is added to the coated plates. After an 1 h incubation at rt and several washing steps, an integrin specific antibody is added for 1 h at rt. After three washing steps the secondary peroxidase-labeled antibody is added to the plate. After an 1 h incubation at RT the plate is developed by quick addition of SeramunBlau (50 μL per well, Seramun Diagnostic GmbH, Heidesee, Germany) and incubated for 5 min at rt in the dark. The reaction is stopped with 0.3M H₂SO₄ (50 μL/well), and the absorbance was measured at 450 nm with a plate reader. IC₅₀ of each compound is tested in duplicate, and the resulting inhibition curves is analyzed. As a reference standard, Cilengitide is used. The αᵥβ₃ binding of the reference is used as 100% value.

### Example B4: Stability in Rat Plasma

1 mg of the respective test compound of is dissolved in a mixture of 1.5 mL dimethylsulfoxide and 1 ml water. For complete dissolution, the HPLC vial is shaken and treated with an ultrasound. 500 μl of this solution is added to 0.5 mL of rat plasma with vortexing at a temperature of 37° C. Aliquots (10 μL each) are taken at respective time points and analyzed by HPLC to determine the amount of the test compound. All data is given as percent area of the initial compound at to.

Method for measurement of stability in buffer: 0.15 mg of the test compound are solved in 0.1 ml dimethylsulfoxide and 0.4 ml acetonitrile. For complete dissolution the HPLC vial with the sample solution is shaken and sonicated. Then 1.0 ml of the respective buffer solution is added and the sample is vortexed. The sample solution is analysed by HPLC to determine the amount of the test compound and up to two byproducts at a particular time over a period of 24 h at 37° C. to values result from a sample immediately taken after vortexing with buffer at RT. The peak areas (in percentage) are used for quantification.

### Example B5: Lactone Ring Stability

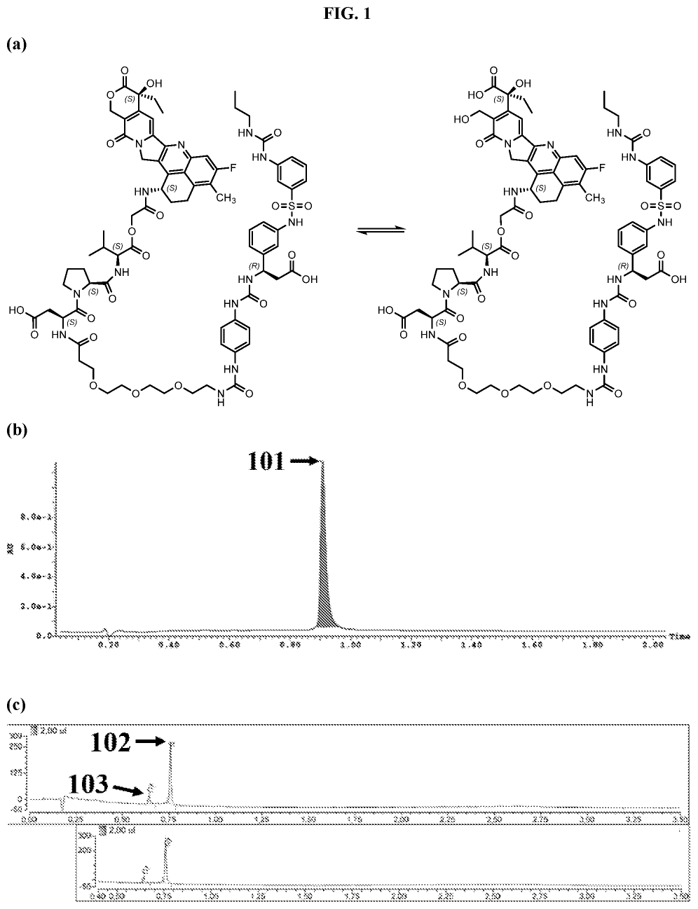

pH dependent ring-opening by hydrolysis of the lactone ring is an issue in the camptothecin class of agents. It is a pH dependent equilibrium between closed lactone form and ring-opened hydroxy acid form which is shifted to the closed ring at acidic pH and to the opened form under basic conditions. Only the lactone form is active. As shown in FIG. 1 panel (a) the lactone ring of Compound 1 may undergo ring-opening by hydrolysis, resulting in the inactive product. Under acidic conditions (FIG. 1 panel (b)), only the closed form of the lactone (101) is observed. However, under basic conditions (FIG. 1 panel (c)), both the closed lactone ring (102) and hydrolized ring-opened form (103) are detected in the UV track of LC-MS. The closed and hydrolyzed open forms are differentiable by mass using the LC-MS methods described herein.

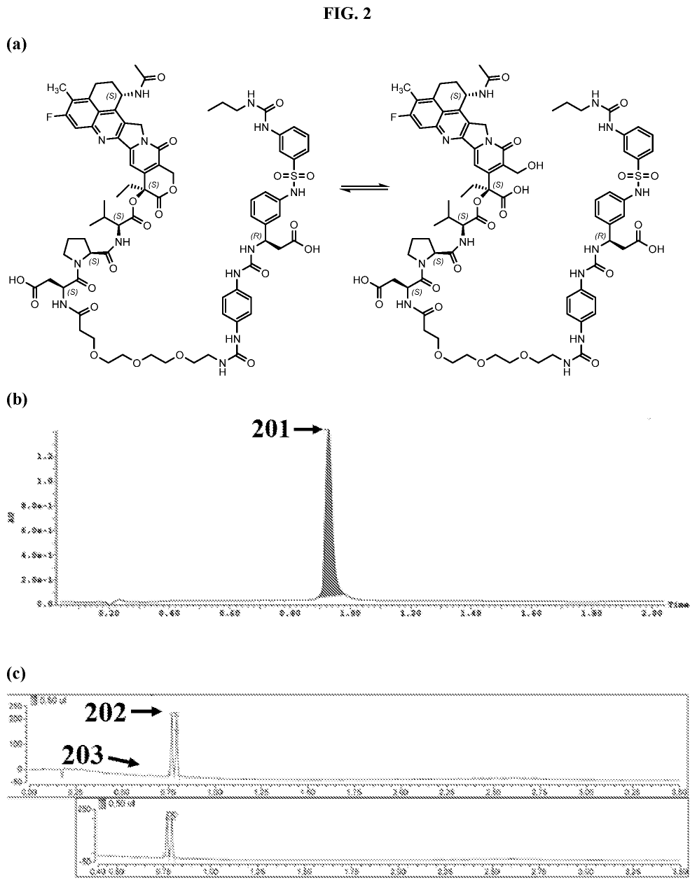

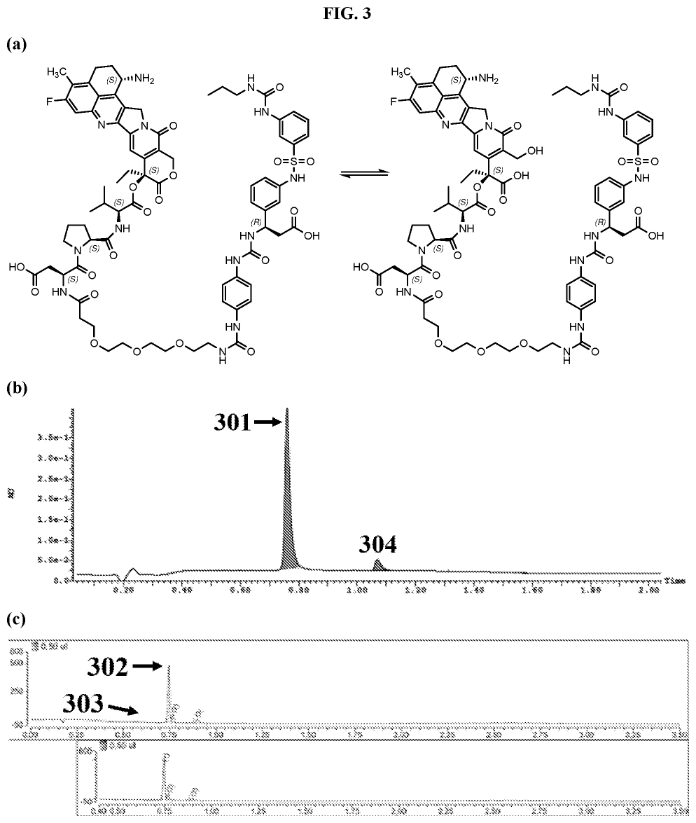

As shown in FIGS. 2 and 3 for the exemplified for Compound 2 and Compound 4 respectively, linker attachment via acylation of the hydroxy group at the lactone ring (e.g. Compounds 2-6 and Compounds 13-15) stabilizes the closed lactone form. Panel (a) of FIG. 2 and FIG. 3 shows the equilibrium between closed and open forms of the lactone rings for Compound 2 and Compound 4 respectively. Compound 2 is detectable under acidic LC-MS conditions (FIG. 2 panel (b)) in only the closed form (201). Under basic conditions (FIG. 2 panel (c)), two peaks (202) were detected, both of which had the same mass, corresponding to the closed form of Compound 2. These peaks were assigned as rotamers of the closed form of Compound 2. No open form (203) was detected under basic conditions, showing the superior stability when the hydroxy group at the lactone ring is acylated. Compound 4 showed enhanced stability similar to Compound 2. Under acidic conditions (FIG. 3 panel (b)), only the closed form (301) was detected by UV and mass spectrometry. A second peak (304) was detected by UV spectroscopy, however there was no corresponding mass detected. Under basic conditions (FIG. 3 panel (c)), a major peak (302) and the adjacent shoulder peak were detected by UV spectroscopy and corresponded to mass spectrometry peaks consistent with the closed form of Compound 4. As with Compound 2, no open form (303) was detected by LC-MS under acidic or basic conditions.

This is in contrast to Compound 1 with the unmodified lactone ring and the linker attached to the hydroxyacetylamide in 1-position. Compound 1 remains stable in LC-MS when the acidic eluent is used, but significant hydrolysis of the lactone ring is observed with the basic eluent.

Method for acidic eluent: System MS: Waters TOF instrument; System UPLC: Waters Acquity I-CLASS; Column: Waters Acquity UPLC HSS T3 1.8 μm 50×1 mm; Eluent A: 1 L Water+0.100 ml 99% Formic acid (pH 3.4); Eluent B: 1 L Acetonitrile+0.100 ml 99% Formic acid; Gradient: 0.0 min 90% A→1.2 min 5% A→2.0 min 5% A Oven: 50° C.; Flow: 0.40 ml/min; UV-Detection: 210 nm.

Method for basic eluent: System MS: Thermo Scientific FT-MS; System UHPLC+: Thermo Scientific UltiMate 3000; Column: Waters, BEH c18 1.7μ, 2.1×50 mm, Eluent A: 1 L Water+1.0 mL (25% Ammonia) (pH of eluent A=10.2); Eluent B: 1 L Acetonitrile; Gradient: 0.0 min 5% B→2.5 min 95% B→3.5 min 95% B; Oven: 50° C.; Flow: 0.90 ml/min; UV-Detection: 210 nm/Optimum Integration Path 210-300 nm.

### Example B6: In Vitro Tests for Determining Cellular Permeability

Caco-2: The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells were cultured for 15-16 days on 24-well filter plates. For the determination of permeation, the respective test substance was applied in a HEPES buffer to the cells either apically (A) or basally (B) and incubated for 2 hours. After 0 hours and after 2 hours, samples were taken from the cis and trans compartments. The samples were separated by HPLC (Agilent 1200, Böblingen, Germany) using reverse phase columns. The HPLC system was coupled via a Turbo Ion Spray Interface to a Triple Quadropol mass spectrometer API 4000 (AB SCIEX Deutschland GmbH, Darmstadt, Germany). The permeability was evaluated on the basis of a Papp value, which was calculated using the formula published by Schwab et al. [D. Schwab et al., J. Med. Chem. 46, 1716-1725 (2003)]. A substance was classified as actively transported when the ratio of Papp (B-A) to Papp (A-B) (efflux ratio) was >2 or <0.5. Compounds with an efflux ratio of 0.5-2 are considered not actively transported. In Table 5, reference compounds are provided for comparison. Ref 1 is exatecan mesylate, Ref 2 is DXd, and Ref 3 is 10,11-methylenedioxy-camptothecin (FL118).

TABLE 5
Caco-2 Transport data of test compounds

Caco-2 Flux

| Compound | Papp A-B [nm/s] | Efflux ratio |
|---|---|---|
| 7 | AA | A |
| 8 | BB | A |
| 9 | AA | A |
| 16 | CC | B |
| 17 | CC | B |
| Ref 1 | CC | B |
| Ref 2 | AA | A |
| Ref 3 | CC | B |

AA: 1-9
BB: 10-99
CC: 100-999
A: actively transported
B: not actively transported

P-glycoprotein (P-gp) assay: Many tumor cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile. The substrate properties of a substance for P-gp (ABCB1) are determined by means of a flux assay using LLC-PKl cells which overexpress P-gp (L-MDRl cells) [A. H. Schinkel et al., J. Clin. Invest. 96, 1698-1705 (1995)]. For this purpose, the LLC-PKl cells or L-MDRl cells are cultured on 96-well filter plates for 3-4 days. For determination of the permeation, the respective test substance, alone or in the presence of an inhibitor (such as ivermectin or verapamil, for example), is applied in a HEPES buffer to the cells either apically (A) or basally (B) and incubated for 2 hours. After 0 hours and after 2 hours, samples are taken from the cis and trans compartments. The samples are separated by HPLC using reverse phase columns. The HPLC system is coupled via a Turbo Ion Spray Interface to a Triple Quadropol mass spectrometer API 3000 (Applied Biosystems Applera, Darmstadt, Germany). The permeability is evaluated on the basis of a Papp value, which is calculated using the formula published by Schwab et al. [D. Schwab et al., J. Med. Chem. 46, 1716-1725 (2003)]. A substance is classified as P-gp substrate when the efflux ratio of Papp (B-A) to Papp (A-B) is >2.

### Example B7: Pharmacokinetics

Male rats (n=3) were given an intravenous 0.5 or 2.0 mg/kg bolus dose of test article dissolved in plasma with 1% DMSO and 2% ethanol. Serial blood samples were collected up to 24 hours post-dose. Plasma was harvested from blood samples and stored frozen until sample analysis. Test article plasma concentrations were analyzed by LC/MS/MS. Pharmacokinetic parameters were calculated using non-compartmental analysis.

### Example B8: In Vivo Xenotransplantation Studies

The anti-tumor activities of the test compounds were examined in a SCLC mouse xenograft model. For this purpose, immunocompromised mice were implanted subcutaneously with tumor cells or tumor fragments. At a mean tumor size of 20-40 mm², animals were randomized, split into treatment and control groups (n=10 animals/group), and treatment started with vehicle only or example C4b or C5 (formulation: phosphate buffered saline (“PBS”); application route: intravenously into the tail vein (“i.v.”)). Intravenous treatments were performed on two consecutive days once daily followed by five days of drug holiday without any treatment. The tumor size and the body weight were determined twice weekly. The tumor area was detected by means of an electronic caliper [length (mm)×width (mm)]. The experimental groups were ended after three 3 weeks of treatment. In vivo anti-tumor efficacy is presented as T/C ratio of mean tumor area measured for treatment and control group on the last day at which the vehicle control remained in study (Treatment/Control; mean tumor area of treatment group/mean tumor area of control group. A compound having a T/C below 0.5 is defined as active (i.e., effective). Statistical analysis was assessed using GraphPadPrizm software. An unpaired t-test was performed versus the control group.

We claim:

1. A compound, or a pharmaceutically acceptable salt thereof, comprising a hexacyclic cytotoxic or cytostatic ligand (CT) conjugated to one or more integrin binders (IN) via a linker.

2. The compound of claim 1, or a pharmaceutically acceptable salt thereof, wherein the linker is enzymatically-cleavable.

3. The compound of claim 1 or 2, or a pharmaceutically acceptable salt thereof, having a structure represented by Formula (A), or Formula (C):
CT-EL-L¹-A¹(L²-IN)(L³-IN) Formula (A)
CT-EL-L⁵-IN Formula (C)
wherein:
CT is a hexacyclic cytotoxic or cytostatic ligand;
EL is an enzymatically-cleavable linker; optionally further comprising a self-immolative group (SIL);
each L¹, L², L³, and L⁵ is independently a bivalent linker;
A¹ is a trivalent linker; and
IN is, in each instance, independently, an integrin binder.

4. The compound of any one of claims 1-3, or a pharmaceutically acceptable salt thereof, wherein the compound is cleaved by neutrophil elastase.

5. The compound of claim 3 or 4, or a pharmaceutically acceptable salt thereof, wherein EL is an enzymatically-cleavable peptide linker of the formula: L-Asp-L-Pro-L-Ala, L-Asn-L-Pro-L-Ala, L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-.

6. The compound of any one of claims 1-5, or a pharmaceutically acceptable salt thereof, having the structure of Formula (I-A) or Formula (I-C): 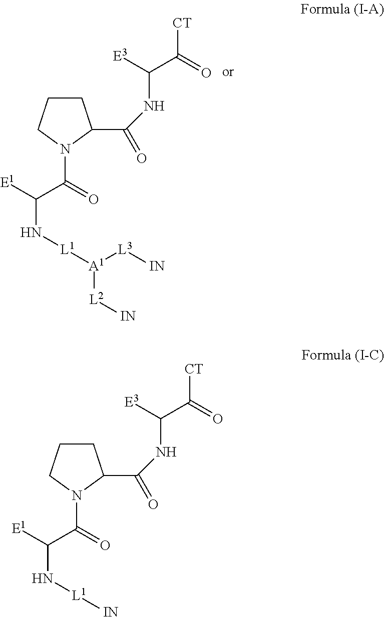
or a stereoisomer or mixture of stereoisomers thereof, wherein:
CT is a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂CH₂C(O)OH; —CH₂C(O)OR¹; or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each L¹, L², L³, and L⁵ is independently a bivalent linker;
R¹ is a substituted or unsubstituted C₁₋₁₂ alkyl; wherein if R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and
IN is, in each instance, independently, an integrin binder.

7. The compound of any one of claims 1-6, or a pharmaceutically acceptable salt thereof, or a stereoisomer or mixture of stereoisomers thereof, wherein IN is, in each instance, a non-peptide integrin binder.

8. The compound of any one of claims 1-7, or a pharmaceutically acceptable salt thereof, or a stereoisomer or mixture of stereoisomers thereof, wherein IN is, in each instance, a small molecule integrin-binding moiety.

9. The compound of any one of claims 1-8, or a pharmaceutically acceptable salt thereof, or a stereoisomer or mixture of stereoisomers thereof, wherein IN is, in each instance, an αᵥβ₃ integrin binder.

10. The compound of any one of claims 1-9, or a pharmaceutically acceptable salt thereof, or a stereoisomer or mixture of stereoisomers thereof, wherein IN, in each instance, has the structure: 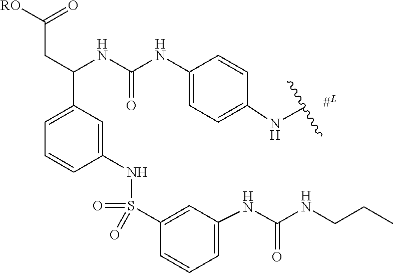
or a pharmaceutically acceptable salt thereof, or a stereoisomer thereof, wherein:
#^{L} denotes a bond to L¹, L², L³, L⁵, or L⁶; and
R is hydrogen or a substituted or unsubstituted C₁₋₁₂ alkyl.

11. The compound of any one of claims 1-10, or a pharmaceutically acceptable salt thereof, or a stereoisomer or mixture of stereoisomers thereof, having a structure of Formula (II-A), or Formula (II-C): 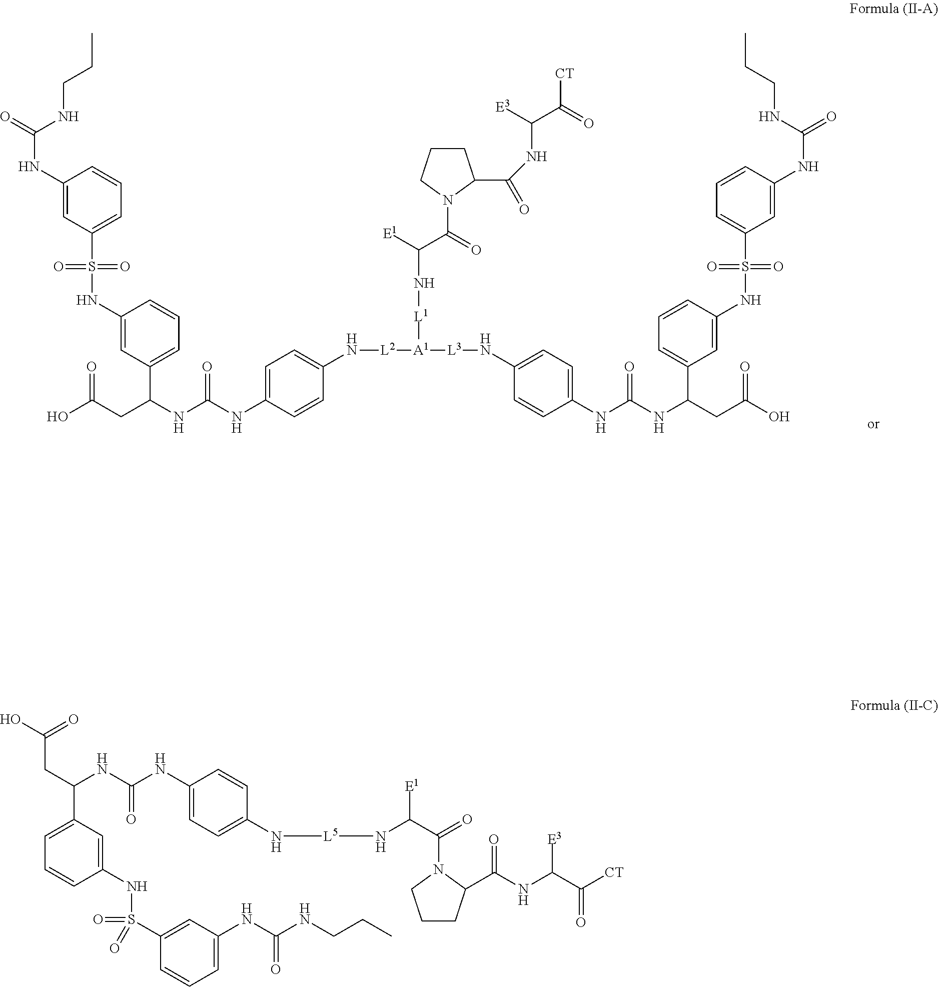
wherein:
CT is a hexacyclic cytotoxic or cytostatic ligand;
E¹ is hydrogen, —CH₃, —CH₂CH₃, —CH₂CH₂CH₃, —CH₂C(O)NH₂, —CH₂C(O)OH, —CH₂CH₂C(O)OH; —CH₂C(O)OR¹; or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each L¹, L², L³, and L⁵ is each independently a bivalent linker; and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and IN is an integrin binder.

12. The compound of any one of claims 1-11, or a pharmaceutically acceptable salt thereof, or a stereoisomer or mixture of stereoisomers thereof, wherein CT is: 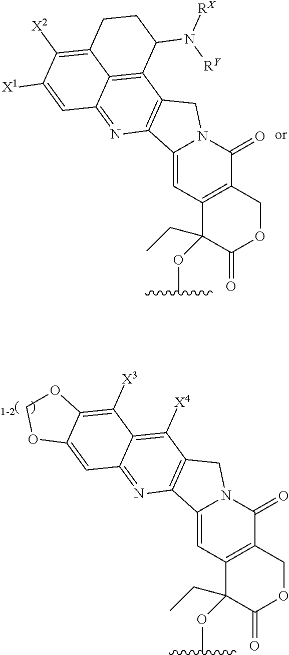
wherein:
X¹ is —F, —Cl, or —CH₃;
X² is —F, —Cl, or —CH₃;
X³ is hydrogen; and
X⁴ is hydrogen, C₁₋₃ alkyl, or C₁₋₃ alkylamine;
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
wherein EL² is an enzymatically-cleavable linker; and
R^{Y} is hydrogen, C₁₋₆ alkyl, —C(O)C₁₋₆ alkyl, —C(O)C₁₋₆ haloalkyl, —C(O)C₁₋₆ alkyl-OH, or —C(O)C₁₋₆ alkyl-OCH₃.

13. The compound of any one of claims 1-12, or a pharmaceutically acceptable salt thereof, or a stereoisomer or mixture of stereoisomers thereof, having the structure of Formula (III-A), Formula (III-C), Formula (IV-A), or Formula (IV-C): 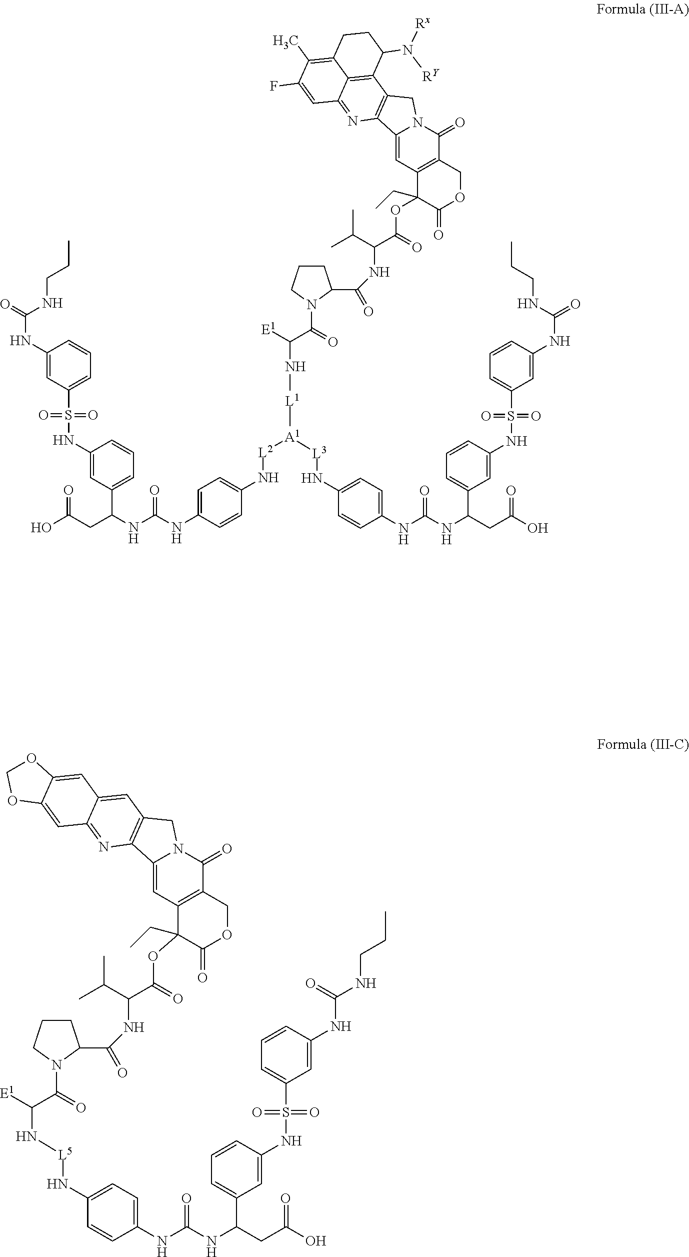 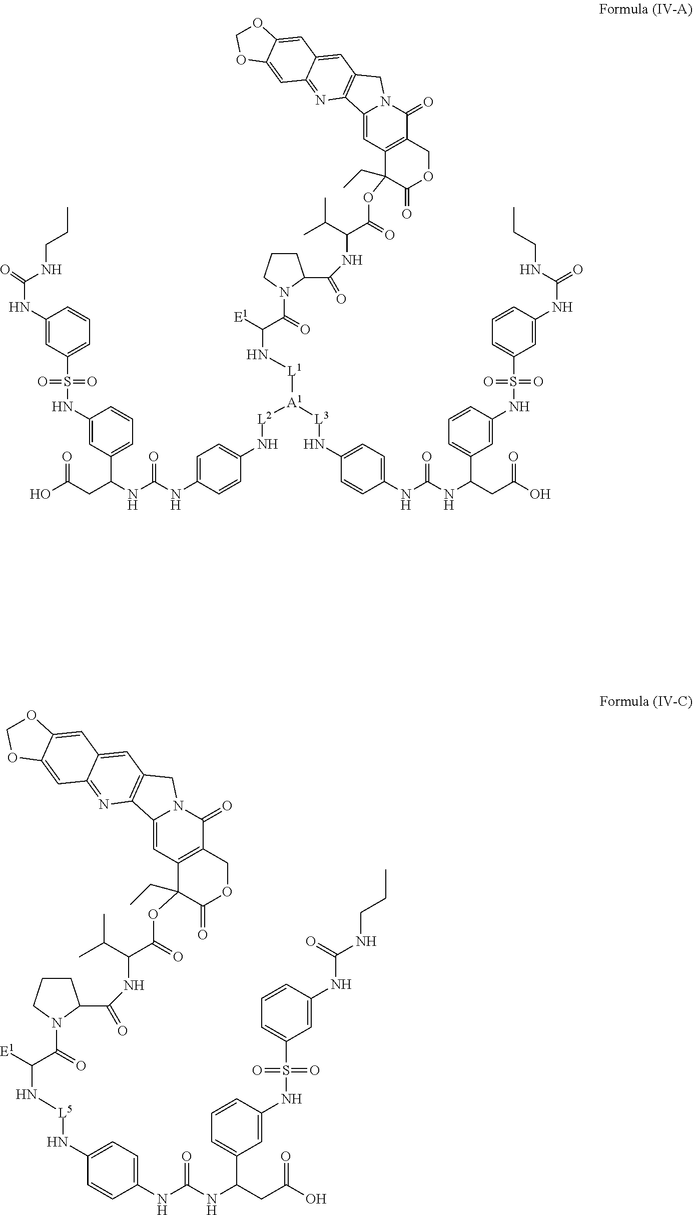
or a pharmaceutically acceptable salt thereof, or a stereoisomer or mixture of stereoisomers thereof; wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
wherein EL² is a legumain-cleavable linker;
R^{Y} is hydrogen, C₁₋₆ alkyl, —C(O)C₁₋₆ alkyl, or —C(O)C₁₋₆ haloalkyl;
E¹ is hydrogen, —CH₂C(O)NH₂ or —CH₂C(O)OH; and
A¹ is a trivalent linker; and
each L¹, L², L³, and L⁵ is independently a bivalent linker.

14. The compound of any one of claims 1-13, or a pharmaceutically acceptable salt thereof, or a stereoisomer or mixture of stereoisomers thereof, wherein each of L¹, L², L³, and L⁵ is independently a substituted or unsubstituted C₁₋₆₀ alkyl or 1-60 heteroalkyl that is optionally substituted or interrupted by one or more groups, each independently selected from the group consisting of: —C(O)—, —C(O)NH—, —C(O)N(CH₃)—, —C(O)O—, —NH—, —N(CH₃)—, —NHC(O)—, —N(CH₃)C(O)—, —NHC(O)NH—, —NHS(O)₂NH—, —NHS(O)₂NHC(O)—, —NHS(O)₂NHC(O)O—, —O—, —S—, —S(O)—, —S(O)₂—, —S(O)₂NH—, —S(O)₂NHC(O)—, —S(O)₂NHC(O)NH—, —S(O)₂NHC(O)O—, heterocyclyl, and heteroaryl; or any combination thereof.

15. The compound of any one of claims 1-14, or a pharmaceutically acceptable salt thereof, or a stereoisomer or mixture of stereoisomers thereof, wherein:
R^{X} is hydrogen, —C₁₋₆ alkyl, —C(O)CH₃, —C(O)CF₃, —C(O)OBn, —C(O)CH₂(pyridine), -Asn-C(O)CH₂(pyridine), or -EL²-C(O)CH₂-(pyridine);
EL² is -(L-Ala)(L-Ala)(L-Asn)- or -(L-Ala)(NMe-L-Ala)(L-Asn)-;
R^{Y} is hydrogen, —C₁₋₆ alkyl, or —C(O)C₁₋₆ alkyl;
E¹ is hydrogen, —CH₂C(O)NH₂, or —CH₂C(O)OH;
A¹ is a substituted or unsubstituted amino acid; and
L¹, L², L³, and L⁵ are each independently selected from (i), (ii), or (iii), and (iv):
(i) —C(O)—,
(ii) —(CO)ₘ(CH₂)ₙ(OC₂₋₆ alkyl)ₒ(NH)ₚ(CO)_{q}—,
(iii) —(CO)ᵣ(CH₂)ₛ(NR¹¹C₂₋₆ alkyl)ₜ(NR¹¹)ᵤ(CO)ᵥ—; or
(iv) —(CO)ᵣ—(CH₂)ₛ—(NR¹⁰C(O)C₁₋₆ alkyl)ₜ-(NR¹¹)ᵤ—(CO)ᵥ—;
wherein:
R¹⁰ is, in each instance, independently hydrogen or C₁₋₃ alkyl;
R¹¹ is, in each instance, independently hydrogen or C₁₋₃ alkyl;
m is, in each instance, independently 0 or 1;
n is in each instance, independently an integer from 0 to 10;
is in each instance, independently an integer from 0 to 10;
p is in each instance, independently 0 or 1;
q is in each instance, independently 0 or 1;
r is in each instance, independently 0 or 1;
s is in each instance, independently an integer from 0 to 10;
t is in each instance, independently an integer from 0 to 10;
u is in each instance, independently 0 or 1; and
v is in each instance, independently 0 or 1.

16. The compound of any one of claims 1-15, or a pharmaceutically acceptable salt thereof, or a stereoisomer or mixture of stereoisomers thereof, wherein:
R^{X} is hydrogen, —C₁₋₄ alkyl, —C(O)CH₃, —C(O)CF₃, or —C(O)OBn;
R^{Y} is hydrogen, —C₁₋₄ alkyl, or —C(O)C₁₋₄ alkyl;
E¹ is hydrogen, —CH₂C(O)OH, or —CH₂C(O)NH₂;
A¹ is a substituted or unsubstituted amino acid that is: L-Lys, L-Glu, or L-Gln;
L¹ is —C(O)—C₂₋₄ alkyl-[O—C₂₋₆ alkyl]₁₋₈-NH,
—C(O)—C₁₋₄ alkyl-[N(CH₃)—C₁₋₆ alkyl]₁₋₈-NH—,
—C(O)—C₁₋₄ alkyl-[N(CH₃)—C₁₋₆ alkyl]₁₋₈-N(CH₃)—, or
—C(O)—C₁₋₄ alkyl-[N(CH₃)C(O)—C₁₋₆ alkyl]₁₋₈-N(CH₃)—; and
each L², L³, and L⁵ is independently:
—C(O)—C₂₋₄ alkyl-[O—C₂₋₆ alkyl]₁₋₈-NHC(O),
—C(O)—C₁₋₄ alkyl-[N(CH₃)—C₁₋₆ alkyl]₁₋₈-NHC(O)—,
—C(O)—C₁₋₄ alkyl-[N(CH₃)—C₁₋₆ alkyl]₁₋₈-N(CH₃)C(O)—, or
—C(O)—C₁₋₄ alkyl-[N(CH₃)C(O)—C₁₋₆ alkyl]₁₋₈-N(CH₃)C(O)—.

17. The compound of any one of claims 1-16, or a pharmaceutically acceptable salt thereof, or a stereoisomer or mixture of stereoisomers thereof, wherein:
R^{X} is hydrogen, —CH₃, —CH₂CH₃, —C(O)CH₃, or —C(O)OBn;
R^{Y} is hydrogen, —CH₃, or —CH₂CH₃;
E¹ is —CH₂C(O)OH or —CH₂C(O)NH₂;
L¹ is a bivalent linker having the structure: 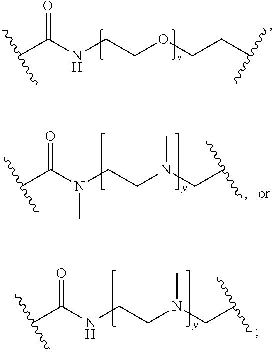
A¹ is a trivalent linker having the structure: 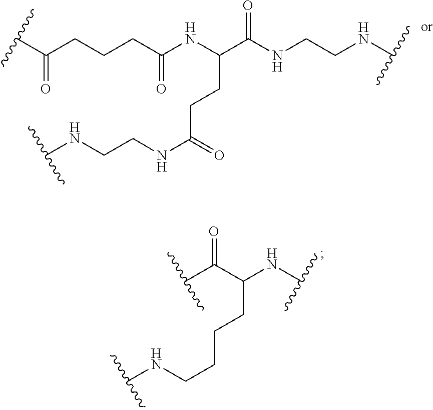 [SMILES: *NCCNC(=O)CCC(NC(=O)CCCC(*)=O)C(=O)NCCN* ; *NCCCCC(N*)C(*)=O]
each of L², L³, and L⁵ is a bivalent linker having the structure: 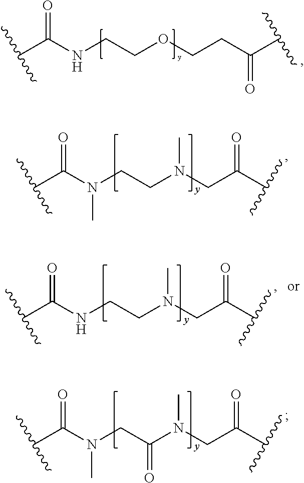
wherein in each instance, each y is independently 1, 2, 3, 4, 5, 6, 7, or 8.

18. The compound of any one of claims 1-17, or a pharmaceutically acceptable salt thereof, or a stereoisomer or mixture of stereoisomers thereof, wherein CT is:

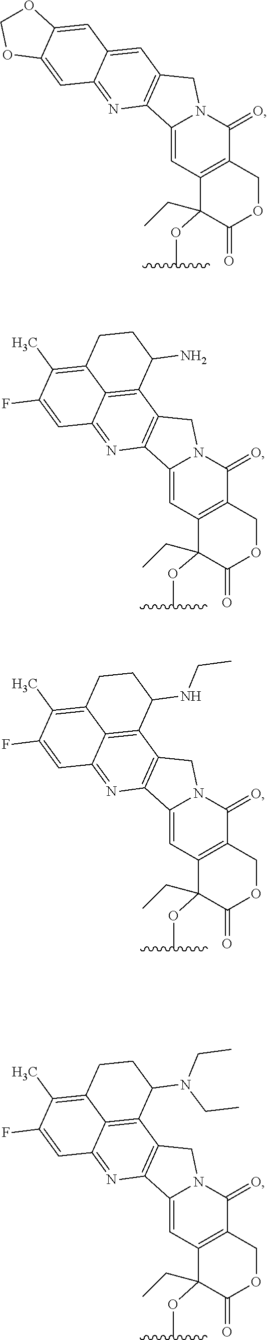 [SMILES: *OC1(CC)C(=O)OCc2c1cc1n(c2=O)Cc2cc3cc4c(cc3nc2-1)OCO4 ; *OC1(CC)C(=O)OCc2c1cc1n(c2=O)Cc2c-1nc1cc(F)c(C)c3c1c2C(N)CC3 ; *OC1(CC)C(=O)OCc2c1cc1n(c2=O)Cc2c-1nc1cc(F)c(C)c3c1c2C(NCC)CC3 ; *OC1(CC)C(=O)OCc2c1cc1n(c2=O)Cc2c-1nc1cc(F)c(C)c3c1c2C(N(CC)CC)CC3]

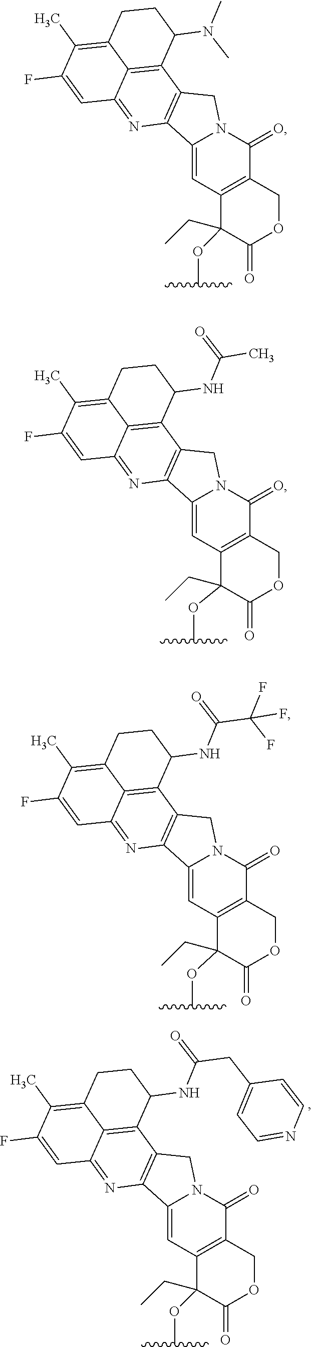

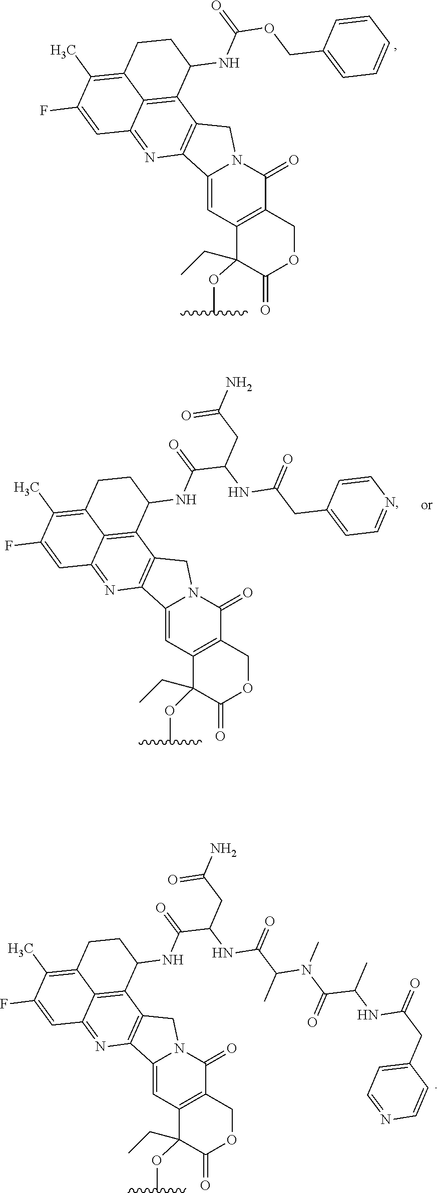 [SMILES: *OC1(CC)C(=O)OCc2c1cc1n(c2=O)Cc2c-1nc1cc(F)c(C)c3c1c2C(NC(=O)OCc1ccccc1)CC3 ; *OC1(CC)C(=O)OCc2c1cc1n(c2=O)Cc2c-1nc1cc(F)c(C)c3c1c2C(NC(=O)C(CC(N)=O)NC(=O)Cc1ccncc1)CC3 ; *OC1(CC)C(=O)OCc2c1cc1n(c2=O)Cc2c-1nc1cc(F)c(C)c3c1c2C(NC(=O)C(CC(N)=O)NC(=O)C(C)N(C)C(=O)C(C)NC(=O)Cc1ccncc1)CC3]

19. The compound of any one of claims 1-18, or a pharmaceutically acceptable salt thereof, or a stereoisomer or mixture of stereoisomers thereof, wherein CT is:

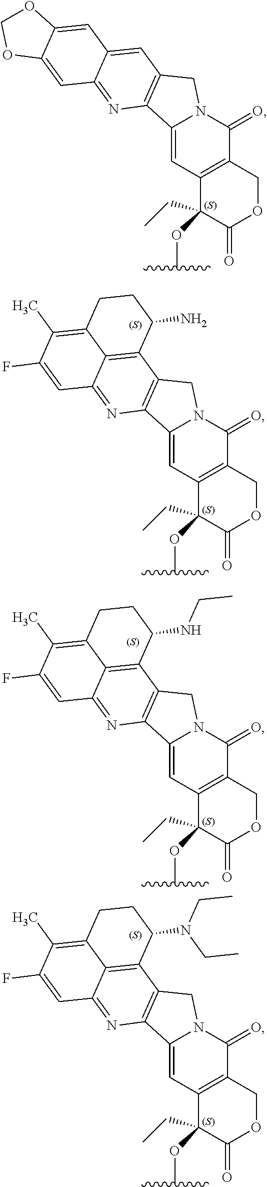

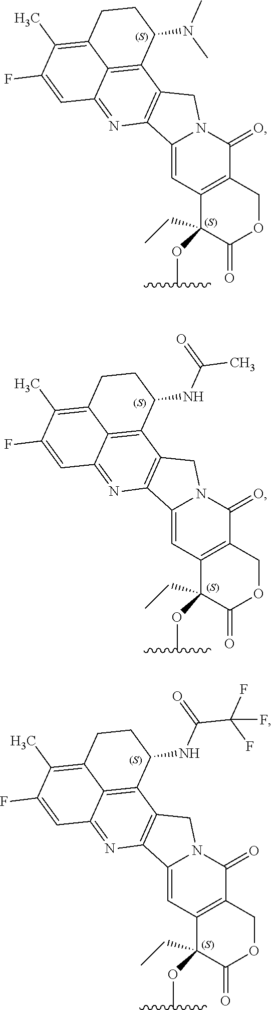

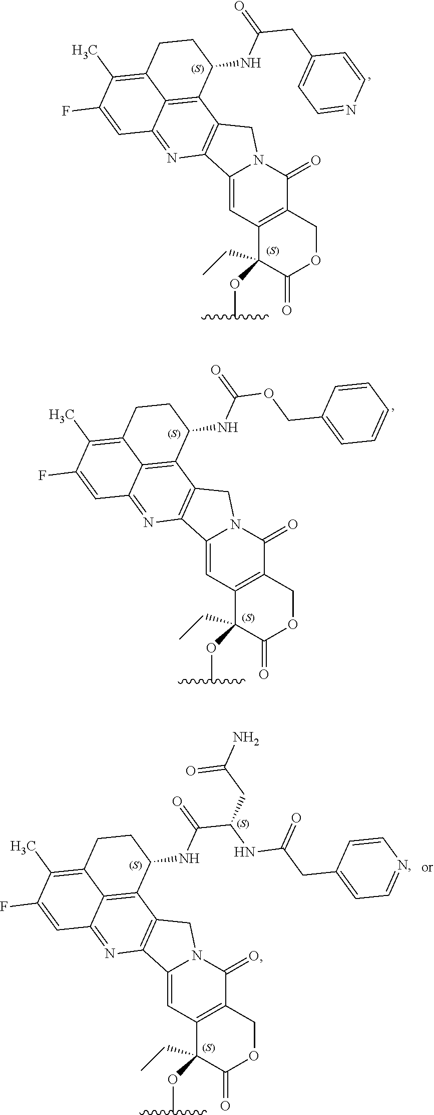

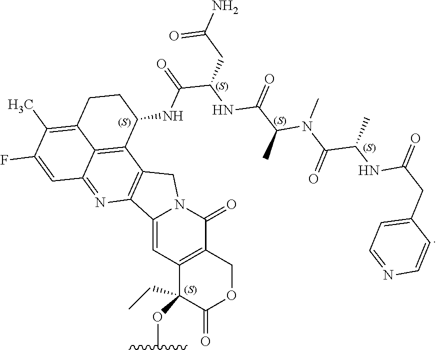

20. The compound of any one of claims 1-18, or a pharmaceutically acceptable salt thereof, or a stereoisomer or mixture of stereoisomers thereof, that is: 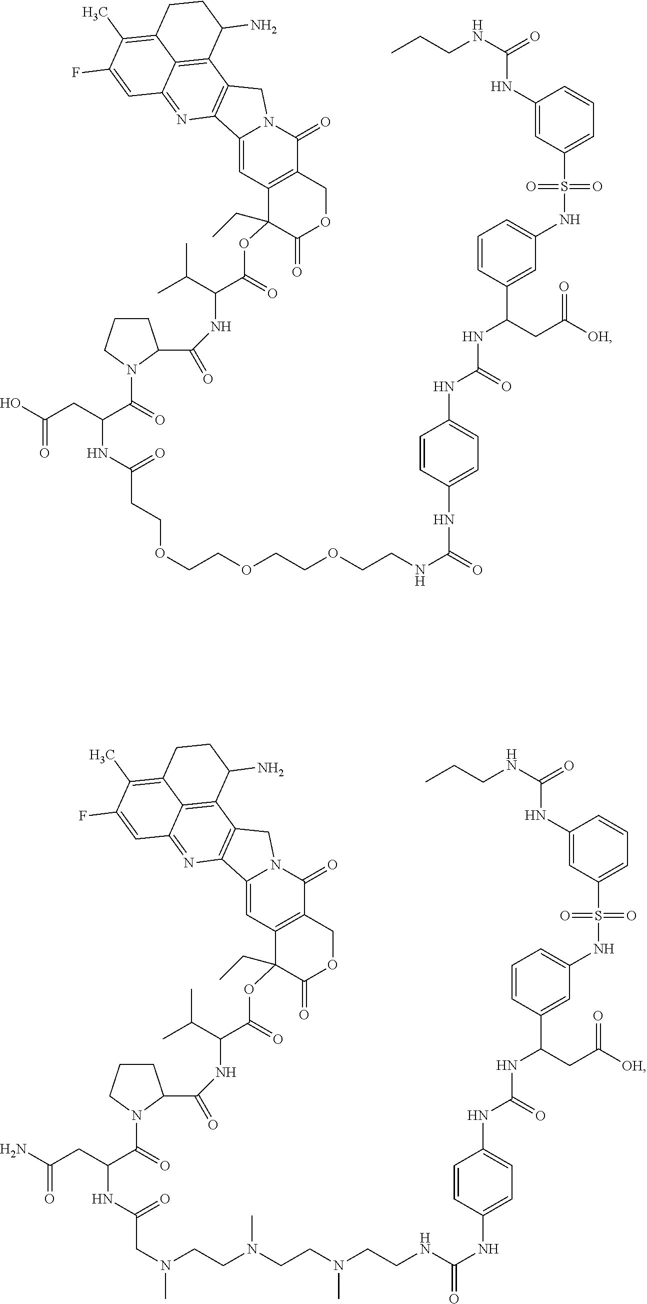 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(N)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(N)CC6)C(C)C)cc3)c2)c1] 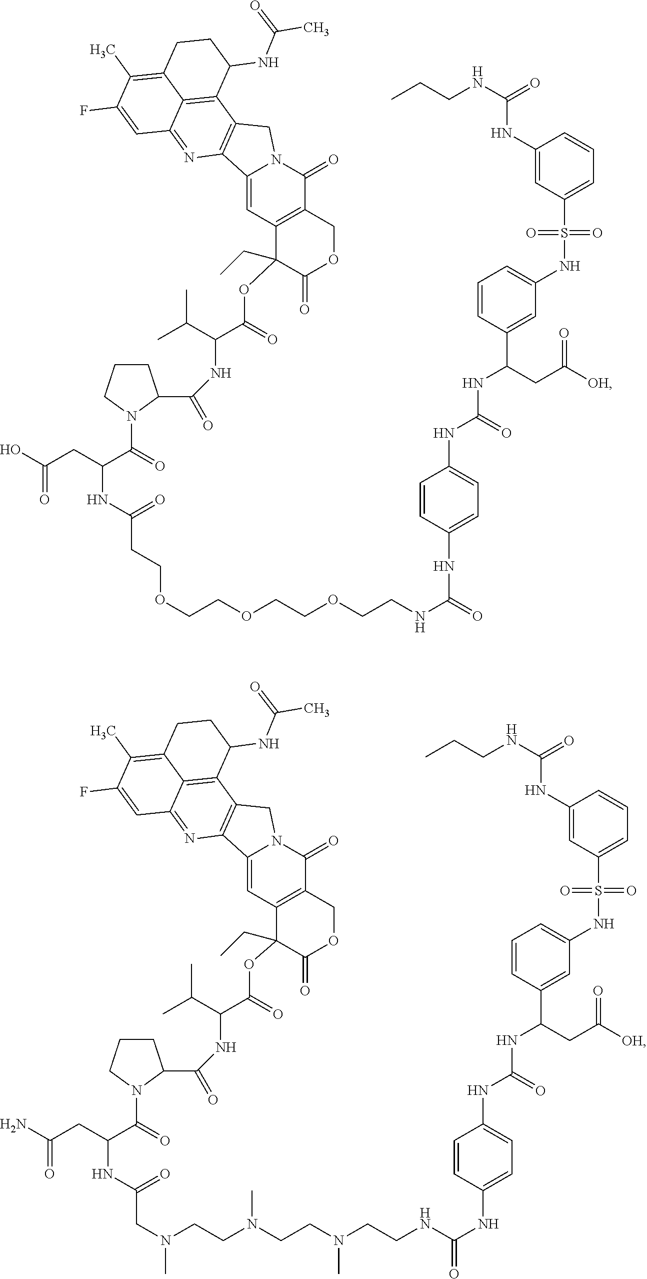 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(NC(C)=O)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(NC(C)=O)CC6)C(C)C)cc3)c2)c1] 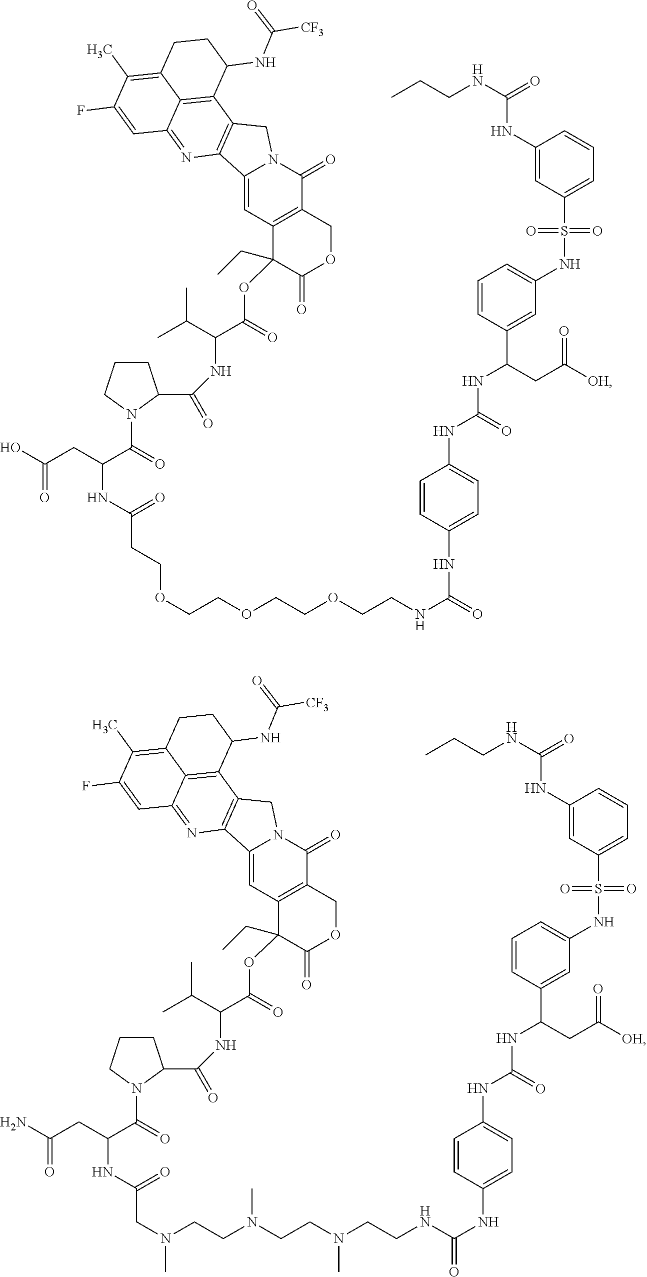 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(NC(=O)C(F)(F)F)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(NC(=O)C(F)(F)F)CC6)C(C)C)cc3)c2)c1] 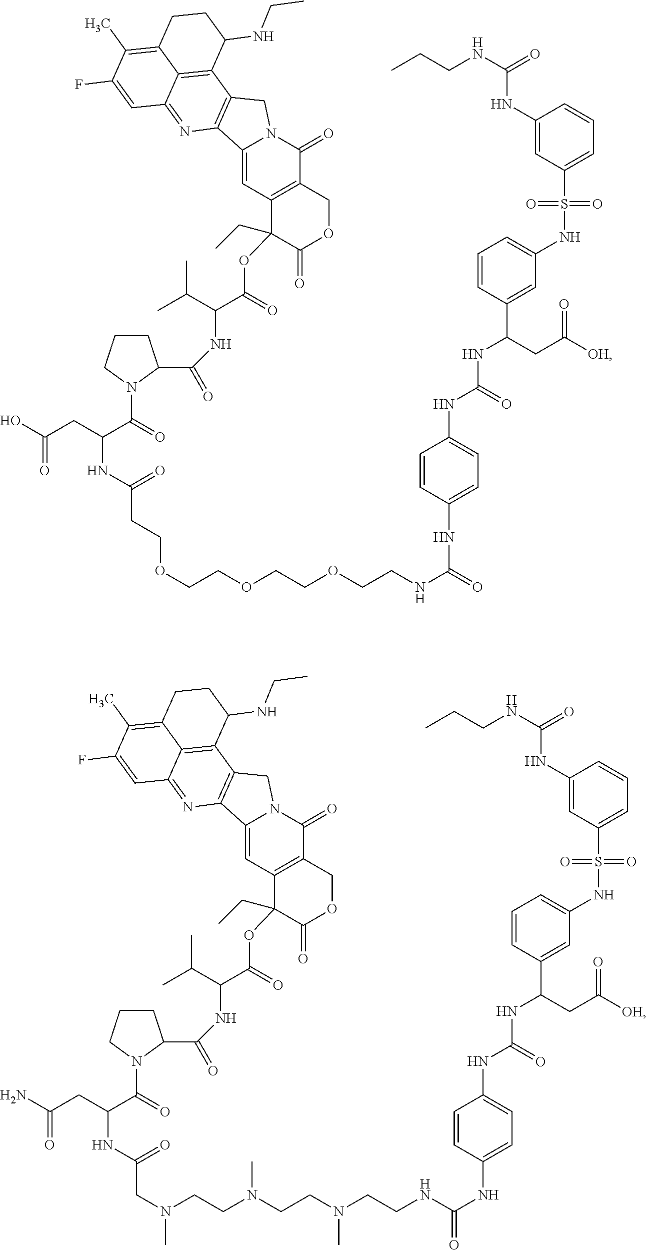 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(NCC)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(NCC)CC6)C(C)C)cc3)c2)c1] 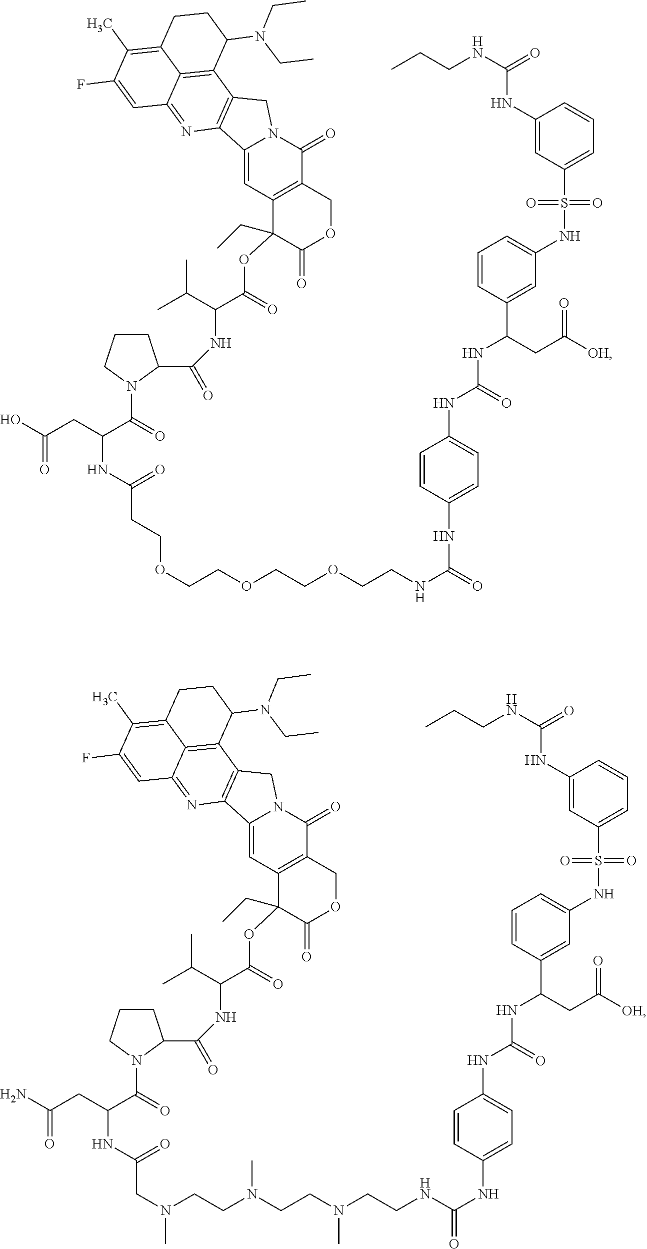 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(N(CC)CC)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(N(CC)CC)CC6)C(C)C)cc3)c2)c1] 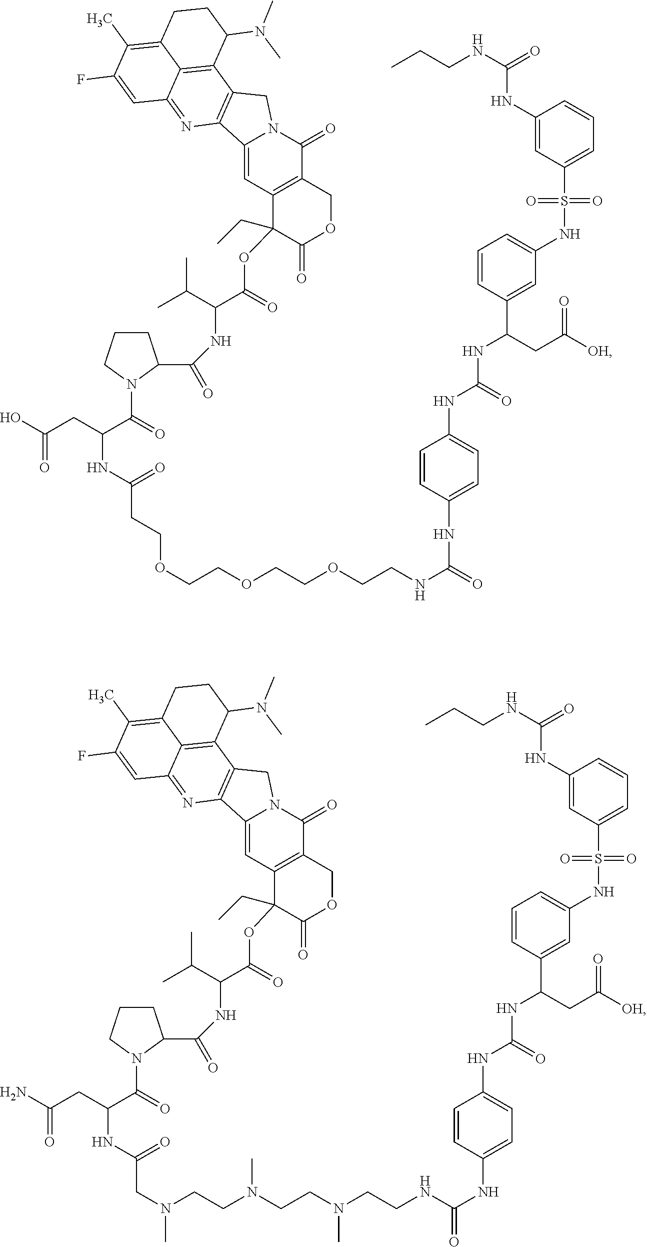 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(N(C)C)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(N(C)C)CC6)C(C)C)cc3)c2)c1] 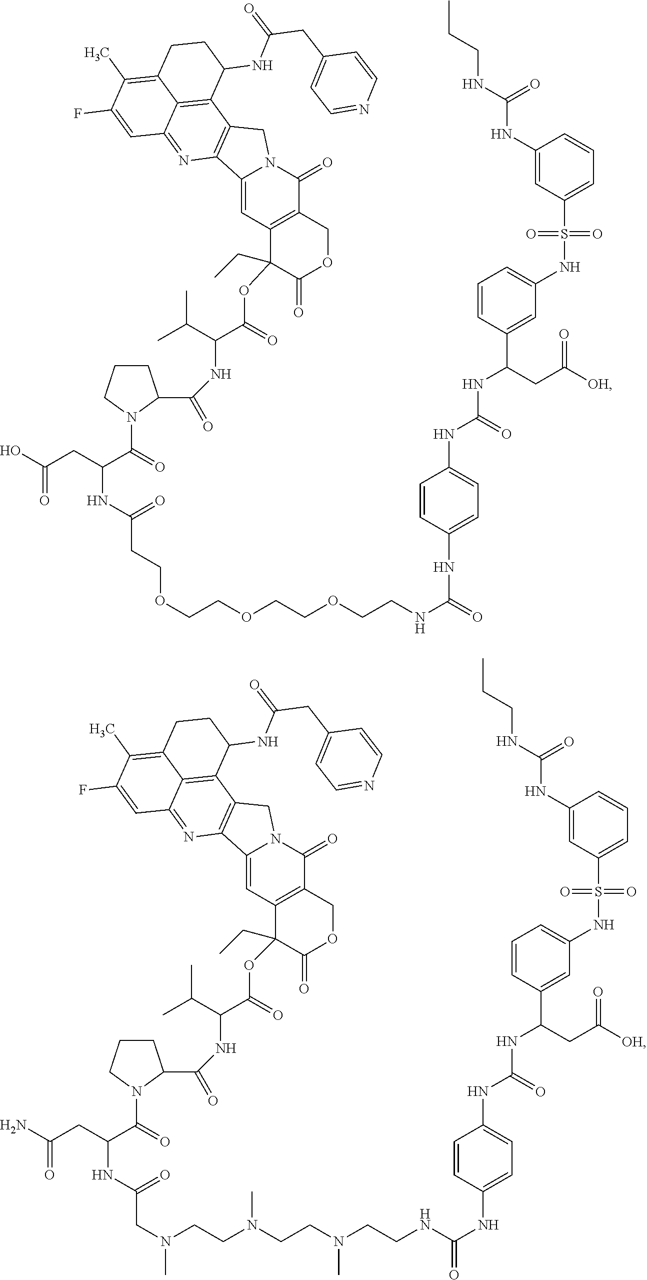 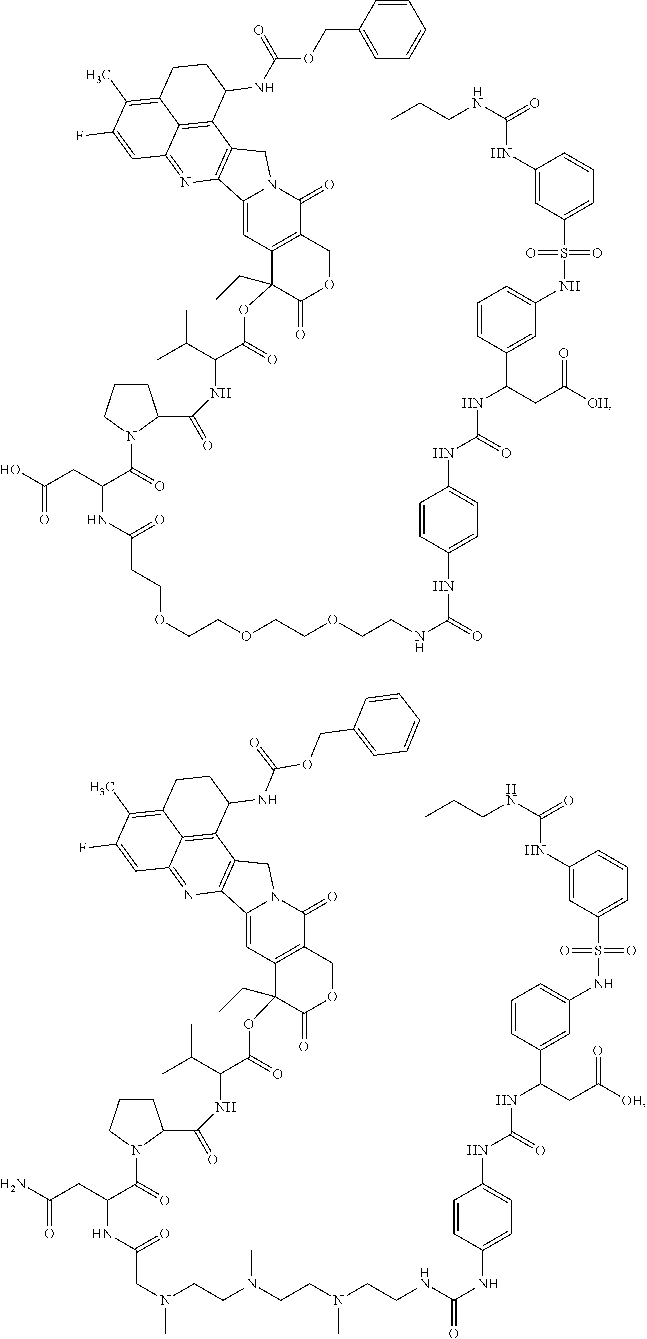 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(NC(=O)OCc4ccccc4)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(NC(=O)OCc4ccccc4)CC6)C(C)C)cc3)c2)c1] 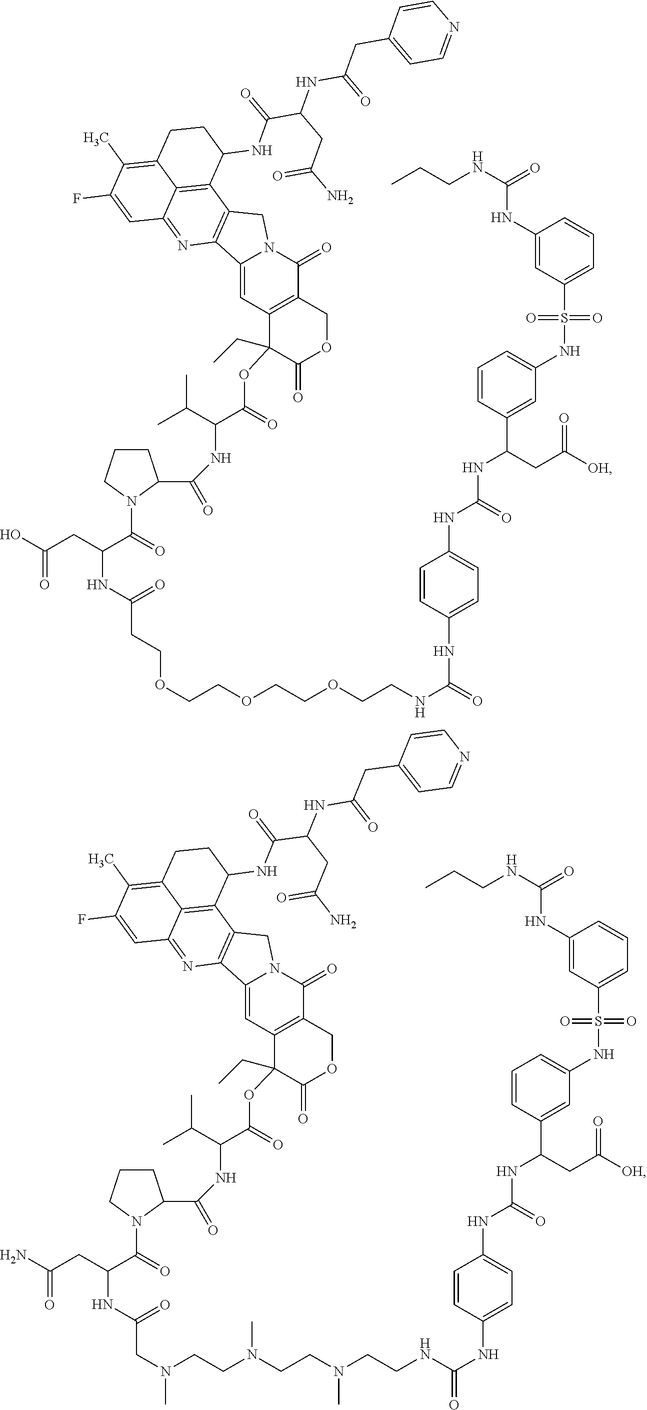 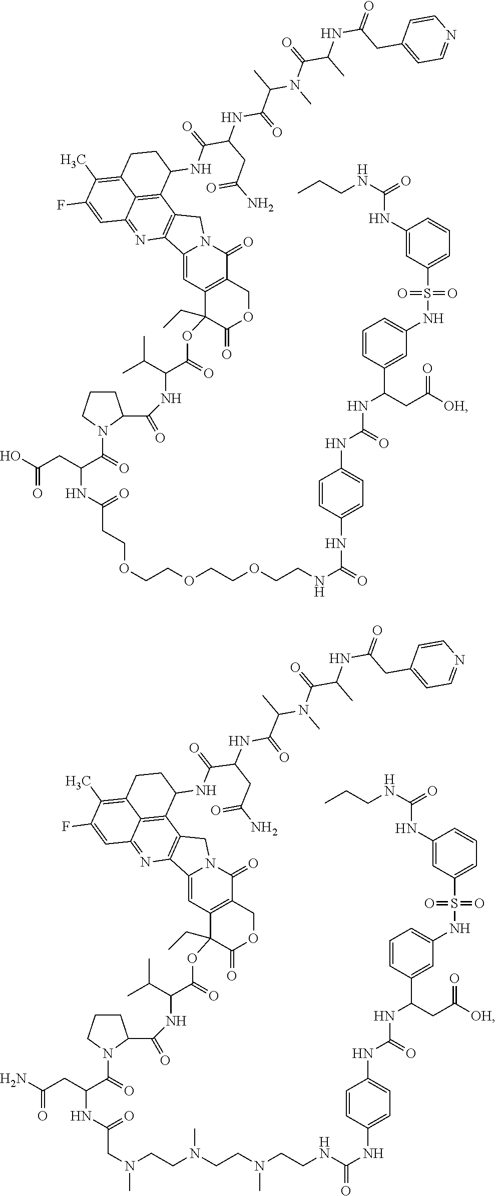 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4cc(F)c(C)c6c4c5C(NC(=O)C(CC(N)=O)NC(=O)C(C)N(C)C(=O)C(C)NC(=O)Cc4ccncc4)CC6)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4cc(F)c(C)c6c4c5C(NC(=O)C(CC(N)=O)NC(=O)C(C)N(C)C(=O)C(C)NC(=O)Cc4ccncc4)CC6)C(C)C)cc3)c2)c1] 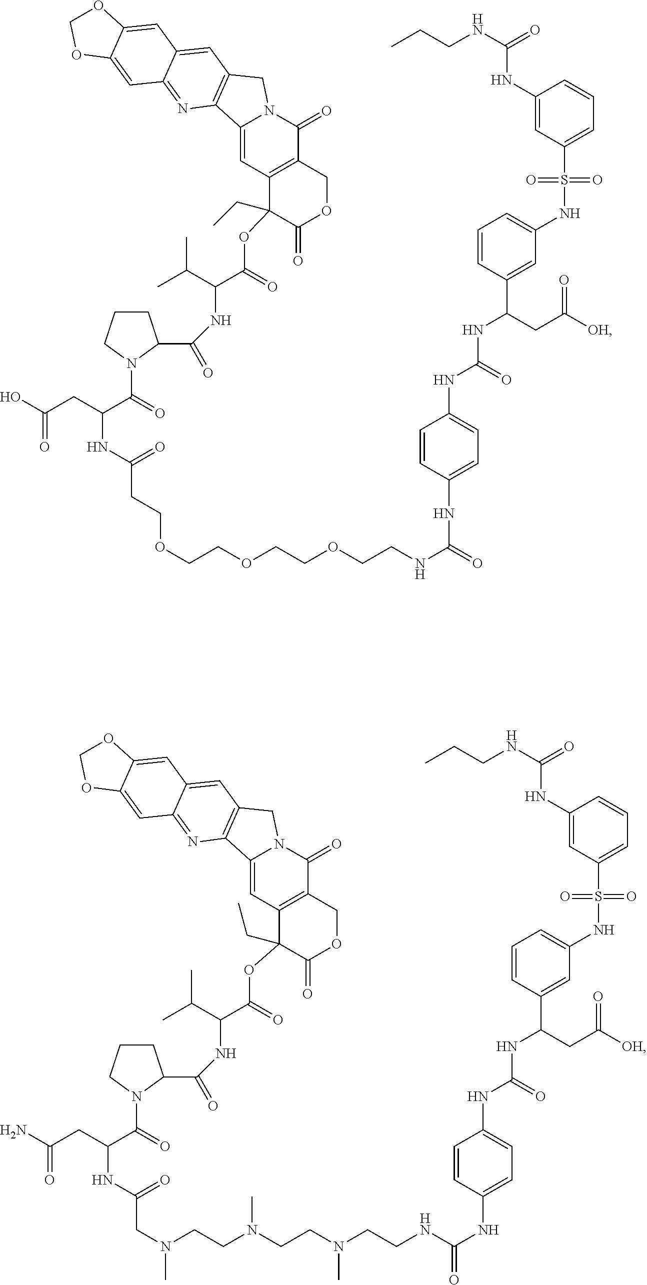 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5cc6cc7c(cc6nc5-4)OCO7)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5cc6cc7c(cc6nc5-4)OCO7)C(C)C)cc3)c2)c1] 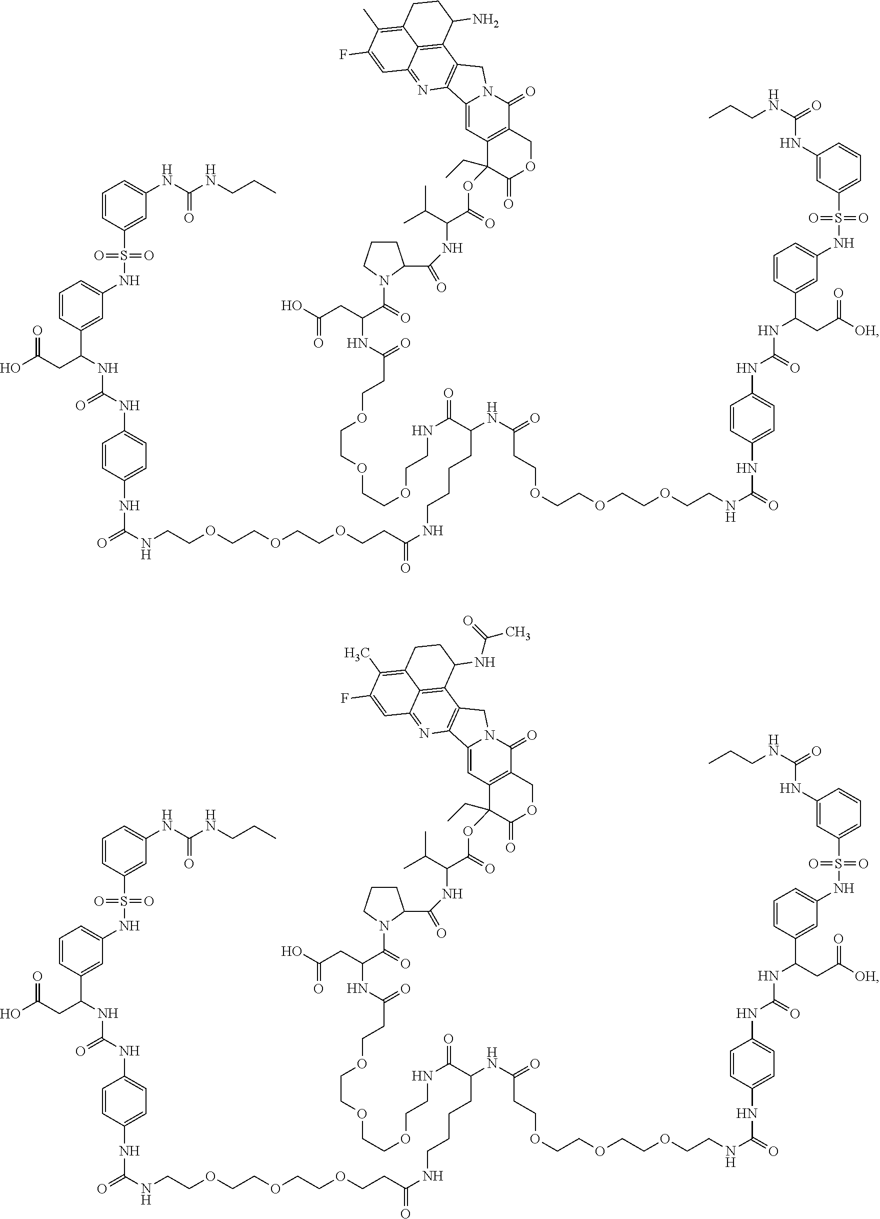 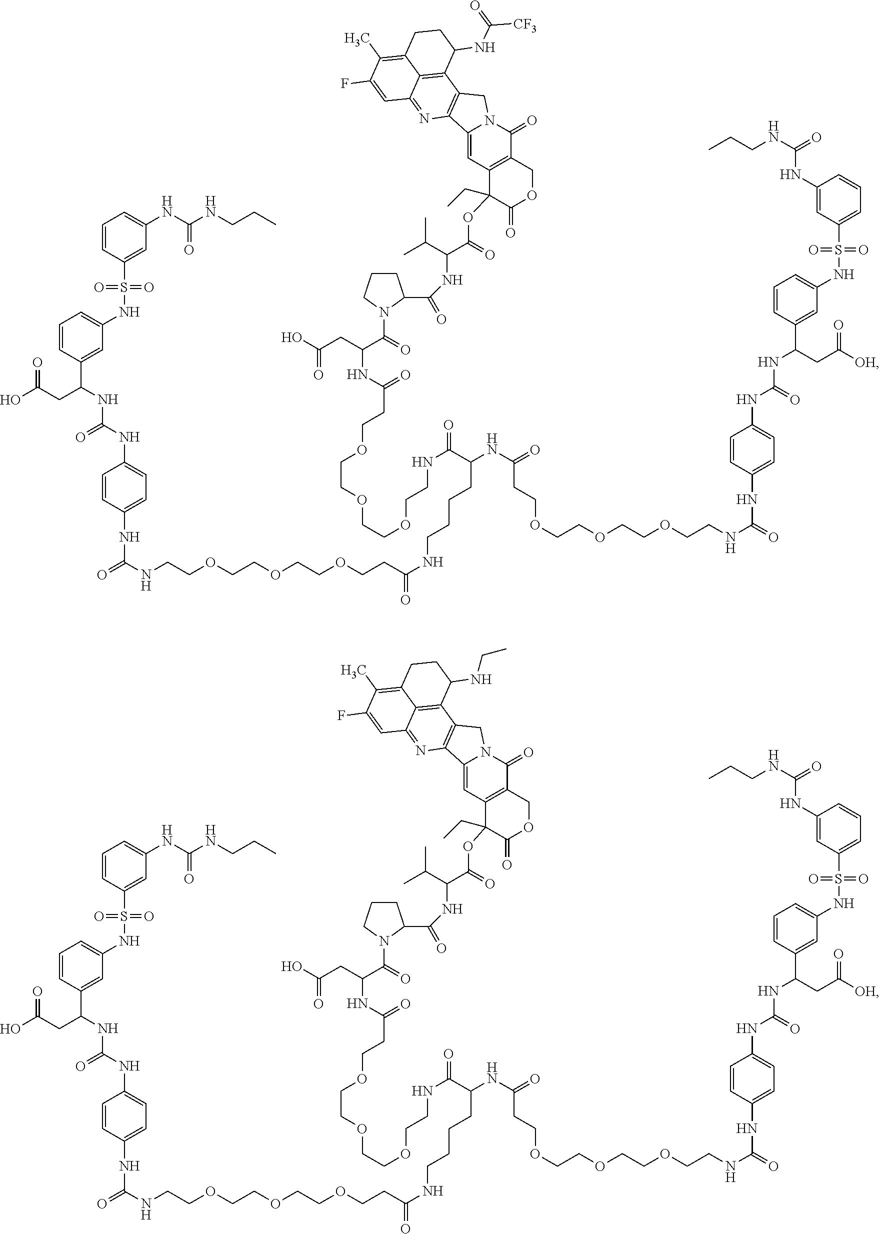 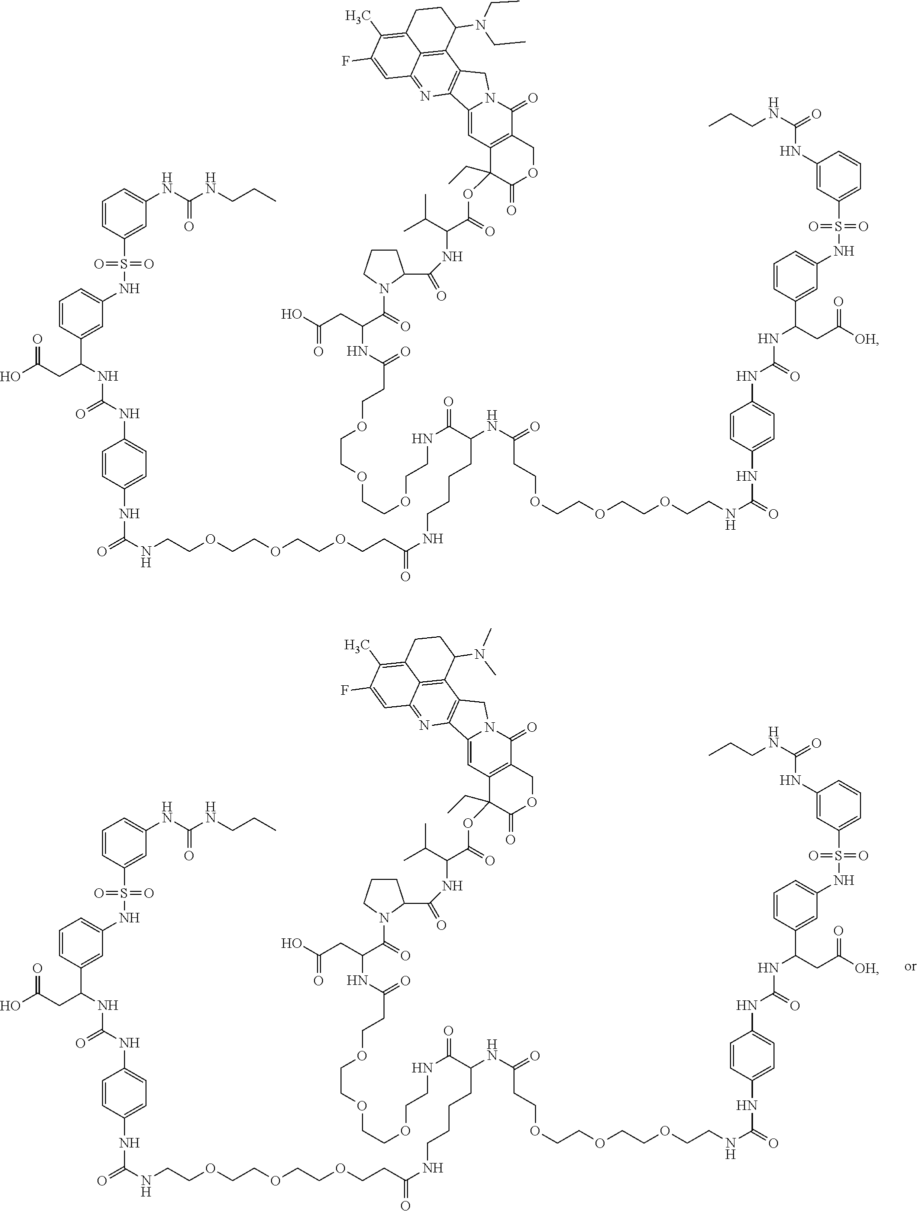 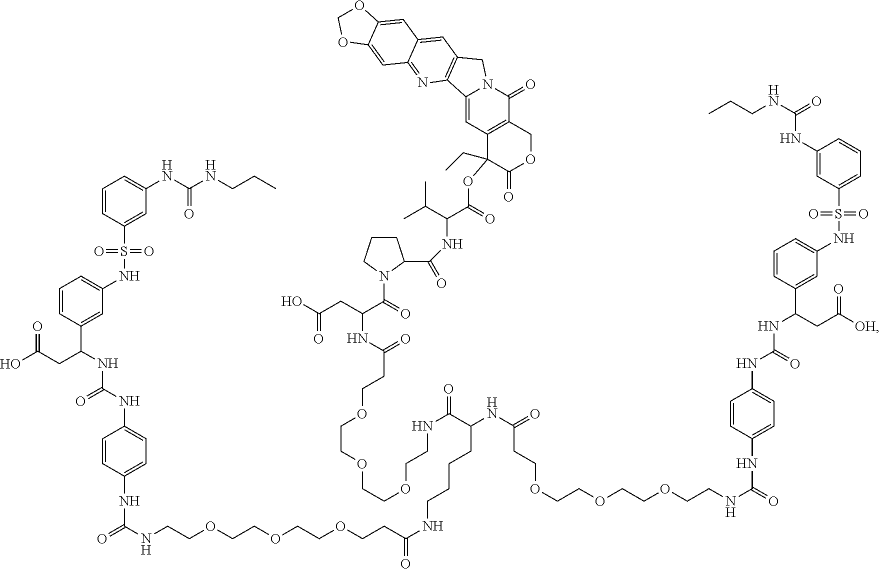 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCCCC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCOCCOCCOCCC(=O)NC(CC(=O)O)C(=O)N4CCCC4C(=O)NC(C(=O)OC4(CC)C(=O)OCc5c4cc4n(c5=O)Cc5cc6cc7c(cc6nc5-4)OCO7)C(C)C)cc3)c2)c1]
or a pharmaceutically acceptable salt thereof, or a stereoisomer or mixture of stereoisomers thereof.

21. The compound of any one of claims 1-20, or a pharmaceutically acceptable salt thereof, or a stereoisomer or mixture of stereoisomers thereof, that is:

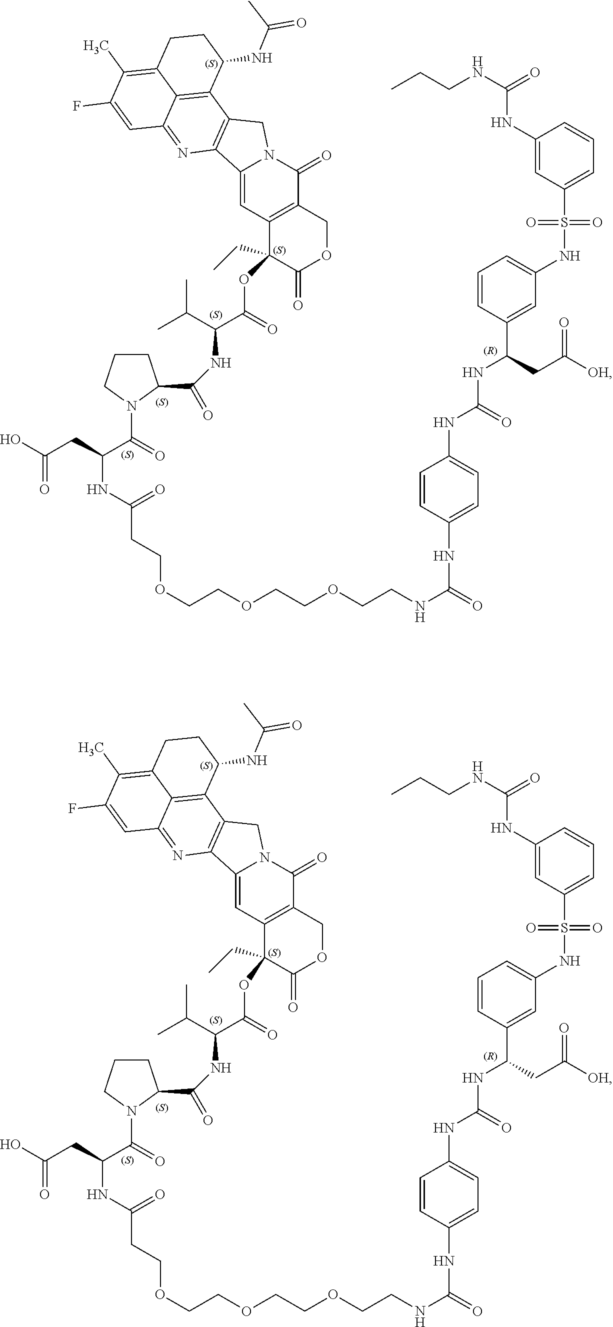

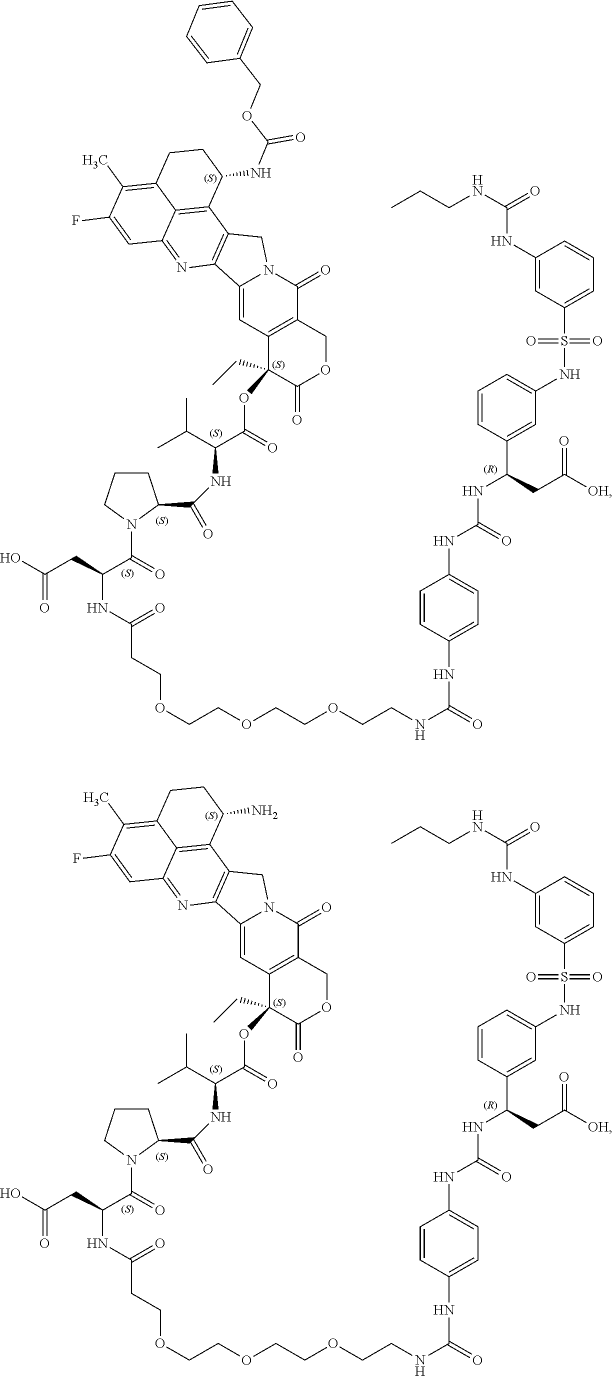

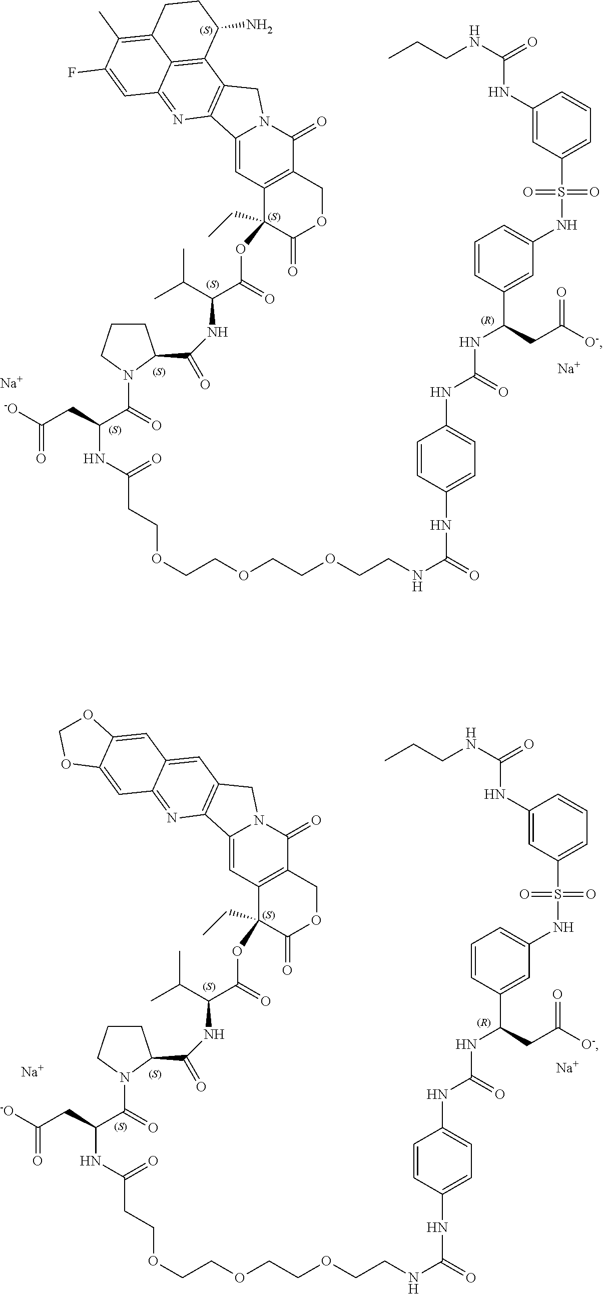

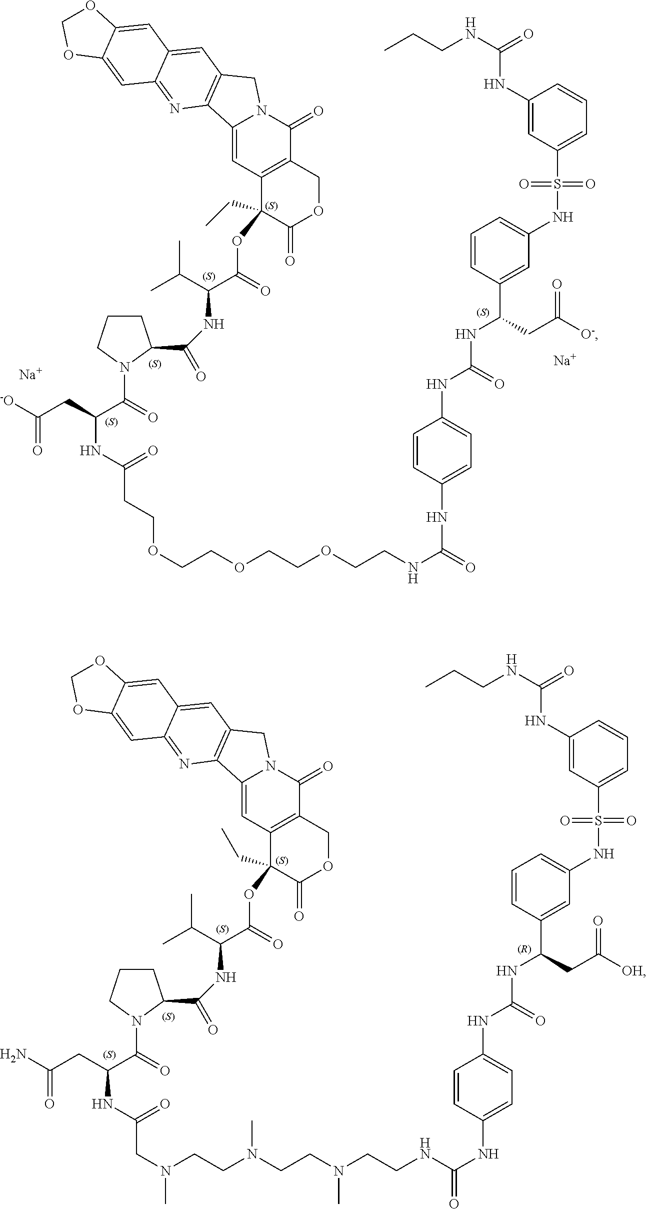

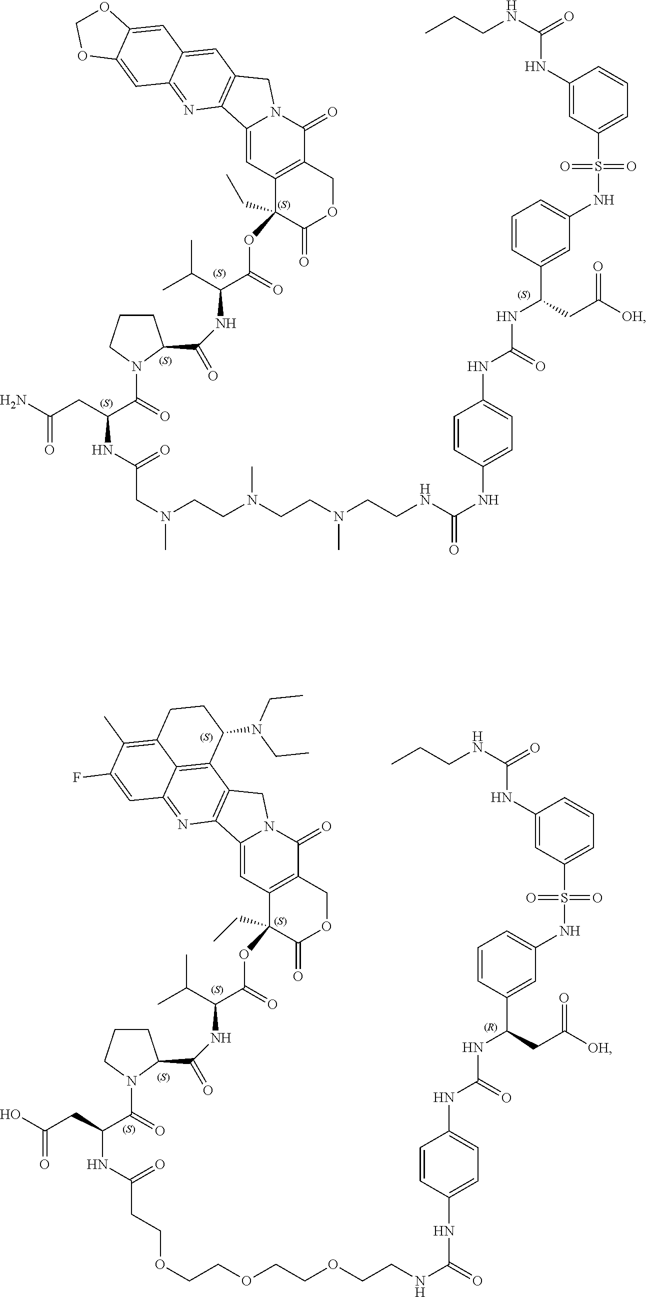

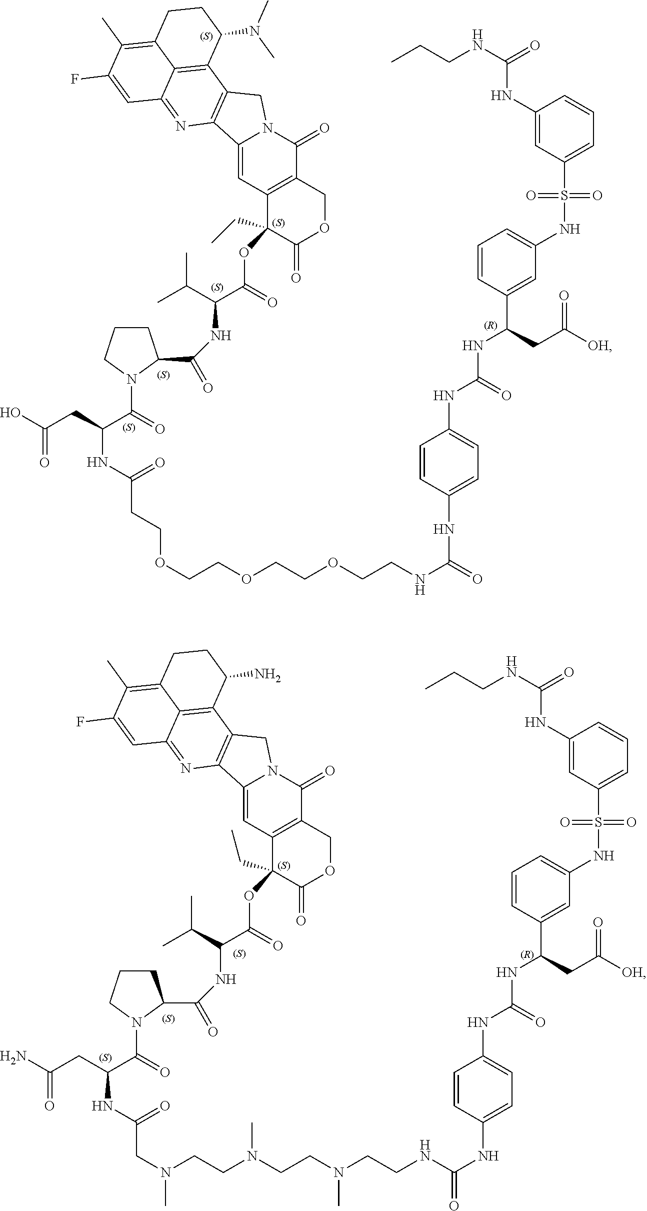

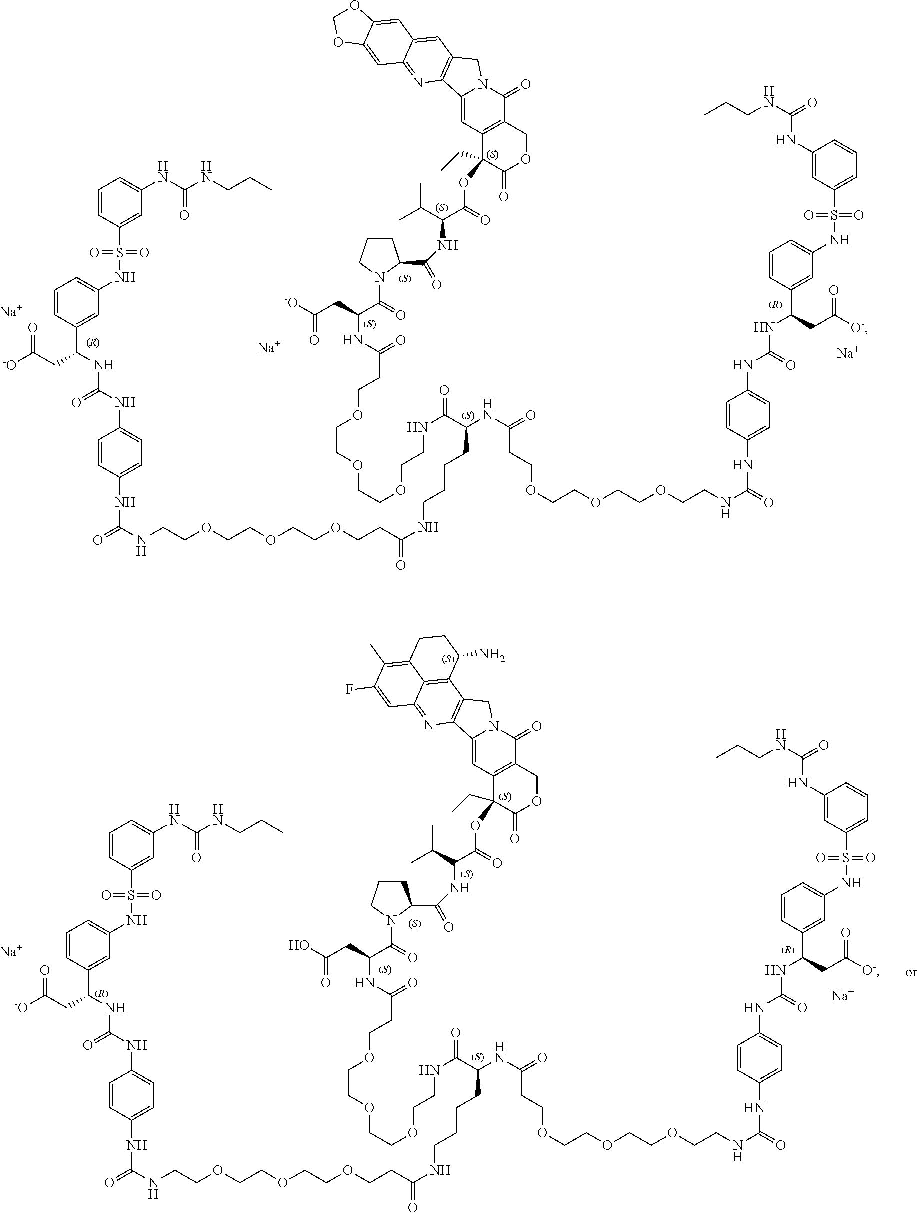

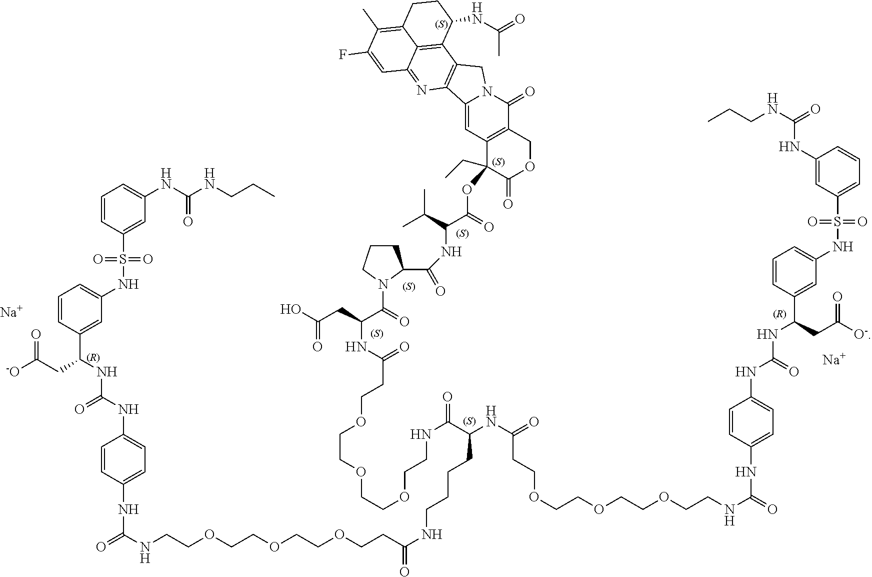

22. A compound, or a pharmaceutically acceptable salt thereof, or a stereoisomer or mixture of stereoisomers thereof, of Formula (X): 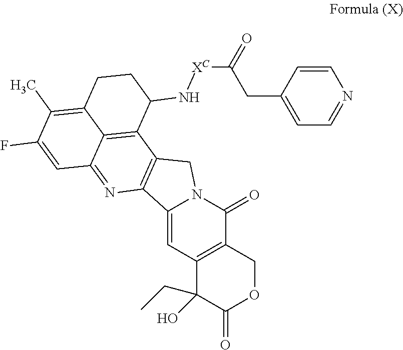
wherein:
X^{C} is a bond,
(-L-Asn-)-,
(-L-Asp)-(L-Pro)-(L-Val-)-,
(-L-Asn)-(L-Pro)-(L-Val-)-,
(-L-Ala)-(L-Ala)-(L-Asn-)-, or
(-L-Ala)-(L-N-Me-Ala)-(L-Asn-)-.

23. The compound of claim 22, or a pharmaceutically acceptable salt thereof, or a stereoisomer or mixture of stereoisomers thereof; wherein X^{C} is a bond, (-L-Asn-), or (-L-Ala)-(L-N-Me-Ala)-(L-Asn-).

24. The compound of claim 22 or 23, or a pharmaceutically acceptable salt thereof, or a stereoisomer or mixture of stereoisomers thereof, that is: 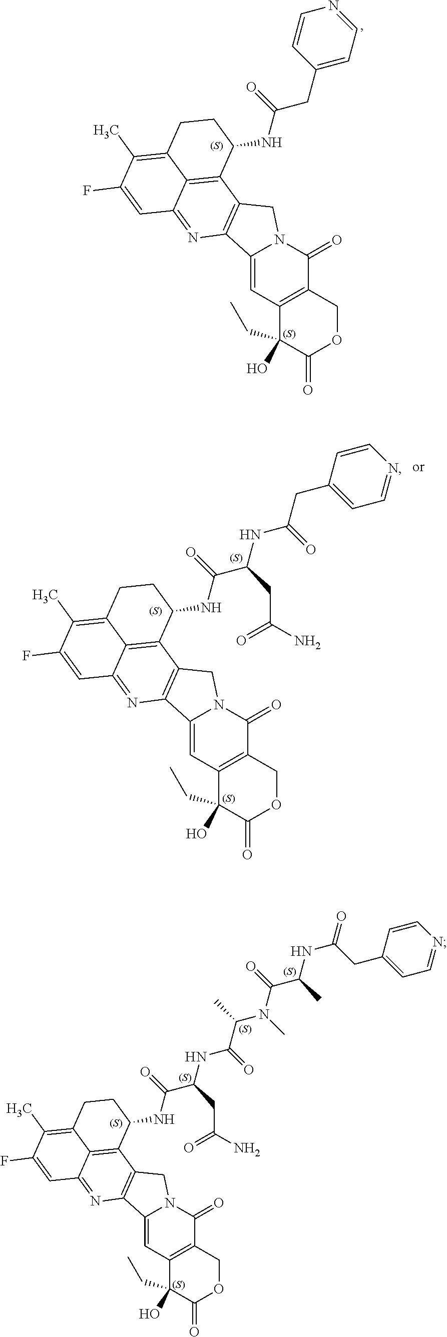
or a pharmaceutically acceptable salt thereof, or a stereoisomer or mixture of stereoisomers thereof.

25. A compound, or a pharmaceutically acceptable salt thereof, or a stereoisomer or mixture of stereoisomers thereof, that is: 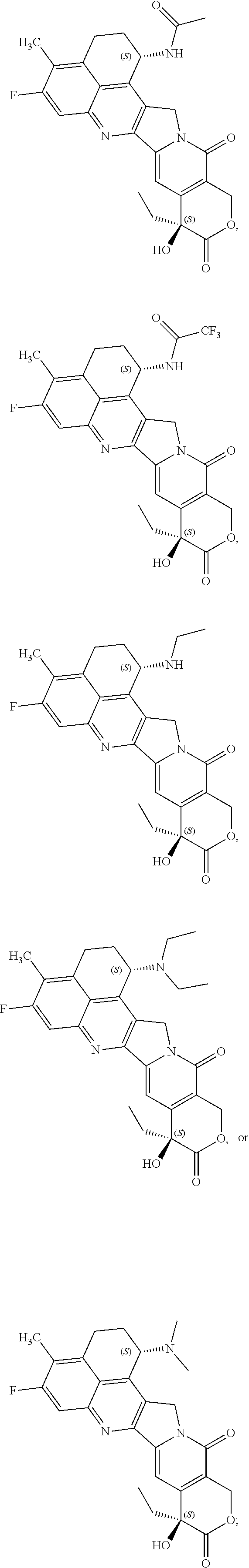
or a pharmaceutically acceptable salt thereof.

26. A pharmaceutical composition comprising a compound of any one of claims 1-25, or a pharmaceutically acceptable salt thereof; or a stereoisomer or mixture of stereoisomers thereof; and a pharmaceutically acceptable excipient.

27. The compound of any one of claims 1-25, or a pharmaceutically acceptable salt thereof; or a stereoisomer or mixture of stereoisomers thereof, for use in the manufacture of a medicament for treating a disease or disorder.

28. The compound of any one of claims 1-24, or a pharmaceutically acceptable salt thereof; or a stereoisomer or mixture of stereoisomers thereof, for use as a medicament.

29. The compound of any one of claims 1-24, or a pharmaceutically acceptable salt thereof; or a stereoisomer or mixture of stereoisomers thereof, for use in treating a disease or disorder.

30. The compound for use of claim 29, or a pharmaceutically acceptable salt thereof, or a stereoisomer or mixture of stereoisomers thereof, wherein the disease or disorder is a hyperproliferative disorder.

31. The compound for use of claim 29, or a pharmaceutically acceptable salt thereof, or a stereoisomer or mixture of stereoisomers thereof, wherein the disease or disorder is an autoimmune disorder.

32. The compound for use of claim 29, or a pharmaceutically acceptable salt thereof, or a stereoisomer or mixture of stereoisomers thereof, wherein the disease or disorder is an ophthalmological disease or disorder.

33. The compound for use of claim 32, or a pharmaceutically acceptable salt thereof, or a stereoisomer or mixture of stereoisomers thereof, wherein the ophthalmological disease or disorder is macular degeneration.

34. The compound for use of claim 29, or a pharmaceutically acceptable salt thereof, or a stereoisomer or mixture of stereoisomers thereof, wherein the disease or disorder is a skin disorder.

35. The compound for use of claim 34, or a pharmaceutically acceptable salt thereof, or a stereoisomer or mixture of stereoisomers thereof, wherein the skin disorder is psoriasis.

36. The compound for use of claim 30, or a pharmaceutically acceptable salt thereof, or a stereoisomer or mixture of stereoisomers thereof, wherein the hyperproliferative disorder is a cancer (e.g., an invasive and/or metastatic cancer).

37. The compound for use of claim 36, or a pharmaceutically acceptable salt thereof, or a stereoisomer or mixture of stereoisomers thereof, wherein the cancer is of the breast, respiratory tract, brain, reproductive organs, digestive tract, urinary tract, eye, liver, skin, head and neck, thyroid, parathyroid or a metastasis thereof.

38. The compound for use of claim 37, or a pharmaceutically acceptable salt thereof, or a stereoisomer or mixture of stereoisomers thereof, wherein the cancer of the breast is invasive ductal carcinoma, invasive lobular carcinoma, ductal carcinoma in situ, lobular carcinoma in situ, inflammatory breast cancer, basal breast cancer, or triple negative breast cancer.

39. The compound for use of claim 37, or a pharmaceutically acceptable salt thereof, or a stereoisomer or mixture of stereoisomers thereof, wherein the cancer of the respiratory tract is small-cell, non-small-cell lung carcinoma, bronchial adenoma, or pleuropulmonary blastoma.

40. The compound for use of claim 37, or a pharmaceutically acceptable salt thereof, or a stereoisomer or mixture of stereoisomers thereof, wherein the cancer of the brain is a brain stem glioma, hypothalamic glioma, glioblastoma, cerebellar astrocytoma, cerebral astrocytoma, medulloblastoma, ependymoma, neuroectodermal tumor, or pineal tumor.

41. The compound for use of claim 37, or a pharmaceutically acceptable salt thereof, or a stereoisomer or mixture of stereoisomers thereof, wherein the cancer of the reproductive organs is a prostate cancer, testicular cancer, endometrial cancer, cervical cancer, ovarian cancer, vaginal cancer, vulvar cancer, or a sarcoma of the uterus.

42. The compound for use of claim 37, or a pharmaceutically acceptable salt thereof, or a stereoisomer or mixture of stereoisomers thereof, wherein the cancer of the digestive tract is anal cancer, colon cancer, colorectal cancer, esophageal cancer, gallbladder cancer, gastric cancer, pancreatic cancer, rectal cancer, small intestinal cancer, or salivary gland cancer.

43. The compound for use of claim 37, or a pharmaceutically acceptable salt thereof, or a stereoisomer or mixture of stereoisomers thereof, wherein the cancer of the urinary tract is bladder cancer, penile cancer, kidney cancer, renal pelvis cancer, ureter cancer, or urethral cancer.

44. The compound for use of claim 37, or a pharmaceutically acceptable salt thereof, or a stereoisomer or mixture of stereoisomers thereof, wherein the cancer of the eye is intraocular melanoma or retinoblastoma.

45. The compound for use of claim 37, or a pharmaceutically acceptable salt thereof, or a stereoisomer or mixture of stereoisomers thereof, wherein the cancer of the liver is to hepatocellular carcinoma, liver cell carcinoma with or without fibrolamellar variant, cholangiocarcinoma, intrahepatic bile duct carcinoma, or mixed hepatocellular cholangiocarcinoma.

46. The compound for use of claim 37, or a pharmaceutically acceptable salt thereof, or a stereoisomer or mixture of stereoisomers thereof, wherein the cancer of the skin is squamous cell carcinoma, Kaposi's sarcoma, malignant melanoma, Merkel cell skin cancer, or non-melanoma skin cancer.

47. The compound for use of claim 37, or a pharmaceutically acceptable salt thereof, or a stereoisomer or mixture of stereoisomers thereof, wherein the cancer of the head-and-neck is laryngeal cancer, hypopharyngeal cancer, nasopharyngeal cancer, oropharyngeal cancer, or a lip and oral cavity cancer.

48. The compound for use of claim 36, or a pharmaceutically acceptable salt thereof, or a stereoisomer or mixture of stereoisomers thereof, wherein the cancer is a sarcoma.

49. The compound for use of claim 48, or a pharmaceutically acceptable salt thereof, or a stereoisomer or mixture of stereoisomers thereof, wherein the sarcoma is Ewing sarcoma, osteosarcoma, or fibrosarcoma.

50. A method of treating a disease or disorder in a subject, comprising administering to the subject a therapeutically effective amount of a compound of any one of claims 1-24, or a pharmaceutically acceptable salt thereof, or a stereoisomer or mixture of stereoisomers thereof.

51. The method of claim 50, wherein the disease or disorder is a hyperproliferative disorder.

52. The method of claim 50, wherein the disease or disorder is a cancer.

53. The method of claim 52, wherein the cancer is of the breast, respiratory tract, brain, reproductive organs, digestive tract, urinary tract, eye, liver, skin, head and neck, thyroid, parathyroid or a metastasis thereof.

54. The method of claim 53, wherein the cancer is breast cancer, colon cancer, renal cancer, or lung cancer.
